(19) United States
(12) Patent Application Publication
(10) Pub. No.: US 2024/0336621 A1
(43) Pub. Date: Oct. 10, 2024

(54) MATERIALS FOR ORGANIC ELECTROLUMINESCENT DEVICES

(71) Applicant: MERCK PATENT GMBH, DARMSTADT (DE)

(72) Inventors: AMIR HOSSAIN PARHAM, FRANKFURT AM MAIN (DE); CHRISTIAN EHRENREICH, DARMSTADT (DE)

(21) Appl. No.: 18/742,755

(22) Filed: Jun. 13, 2024

Related U.S. Application Data
(63) Continuation of application No. PCT/EP2022/085369, filed on Dec. 12, 2022.

(30) Foreign Application Priority Data
Dec. 13, 2021 (EP) 21214069.3

(51) Int. Cl. C07D 487/14; C07D 471/22; C07D 493/14; C07D 495/14; C07D 519/00; C07F 5/02; H10K 85/60

(52) U.S. Cl. CPC C07D 487/14; C07D 471/22; C07D 493/14; C07D 495/14; C07D 519/00; C07F 5/027; H10K 85/622; H10K 85/654; H10K 85/6572; H10K 85/6574; H10K 85/6576; H10K 85/658

(57) ABSTRACT

A compound according to formula (1) as described herein is used for organic electronic devices, especially organic electroluminescent devices, which may be used in electronic devices, especially organic electroluminescent devices.

### CROSS-REFERENCE TO RELATED APPLICATIONS

This application is a Continuation under 35 USC § 111(a) of International Patent Application No. PCT/EP2022/085369 filed Dec. 12, 2022, which claims priority to the EP Application No. 21214069.3 filed on Dec. 13, 2021. The entire contents of these applications are incorporated herein by reference in their entirety.

The present invention describes compounds, especially for use in electronic devices. The invention further relates to a process for preparing the compounds of the invention and to electronic devices comprising these compounds.

### BACKGROUND

The structure of organic electroluminescent devices in which organic semiconductors are used as functional materials is described, for example, in U.S. Pat. Nos. 4,539,507, 5,151,629, EP 0676461, WO 98/27136 and WO 2010/151006 A1. Emitting materials used are frequently organometallic complexes which exhibit phosphorescence. For quantum-mechanical reasons, up to four times the energy efficiency and power efficiency is possible using organometallic compounds as phosphorescent emitters. In general terms, there is still a need for improvement in electroluminescent devices, especially also in electroluminescent devices which exhibit phosphorescence, for example with regard to efficiency, operating voltage and lifetime. Also known are organic electroluminescent devices comprising fluorescent emitters or emitters that exhibit TADF (thermally activated delayed fluorescence).

According to the prior art, lactams according to WO2013/064206 or lactones according to WO2015/106789 are among the matrix materials used for phosphorescent emitters or as electron transport materials.

The properties of organic electroluminescent devices are not only determined by the emitters used. Also of particular significance here are especially the other materials used, such as matrix materials, hole blocker materials, electron transport materials, hole transport materials and electron or exciton blocker materials. Improvements to these materials can lead to distinct improvements to electroluminescent devices.

In general terms, in the case of these materials, for example for use as matrix materials, hole transport materials or electron transport materials, there is still a need for improvement, particularly in relation to the lifetime, but also in relation to the efficiency and operating voltage of the device. Moreover, OLEDs containing the compounds are supposed to have high color purity.

It is an object of the present invention to provide compounds which are suitable for use as matrix materials or charge transport materials in an organic electronic device, especially in an organic electroluminescent device, and which lead to good device properties when used in this device, and to provide the corresponding electronic device.

It is a particular object of the present invention to provide compounds which lead to a high lifetime, good efficiency and low operating voltage.

A further object of the present invention can be considered that of providing compounds suitable for use in phosphorescent or fluorescent electroluminescent devices, especially as a matrix material. A particular object of the present invention is that of providing matrix materials suitable for red-, yellow- and green-phosphorescing electroluminescent devices.

### DETAILED DESCRIPTION OF THE INVENTION

It has been found that, surprisingly, these objects are achieved by particular compounds that are described in detail hereinafter, and these have lower operating voltage, higher efficiency and/or longer lifetime compared to materials from the prior art. The use of the compounds leads to very good properties of organic electronic devices, especially of organic electroluminescent devices, especially with regard to lifetime, efficiency and operating voltage. The present invention therefore provides electronic devices, especially organic electroluminescent devices, comprising such compounds.

The present invention therefore provides a compound of formula (1)

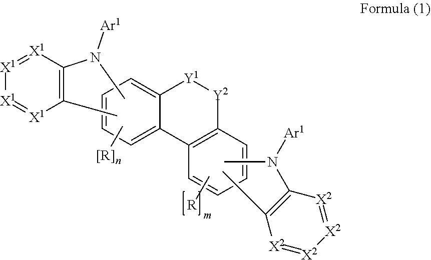

where the symbols used are as follows:
X¹ is the same or different at each instance and is CR or N, with the proviso that not more than two X¹ groups are N;
X² is the same or different at each instance and is CR or N, with the proviso that not more than two X² groups are N;
Y¹ is the same or different at each instance and is C═O, C═S, BR^{a}, NR^{a}, S, O, S═O, SO₂, PR^{a} or POR^{a}; preferably C═O, C═S or NR^{a};
Y² is the same or different at each instance and is C═O, C═S, BR^{a}, NR^{a}, S, O, S═O, SO₂, PR^{a} or POR^{a}; preferably C═O, C═S or NR^{a}; where:
   Y¹ is not the same as Y²; where preferably exactly one Y¹ or Y² is C═O or C═S and the other Y¹ or Y² is NR^{a};
Ar¹ is the same or different at each instance and is an aromatic or heteroaromatic ring system which has 5 to 60 aromatic ring atoms and may be substituted in each case by one or more R¹ radicals; where one Ar¹ radical together with an R or R^{a} radical may form a ring system that may be substituted by one or more R¹ radicals;
R, R^{a} is the same or different at each instance and is H, D, F, Cl, Br, I, CN, NO₂, N(Ar^{a})₂, N(R¹)₂, C(═O)N(Ar^{a})₂, C(═O)N(R¹)₂, C(Ar^{a})₃, C(R¹)₃, Si(Ar^{a})₃, Si(R¹)₃, B(Ar^{a})₂, B(R¹)₂, C(═O)Ar^{a}, C(═O)R¹, P(═O)(Ar^{a})₂, P(═O)(R¹)₂, P(Ar^{a})₂, P(R¹)₂, S(═O)Ar^{a}, S(═O)R¹, S(═O)₂Ar^{a}, S(═O)₂R¹, OSO₂Ar^{a}, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may be substituted in each case by one or more R¹ radicals, where one or more nonadjacent CH₂ groups may be replaced by R¹C═CR¹, C≡C, Si(R¹)₂, C═O, C═S, C═Se, C═NR¹, C(═O)O, C(═O)NR¹, NR¹, P(═O)(R¹), O, S, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two or more R and/or R^{a} radicals together may form an aliphatic, heteroaliphatic, aromatic or heteroaromatic ring system which may be substituted by one or more R¹ radicals; or an R or R^{a} radical together with an Ar¹ radical may form a ring system that may be substituted by one or more R¹ radicals;
n, m is the same or different at each instance and is 0, 1 or 2;
Ar^{a} is the same or different at each instance and is an aromatic or heteroaromatic ring system which has 5 to 60 aromatic ring atoms and may be substituted by one or more R¹ radicals; at the same time, it is possible for two Ar^{a} radicals bonded to the same carbon atom, silicon atom, nitrogen atom, phosphorus atom or boron atom also to be joined together via a bridge by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹;
R¹ is the same or different at each instance and is H, D, F, Cl, Br, I, CN, NO₂, N(R²)₂, C(═O)R², P(═O)(R²)₂, P(R²)₂, B(R²)₂, C(R²)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by R²C═CR², C≡C, Si(R²)₂, C═O, C═S, C═Se, C═NR², C(═O)O, C(═O)NR², NR², P(═O)(R²), O, S, SO or SO₂ and where one or more hydrogen atoms may be replaced by D, F, C, Br, I, CN or NO₂, or an aromatic or heteroaromatic ring system which has 5 to 60 aromatic ring atoms, each of which may be substituted by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, where two or more R¹ radicals together may form an aliphatic, heteroaliphatic, aromatic or heteroaromatic ring system that may be substituted by one or more R² radicals;
R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, C, Br, I or CN and which may be substituted by one or more alkyl groups each having 1 to 4 carbon atoms; at the same time, two or more, preferably adjacent substituents R² together may form a ring system.

The present compounds may preferably be used as active compound in electronic devices. Active compounds are generally the organic or inorganic materials introduced between anode and cathode, for example in an organic electronic device, especially in an organic electroluminescent device, for example charge injection, charge transport or charge blocker materials, but especially matrix materials. Preference is given here to organic materials.

Adjacent carbon atoms in the context of the present invention are carbon atoms bonded directly to one another. In addition, “adjacent radicals” in the definition of the radicals means that these radicals are bonded to the same carbon atom or to adjacent carbon atoms. These definitions apply correspondingly, inter alia, to the terms “adjacent groups” and “adjacent substituents”.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

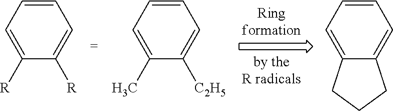

In addition, however, the abovementioned wording shall also be understood to mean that, if one of the two radicals is hydrogen, the second radical binds to the position to which the hydrogen atom was bonded, forming a ring. This will be illustrated by the following scheme:

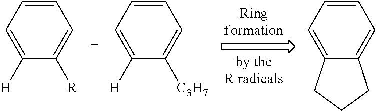

A fused aryl group, a fused aromatic ring system or a fused heteroaromatic ring system in the context of the present invention is a group in which two or more aromatic groups are fused, i.e. annelated, to one another along a common edge, such that, for example, two carbon atoms belong to the at least two aromatic or heteroaromatic rings, as, for example, in naphthalene. By contrast, for example, fluorene is not a fused aryl group in the context of the present invention, since the two aromatic groups in fluorene do not have a common edge. Corresponding definitions apply to heteroaryl groups and to fused ring systems which may but need not also contain heteroatoms.

If two or more, preferably adjacent R, R^{a}, R¹ and/or R² radicals together form a ring system, the result may be a monocyclic or polycyclic, aliphatic, heteroaliphatic, aromatic or heteroaromatic ring system.

An aryl group in the context of this invention contains 6 to 60 carbon atoms, preferably 6 to 40 carbon atoms, more preferably 6 to 30 carbon atoms; a heteroaryl group in the context of this invention contains 2 to 60 carbon atoms, preferably 2 to 40 carbon atoms, more preferably 2 to 30 carbon atoms, and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused aryl or heteroaryl group, for example naphthalene, anthracene, phenanthrene, quinoline, isoquinoline, etc.

An aromatic ring system in the context of this invention contains 6 to 60 carbon atoms, preferably 6 to 40 carbon atoms, more preferably 6 to 30 carbon atoms, in the ring system. A heteroaromatic ring system in the context of this invention contains 1 to 60 carbon atoms, preferably 1 to 40 carbon atoms, more preferably 1 to 30 carbon atoms, and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for a plurality of aryl or heteroaryl groups to be interrupted by a nonaromatic unit (preferably less than 10% of the atoms other than H), for example a carbon, nitrogen or oxygen atom or a carbonyl group. For example, systems such as 9,9′-spirobifluorene, 9,9-diarylfluorene, triarylamine, diaryl ethers, stilbene, etc. shall thus also be regarded as aromatic ring systems in the context of this invention, and likewise systems in which two or more aryl groups are interrupted, for example, by a linear or cyclic alkyl group or by a silyl group. In addition, systems in which two or more aryl or heteroaryl groups are bonded directly to one another, for example biphenyl, terphenyl, quaterphenyl or bipyridine, shall likewise be regarded as an aromatic or heteroaromatic ring system.

A cyclic alkyl, alkoxy or thioalkoxy group in the context of this invention is understood to mean a monocyclic, bicyclic or polycyclic group.

In the context of the present invention, a C1- to C20-alkyl group in which individual hydrogen atoms or CH₂ groups may also be substituted by the abovementioned groups is understood to mean, for example, the methyl, ethyl, n-propyl, i-propyl, cyclopropyl, n-butyl, i-butyl, s-butyl, t-butyl, cyclobutyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl radicals. An alkenyl group is understood to mean, for example, ethenyl, propenyl, butenyl, pentenyl, cyclopentenyl, hexenyl, cyclohexenyl, heptenyl, cycloheptenyl, octenyl, cyclooctenyl or cyclooctadienyl. An alkynyl group is understood to mean, for example, ethynyl, propynyl, butynyl, pentynyl, hexynyl, heptynyl or octynyl. A C1- to C40-alkoxy group is understood to mean, for example, methoxy, trifluoromethoxy, ethoxy, n-propoxy, i-propoxy, n-butoxy, i-butoxy, s-butoxy, t-butoxy or 2-methylbutoxy.

An aromatic or heteroaromatic ring system which has 5 to 60, preferably 5-40, aromatic ring atoms, more preferably 5 to 30 aromatic ring atoms, and may also be substituted in each case by the abovementioned radicals and which may be joined to the aromatic or heteroaromatic system via any desired positions is understood to mean, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

Preferred compounds in the context of the invention are compounds of the formulae (1-1a) or (1-1 b), where the symbols used have the definition given above.

More preferably, not more than two of the symbols X¹ and X² in total are N; most preferably, not more than one of the symbols X¹ and X² in total is N.

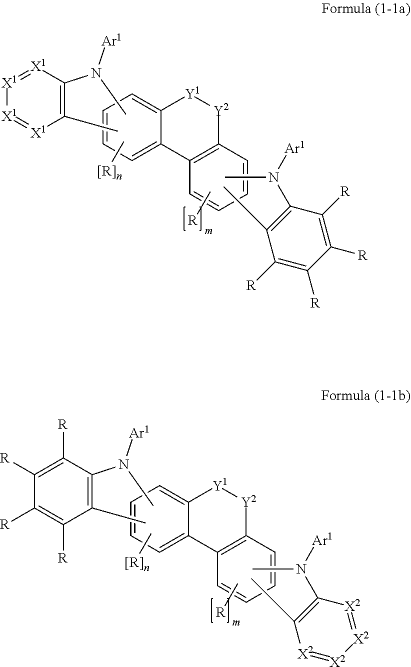

Especially preferred are compounds of the formula (1-1c) where the symbols used have the definition given above.

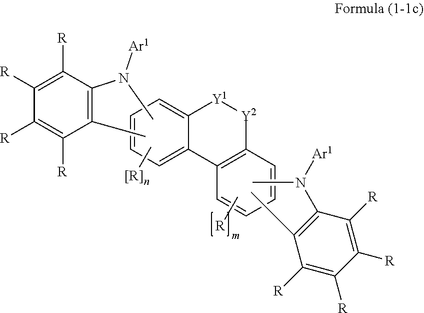

Further preferred compounds are compounds of the formulae (1-2a), (1-2b), (1-2c) and (1-2d), where the symbols used have the definition given above.

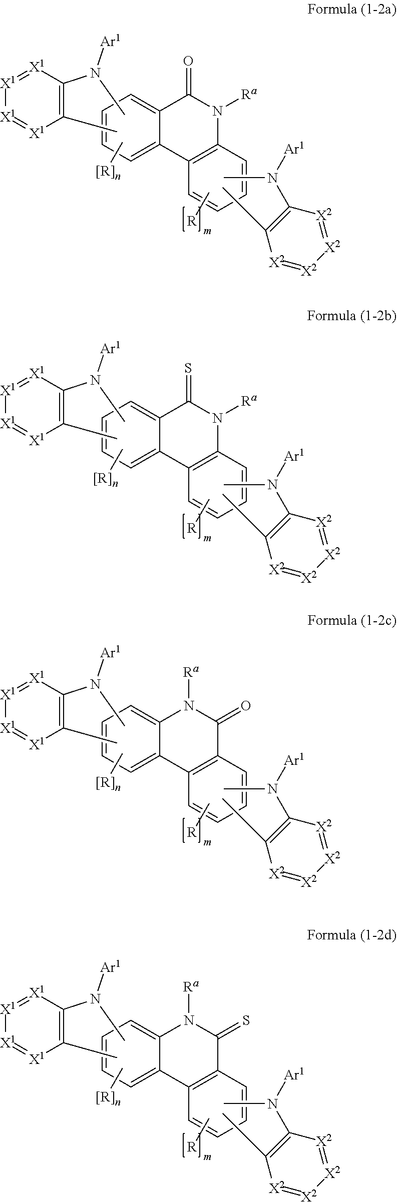

Particular preference is given to compounds of the formulae (1-2a) and (1-2c).

A further preferred embodiment of the invention is the compounds of the formulae (1-3a) and (1-3b), where the symbols used have the definition given above.

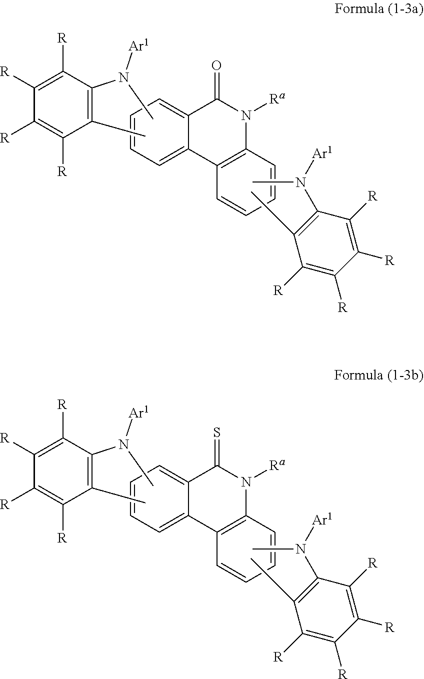

In a further preferred embodiment, the compounds of the invention are selected from compounds of the formulae (2-1a) to (2-36a), where symbols used have the definitions given above.

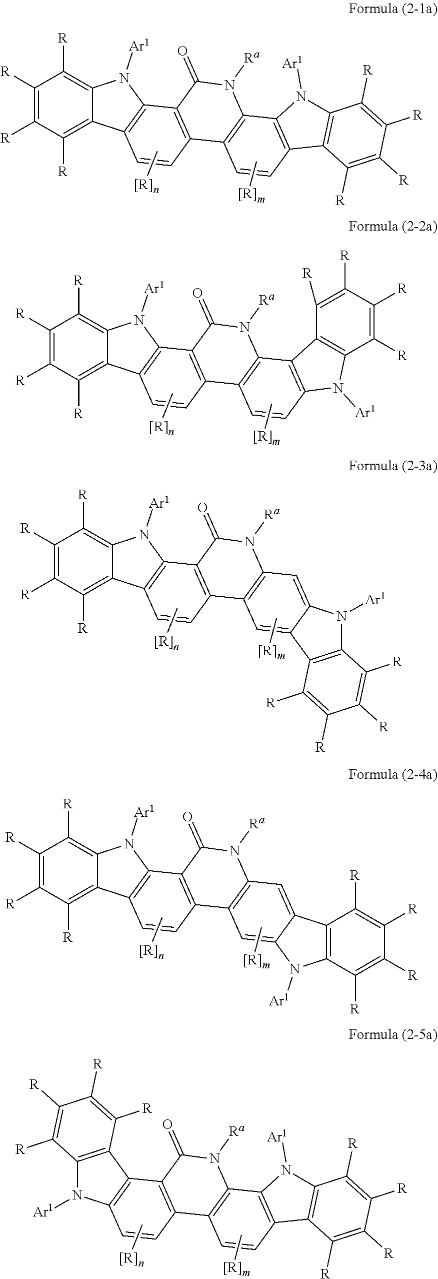

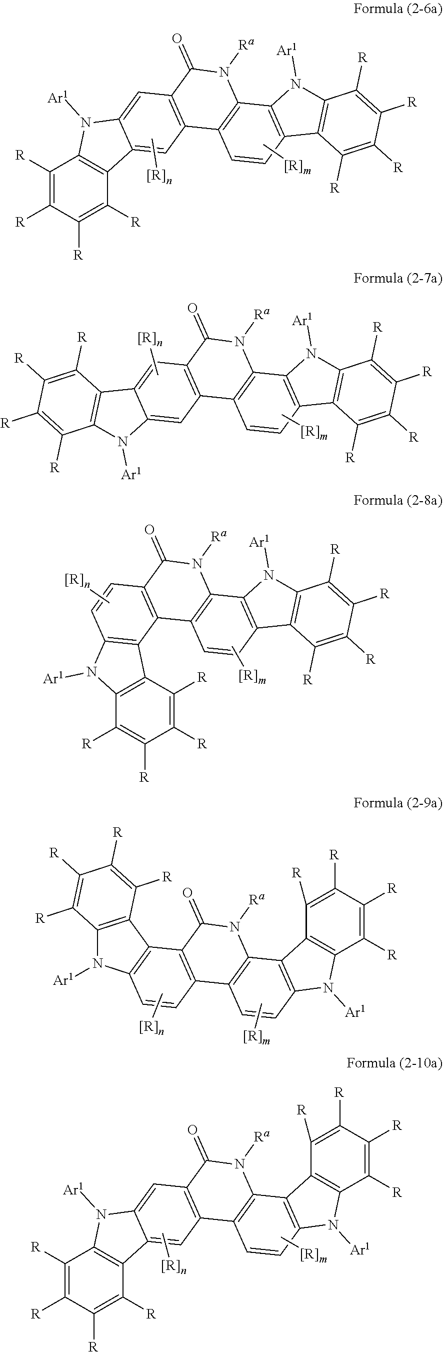

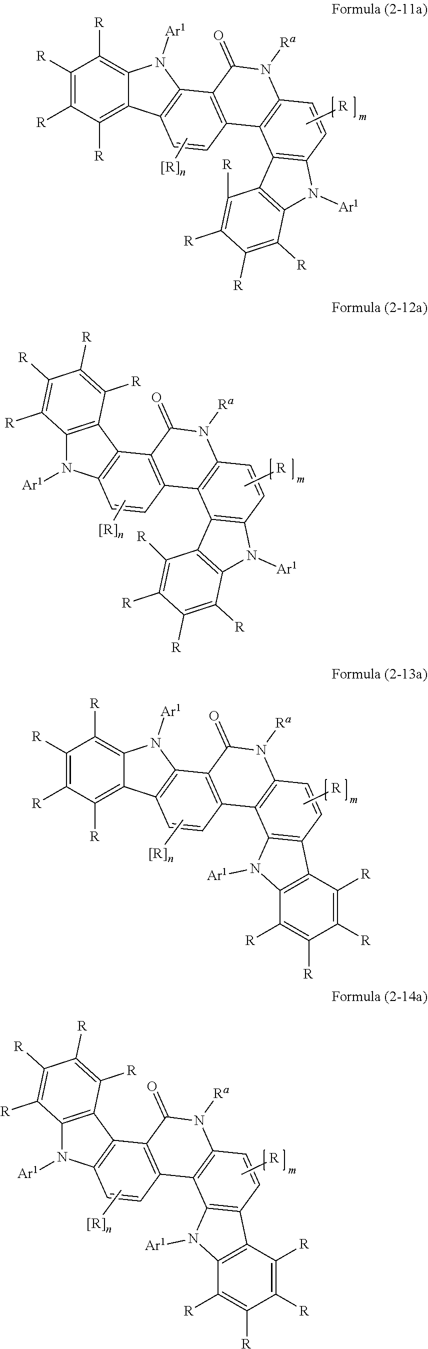

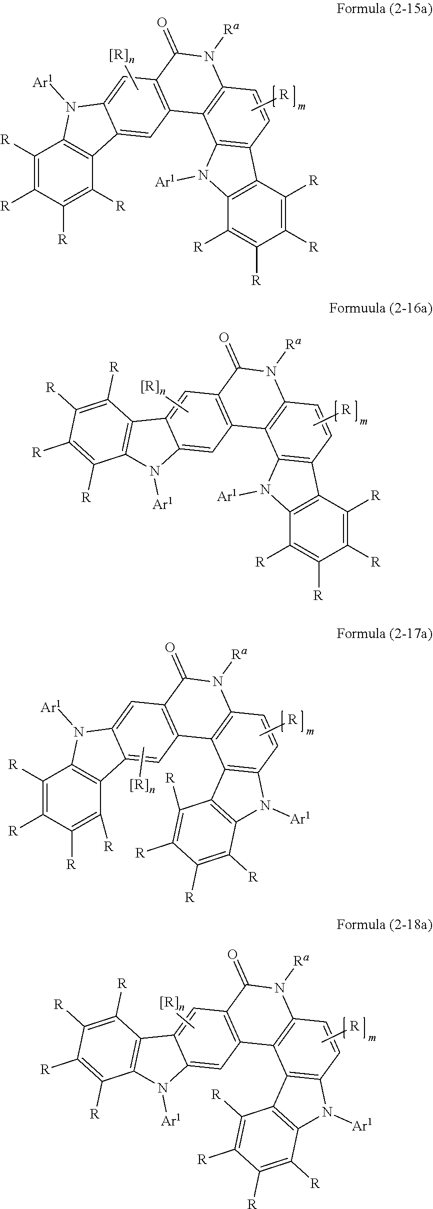

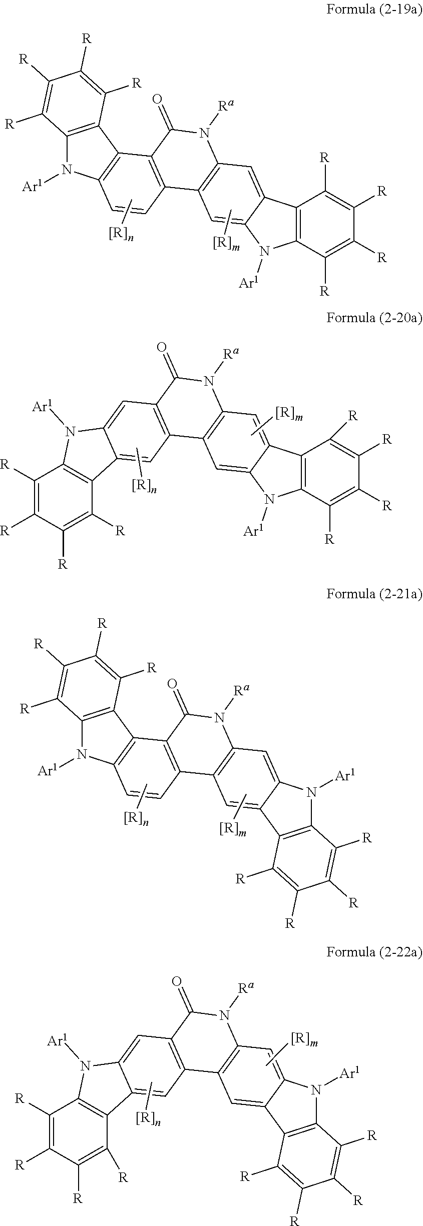

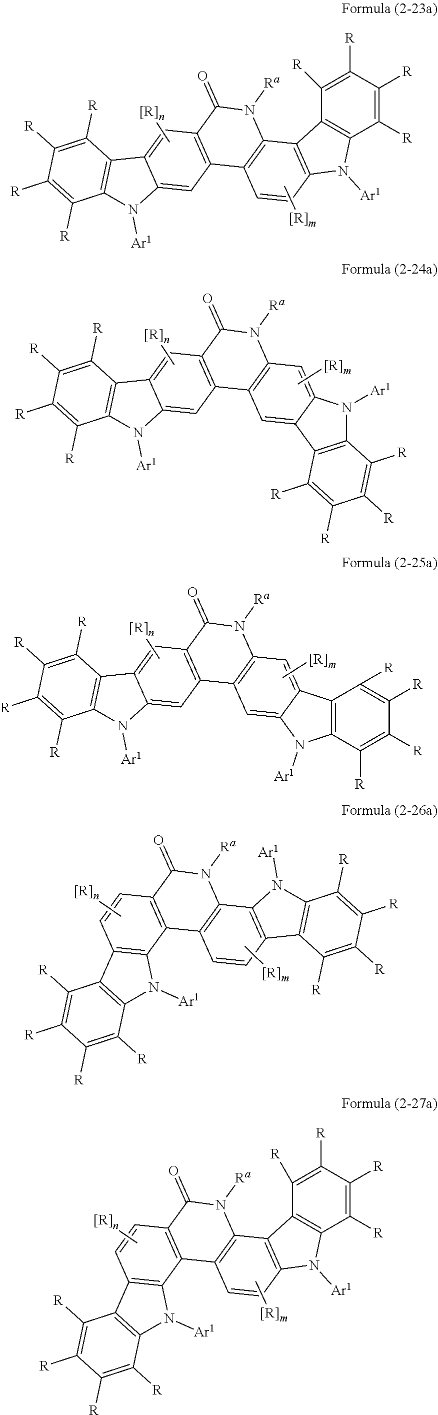

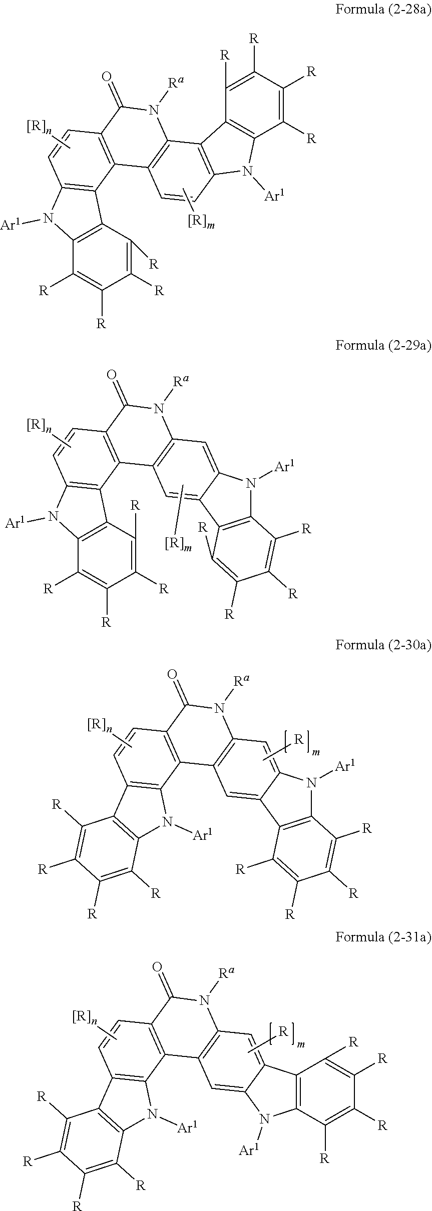

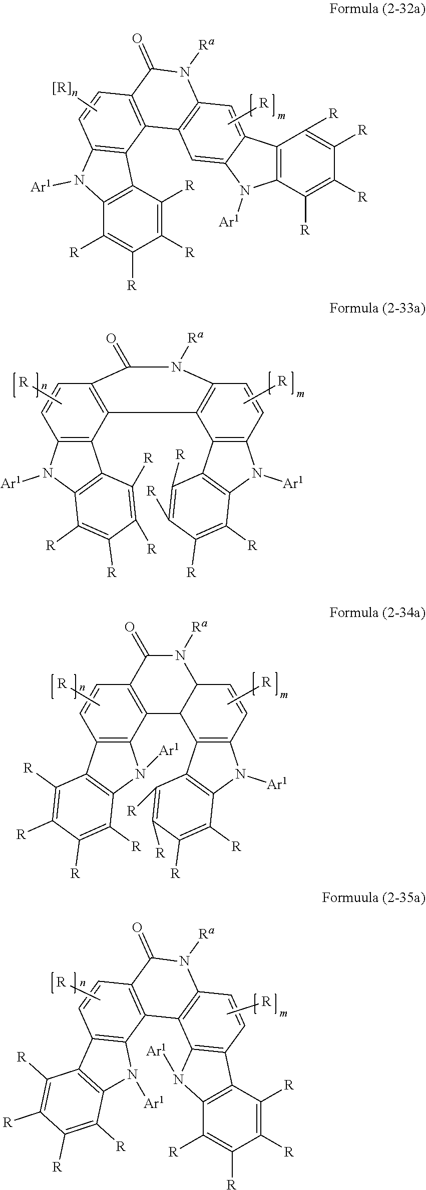

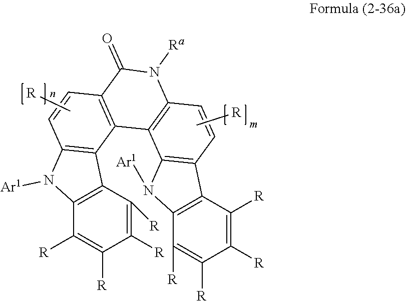

The compounds of the invention are more preferably selected from compounds of the formula (2-9a), (2-10a), (2-21a) or (2-22a).

In a further preferred embodiment of the invention, the compound of the formula (1) or of the preferred embodiments contains not more than two substituents R that are a group other than H or D, and more preferably not more than one substituent R is a group other than H or D. Very particular preference is given here to compounds of the formulae (2-1 b) to (2-72b).

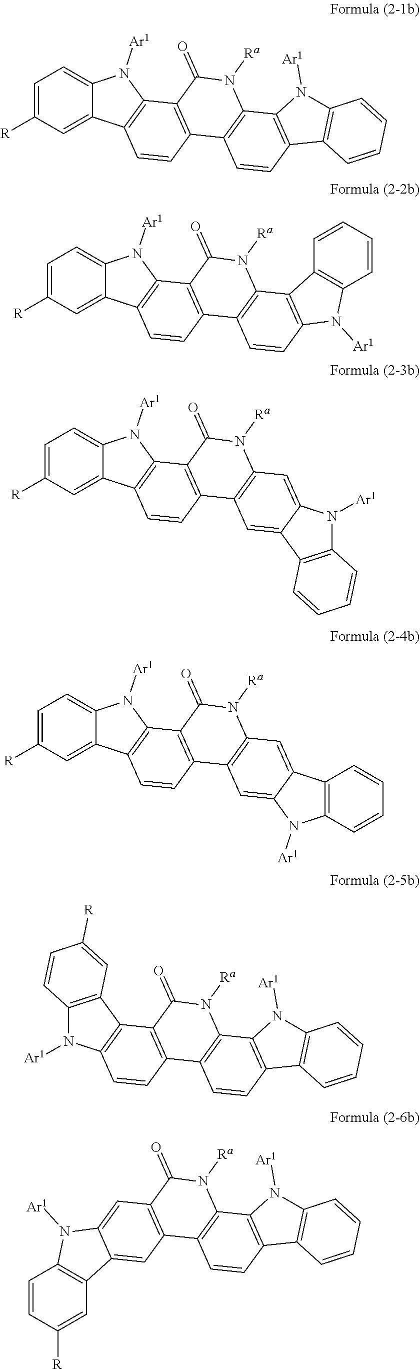

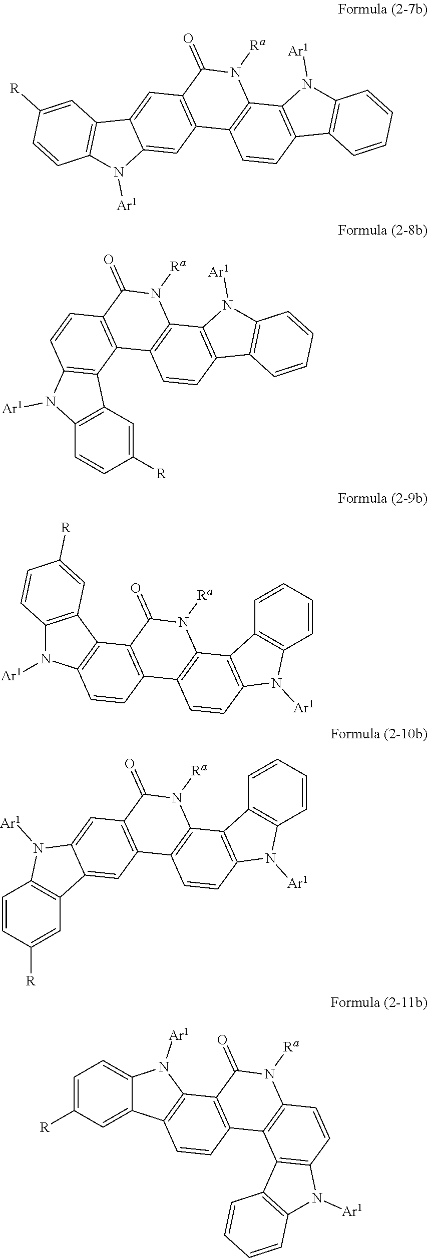

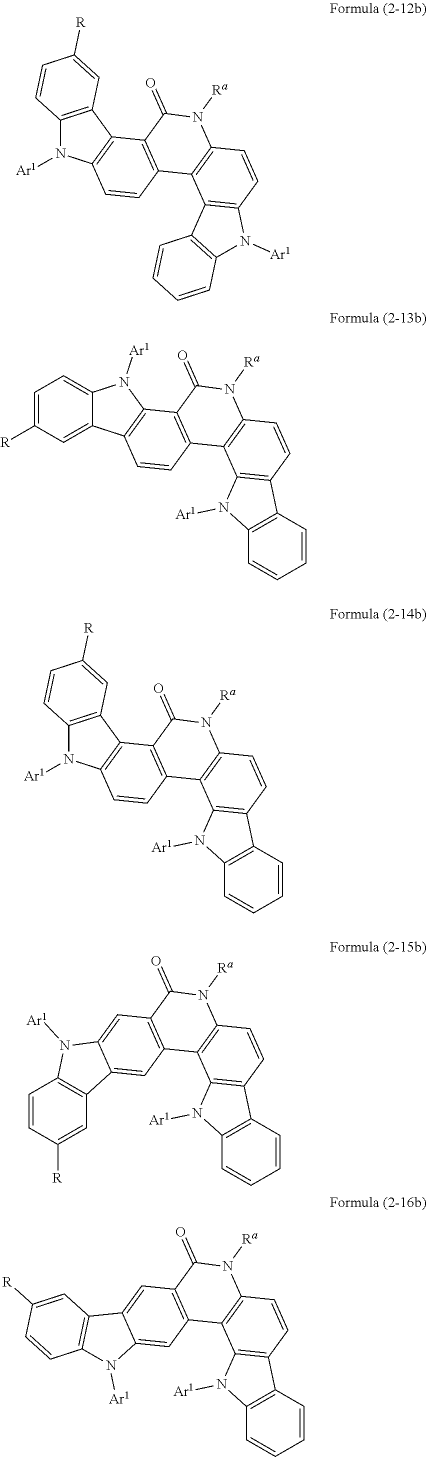

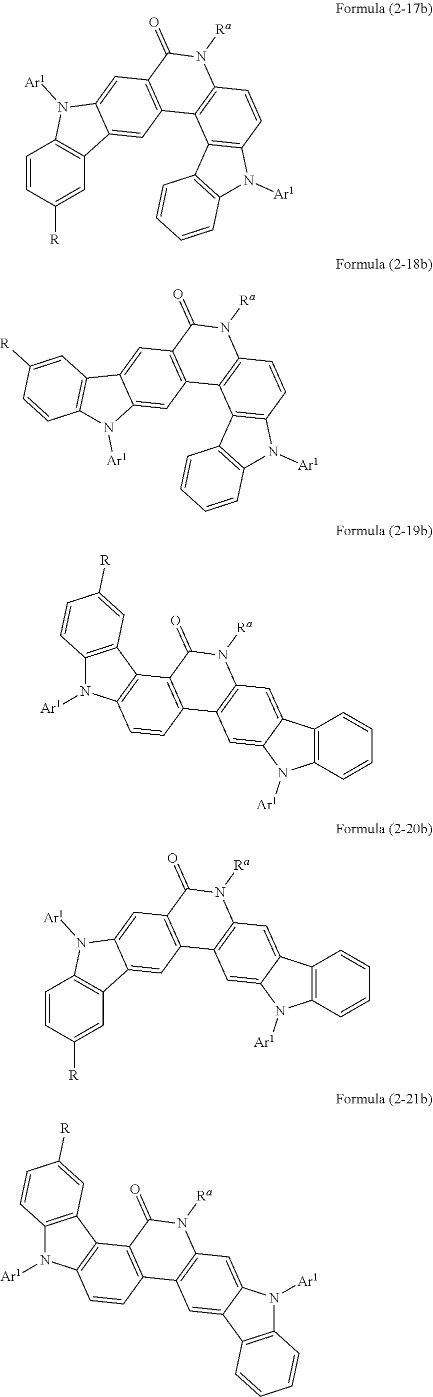

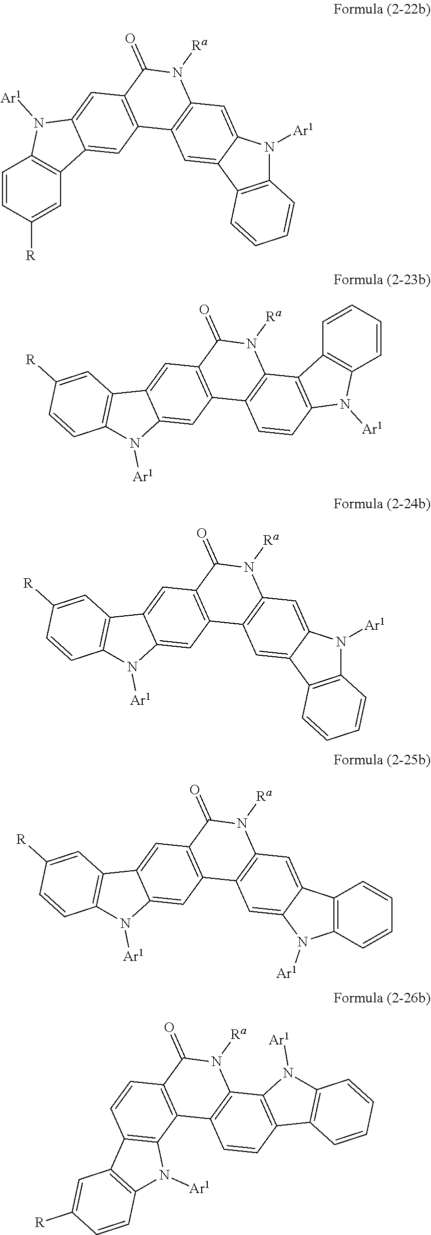

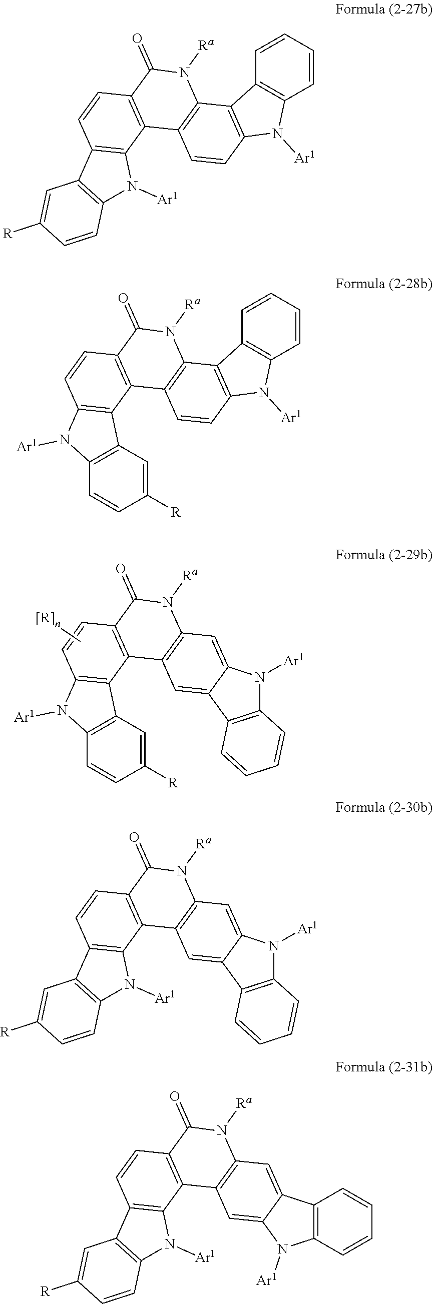

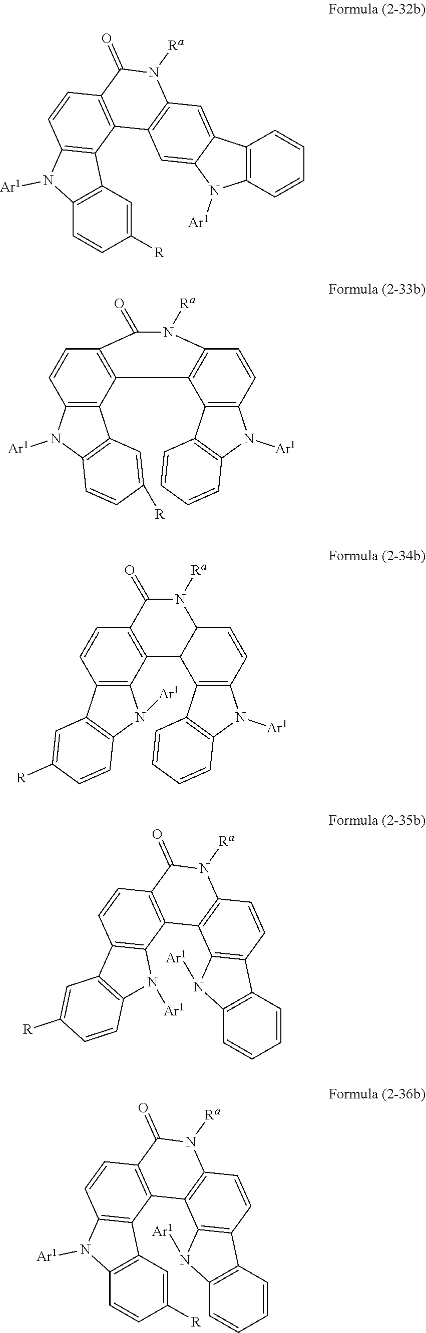

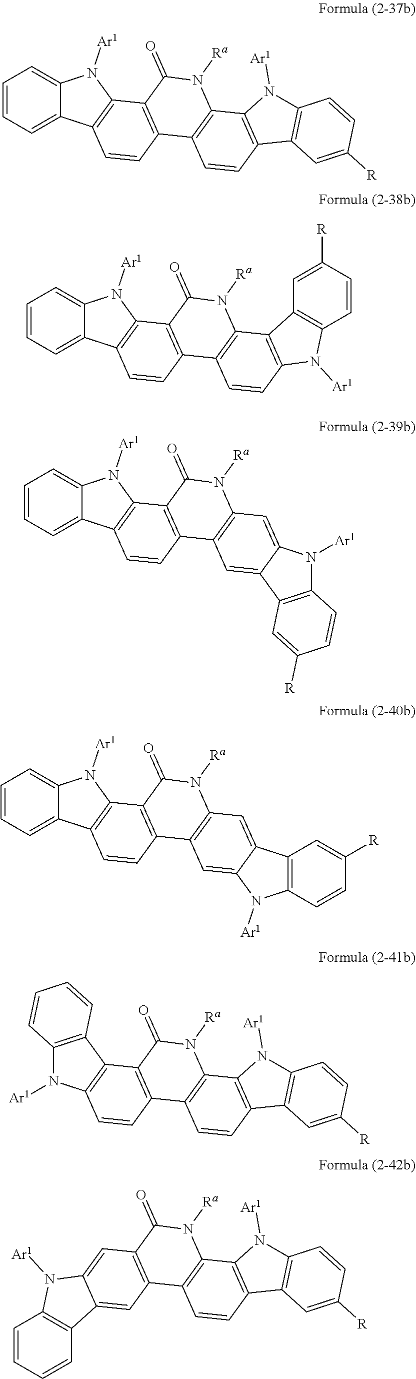

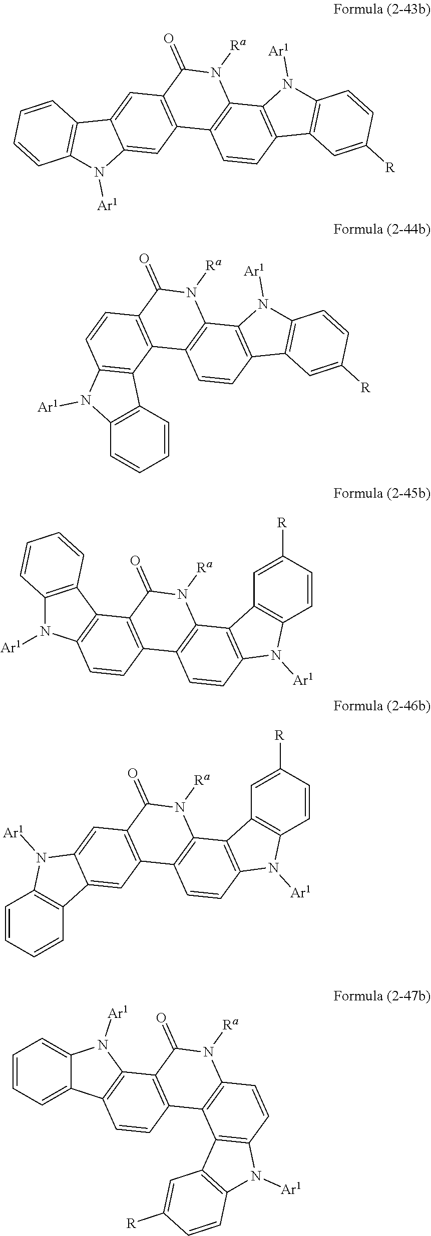

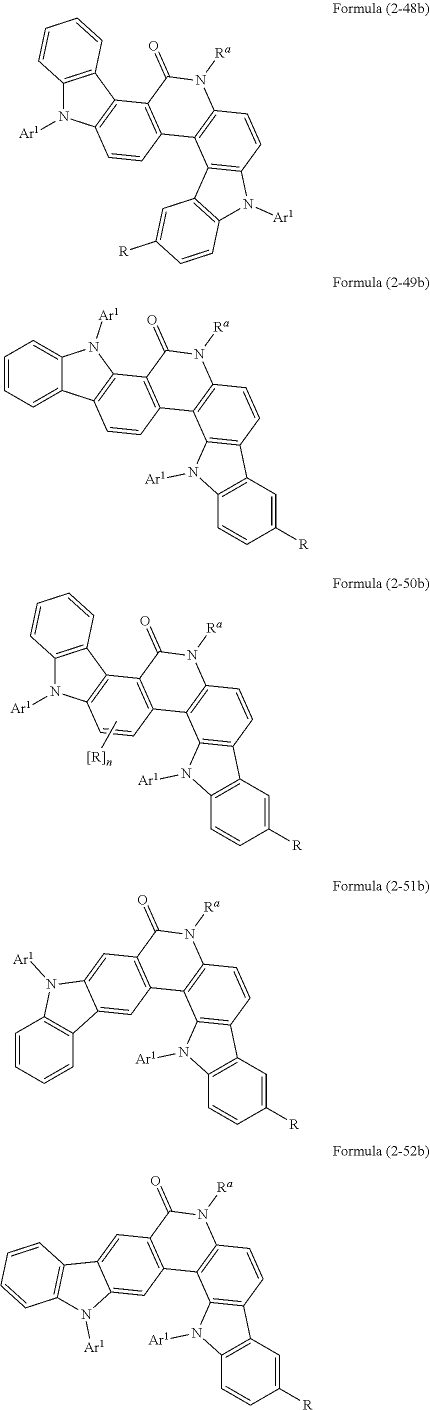

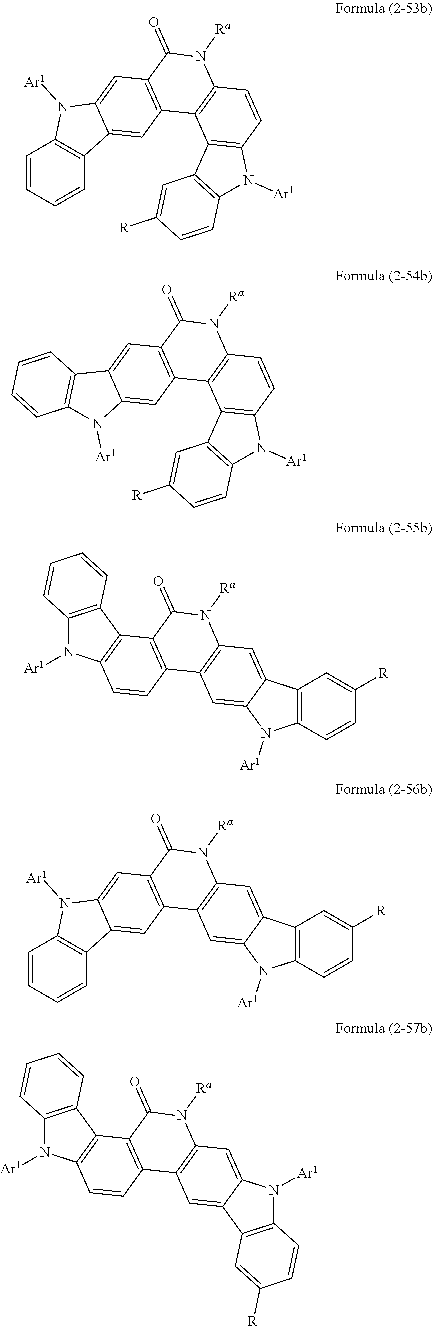

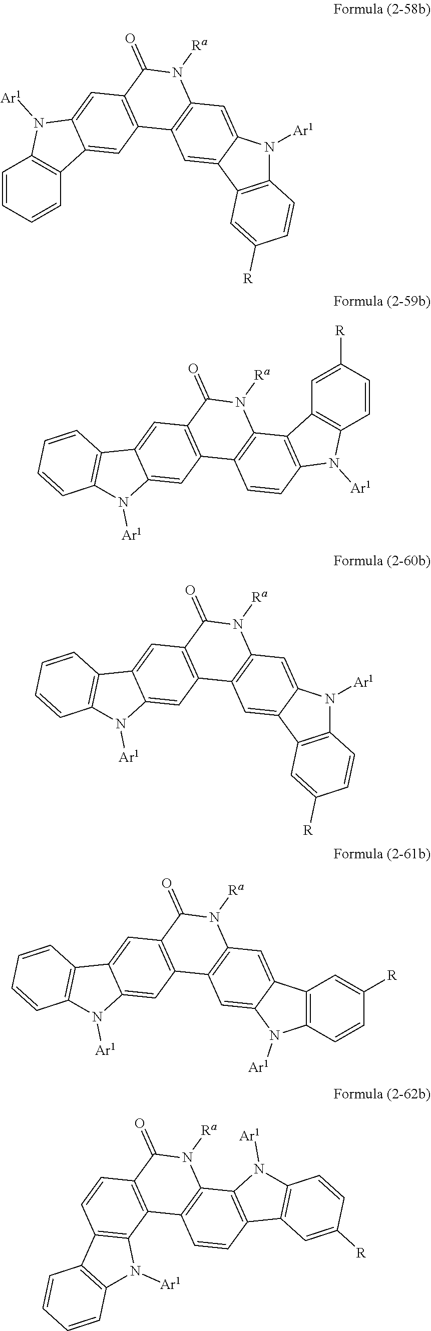

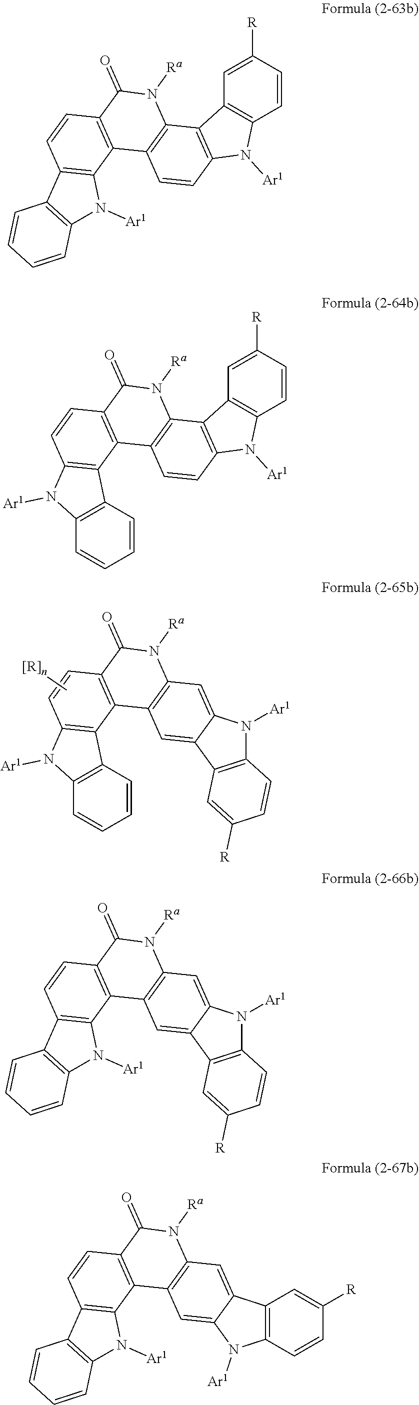

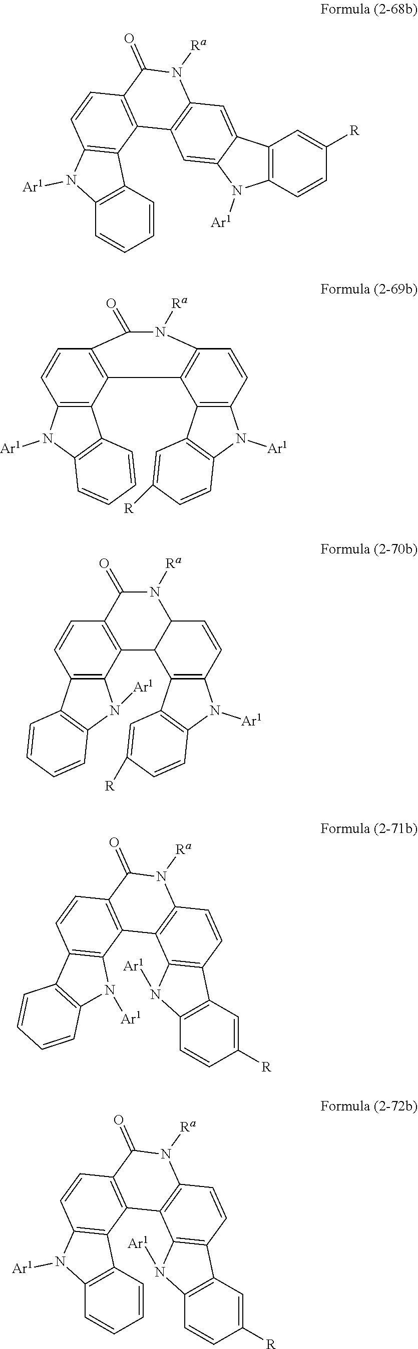

Especially preferred are compounds in which all substituents R are H or D.

In a preferred embodiment of the formula (1), Ar¹ is the same or different at each instance and is an aromatic or heteroaromatic ring system which has 6 to 40 aromatic ring atoms and may be substituted by one or more R¹ radicals, where the R¹ radicals are preferably nonaromatic. More preferably, Ar¹ is the same or different at each instance and is an aromatic or heteroaromatic ring system which has 6 to 24 aromatic ring atoms, especially 6 to 13 aromatic ring atoms, and may be substituted by one or more, preferably nonaromatic, R¹ radicals. When Ar¹ is a heteroaryl group, especially triazine, pyrimidine, quinazoline, quinoxaline or carbazole, preference may also be given to aromatic or heteroaromatic substituents R¹ on this heteroaryl group. Suitable aromatic or heteroaromatic ring systems Ar¹ are the same or different at each instance and are selected from the group consisting of phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R¹ radicals, preferably nonaromatic R¹ radicals. When Ar¹ is a heteroaryl group, especially triazine, pyrimidine, quinazoline, quinoxaline or carbazole, preference may also be given to aromatic or heteroaromatic R¹ radicals on this heteroaryl group.

Ar¹ here is preferably the same or different at each instance and is selected from the groups of the following formulae Ar-1 to Ar-83:

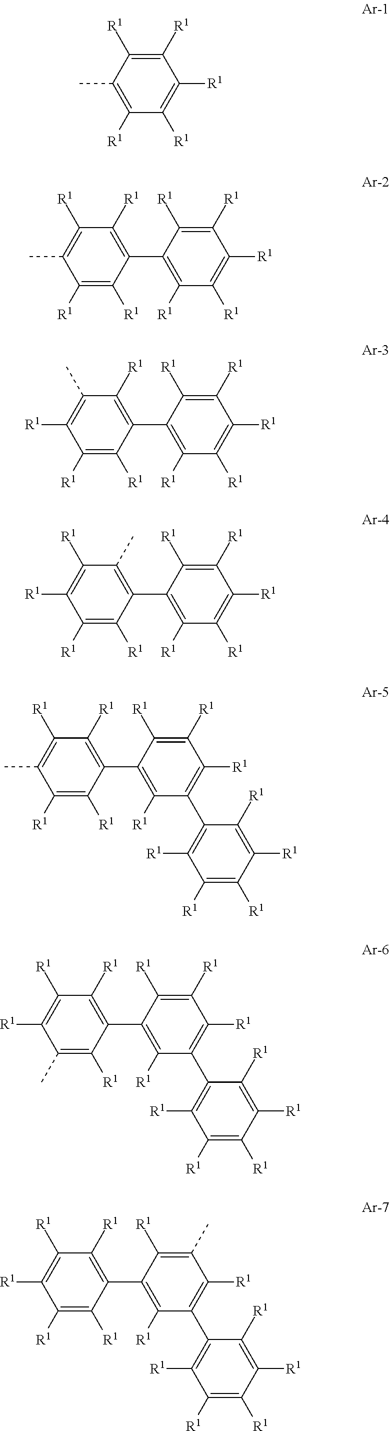

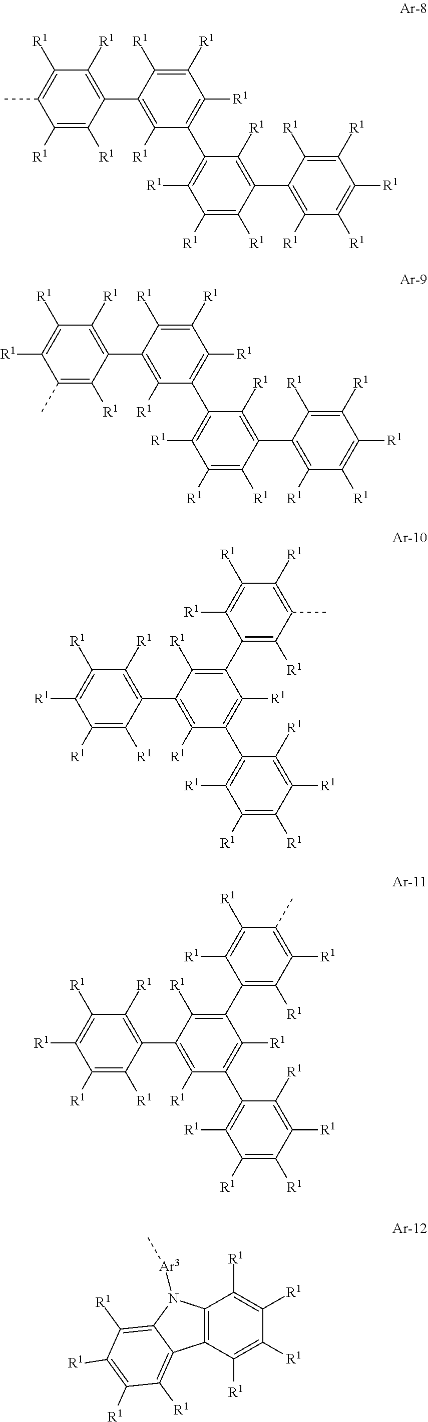

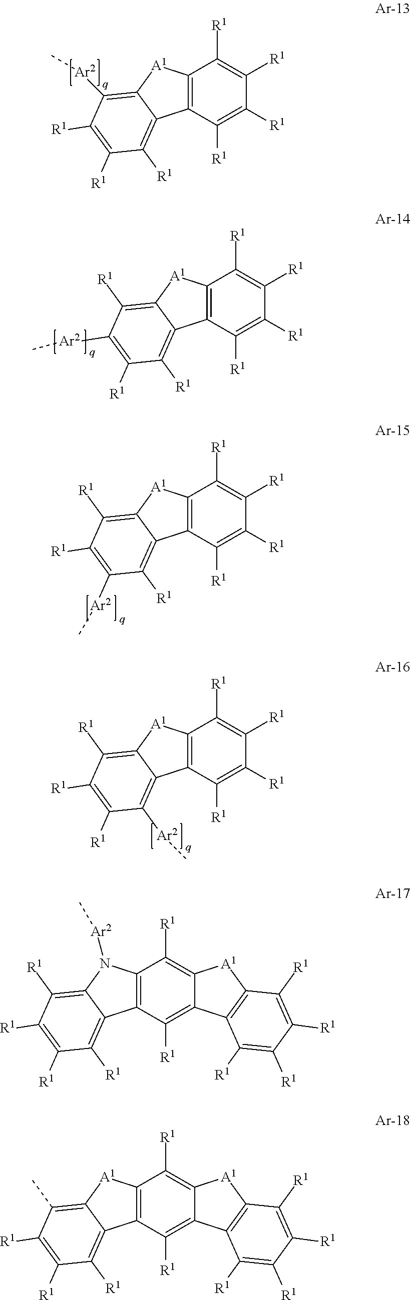

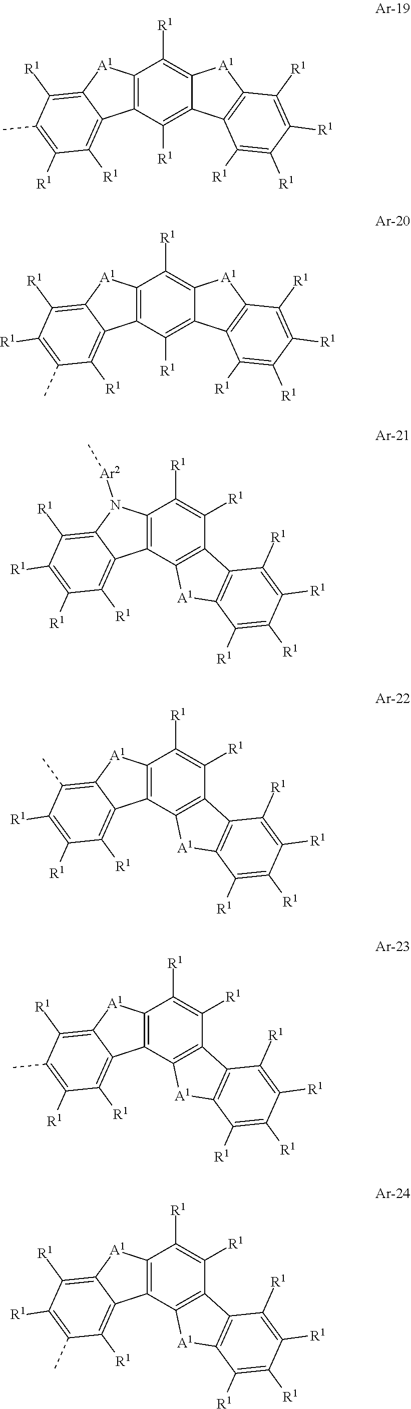

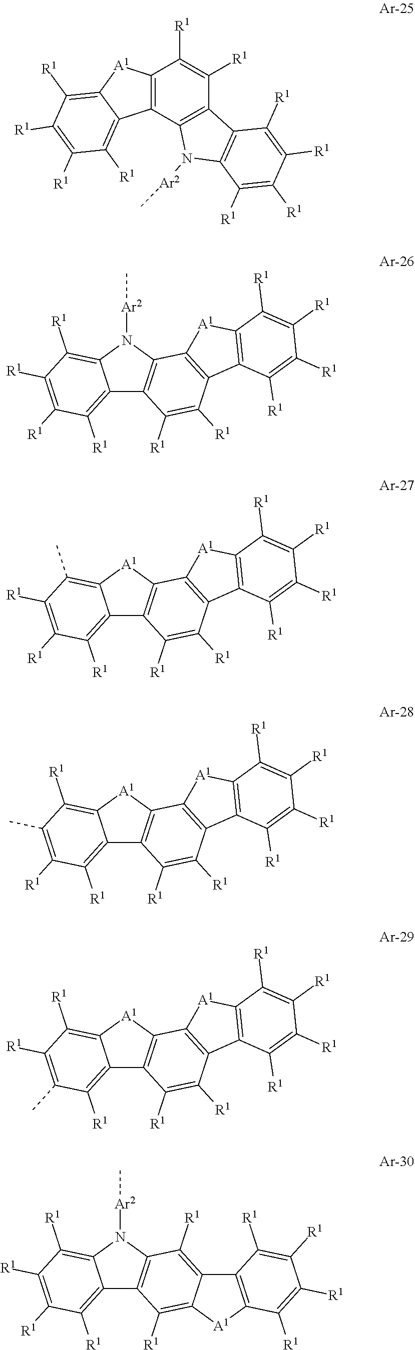

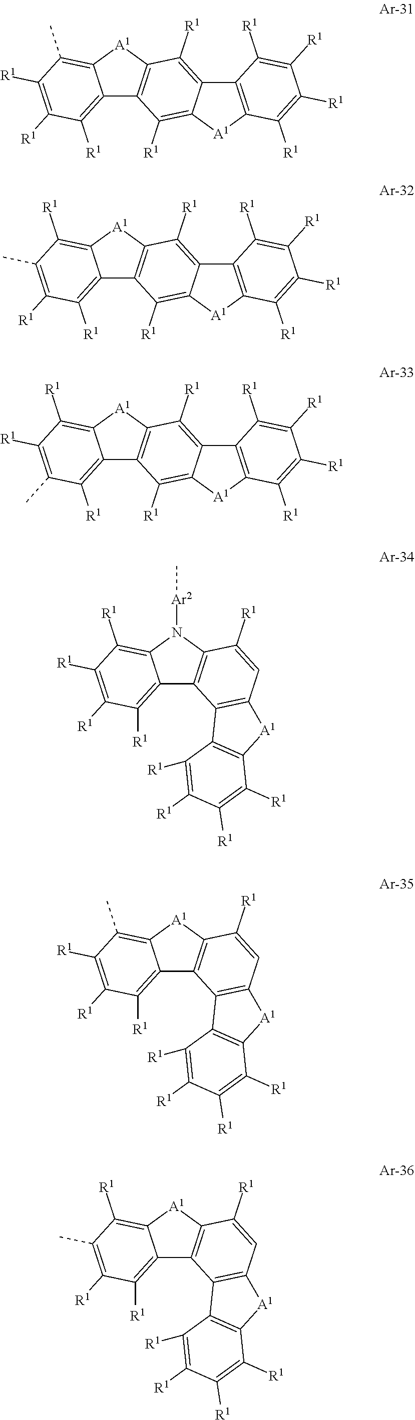

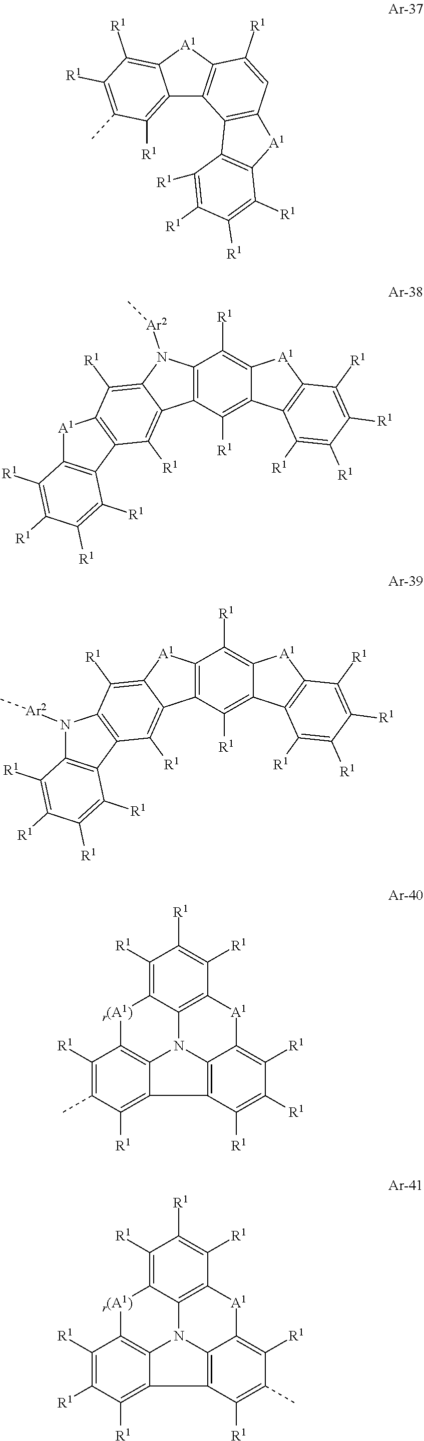

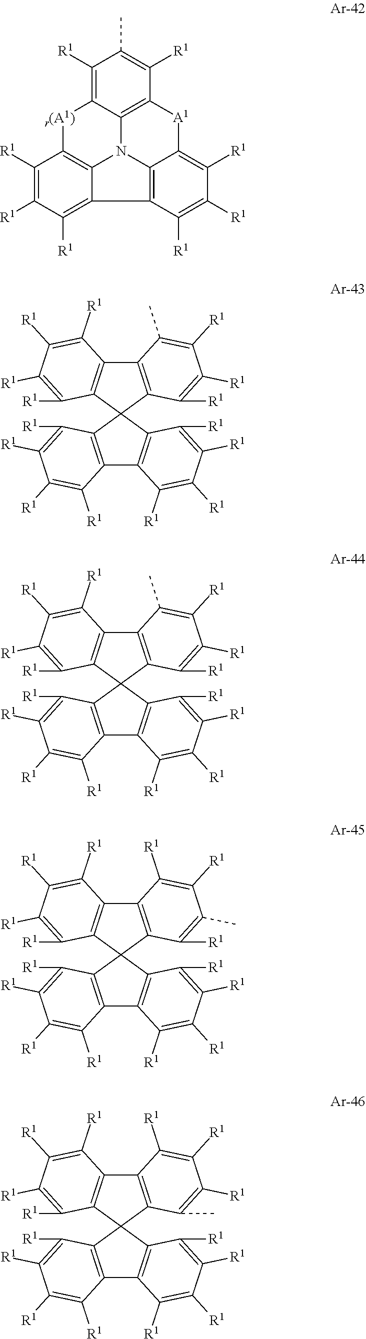

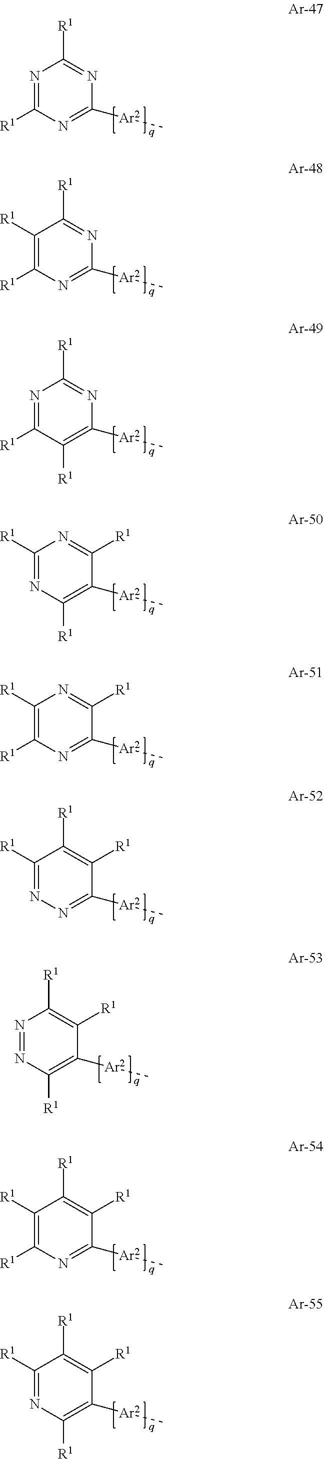

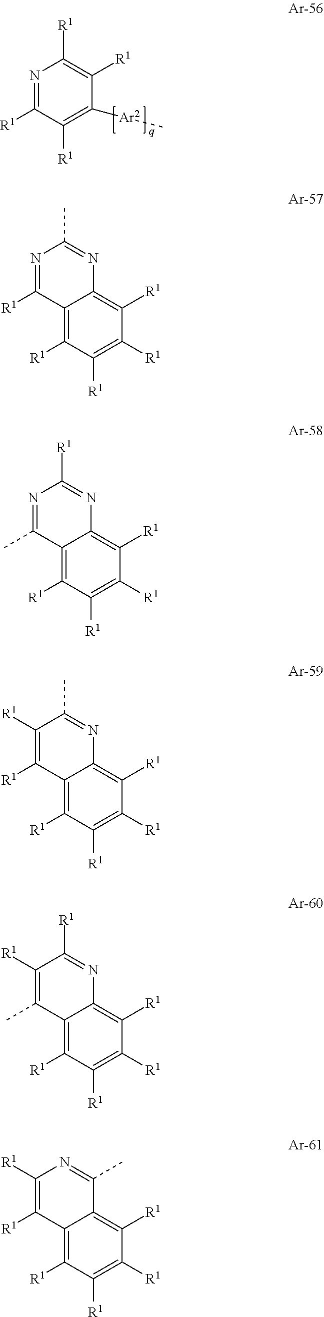

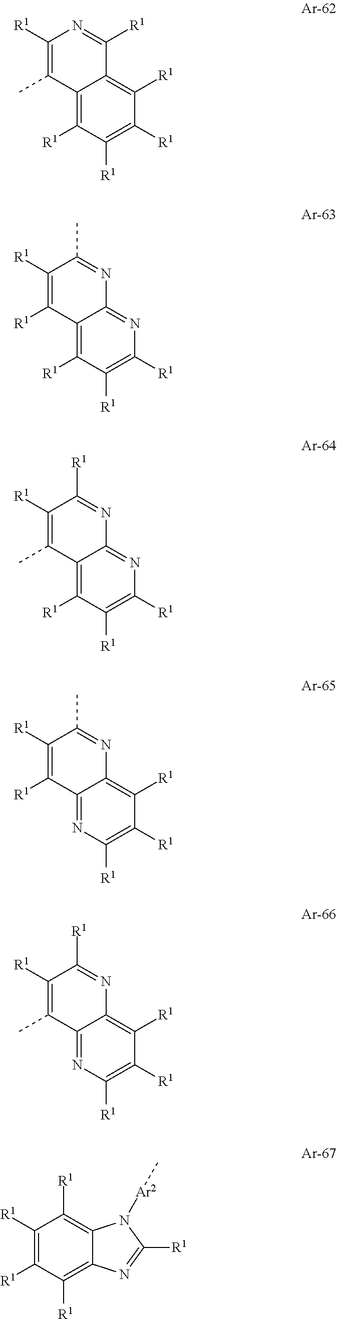

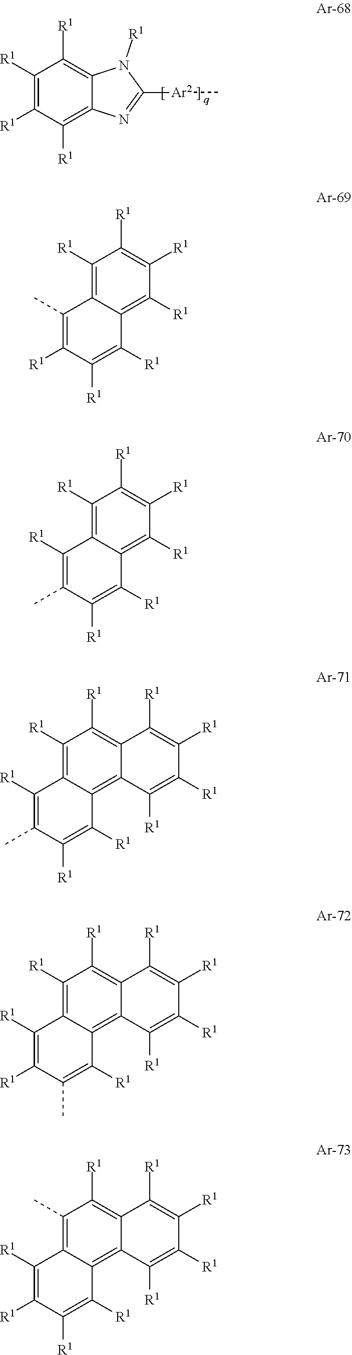

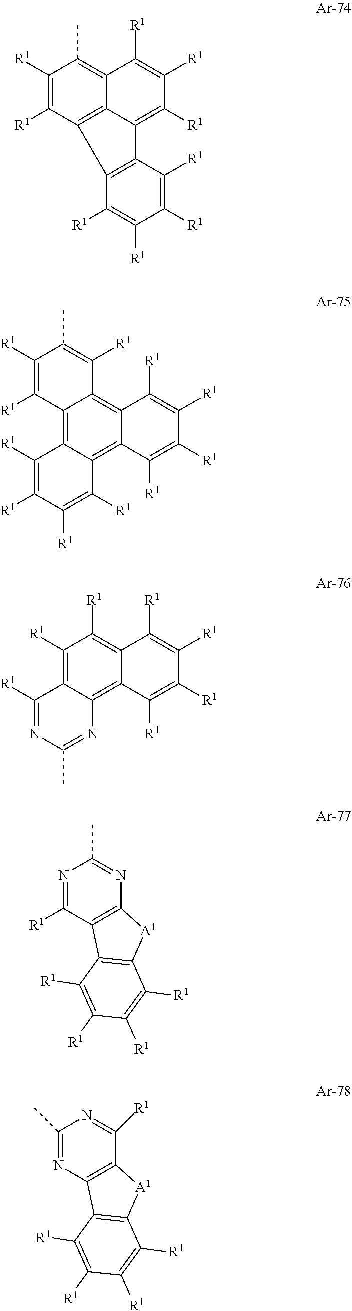

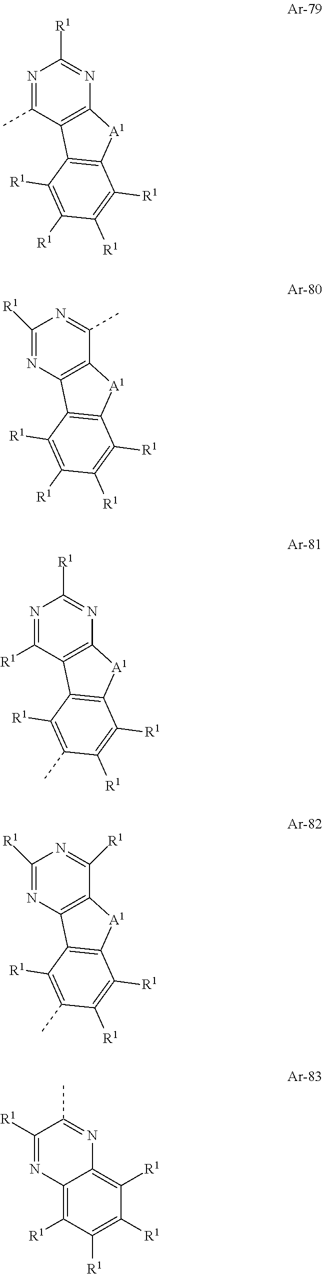

where R¹ has the definitions given above, the dotted bond represents the bond to the nitrogen atom, and in addition:
Ar² is the same or different at each instance and is a bivalent aromatic or heteroaromatic ring system which has 6 to 18 aromatic ring atoms and may be substituted in each case by one or more R¹ radicals;
A¹ is the same or different at each instance and is NR¹, O, S or C(R¹)₂;
r is 0 or 1, where r=0 means that no A¹ group is bonded at this position and R¹ radicals are bonded to the corresponding carbon atoms instead;
q is 0 or 1, where g=0 means that the Ar³ group is absent and that the corresponding aromatic or heteroaromatic group is bonded directly to the nitrogen atom.

When the abovementioned Ar-1 to Ar-83 groups have two or more A¹ groups, possible options for these include all combinations from the definition of A¹. Preferred embodiments in that case are those in which one A¹ group is NR¹ and the other A¹ group is C(R¹)₂ or in which both A¹ groups are NR¹ or in which both A¹ groups are O. In a particularly preferred embodiment of the invention, in Ar¹ groups having two or more A¹ groups, at least one A¹ group is C(R¹)₂ or is NR¹.

When A¹ is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this R¹ substituent is the same or different at each instance and is an aromatic or heteroaromatic ring system which has 6 to 24 aromatic ring atoms, preferably 6 to 12 aromatic ring atoms, and which does not have any fused aryl groups or heteroaryl groups in which two or more aromatic or heteroaromatic 6-membered ring groups are fused directly to one another, and which may also be substituted in each case by one or more R² radicals. Particular preference is given to phenyl, biphenyl, terphenyl and quaterphenyl, and also triphenylene, having bonding patterns as listed above for Ar-1 to Ar-11 and Ar-75, where these structures may be substituted by one or more R² radicals, but are preferably unsubstituted.

When A¹ is C(R¹)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R² radicals. Most preferably, R¹ is a methyl group or a phenyl group. In this case, the R¹ radicals together may also form a ring system, which leads to a spiro system.

In a preferred embodiment of the formula (1), R^{a} is the same or different at each instance and is an aromatic or heteroaromatic ring system which has 6 to 40 aromatic ring atoms and may be substituted by one or more R¹ radicals. In addition, the preferred embodiments specified above for Ar¹ and R¹ are applicable. R^{a} is consequently preferably selected from the formulae Ar-1 to Ar-83.

In a further preferred embodiment of the invention, R is the same or different at each instance and is selected from the group consisting of H, D, F, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R¹ radicals, and where one or more nonadjacent CH₂ groups may be replaced by O, or an aromatic or heteroaromatic ring system which has 6 to 30 aromatic ring atoms and may be substituted in each case by one or more R¹ radicals; at the same time, two or more R¹ radicals together may form an aliphatic, heteroaliphatic, aromatic or heteroaromatic ring system. In a particularly preferred embodiment of the invention, R is the same or different at each instance and is selected from the group consisting of H, D, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R¹ radicals, but is preferably unsubstituted, or an aromatic or heteroaromatic ring system which has 6 to 24 aromatic ring atoms, preferably 6 to 13 aromatic ring atoms, and may be substituted in each case by one or more R¹ radicals, but is preferably unsubstituted.

When R is an aromatic or heteroaromatic ring system, it is preferably selected from the above-depicted structures (Ar-1) to (Ar-83).

In a further preferred embodiment of the invention, R¹ is the same or different at each instance and is selected from the group consisting of H, D, F, CN, OR², a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R² radicals, and where one or more nonadjacent CH₂ groups may be replaced by O, or an aromatic or heteroaromatic ring system which has 6 to 30 aromatic ring atoms and may be substituted in each case by one or more R² radicals; at the same time, two or more R² radicals together may form an aliphatic, heteroaliphatic, aromatic or heteroaromatic ring system. In a particularly preferred embodiment of the invention, R¹ is the same or different at each instance and is selected from the group consisting of H, D, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 24 aromatic ring atoms, preferably 6 to 13 aromatic ring atoms, and may be substituted in each case by one or more R² radicals, but is preferably unsubstituted.

When R¹ is an aromatic or heteroaromatic ring system, it is preferably selected from the above-depicted structures (Ar-1) to (Ar-83).

In a further preferred embodiment of the invention, R² is the same or different at each instance and is H, D, F, an alkyl group having 1 to 4 carbon atoms or an aryl group having 6 to 10 carbon atoms, which may be substituted by an alkyl group having 1 to 4 carbon atoms, but is preferably unsubstituted.

At the same time, the alkyl groups in compounds of the invention which are processed by vacuum evaporation preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

When the compounds of the formula (1) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. It is especially preferable that the R, R¹ and R² groups do not contain any fused aryl or heteroaryl groups in which two or more six-membered rings are fused directly to one another. An exception to this is formed by phenanthrene, triphenylene, quinazoline and quinoxaline, which, because of their higher triplet energy, may be preferable in spite of the presence of fused aromatic six-membered rings.

The abovementioned preferred embodiments may be combined with one another as desired within the restrictions defined in claim 1. In a particularly preferred embodiment of the invention, the abovementioned preferences occur simultaneously.

Examples of suitable compounds according to the above-detailed embodiments are the compounds detailed in the following table:

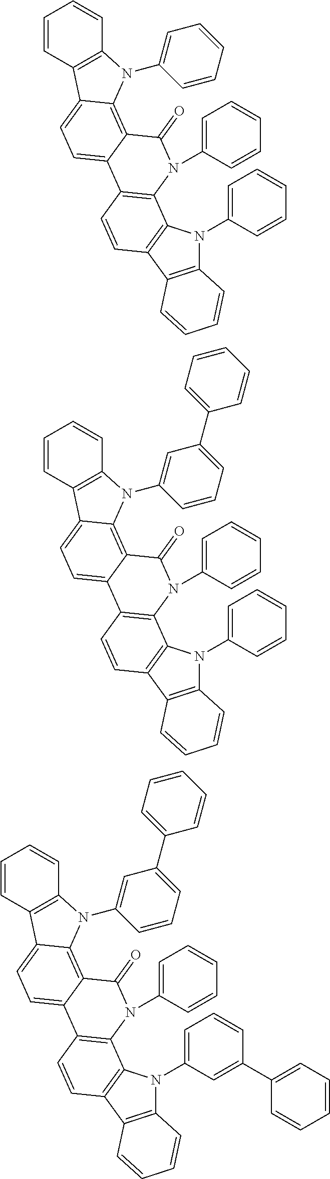

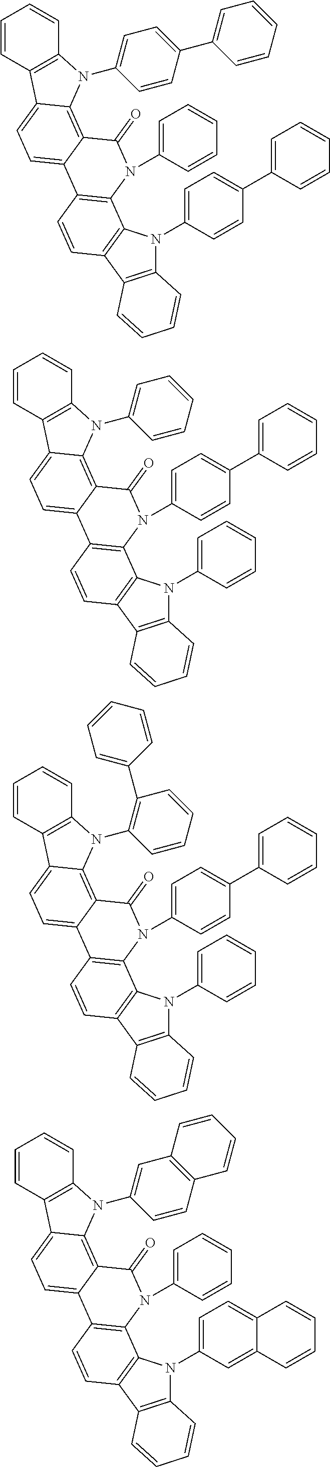

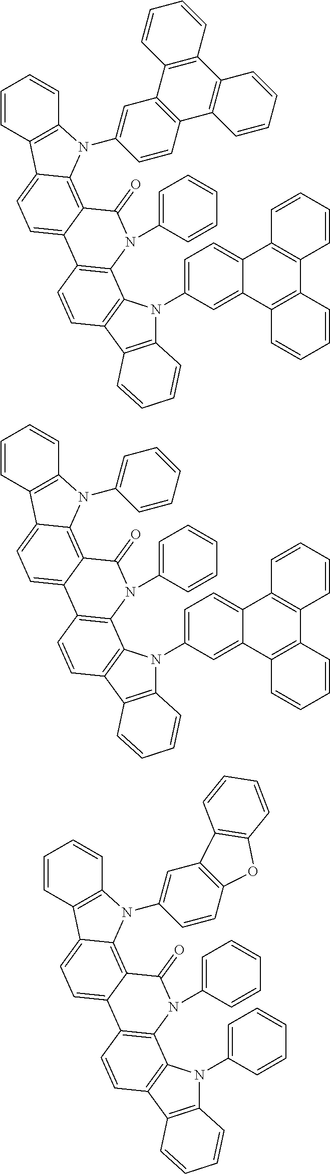

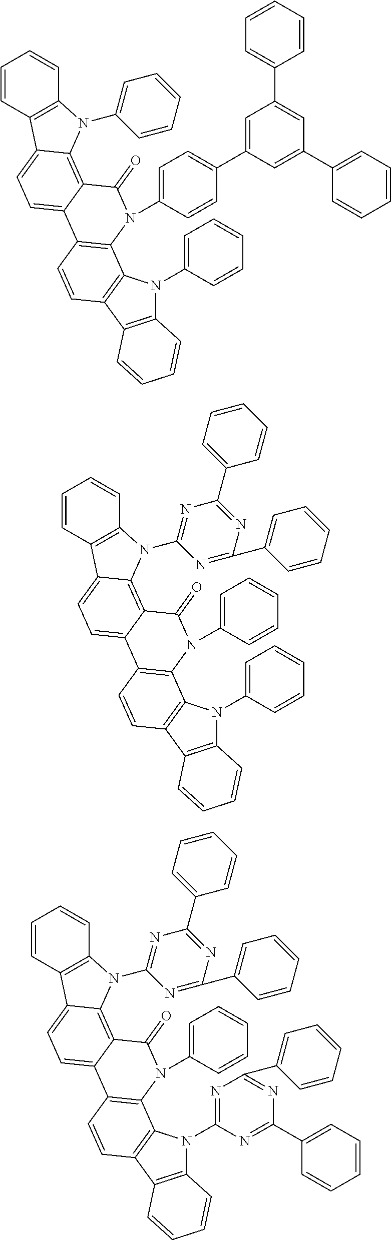

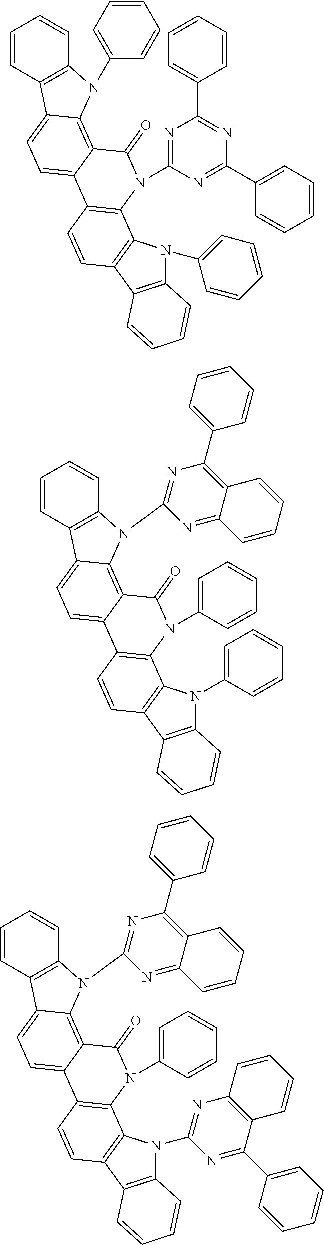

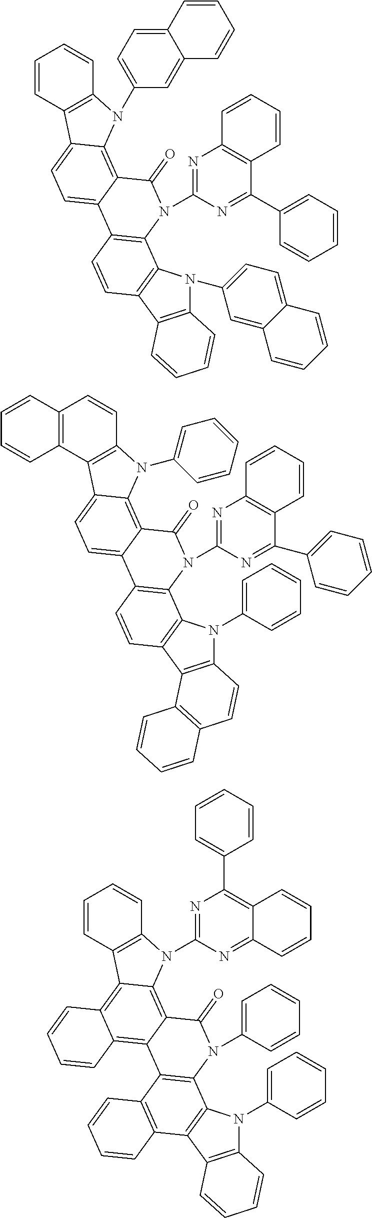

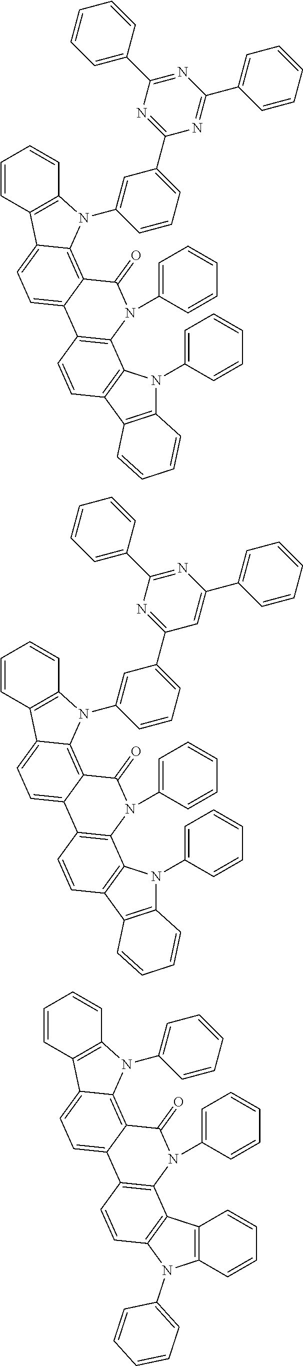

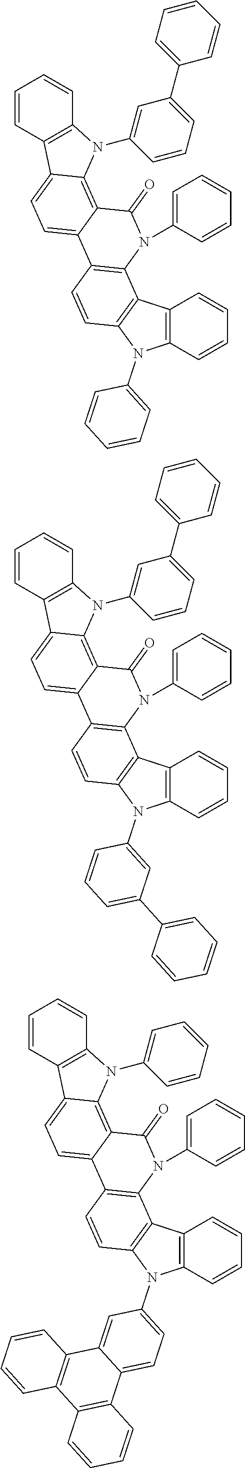

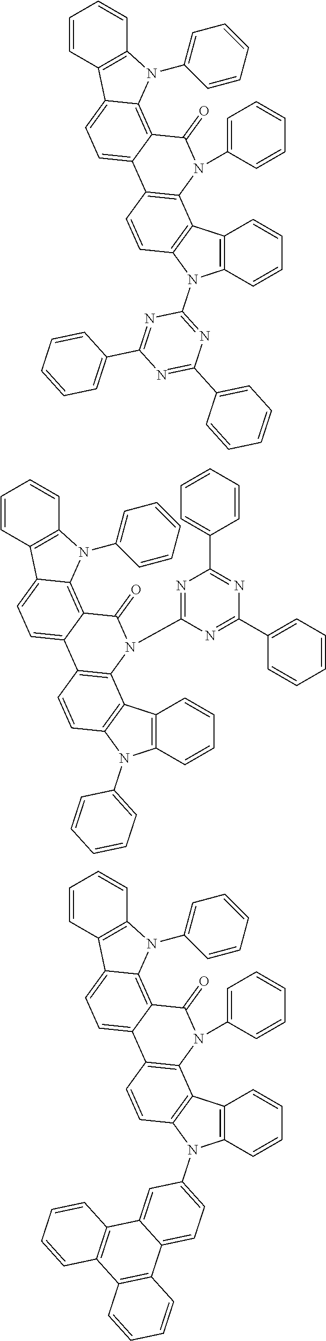

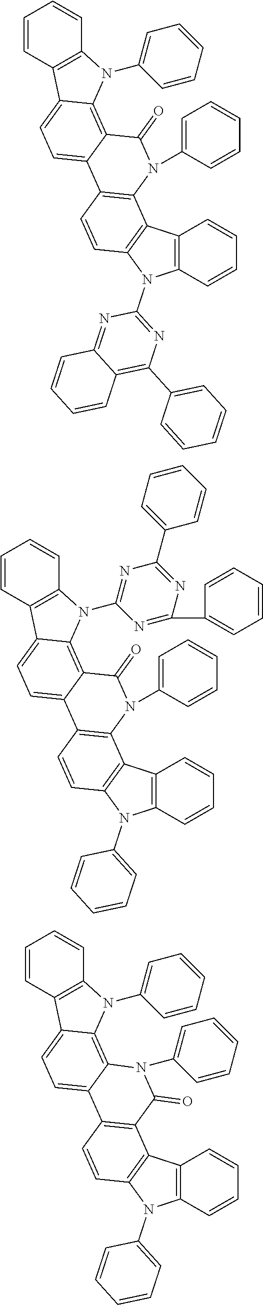

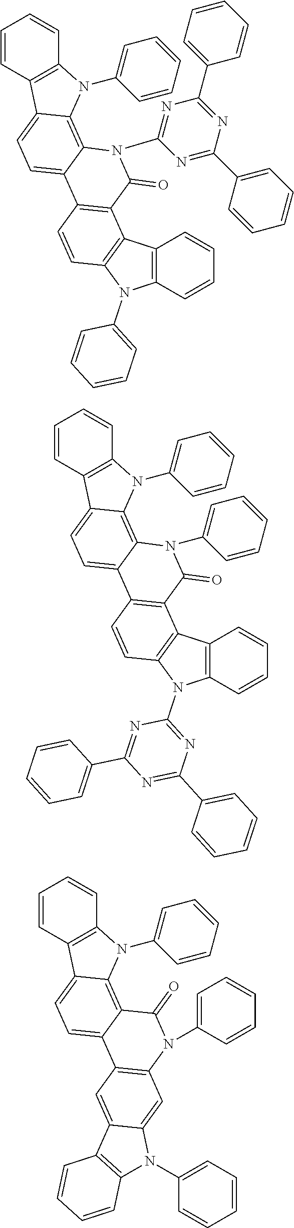

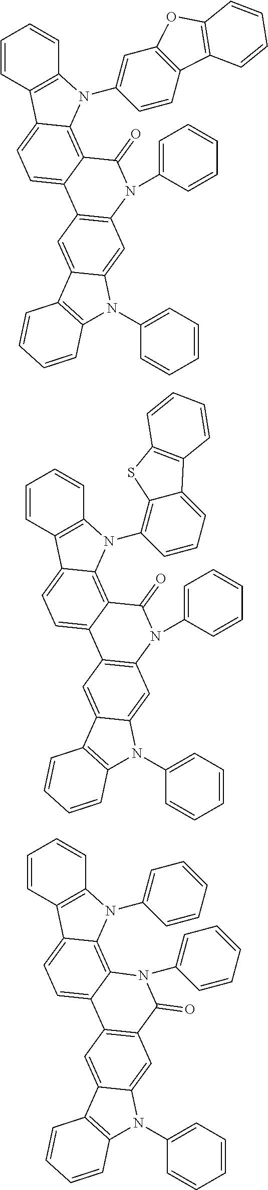

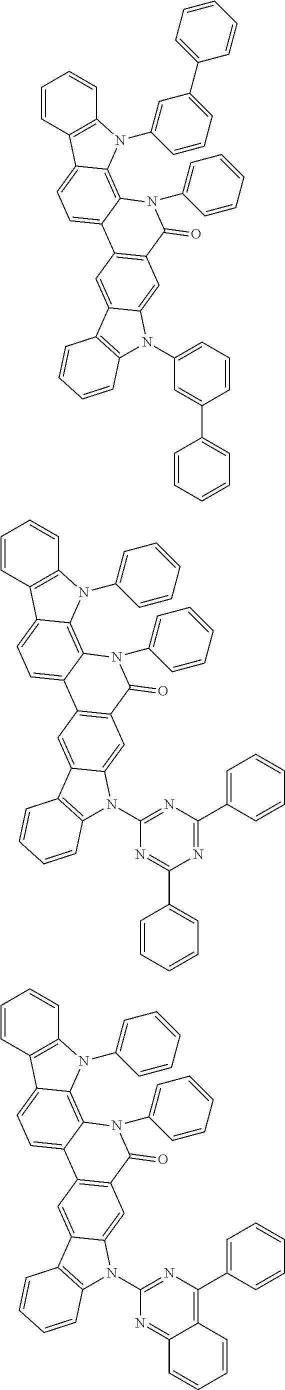

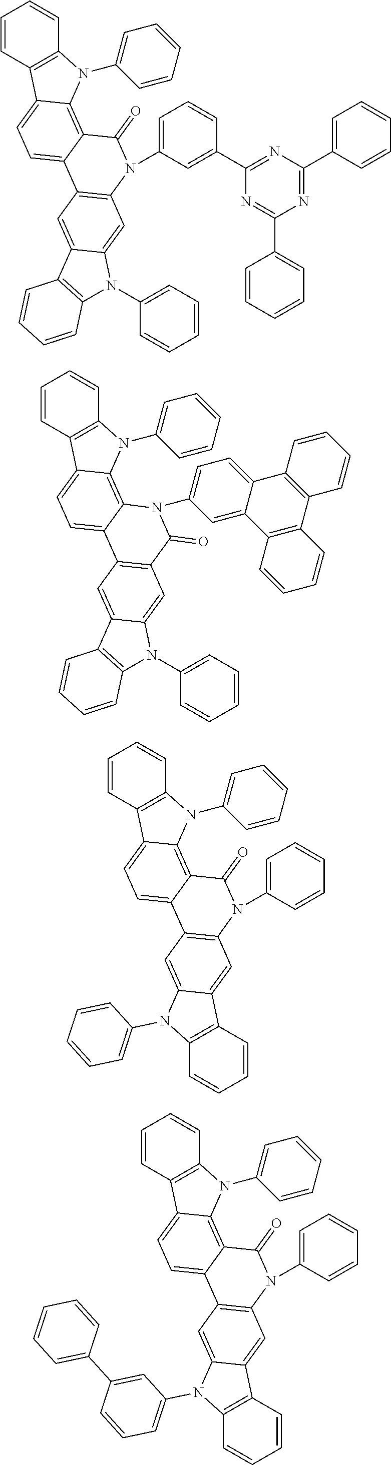

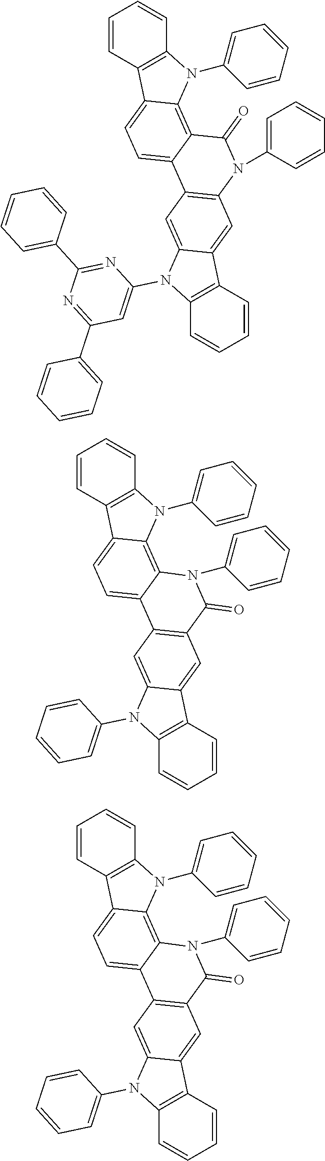

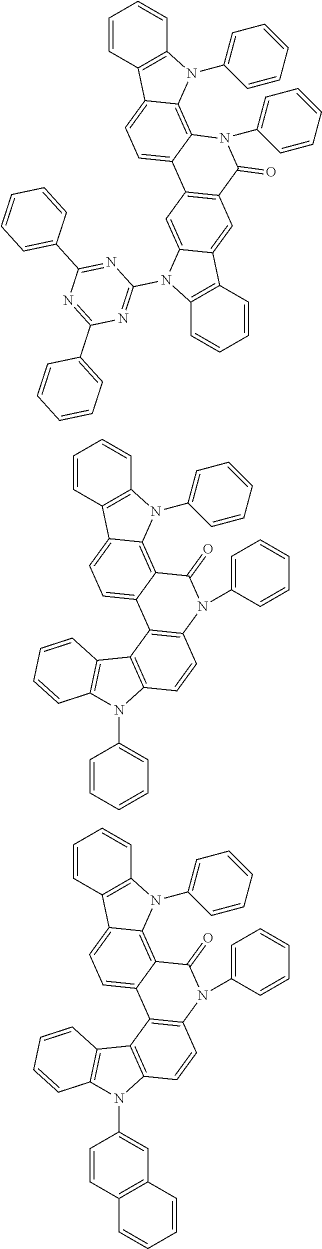

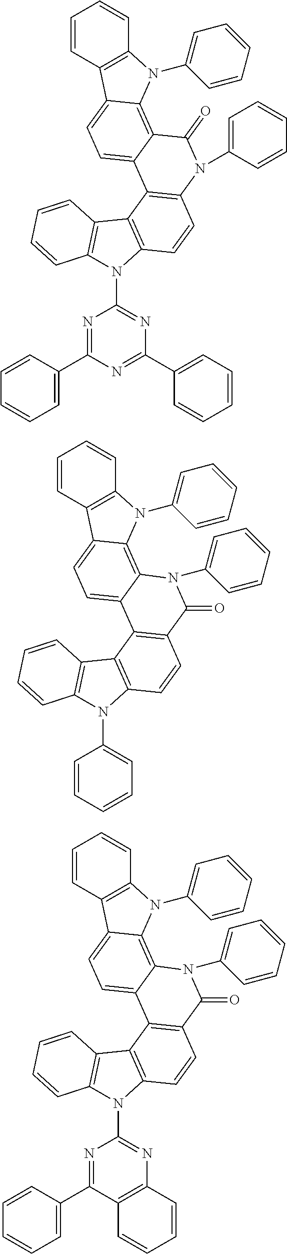

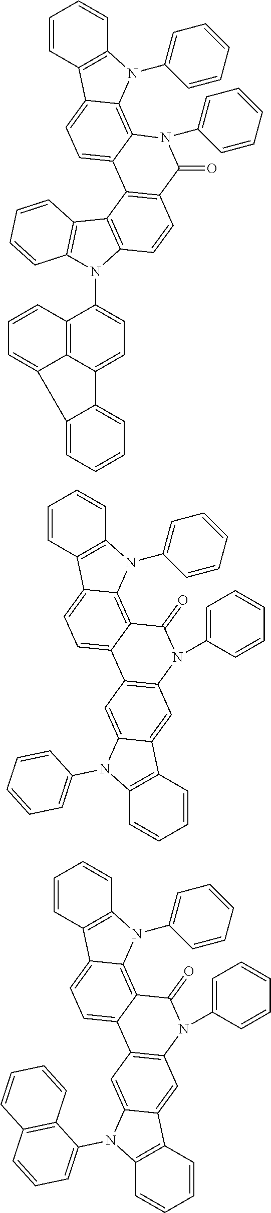

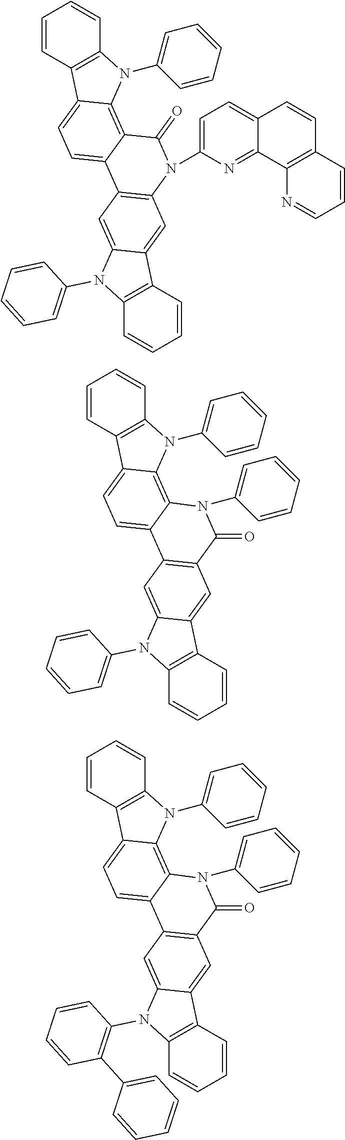

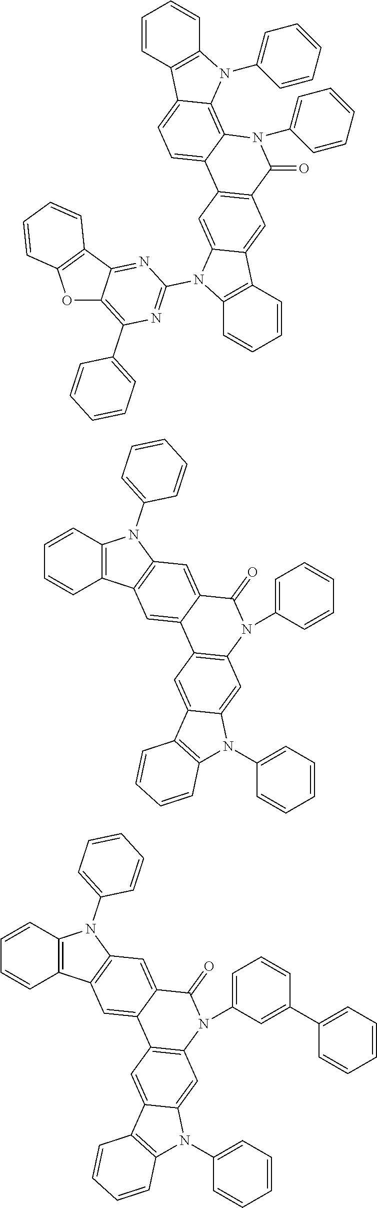

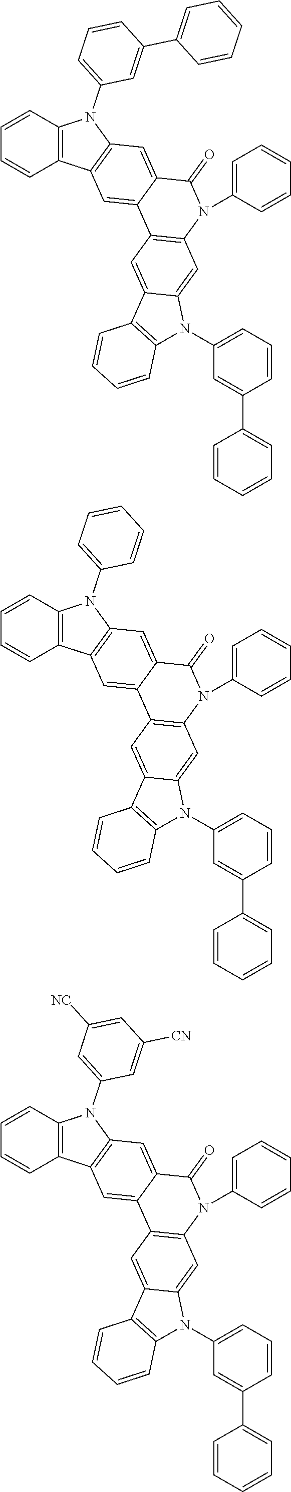 [SMILES: O=c1c2cc3c(cc2c2cc4c5ccccc5n(-c5cccc(-c6ccccc6)c5)c4cc2n1-c1ccccc1)c1ccccc1n3-c1cccc(-c2ccccc2)c1 ; O=c1c2cc3c(cc2c2cc4c5ccccc5n(-c5cccc(-c6ccccc6)c5)c4cc2n1-c1ccccc1)c1ccccc1n3-c1ccccc1 ; N#Cc1cc(C#N)cc(-n2c3ccccc3c3cc4c(cc32)c(=O)n(-c2ccccc2)c2cc3c(cc42)c2ccccc2n3-c2cccc(-c3ccccc3)c2)c1]

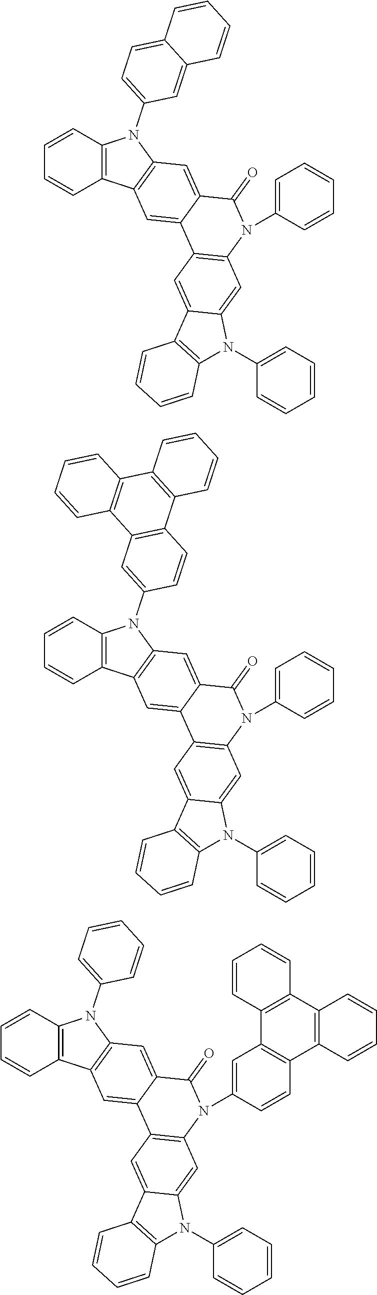

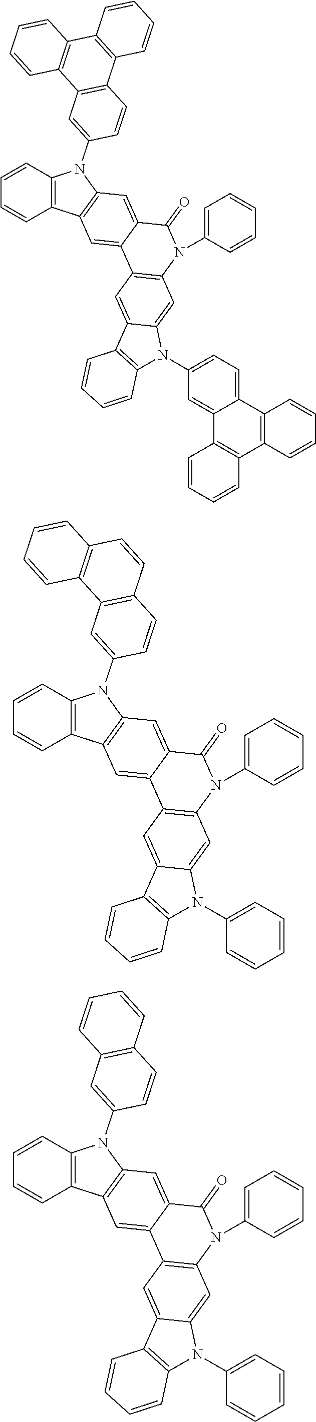

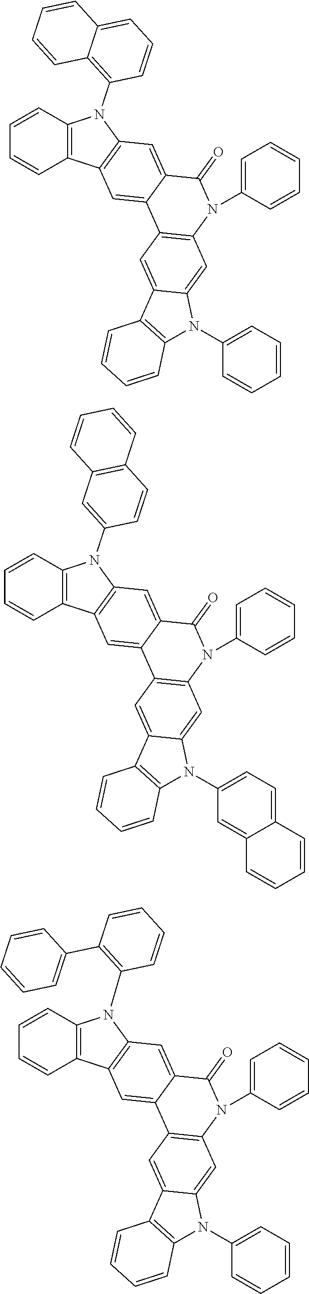

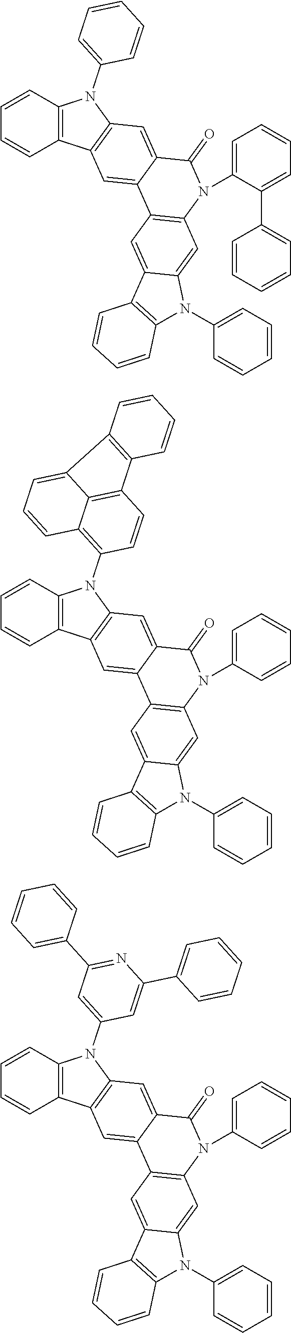

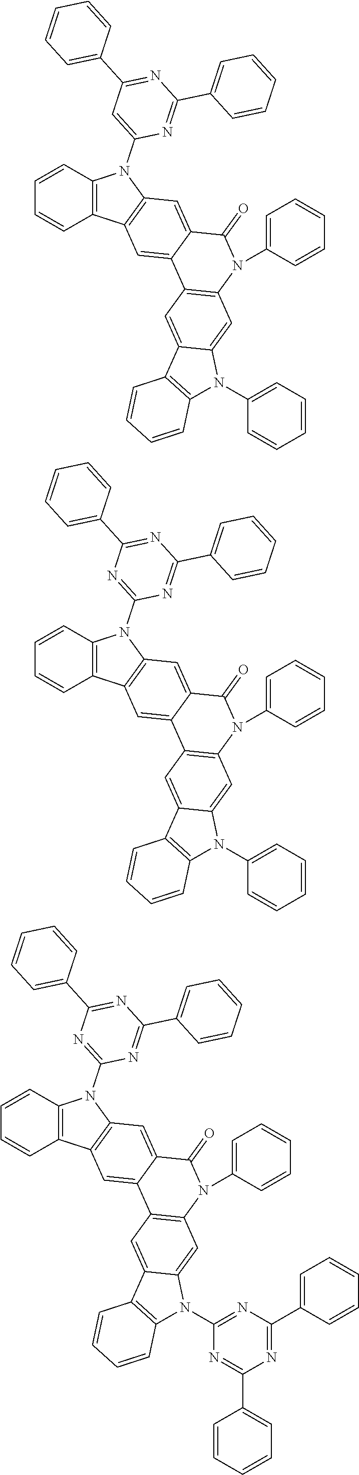 [SMILES: O=c1c2cc3c(cc2c2cc4c5ccccc5n(-c5ccccc5)c4cc2n1-c1ccccc1)c1ccccc1n3-c1cc(-c2ccccc2)nc(-c2ccccc2)n1 ; O=c1c2cc3c(cc2c2cc4c5ccccc5n(-c5ccccc5)c4cc2n1-c1ccccc1)c1ccccc1n3-c1nc(-c2ccccc2)nc(-c2ccccc2)n1 ; O=c1c2cc3c(cc2c2cc4c5ccccc5n(-c5nc(-c6ccccc6)nc(-c6ccccc6)n5)c4cc2n1-c1ccccc1)c1ccccc1n3-c1nc(-c2ccccc2)nc(-c2ccccc2)n1]

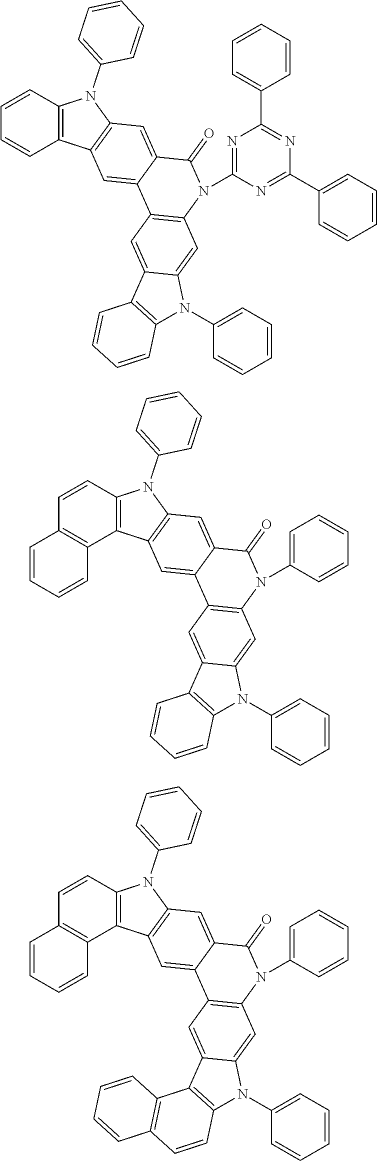

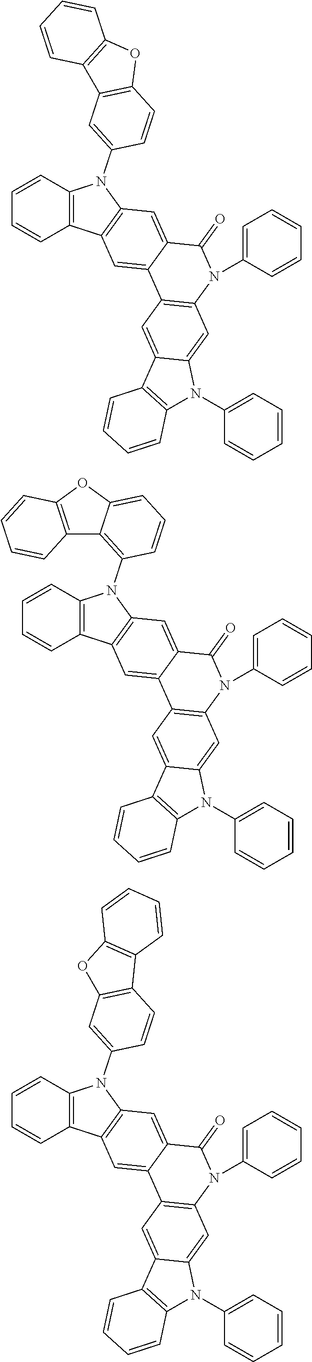

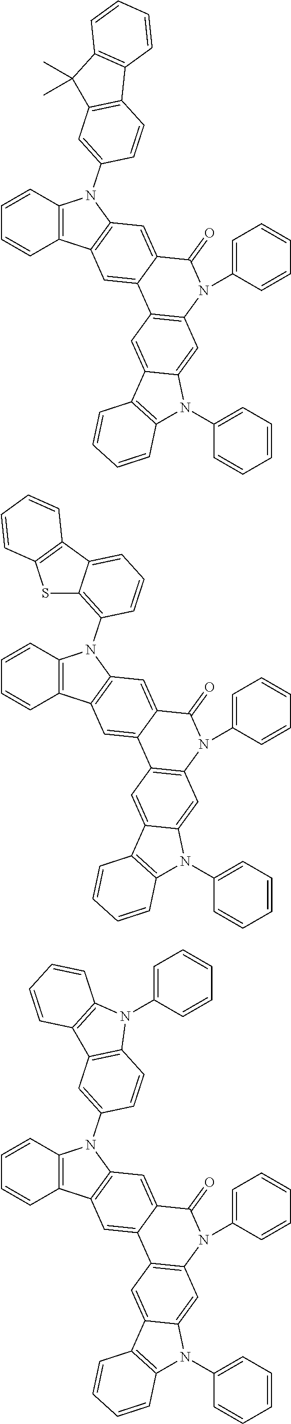 [SMILES: CC1(C)c2ccccc2-c2ccc(-n3c4ccccc4c4cc5c(cc43)c(=O)n(-c3ccccc3)c3cc4c(cc53)c3ccccc3n4-c3ccccc3)cc21 ; O=c1c2cc3c(cc2c2cc4c5ccccc5n(-c5ccccc5)c4cc2n1-c1ccccc1)c1ccccc1n3-c1cccc2c1sc1ccccc12 ; O=c1c2cc3c(cc2c2cc4c5ccccc5n(-c5ccccc5)c4cc2n1-c1ccccc1)c1ccccc1n3-c1ccc2c(c1)c1ccccc1n2-c1ccccc1]

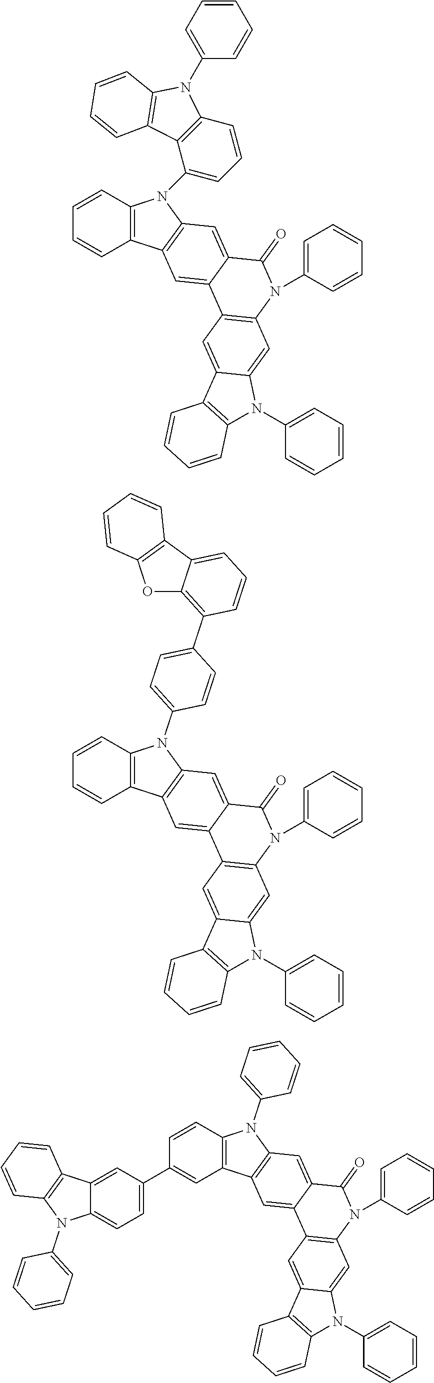

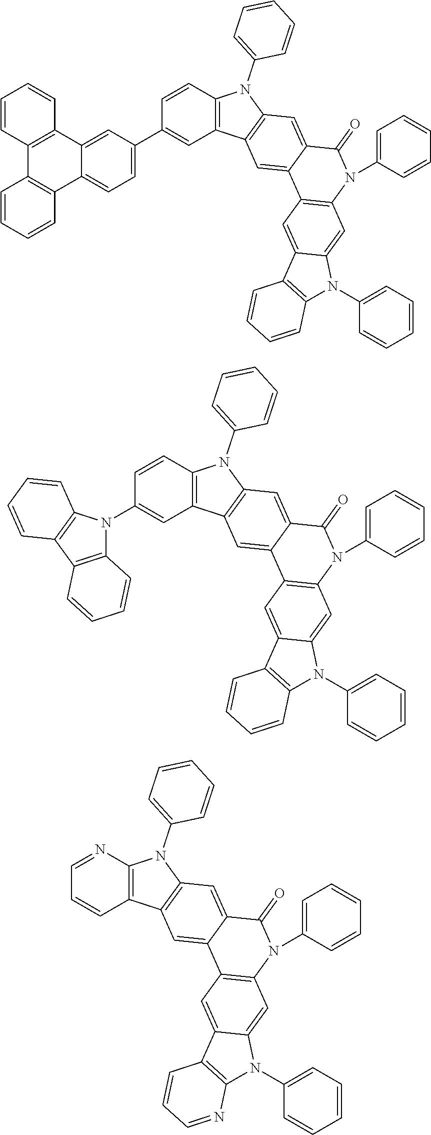

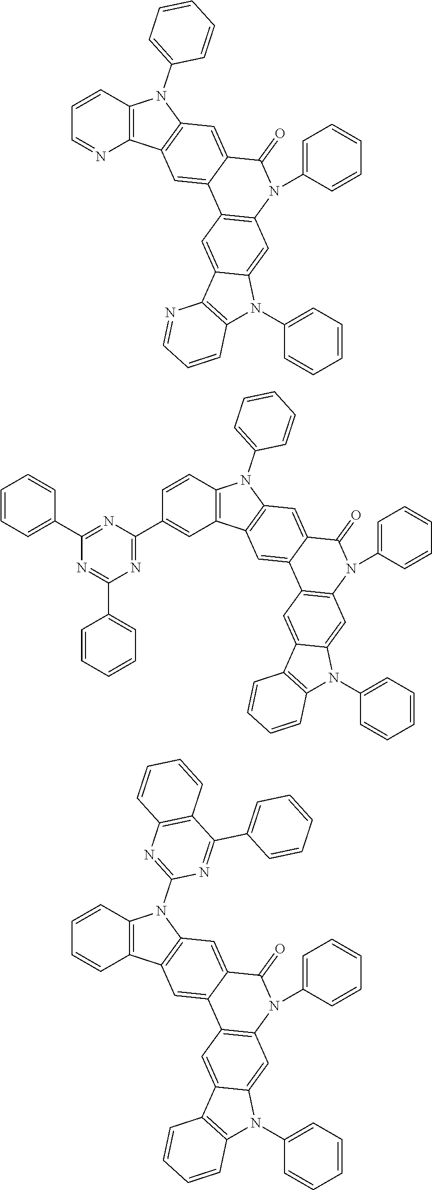

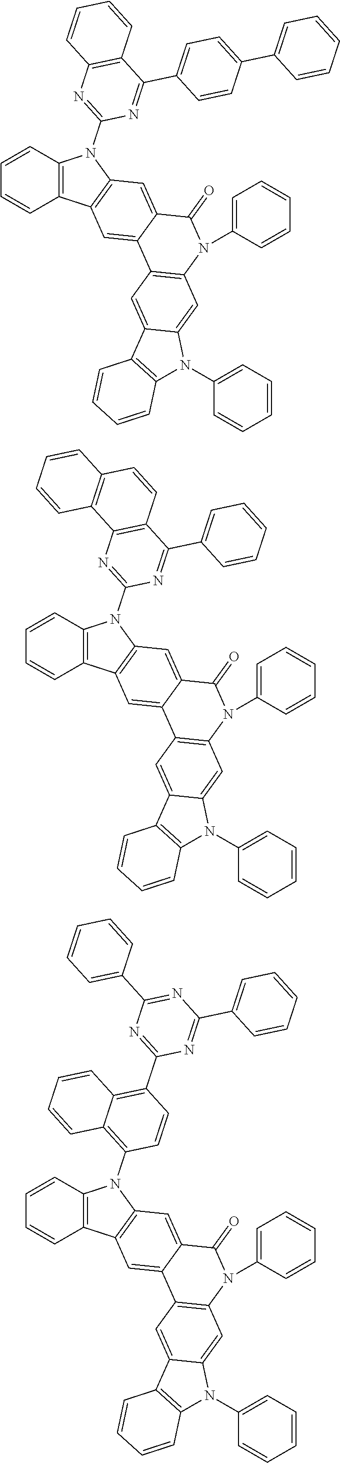

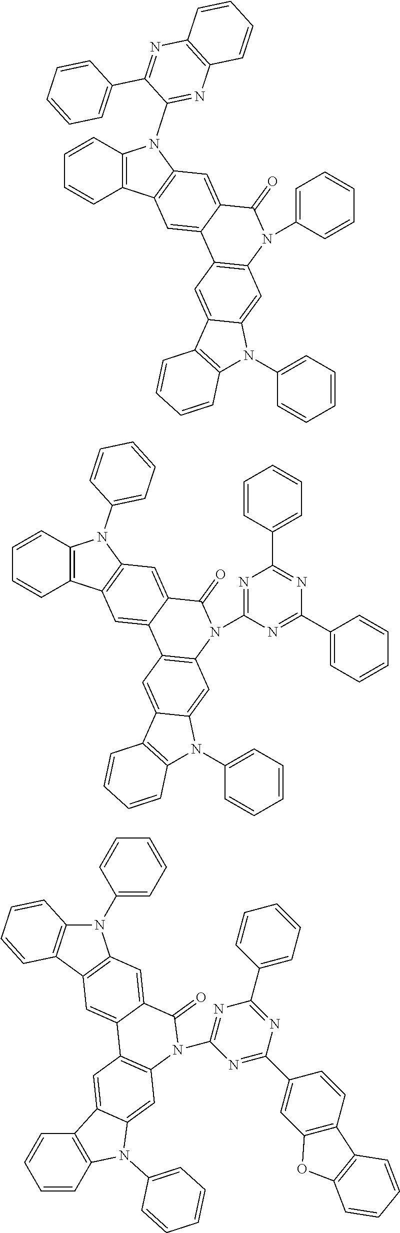

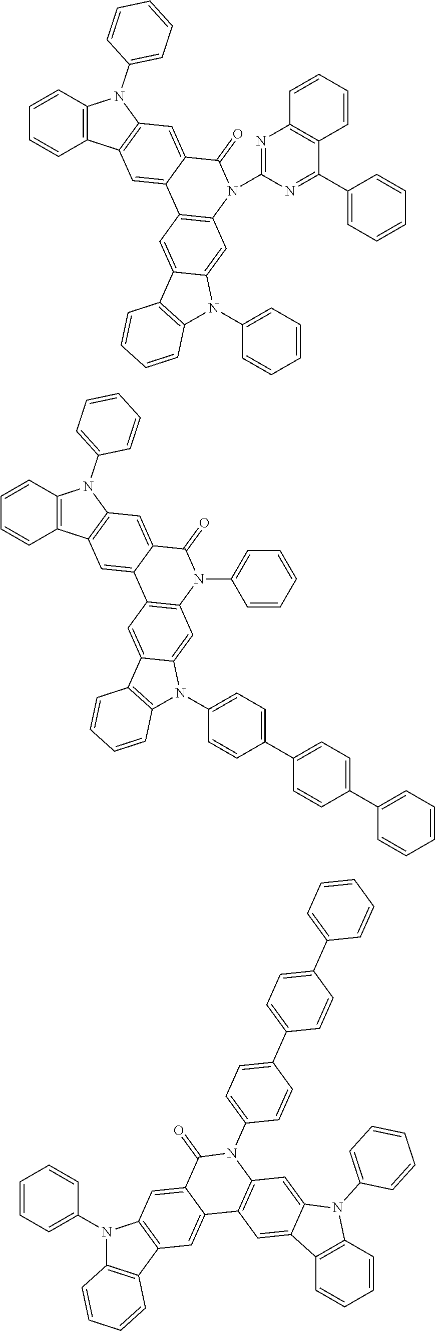

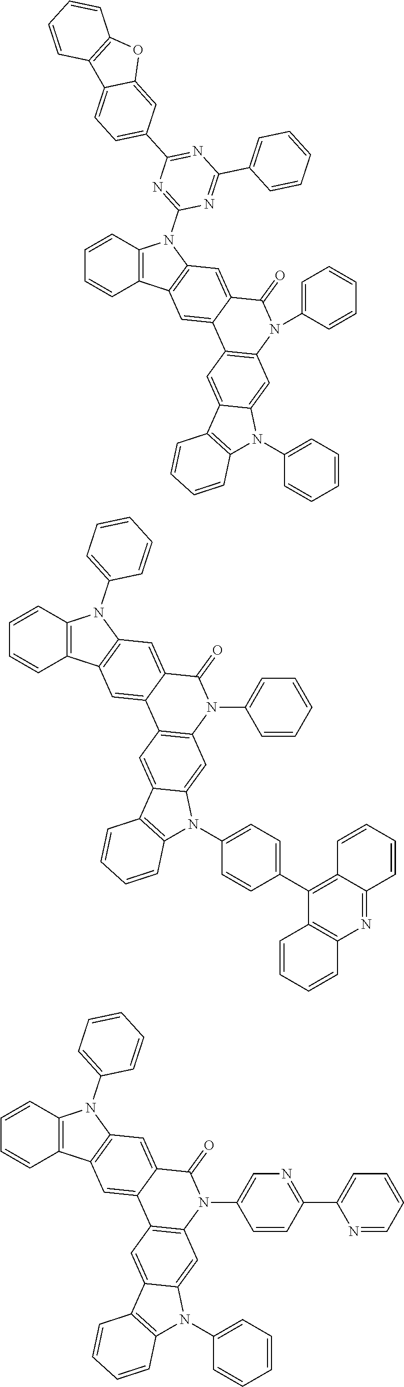 [SMILES: O=c1c2cc3c(cc2c2cc4c5ccccc5n(-c5ccccc5)c4cc2n1-c1ccccc1)c1ccccc1n3-c1nc(-c2ccccc2)nc(-c2ccc3c(c2)oc2ccccc23)n1 ; O=c1c2cc3c(cc2c2cc4c5ccccc5n(-c5ccc(-c6c7ccccc7nc7ccccc67)cc5)c4cc2n1-c1ccccc1)c1ccccc1n3-c1ccccc1 ; O=c1c2cc3c(cc2c2cc4c5ccccc5n(-c5ccccc5)c4cc2n1-c1ccc(-c2ccccn2)nc1)c1ccccc1n3-c1ccccc1]

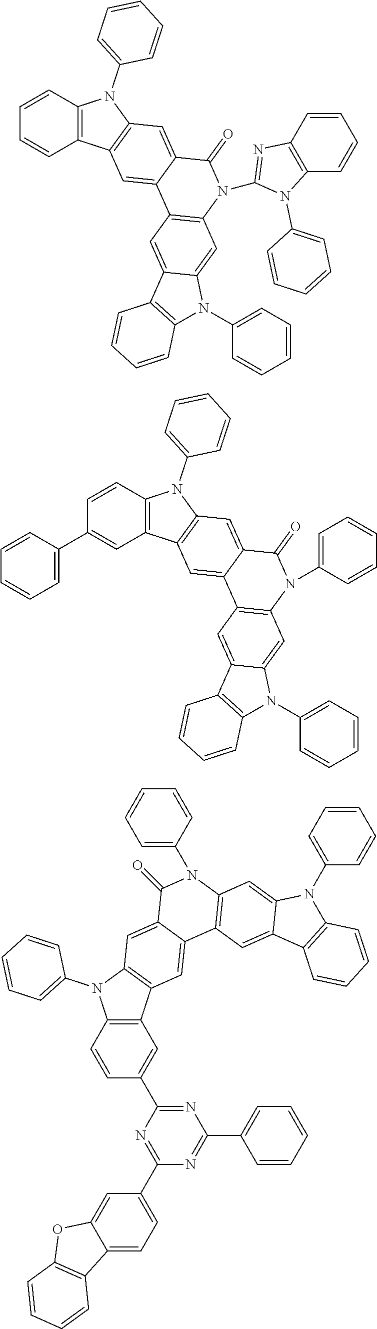

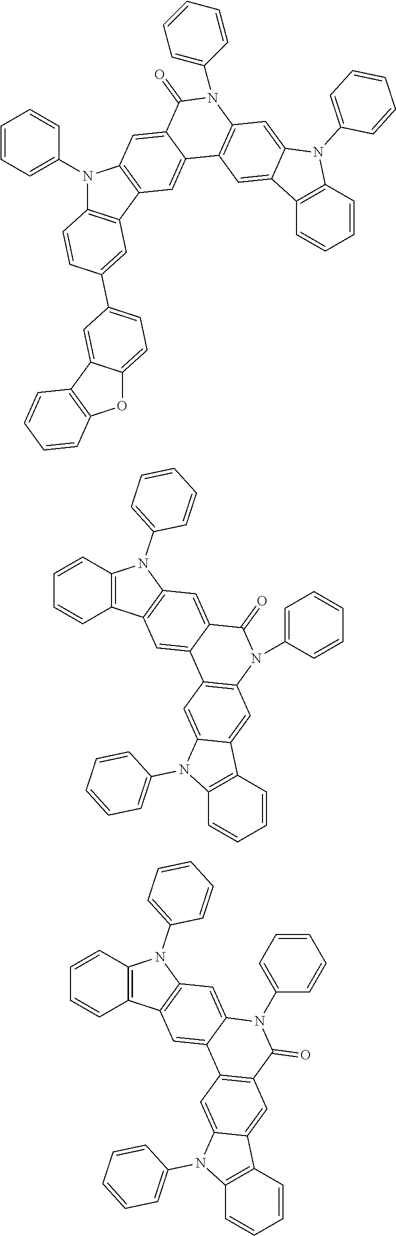

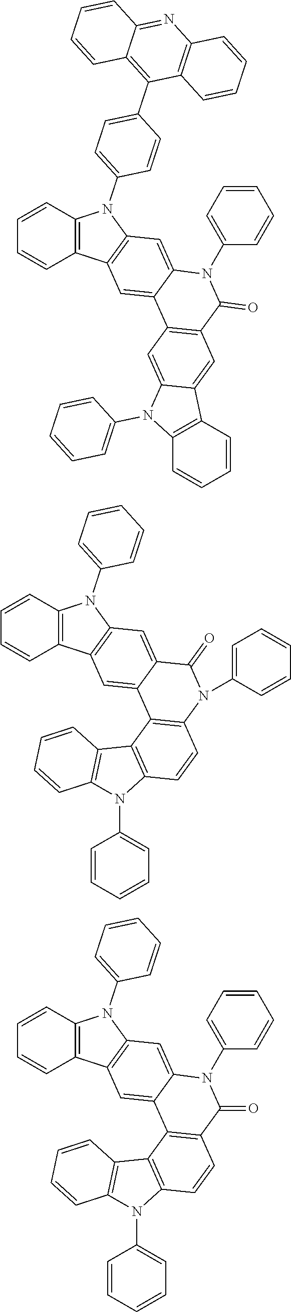

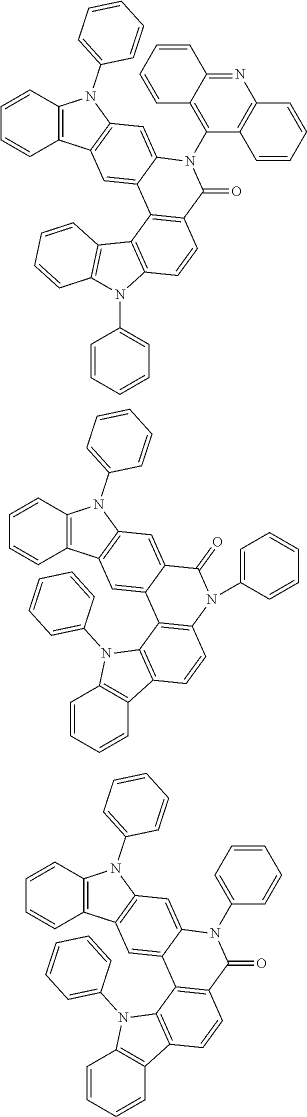

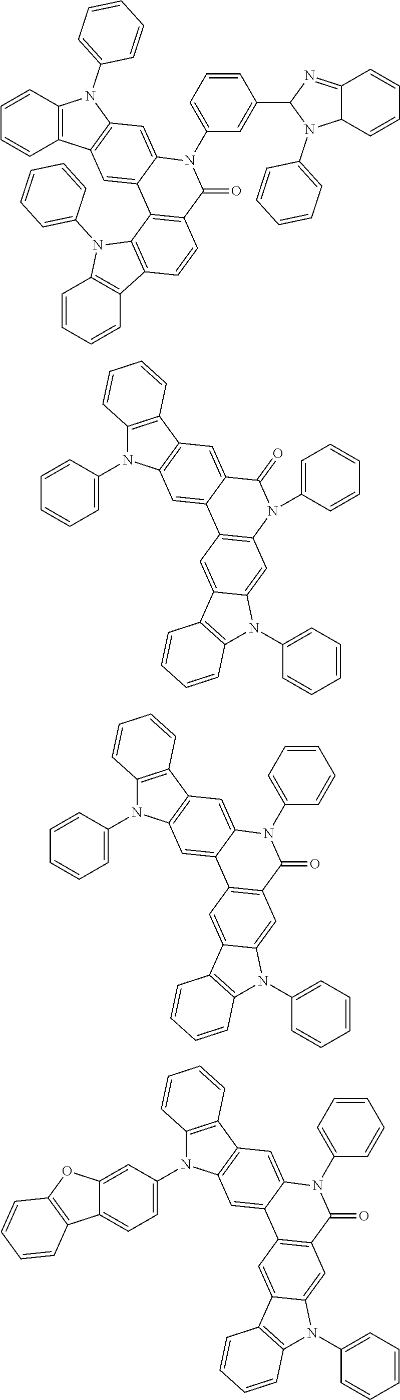

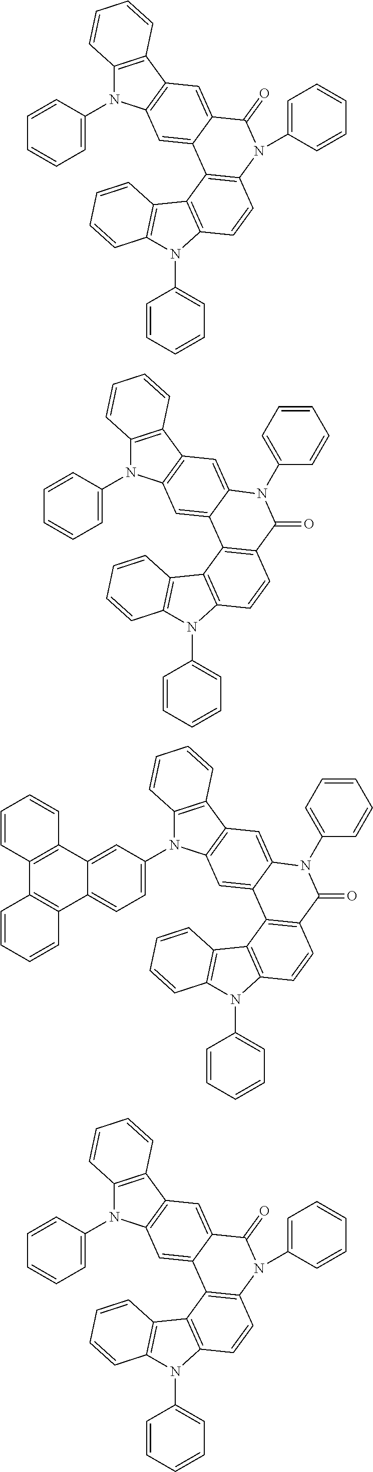

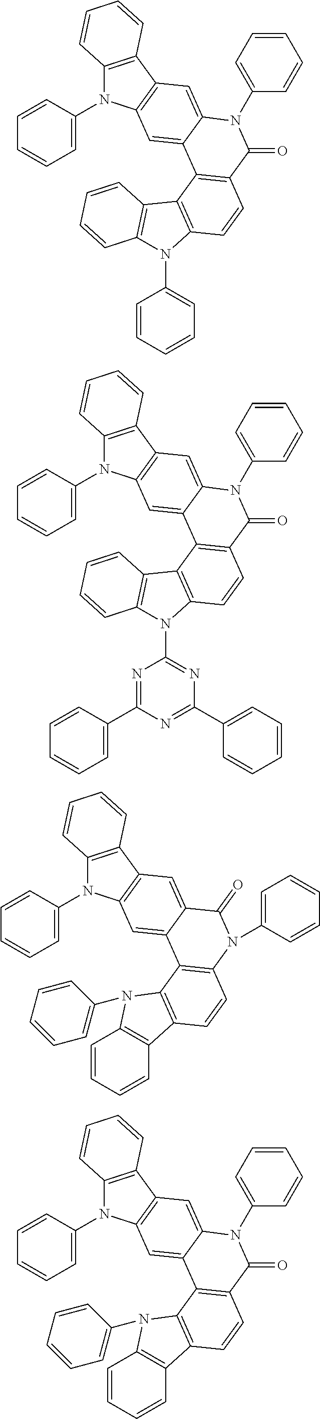

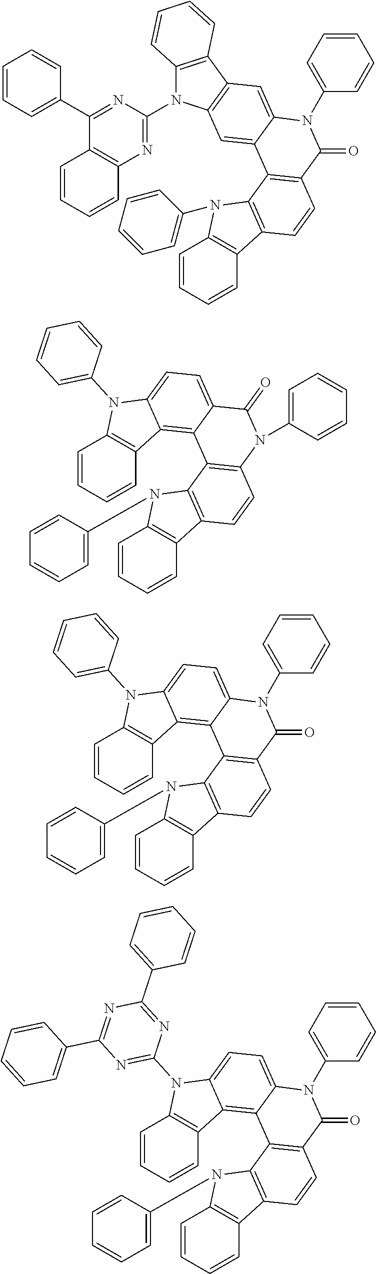

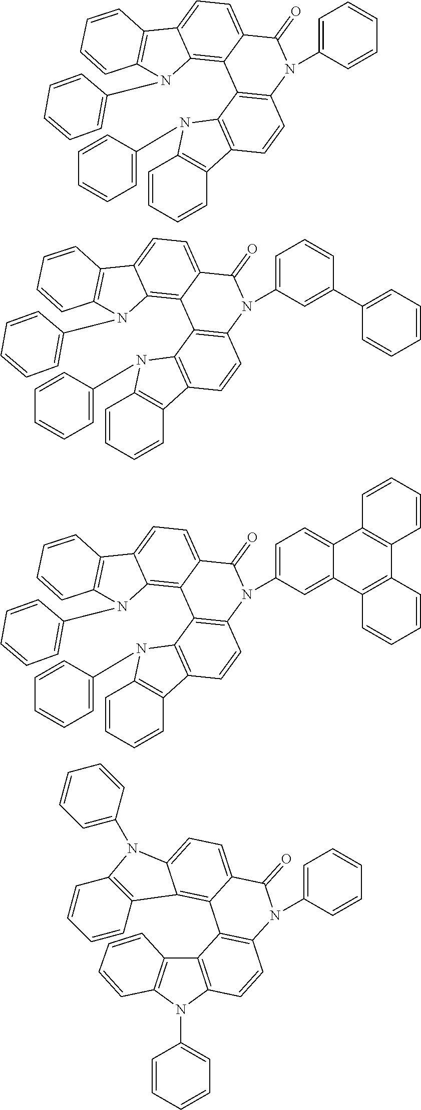

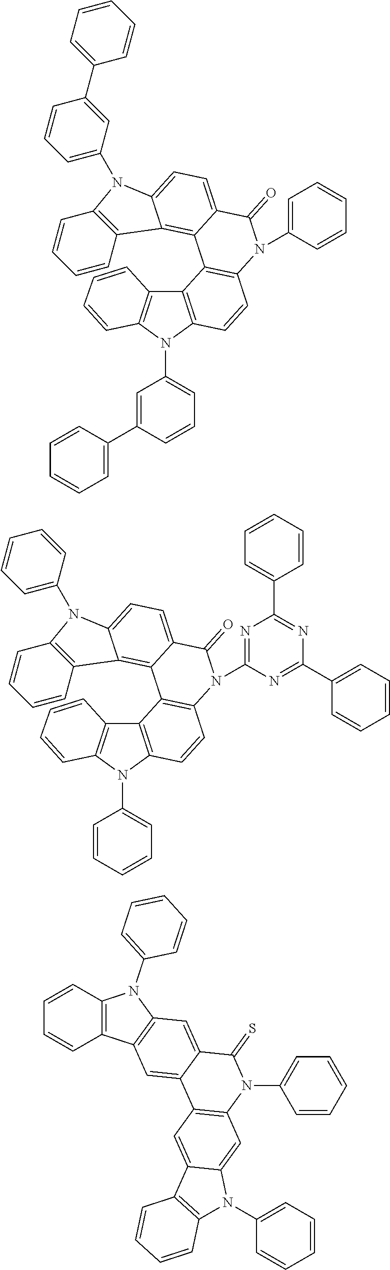

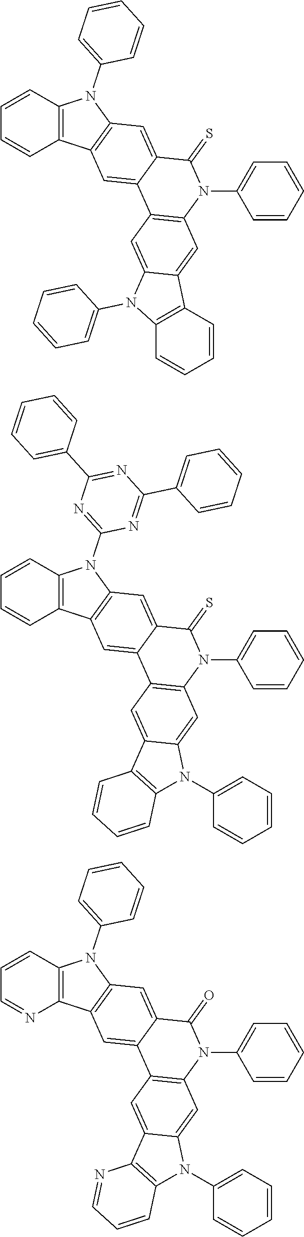

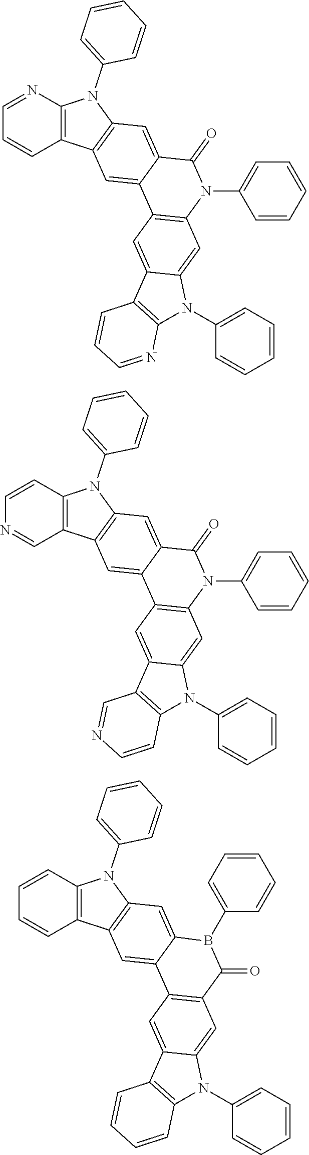

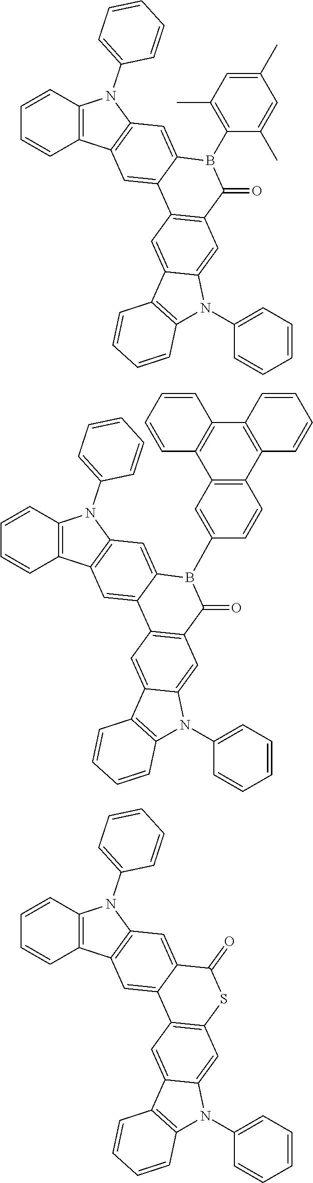

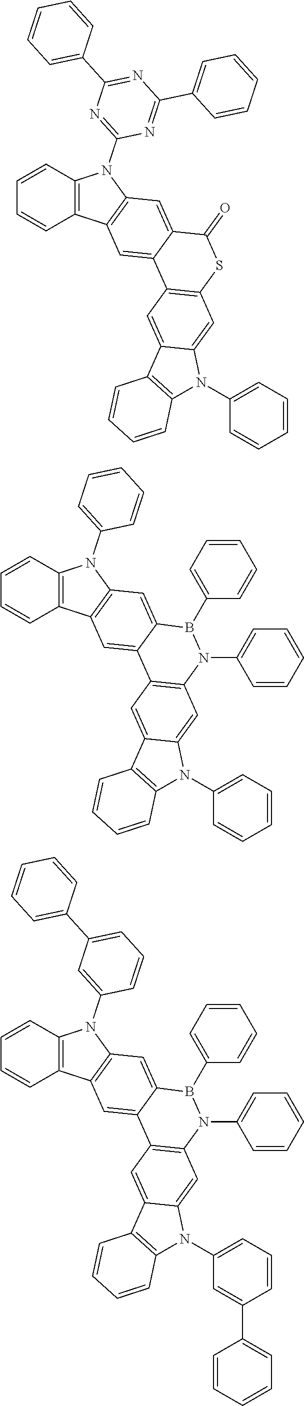

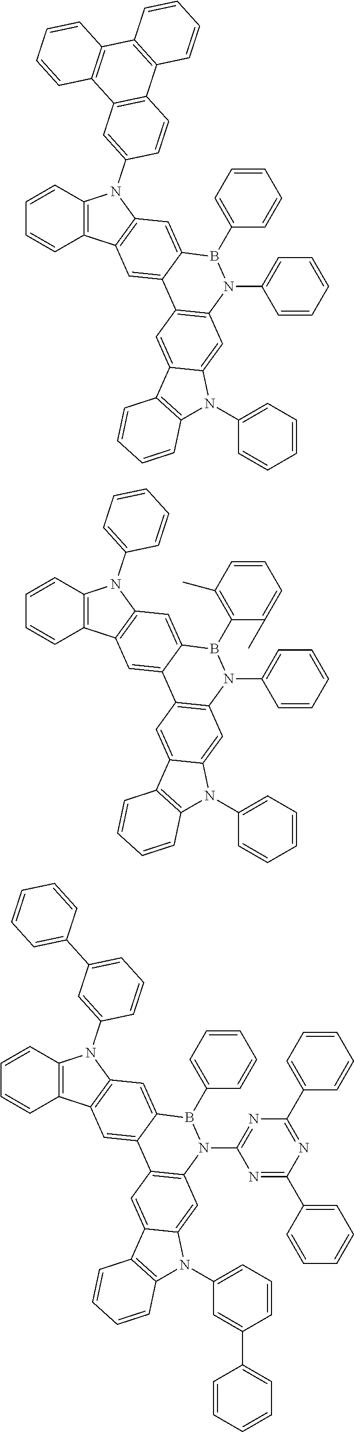

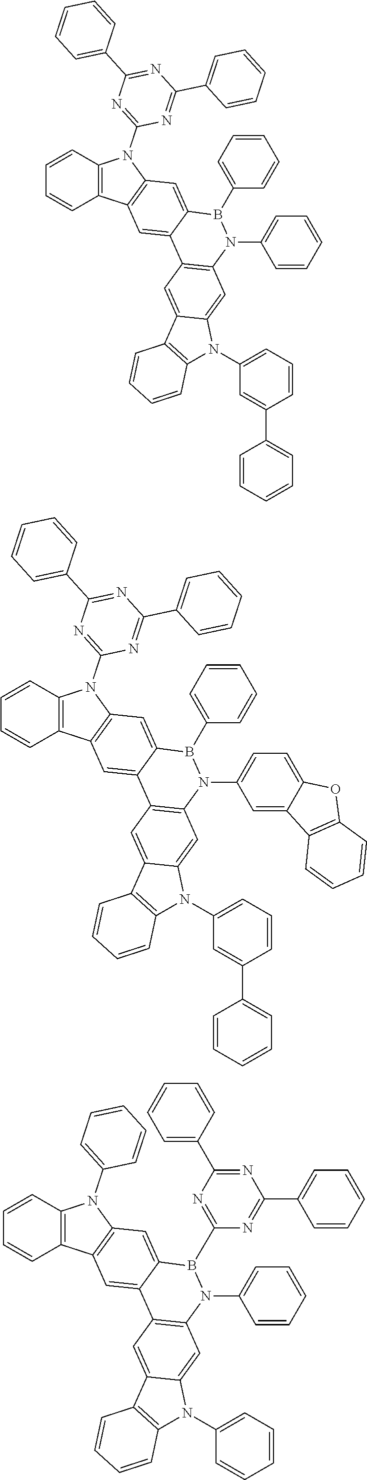

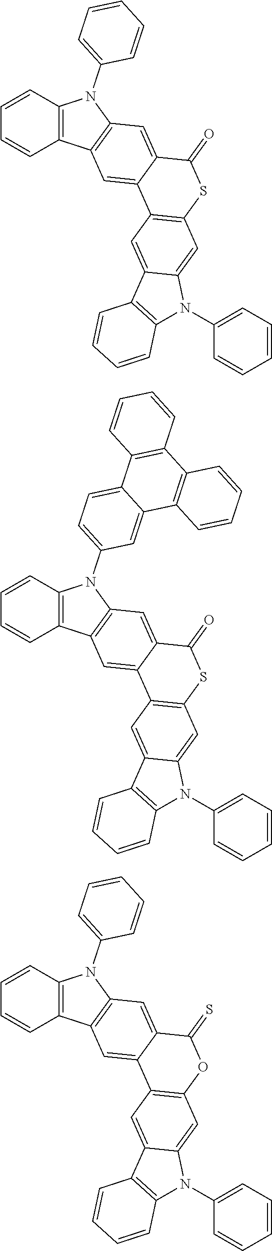

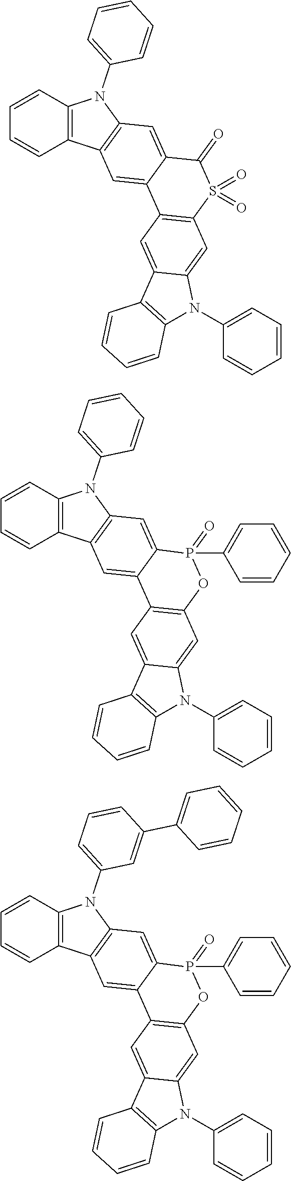

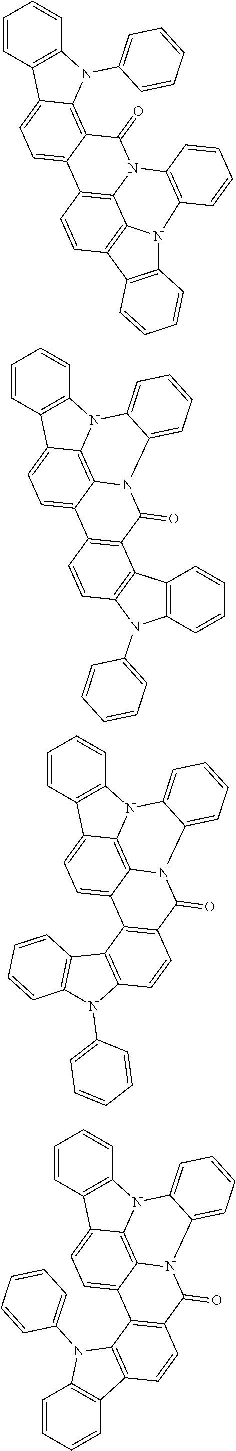

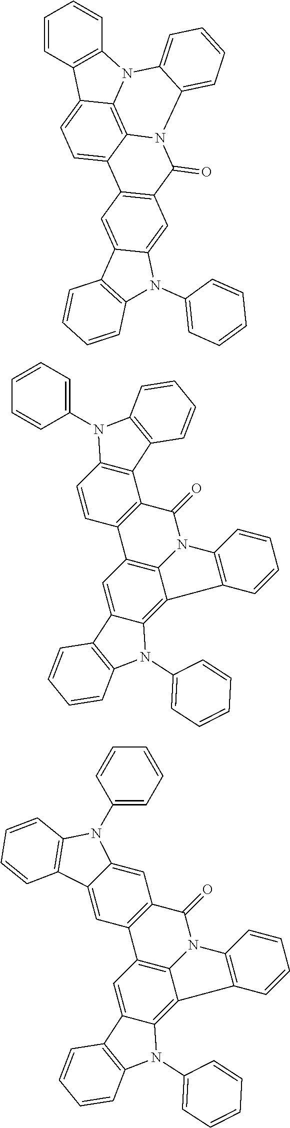

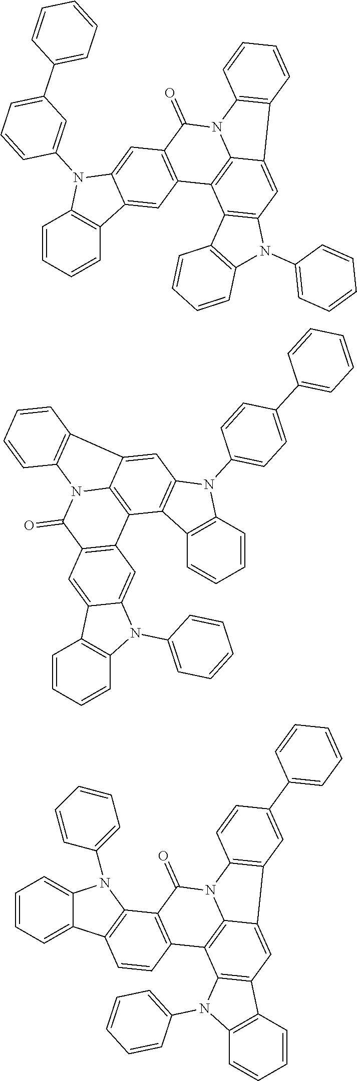

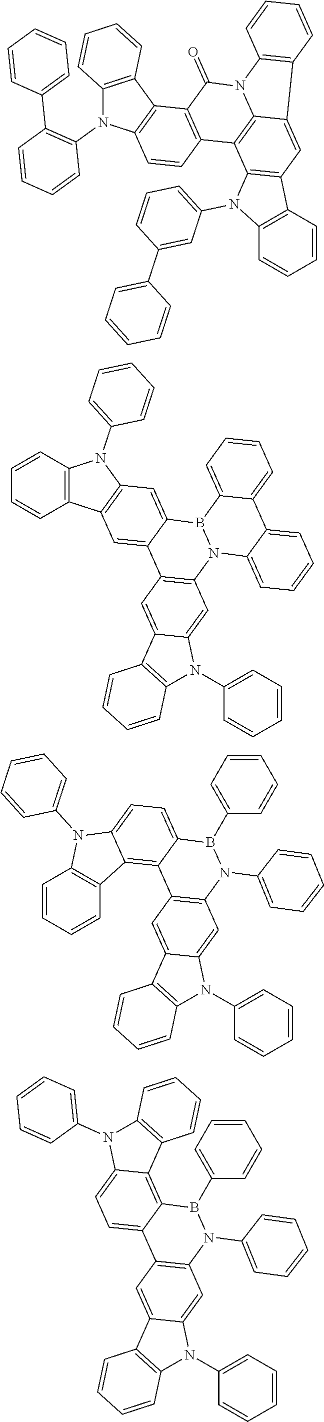

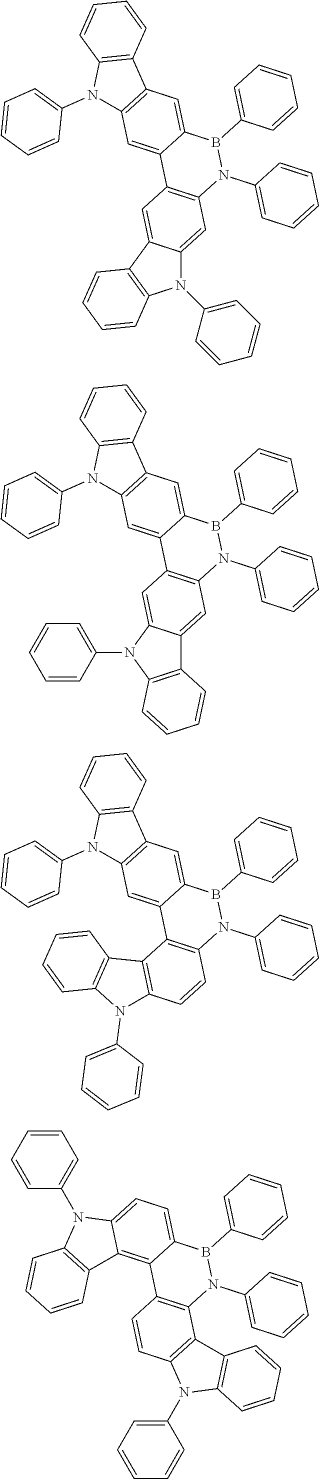

The base structure of the compounds of the invention can be prepared and functionalized by one of the routes outlined in schemes 1, 2 and 3 that follow. In this case, as shown in schemes 1 and 2, proceeding from a bifunctional ketone, the biscarbazole base skeleton can be prepared via a Buchwald coupling and subsequent ring closure reaction with CH activation (scheme 1) or via a Suzuki coupling and subsequent ring closure reaction via a Cadogan reaction, and then functionalized (for example via Buchwald or Ullmann coupling or nucleophilic substitution). Subsequently, the corresponding oxime is first prepared and then the central ring is extended via a rearrangement reaction (Beckmann rearrangement) to give the respective lactam. Subsequently, the central lactam nitrogen (or thiolactam nitrogen) can be functionalized further (for example via Buchwald or Ullmann coupling).

An alternative synthesis route is shown in scheme 3; here, proceeding from the central ring system, the two carbazole units may be fused on via a Buchwald coupling and subsequent ring closure reaction with CH activation and then functionalized (for example via Buchwald or Ullmann coupling or nucleophilic substitution).

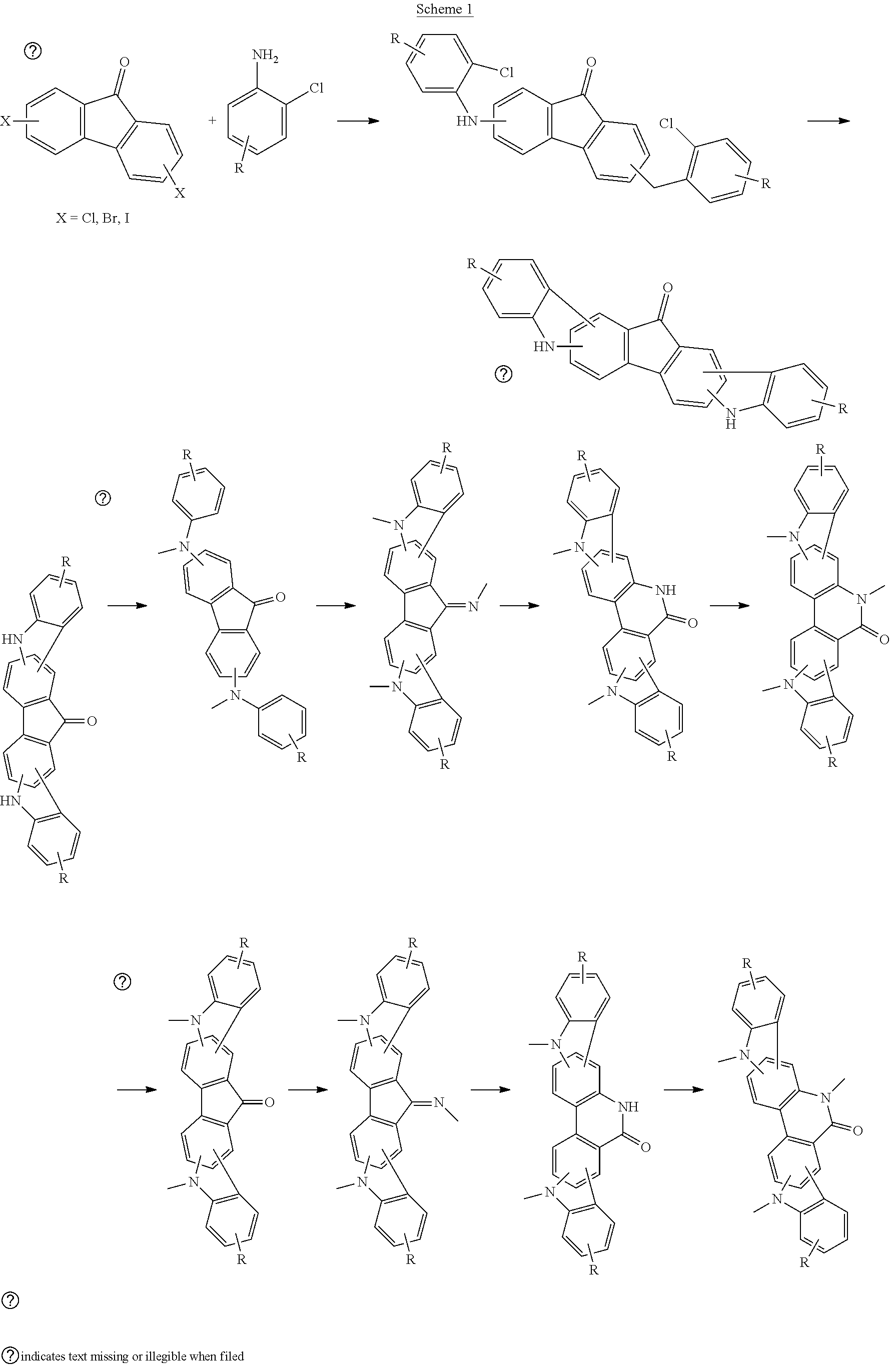

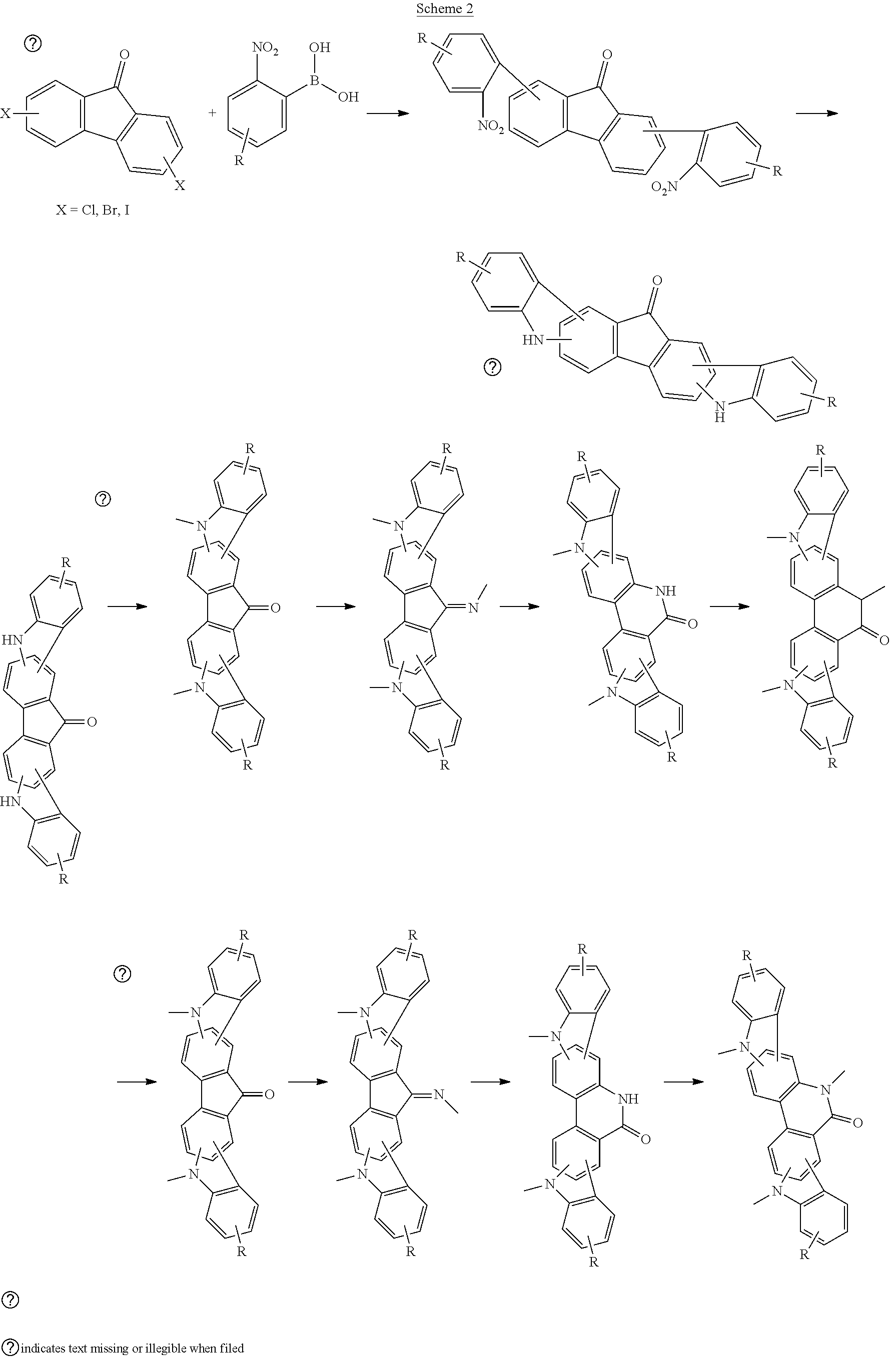

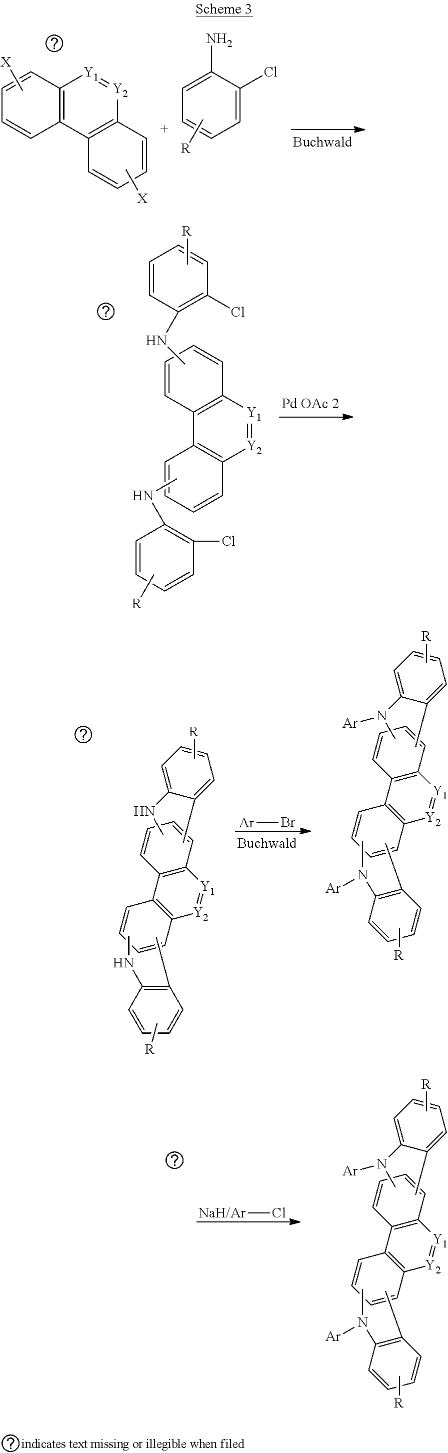

The present invention therefore further provides a process for preparing the compounds of the invention, characterized by the following steps:
(a) synthesizing a biscarbazole base skeleton proceeding from a functionalized fluorenone or the functionalized central ring system via coupling and ring closure reactions;
(b) introducing the substituent Ar¹ by a coupling reaction or nucleophilic substitution;
(c) in the case of the fluorenone precursor, forming the central ring containing Y¹ and Y² via a rearrangement reaction;
(d) introducing the substituent R^{a} for the sequence (a), (b) and (c) by a coupling reaction or nucleophilic substitution.

For the processing of the compounds of formula (1) or the preferred embodiments from the liquid phase, for example by spin-coating or by printing methods, formulations of the compounds of the invention are required. The present invention therefore further provides formulations comprising at least one compound of formula (1) or the preferred embodiments and at least one solvent. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The compounds of the formula (1) or the above-detailed preferred embodiments are used in accordance with the invention in an electronic device, especially in an organic electroluminescent device. The present invention therefore further provides for the use of the compounds of formula (1) or the preferred embodiments in an electronic device, especially in an OLED.

The present invention still further provides an electronic device, especially an organic electroluminescent device, comprising at least one compound of the invention. An electronic device in the context of the present invention is a device comprising at least one layer comprising at least one organic compound. This component may also comprise inorganic materials or else layers formed entirely from inorganic materials.

The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs), more preferably phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem OLED, especially for white-emitting OLEDs.

The compound according to the above-detailed embodiments may be used in different layers, according to the exact structure. Preference is given to an organic electroluminescent device comprising a compound of formula (1) or the above-recited preferred embodiments in an emitting layer as matrix material for phosphorescent or fluorescent emitters or for emitters that exhibit TADF (thermally activated delayed fluorescence), especially as matrix material for phosphorescent emitters. In this case, the organic electroluminescent device may contain an emitting layer, or it may contain a plurality of emitting layers, where at least one emitting layer contains at least one compound of the invention as matrix material. In addition, the compound of the invention may also be used in an electron transport layer and/or in a hole blocker layer.

When the compound is used as matrix material for a phosphorescent compound in an emitting layer, it is preferably used in combination with one or more phosphorescent materials (triplet emitters).

Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the formula (1) or of the preferred embodiments and the emitting compound contains between 99% and 1% by volume, preferably between 98% and 10% by volume, more preferably between 97% and 60% by volume and especially between 95% and 80% by volume of the compound of the formula (1) or of the preferred embodiments, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

A further preferred embodiment of the present invention is the use of the compound of the formula (1) or of the preferred embodiments as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or those in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, or dibenzofuran derivatives, for example according to WO 2015/169412, WO 2016/015810, WO 2016/023608, WO 2017/148564 or WO 2017/148565. It is likewise possible for a further phosphorescent emitter having shorter-wavelength emission than the actual emitter to be present as co-host in the mixture, or a compound not involved in charge transport to a significant extent, if at all, as described, for example, in WO 2010/108579.

In a preferred embodiment of the invention, the materials are used in combination with a further matrix material. Some of the compounds of the formula (1) or the preferred embodiments are electron-rich compounds. This is particularly true of compounds that bear an electron-rich aromatic or heteroaromatic ring system as Ar¹ and/or R^{a} radicals. Preferred co-matrix materials are therefore electron-transporting compounds that are preferably selected from the group of the triazines, pyrimidines, quinazolines, quinoxalines and lactams, or derivatives of these structures.

Preferred triazine, pyrimidine, quinazoline or quinoxaline derivatives that can be used as a mixture together with the compounds of the invention are the compounds of the following formulae (3), (4), (5) and (6):

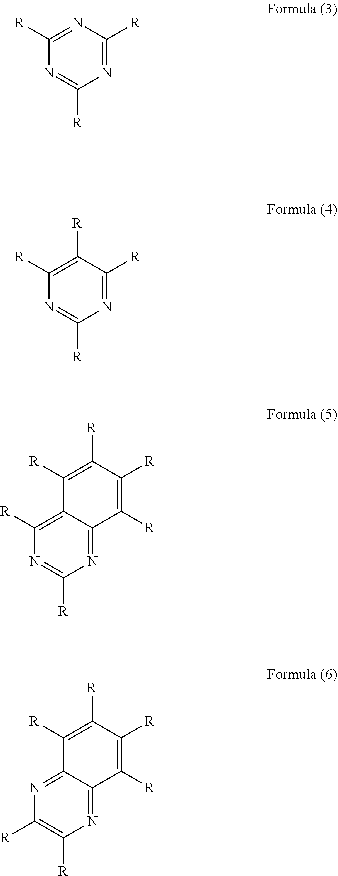

where R has the meanings given above. R is preferably the same or different at each instance and is H, D or an aromatic or heteroaromatic ring system which has 6 to 30 aromatic ring atoms and which may be substituted by one or more R¹ radicals.

Preference is given to the compounds of the following formulae (3a) to (6a):

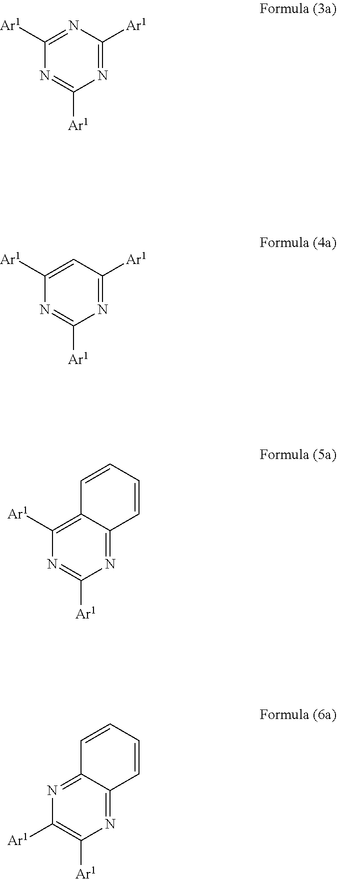

where the symbols used have the definitions given above.

Particular preference is given to the triazine derivatives of the formula (3) or (3a) and the quinazoline derivatives of the formula (6) or (6a), especially the triazine derivatives of the formula (3) or (3a).

In a preferred embodiment of the invention, Ar¹ in the formulae (3a), (4a), (5a) and (6a) is the same or different at each instance and is an aromatic or heteroaromatic ring system which has 6 to 30 aromatic ring atoms, especially 6 to 24 aromatic ring atoms, and may be substituted by one or more R radicals. Suitable aromatic or heteroaromatic ring systems Ar¹ here are the same as set out above as embodiments for Ar¹, especially the structures Ar-1 to Ar-83.

Examples of suitable triazine and pyrimidine compounds that may be used as matrix materials together with the compounds of the invention are the compounds depicted in the following table:

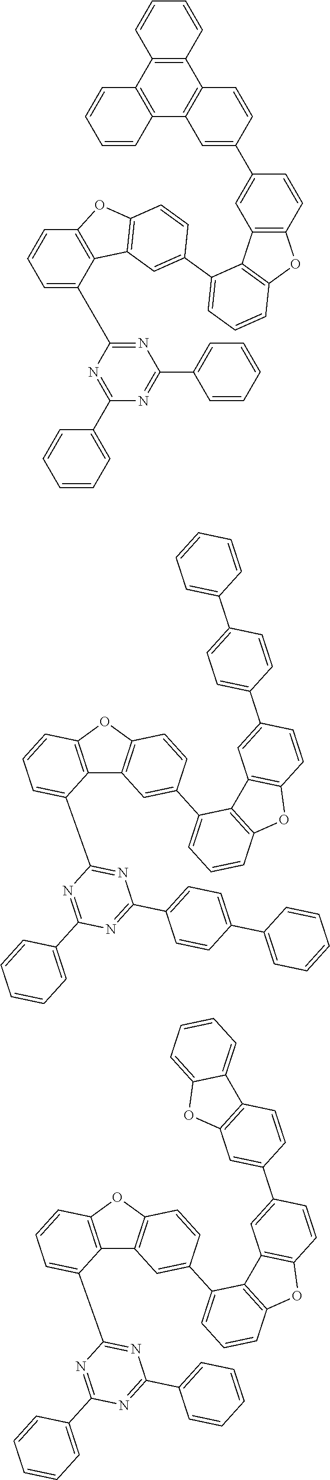 [SMILES: c1ccc(-c2nc(-c3ccccc3)nc(-c3cccc4oc5ccc(-c6cccc7oc8ccc(-c9ccc%10c%11ccccc%11c%11ccccc%11c%10c9)cc8c67)cc5c34)n2)cc1 ; c1ccc(-c2ccc(-c3ccc4oc5cccc(-c6ccc7oc8cccc(-c9nc(-c%10ccccc%10)nc(-c%10ccc(-c%11ccccc%11)cc%10)n9)c8c7c6)c5c4c3)cc2)cc1 ; c1ccc(-c2nc(-c3ccccc3)nc(-c3cccc4oc5ccc(-c6cccc7oc8ccc(-c9ccc%10c(c9)oc9ccccc9%10)cc8c67)cc5c34)n2)cc1]

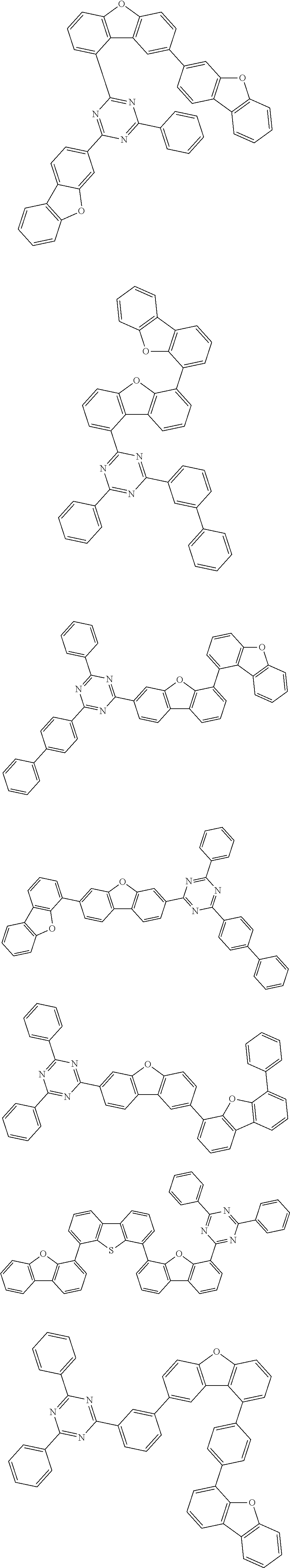

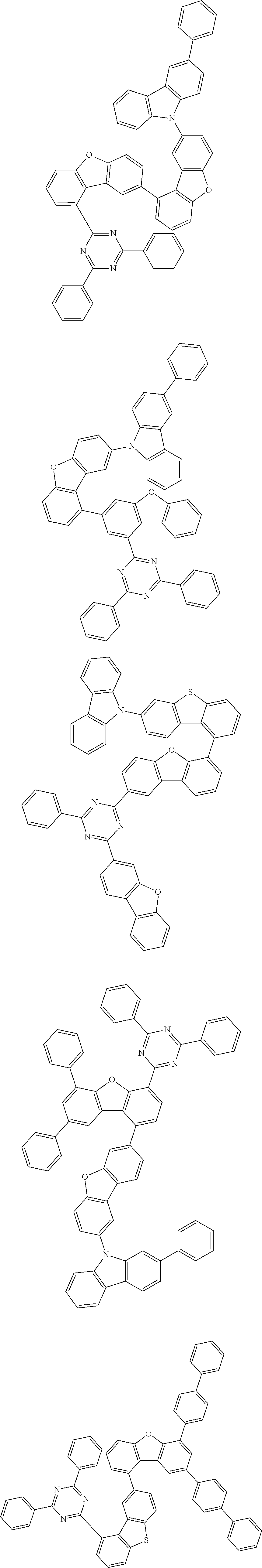

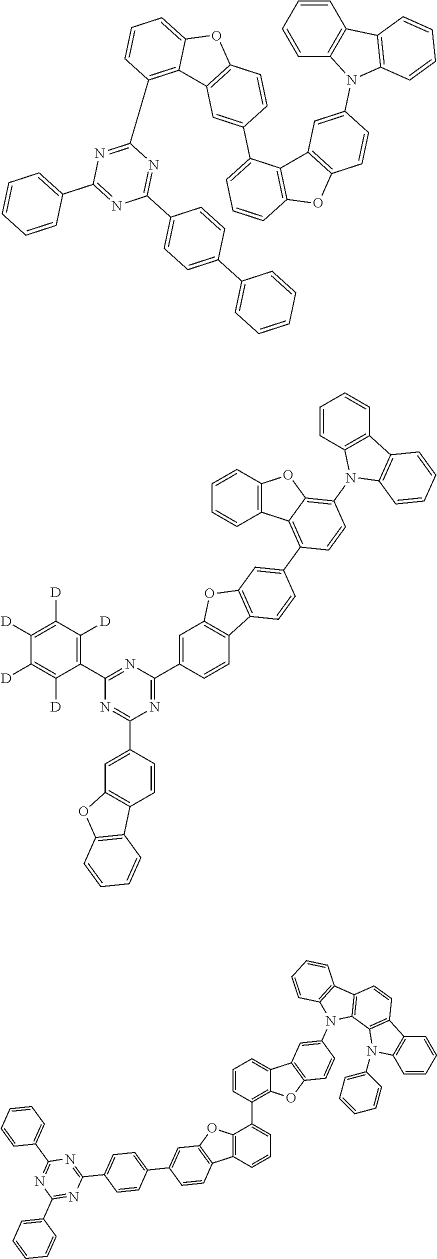 [SMILES: c1ccc(-c2ccc(-c3nc(-c4ccccc4)nc(-c4cccc5oc6ccc(-c7cccc8oc9ccc(-n%10c%11ccccc%11c%11ccccc%11%10)cc9c78)cc6c45)n3)cc2)cc1 ; [2H]c1c([2H])c([2H])c(-c2nc(-c3ccc4c(c3)oc3ccccc34)nc(-c3ccc4c(c3)oc3cc(-c5ccc(-n6c7ccccc7c7ccccc76)c6oc7ccccc7c56)ccc34)n2)c([2H])c1[2H] ; c1ccc(-c2nc(-c3ccccc3)nc(-c3ccc(-c4ccc5c(c4)oc4c(-c6cccc7c6oc6ccc(-n8c9ccccc9c9ccc%10c%11ccccc%11n(-c%11ccccc%11)c%10c98)cc67)cccc45)cc3)n2)cc1]

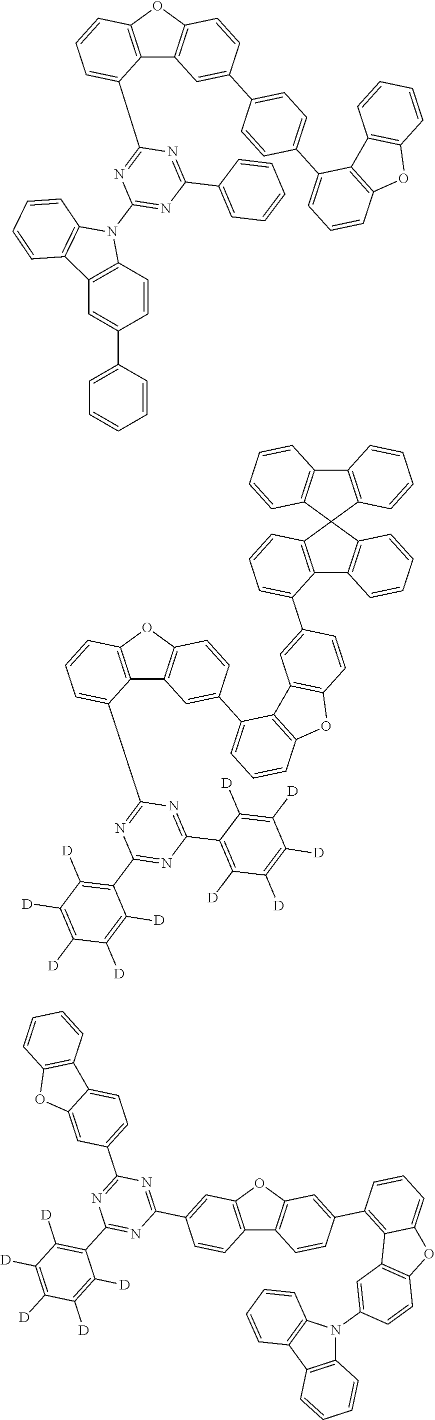 [SMILES: c1ccc(-c2ccc3c(c2)c2ccccc2n3-c2nc(-c3ccccc3)nc(-c3cccc4oc5ccc(-c6ccc(-c7cccc8oc9ccccc9c78)cc6)cc5c34)n2)cc1 ; [2H]c1c([2H])c([2H])c(-c2nc(-c3c([2H])c([2H])c([2H])c([2H])c3[2H])nc(-c3cccc4oc5ccc(-c6cccc7oc8ccc(-c9cccc%10c9-c9ccccc9C%109c%10ccccc%10-c%10ccccc%109)cc8c67)cc5c34)n2)c([2H])c1[2H] ; [2H]c1c([2H])c([2H])c(-c2nc(-c3ccc4c(c3)oc3ccccc34)nc(-c3ccc4c(c3)oc3cc(-c5cccc6oc7ccc(-n8c9ccccc9c9ccccc98)cc7c56)ccc34)n2)c([2H])c1[2H]]

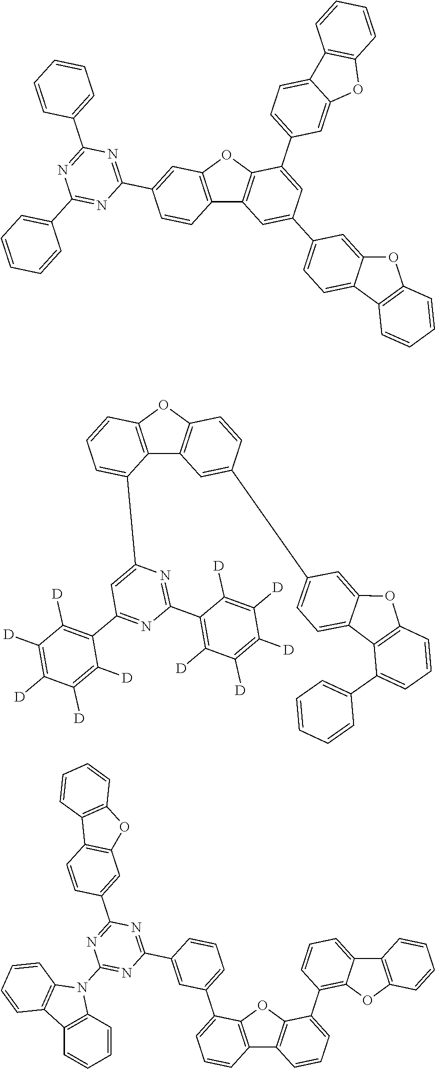 [SMILES: c1ccc(-c2nc(-c3ccccc3)nc(-c3ccc4c(c3)oc3c(-c5ccc6c(c5)oc5ccccc56)cc(-c5ccc6c(c5)oc5ccccc56)cc34)n2)cc1 ; [2H]c1c([2H])c([2H])c(-c2cc(-c3cccc4oc5ccc(-c6ccc7c(c6)oc6cccc(-c8ccccc8)c67)cc5c34)nc(-c3c([2H])c([2H])c([2H])c([2H])c3[2H])n2)c([2H])c1[2H] ; c1cc(-c2nc(-c3ccc4c(c3)oc3ccccc34)nc(-n3c4ccccc4c4ccccc43)n2)cc(-c2cccc3c2oc2c(-c4cccc5c4oc4ccccc45)cccc23)c1]

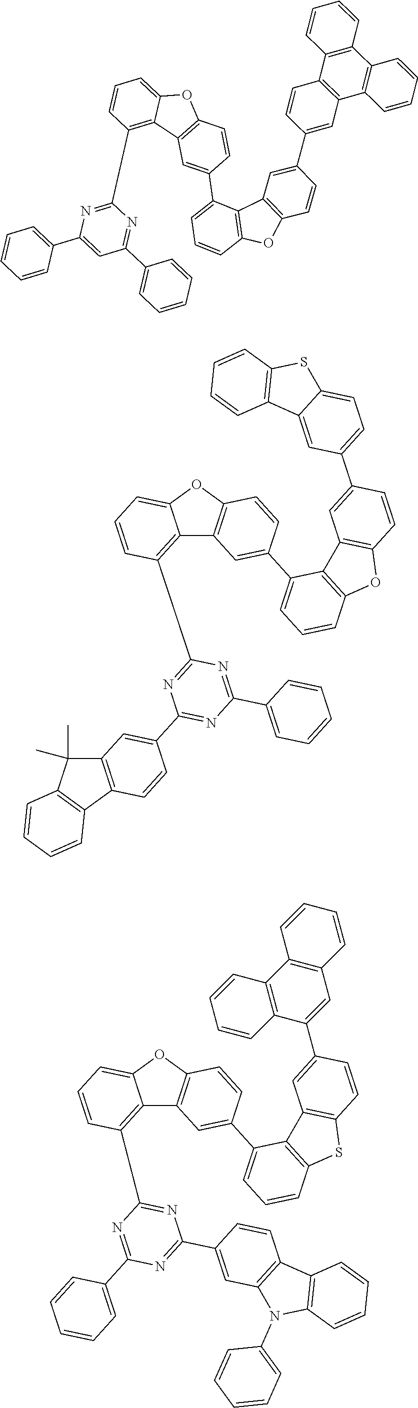 [SMILES: c1ccc(-c2cc(-c3ccccc3)nc(-c3cccc4oc5ccc(-c6cccc7oc8ccc(-c9ccc%10c%11ccccc%11c%11ccccc%11c%10c9)cc8c67)cc5c34)n2)cc1 ; CC1(C)c2ccccc2-c2ccc(-c3nc(-c4ccccc4)nc(-c4cccc5oc6ccc(-c7cccc8oc9ccc(-c%10ccc%11sc%12ccccc%12c%11c%10)cc9c78)cc6c45)n3)cc21 ; c1ccc(-c2nc(-c3ccc4c5ccccc5n(-c5ccccc5)c4c3)nc(-c3cccc4oc5ccc(-c6cccc7sc8ccc(-c9cc%10ccccc%10c%10ccccc9%10)cc8c67)cc5c34)n2)cc1]

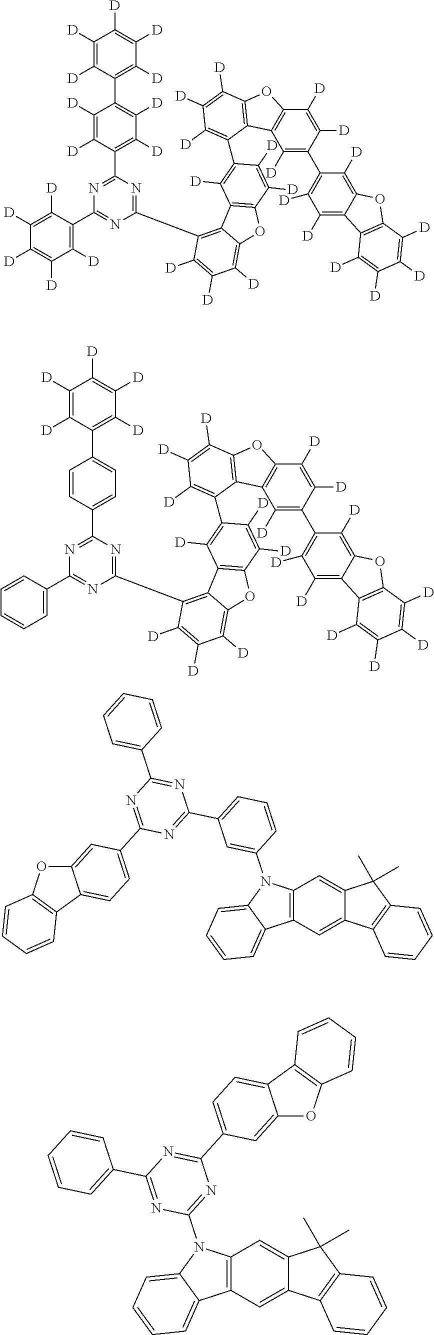

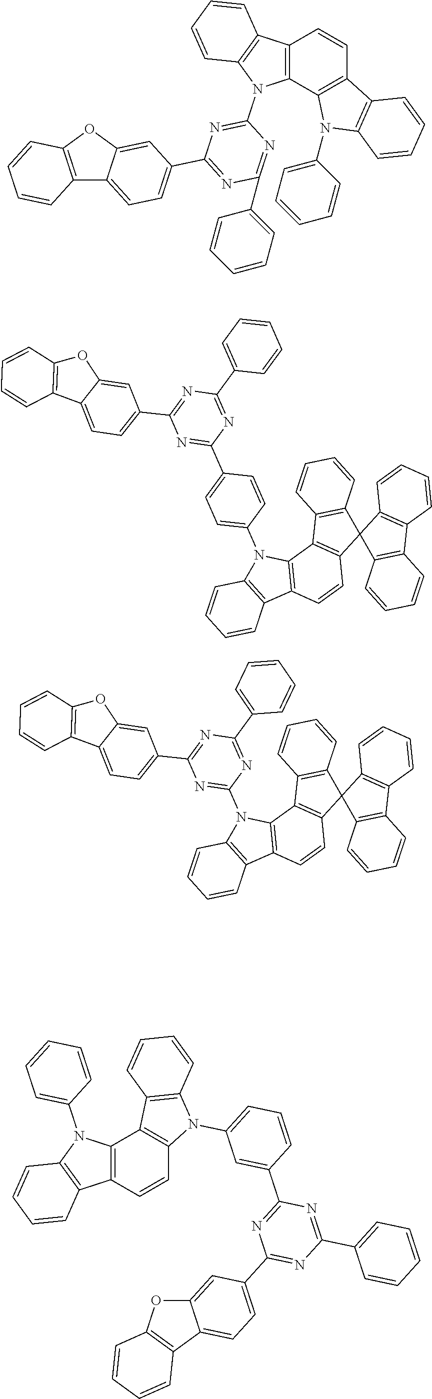

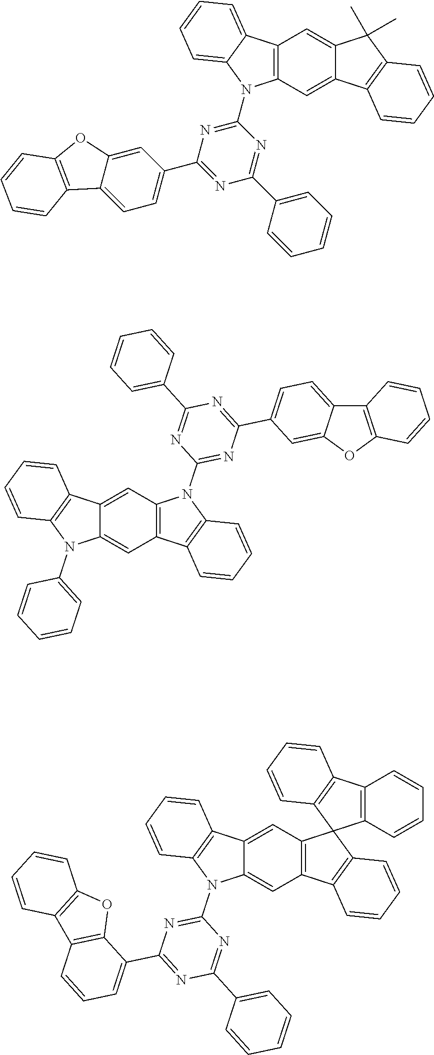 [SMILES: CC1(C)c2ccccc2-c2cc3c(cc21)c1ccccc1n3-c1nc(-c2ccccc2)nc(-c2ccc3c(c2)oc2ccccc23)n1 ; c1ccc(-c2nc(-c3ccc4c(c3)oc3ccccc34)nc(-n3c4ccccc4c4cc5c(cc43)c3ccccc3n5-c3ccccc3)n2)cc1 ; c1ccc(-c2nc(-c3cccc4c3oc3ccccc34)nc(-n3c4ccccc4c4cc5c(cc43)-c3ccccc3C53c4ccccc4-c4ccccc43)n2)cc1]

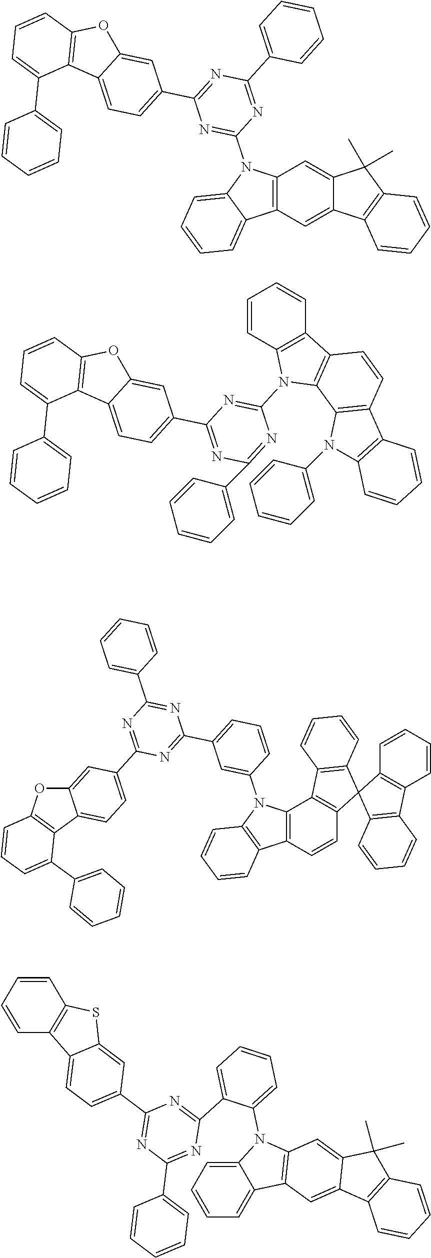 [SMILES: CC1(C)c2ccccc2-c2cc3c4ccccc4n(-c4nc(-c5ccccc5)nc(-c5ccc6c(c5)oc5cccc(-c7ccccc7)c56)n4)c3cc21 ; c1ccc(-c2nc(-c3ccc4c(c3)oc3cccc(-c5ccccc5)c34)nc(-n3c4ccccc4c4ccc5c6ccccc6n(-c6ccccc6)c5c43)n2)cc1 ; c1ccc(-c2nc(-c3cccc(-n4c5ccccc5c5ccc6c(c54)-c4ccccc4C64c5ccccc5-c5ccccc54)c3)nc(-c3ccc4c(c3)oc3cccc(-c5ccccc5)c34)n2)cc1 ; CC1(C)c2ccccc2-c2cc3c4ccccc4n(-c4ccccc4-c4nc(-c5ccccc5)nc(-c5ccc6c(c5)sc5ccccc56)n4)c3cc21]

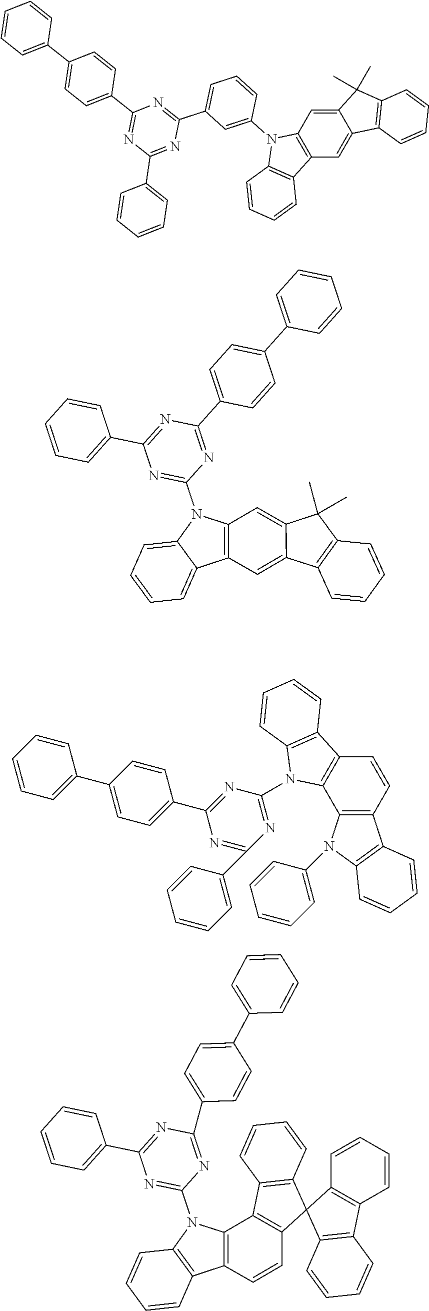

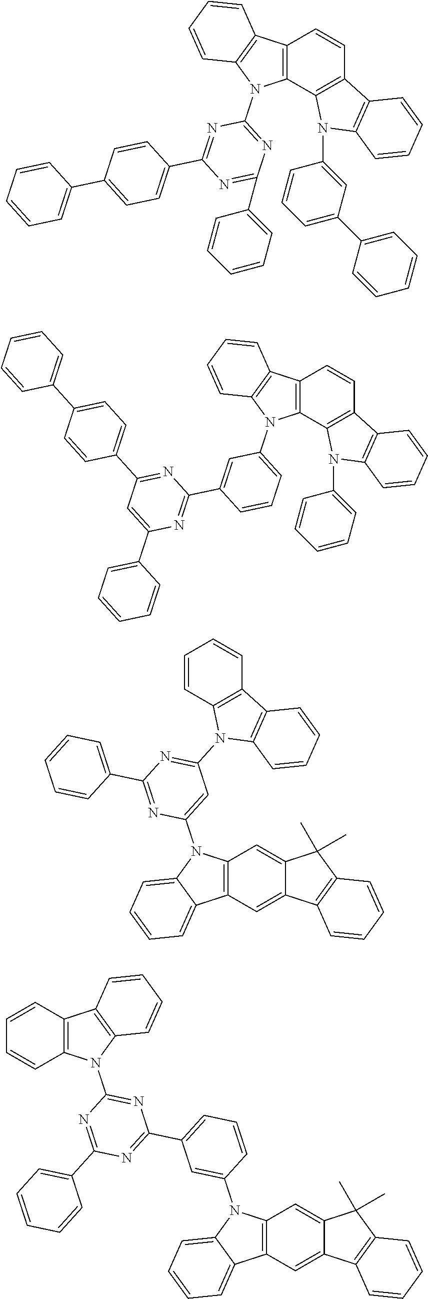

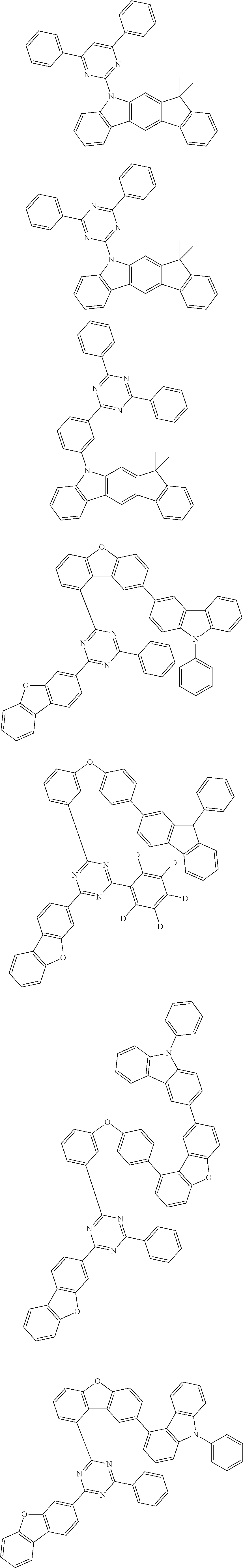

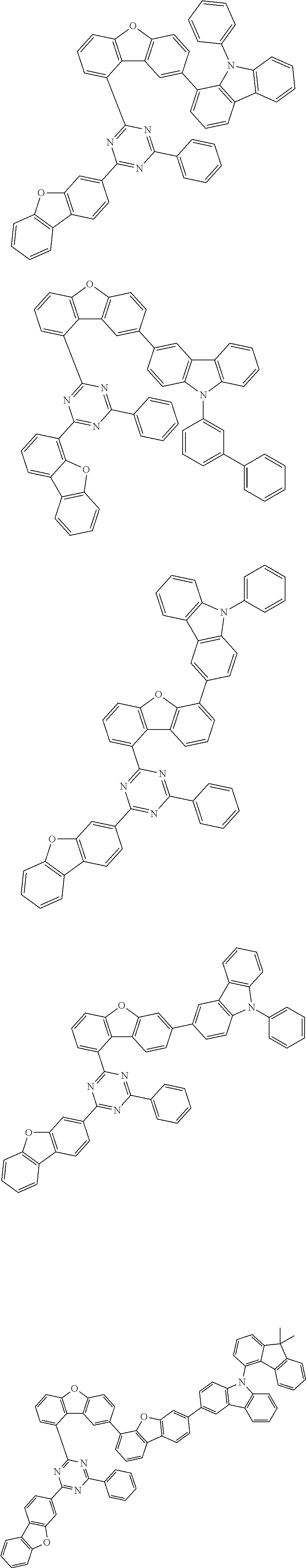

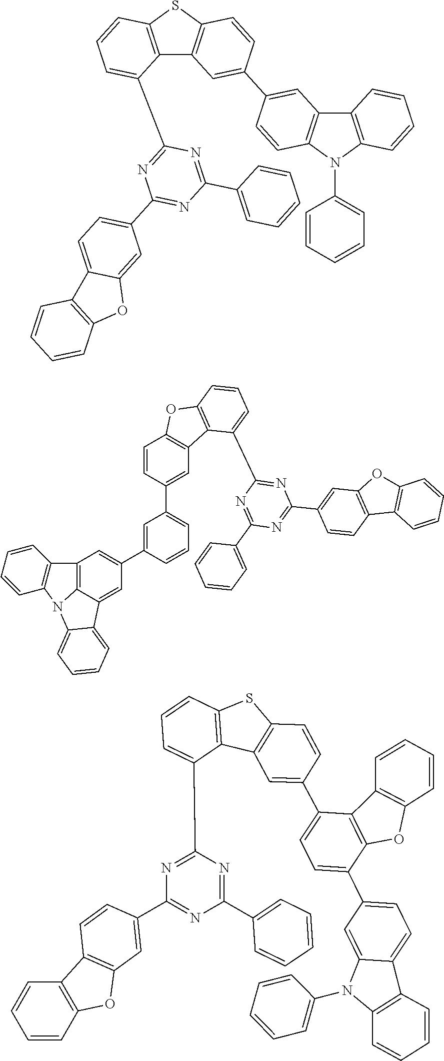 [SMILES: c1ccc(-c2nc(-c3ccc4c(c3)oc3ccccc34)nc(-c3cccc4sc5ccc(-c6ccc7c(c6)c6ccccc6n7-c6ccccc6)cc5c34)n2)cc1 ; c1ccc(-c2nc(-c3ccc4c(c3)oc3ccccc34)nc(-c3cccc4oc5ccc(-c6cccc(-c7cc8c9ccccc9n9c%10ccccc%10c(c7)c89)c6)cc5c34)n2)cc1 ; c1ccc(-c2nc(-c3ccc4c(c3)oc3ccccc34)nc(-c3cccc4sc5ccc(-c6ccc(-c7ccc8c9ccccc9n(-c9ccccc9)c8c7)c7oc8ccccc8c67)cc5c34)n2)cc1]

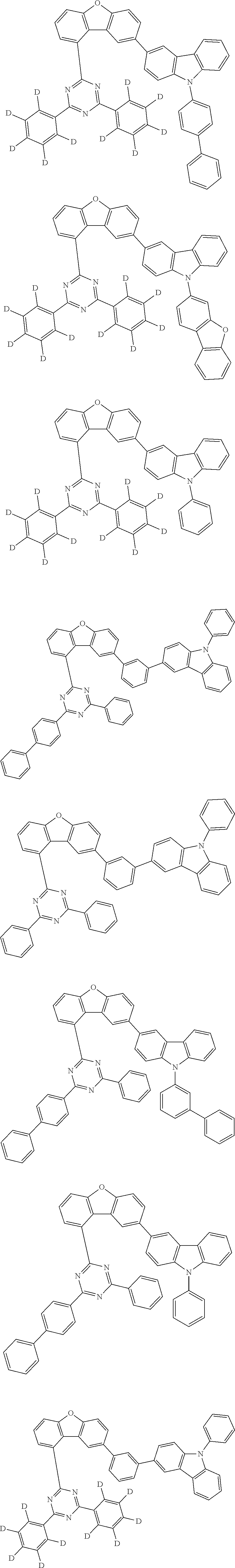

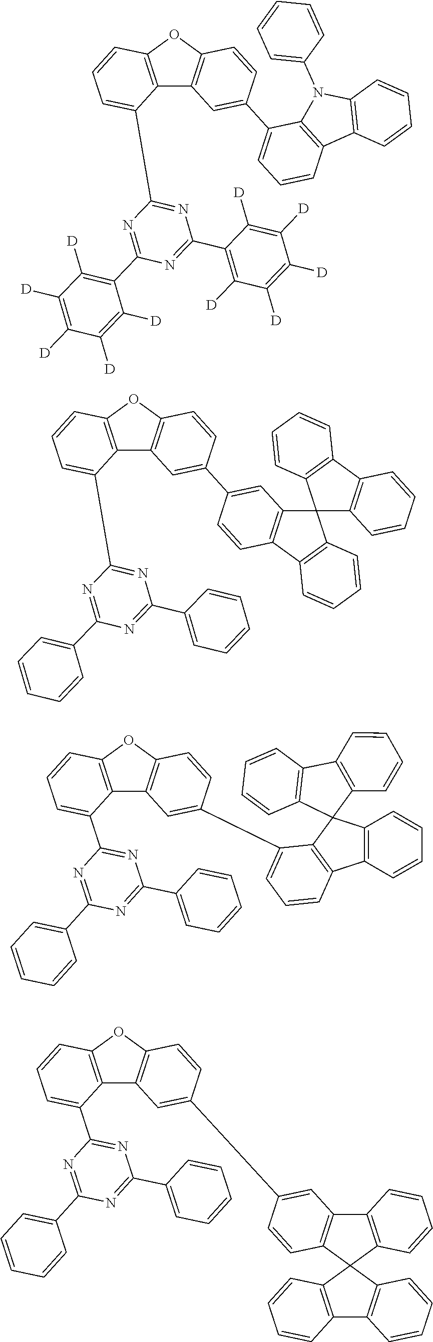

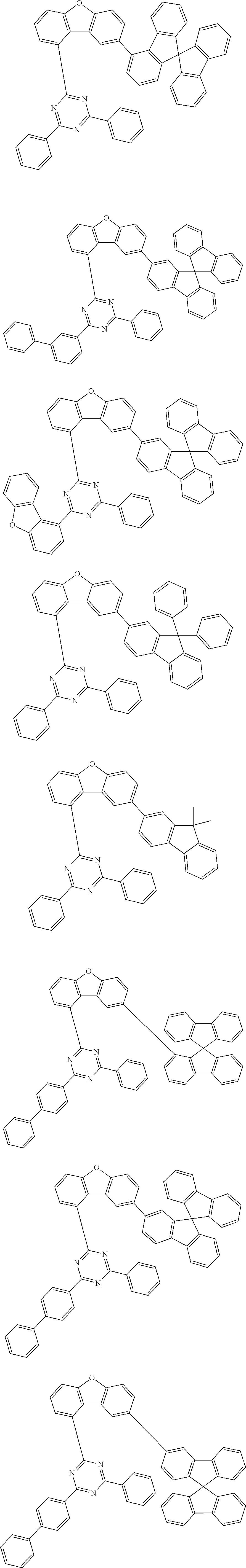

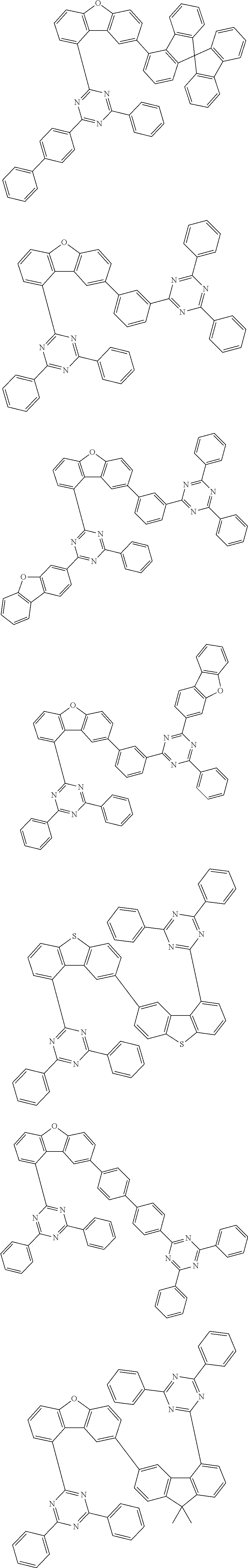

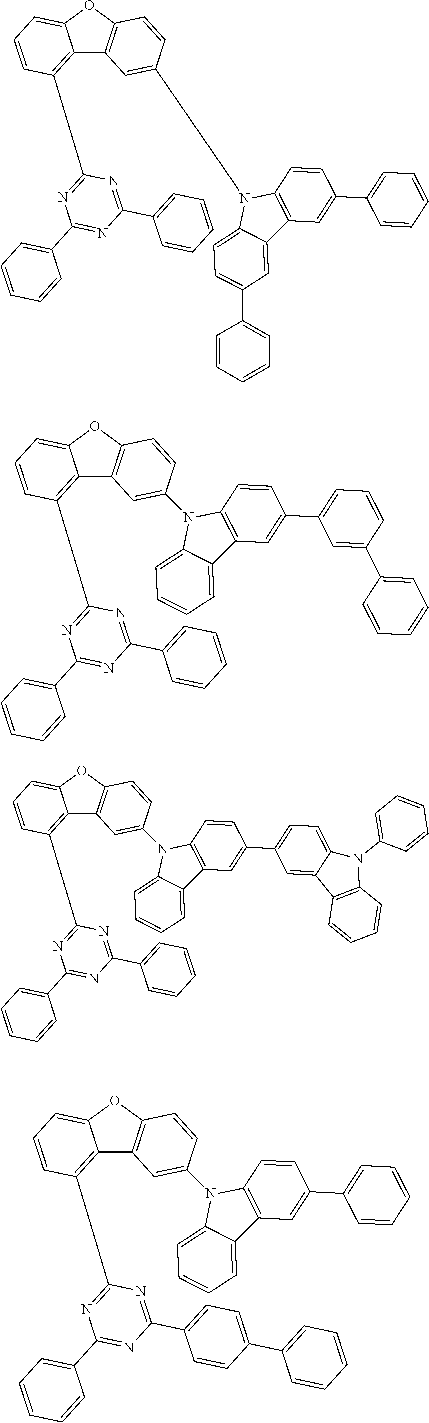

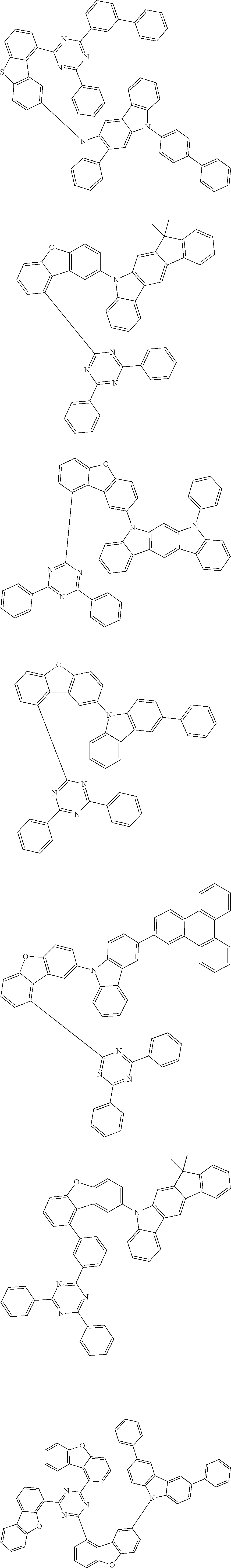

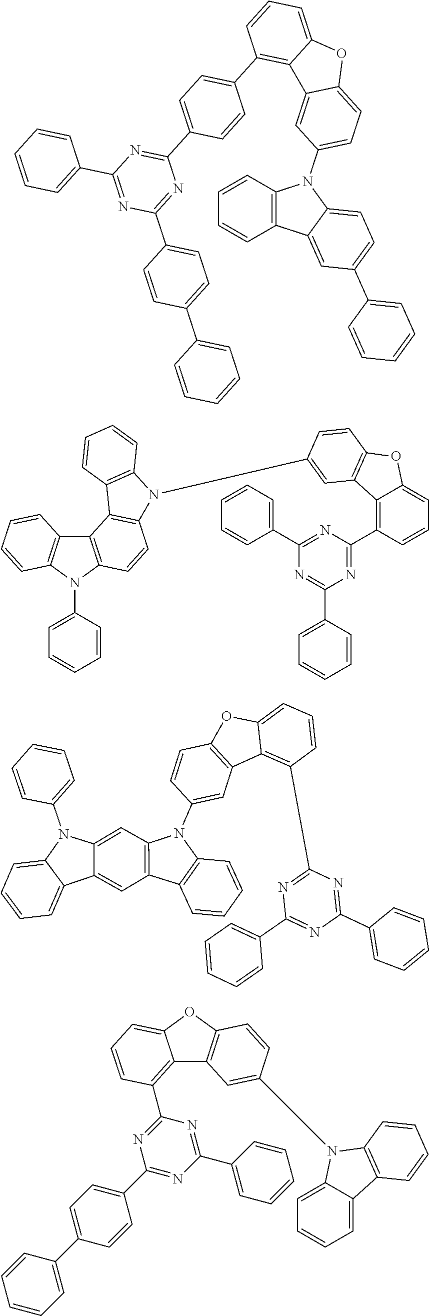

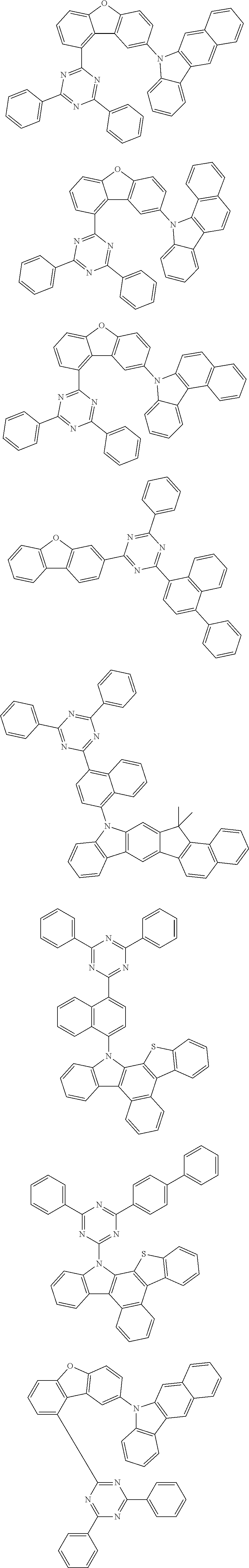

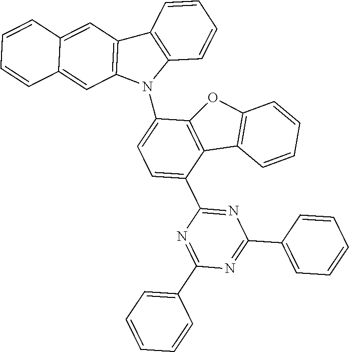 [SMILES: c1ccc(-c2nc(-c3ccccc3)nc(-c3ccc(-n4c5ccccc5c5cc6ccccc6cc54)c4oc5ccccc5c34)n2)cc1]

Examples of suitable quinazoline and quinoxaline derivatives are the structures depicted in the following table;

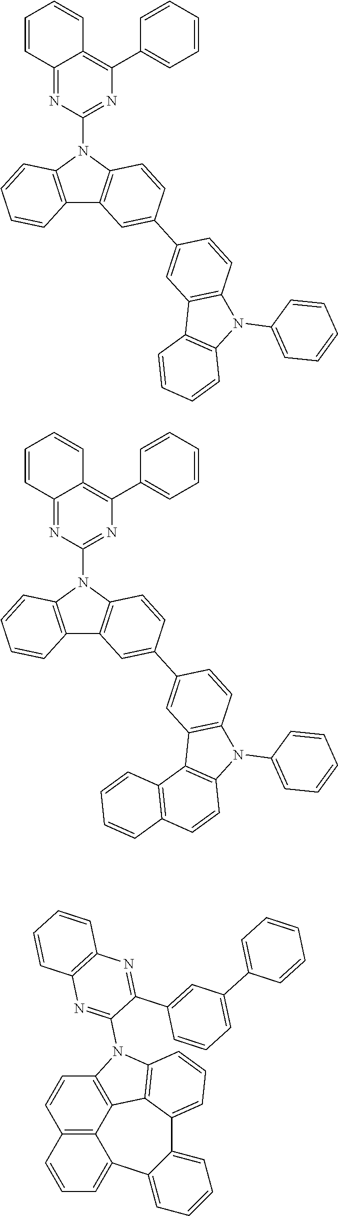

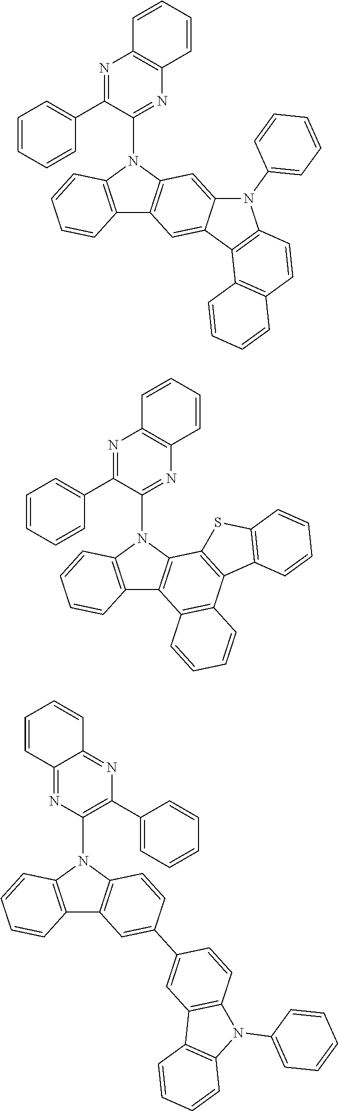 [SMILES: c1ccc(-c2nc3ccccc3nc2-n2c3ccccc3c3cc4c5c6ccccc6ccc5n(-c5ccccc5)c4cc32)cc1 ; c1ccc(-c2nc3ccccc3nc2-n2c3ccccc3c3c4ccccc4c4c5ccccc5sc4c32)cc1 ; c1ccc(-c2nc3ccccc3nc2-n2c3ccccc3c3cc(-c4ccc5c(c4)c4ccccc4n5-c4ccccc4)ccc32)cc1]

Examples of suitable lactams are the structures depicted in the following table:

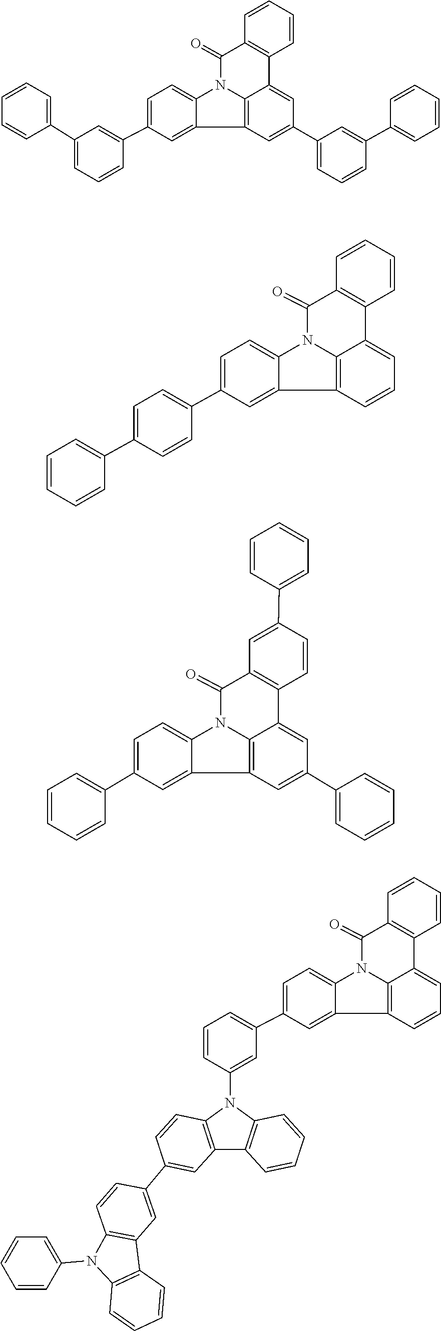 [SMILES: O=c1c2ccccc2c2cc(-c3cccc(-c4ccccc4)c3)cc3c4cc(-c5cccc(-c6ccccc6)c5)ccc4n1c23 ; O=c1c2ccccc2c2cccc3c4cc(-c5ccc(-c6ccccc6)cc5)ccc4n1c23 ; O=c1c2cc(-c3ccccc3)ccc2c2cc(-c3ccccc3)cc3c4cc(-c5ccccc5)ccc4n1c23 ; O=c1c2ccccc2c2cccc3c4cc(-c5cccc(-n6c7ccccc7c7cc(-c8ccc9c(c8)c8ccccc8n9-c8ccccc8)ccc76)c5)ccc4n1c23]

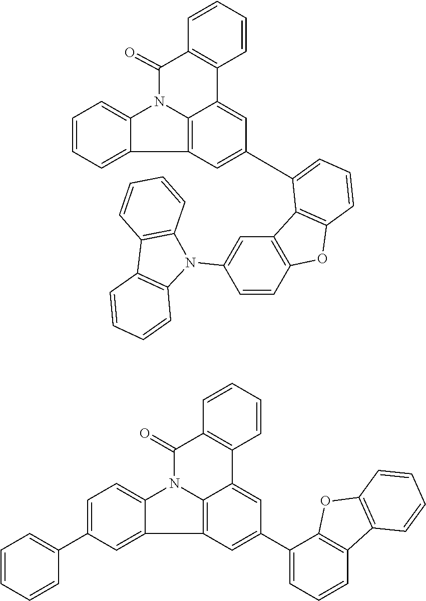 [SMILES: O=c1c2ccccc2c2cc(-c3cccc4oc5ccc(-n6c7ccccc7c7ccccc76)cc5c34)cc3c4ccccc4n1c23 ; O=c1c2ccccc2c2cc(-c3cccc4c3oc3ccccc34)cc3c4cc(-c5ccccc5)ccc4n1c23]

In a further preferred embodiment of the invention, the materials are electron-deficient compounds. This is particularly true of compounds that bear an electron-deficient heteroaromatic ring system as Ar¹ and/or R^{a} radicals. Preferred co-matrix materials are therefore hole-transporting compounds that are preferably selected from the group of the arylamine or carbazole derivatives.

Preferred biscarbazoles are the structures of the following formulae (7) to (13):

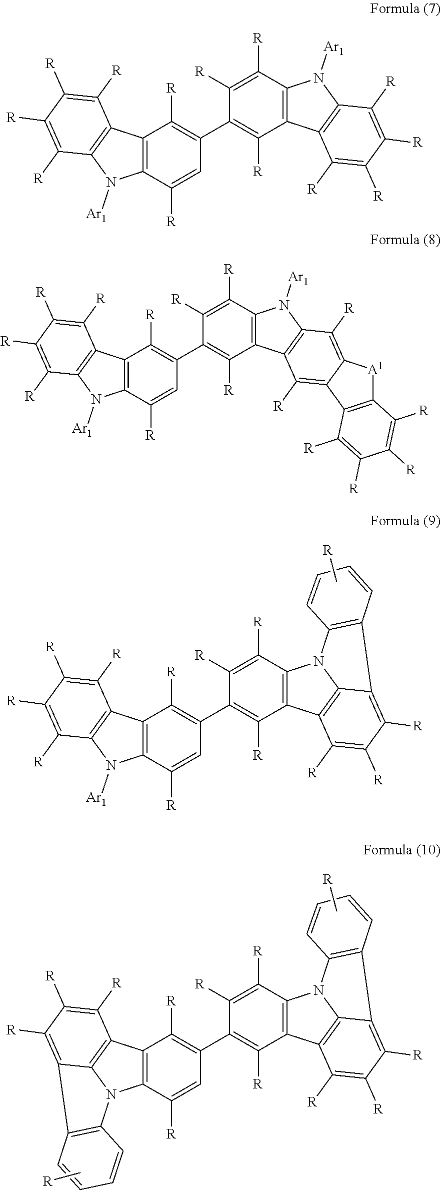

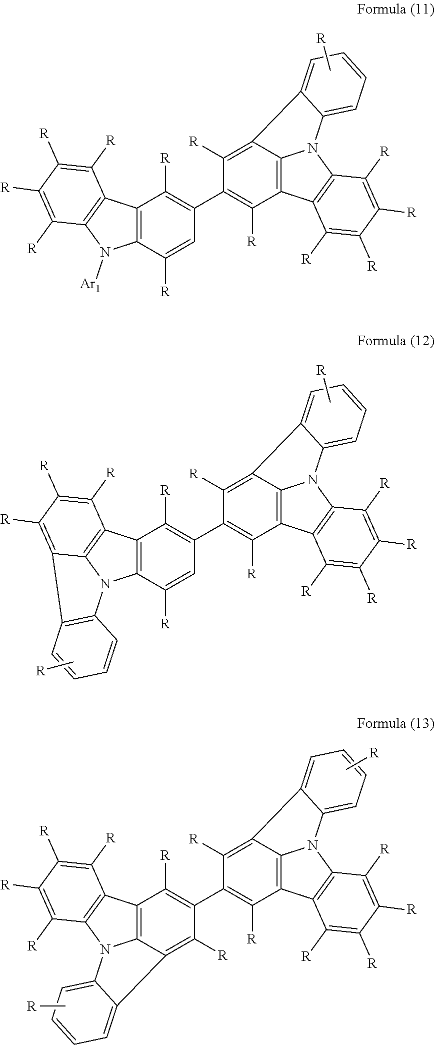

where A¹ has the definitions given above and Ar¹ is the same or different at each instance and is selected from an aromatic or heteroaromatic ring system which has 5 to 40 aromatic ring atoms and may be substituted by one or more R¹ radicals. In a preferred embodiment of the invention, A¹ is CR₂. Preferred embodiments of Ar¹ are the preferred structures detailed above for Ar¹, especially the (Ar-1) to (Ar-83) groups.

Preferred embodiments of the compounds of the formulae (7) to (13) are the compounds of the following formulae (7a) to (13a):

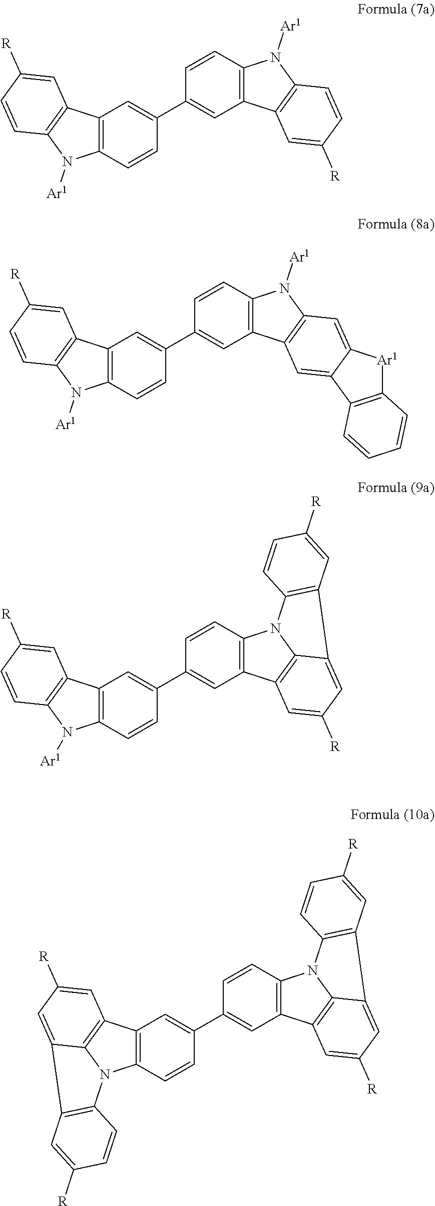

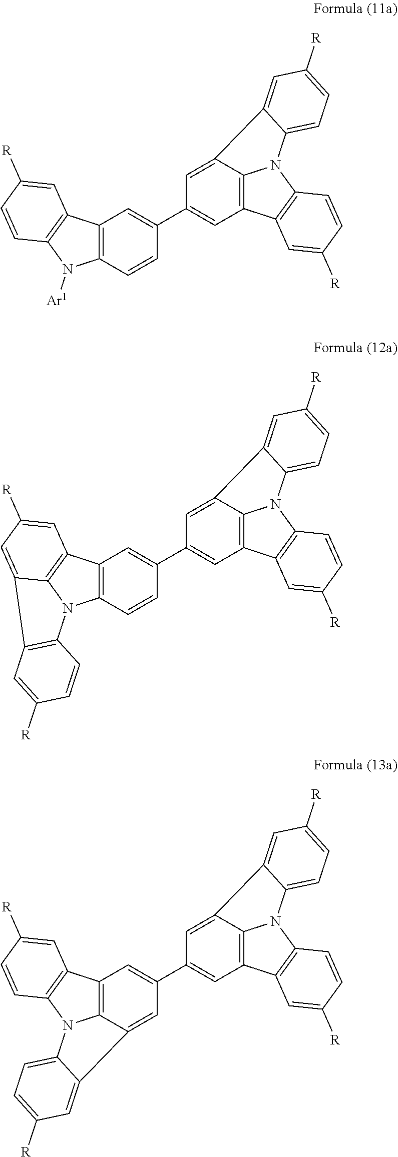

where the symbols used have the definitions given above.

Examples of suitable compounds of formulae (7) to (13) are the compounds depicted below:

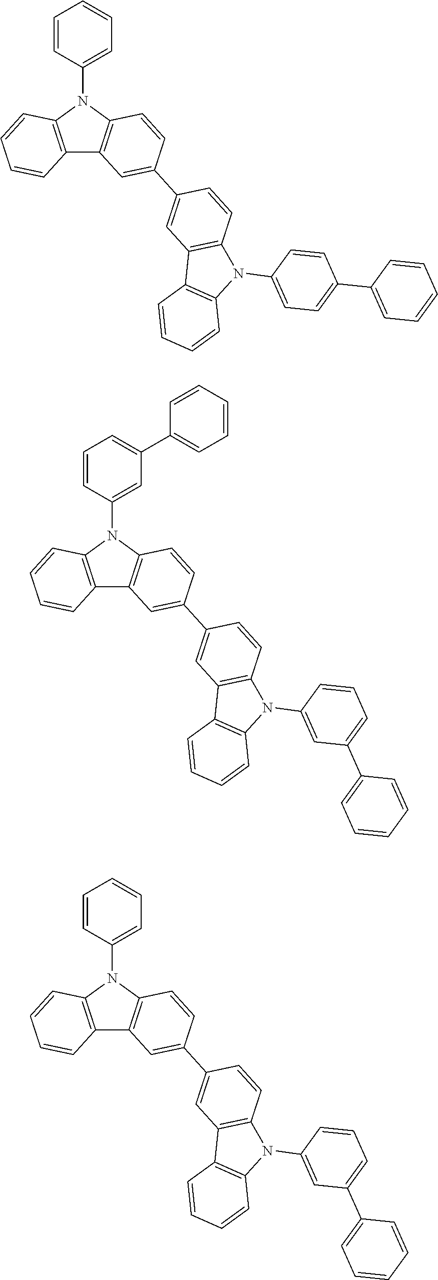

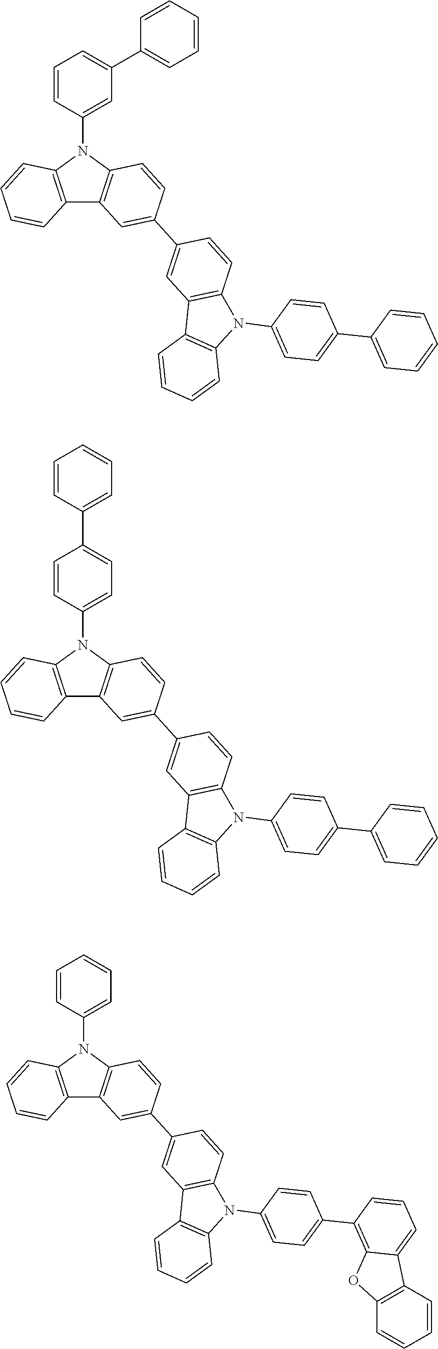 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1]

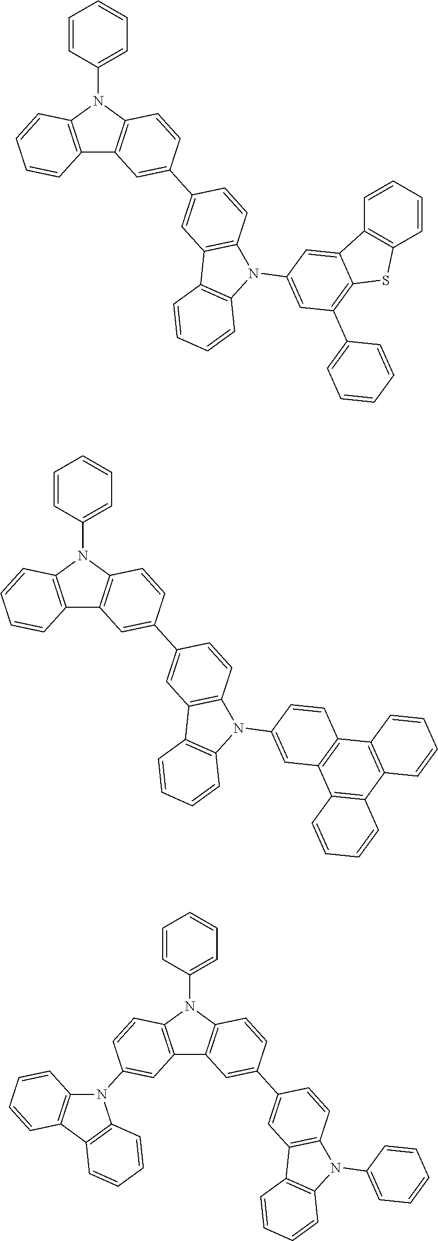 [SMILES: c1ccc(-c2cc(-n3c4ccccc4c4cc(-c5ccc6c(c5)c5ccccc5n6-c5ccccc5)ccc43)cc3c2sc2ccccc23)cc1 ; c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4cc(-n6c7ccccc7c7ccccc76)ccc4n5-c4ccccc4)ccc32)cc1]

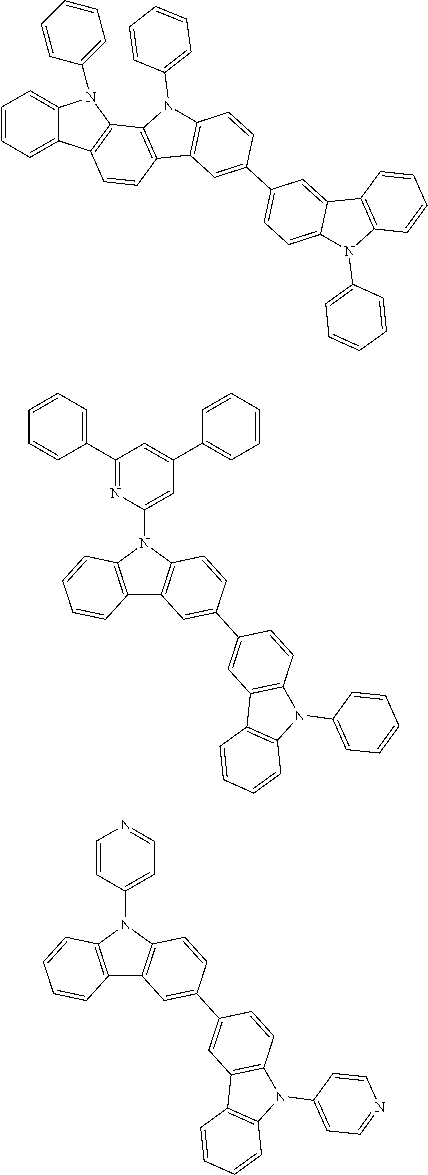 [SMILES: c1ccc(-n2c3ccccc3c3cc(-c4ccc5c(c4)c4ccc6c7ccccc7n(-c7ccccc7)c6c4n5-c4ccccc4)ccc32)cc1 ; c1ccc(-c2cc(-c3ccccc3)nc(-n3c4ccccc4c4cc(-c5ccc6c(c5)c5ccccc5n6-c5ccccc5)ccc43)c2)cc1 ; c1ccc2c(c1)c1cc(-c3ccc4c(c3)c3ccccc3n4-c3ccncc3)ccc1n2-c1ccncc1]

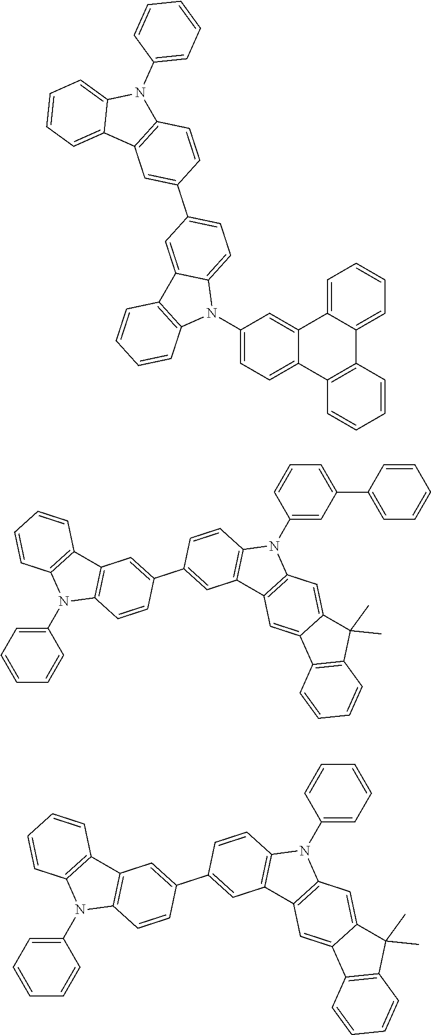

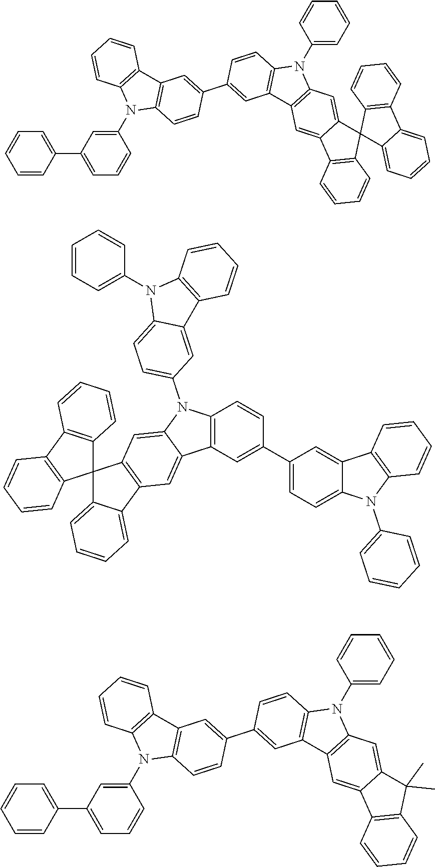 [SMILES: c1ccc(-c2cccc(-n3c4ccccc4c4cc(-c5ccc6c(c5)c5cc7c(cc5n6-c5ccccc5)C5(c6ccccc6-c6ccccc65)c5ccccc5-7)ccc43)c2)cc1 ; c1ccc(-n2c3ccccc3c3cc(-c4ccc5c(c4)c4cc6c(cc4n5-c4ccc5c(c4)c4ccccc4n5-c4ccccc4)C4(c5ccccc5-c5ccccc54)c4ccccc4-6)ccc32)cc1 ; CC1(C)c2ccccc2-c2cc3c4cc(-c5ccc6c(c5)c5ccccc5n6-c5cccc(-c6ccccc6)c5)ccc4n(-c4ccccc4)c3cc21]

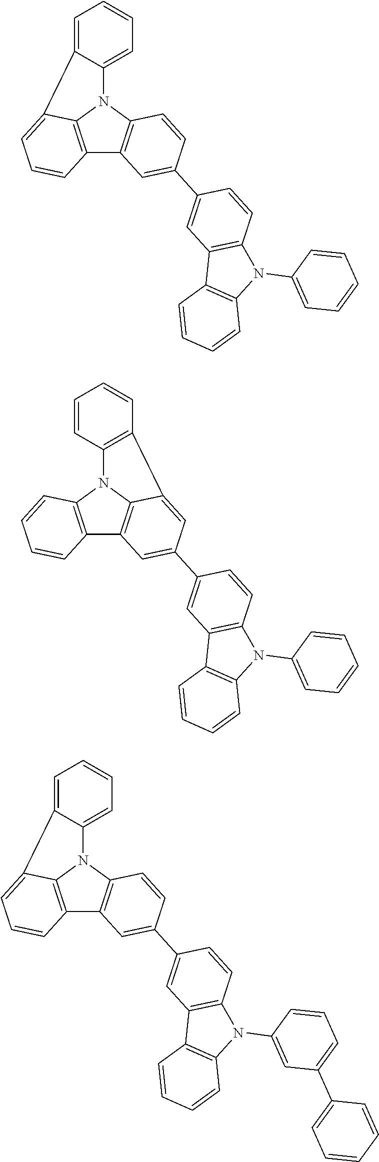 [SMILES: c1ccc(-n2c3ccccc3c3cc(-c4ccc5c(c4)c4cccc6c7ccccc7n5c64)ccc32)cc1 ; c1ccc(-n2c3ccccc3c3cc(-c4cc5c6ccccc6n6c7ccccc7c(c4)c56)ccc32)cc1 ; c1ccc(-c2cccc(-n3c4ccccc4c4cc(-c5ccc6c(c5)c5cccc7c8ccccc8n6c75)ccc43)c2)cc1]

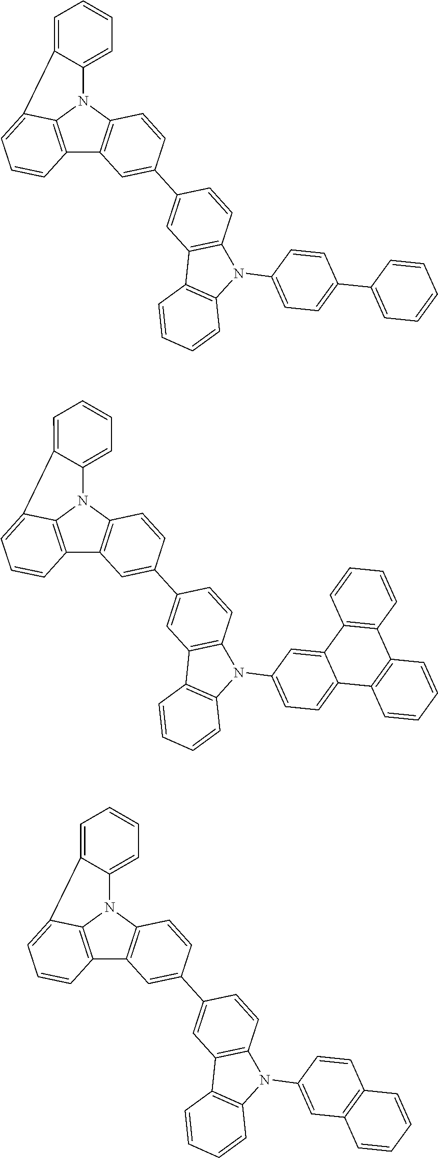 [SMILES: c1ccc(-c2ccc(-n3c4ccccc4c4cc(-c5ccc6c(c5)c5cccc7c8ccccc8n6c75)ccc43)cc2)cc1 ; c1ccc2c(c1)c1ccccc1c1cc(-n3c4ccccc4c4cc(-c5ccc6c(c5)c5cccc7c8ccccc8n6c75)ccc43)ccc21 ; c1ccc2cc(-n3c4ccccc4c4cc(-c5ccc6c(c5)c5cccc7c8ccccc8n6c75)ccc43)ccc2c1]

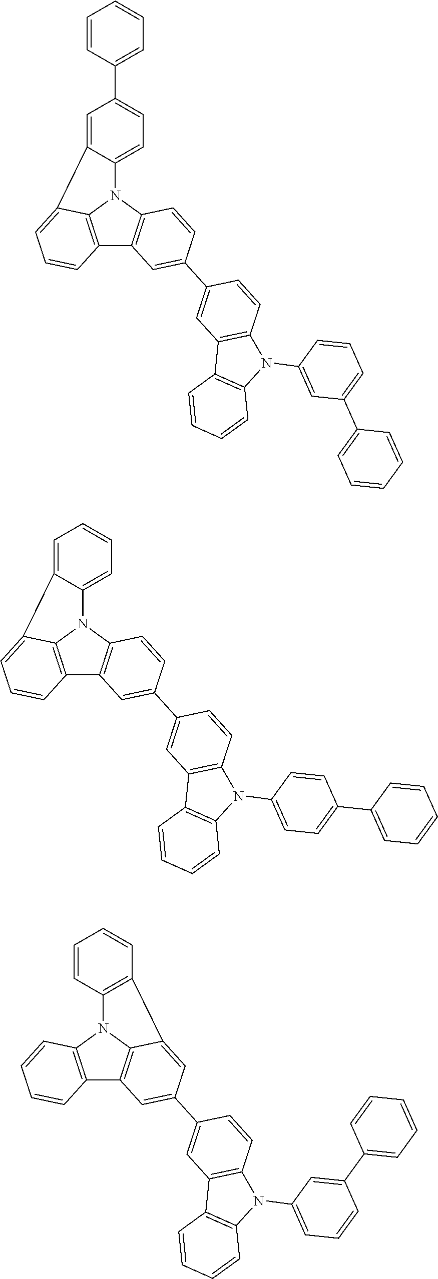 [SMILES: c1ccc(-c2cccc(-n3c4ccccc4c4cc(-c5ccc6c(c5)c5cccc7c8cc(-c9ccccc9)ccc8n6c75)ccc43)c2)cc1 ; c1ccc(-c2ccc(-n3c4ccccc4c4cc(-c5ccc6c(c5)c5cccc7c8ccccc8n6c75)ccc43)cc2)cc1 ; c1ccc(-c2cccc(-n3c4ccccc4c4cc(-c5cc6c7ccccc7n7c8ccccc8c(c5)c67)ccc43)c2)cc1]

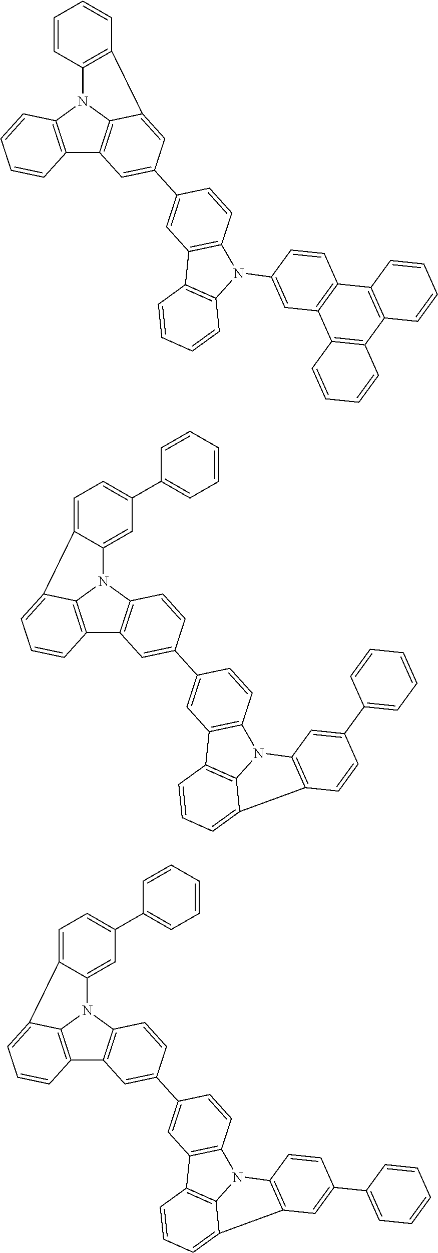 [SMILES: c1ccc2c(c1)c1ccccc1c1cc(-n3c4ccccc4c4cc(-c5cc6c7ccccc7n7c8ccccc8c(c5)c67)ccc43)ccc21 ; c1ccc(-c2ccc3c4cccc5c6cc(-c7ccc8c(c7)c7cccc9c%10ccc(-c%11ccccc%11)cc%10n8c79)ccc6n(c3c2)c54)cc1 ; c1ccc(-c2ccc3c(c2)c2cccc4c5cc(-c6ccc7c(c6)c6cccc8c9ccc(-c%10ccccc%10)cc9n7c68)ccc5n3c24)cc1]

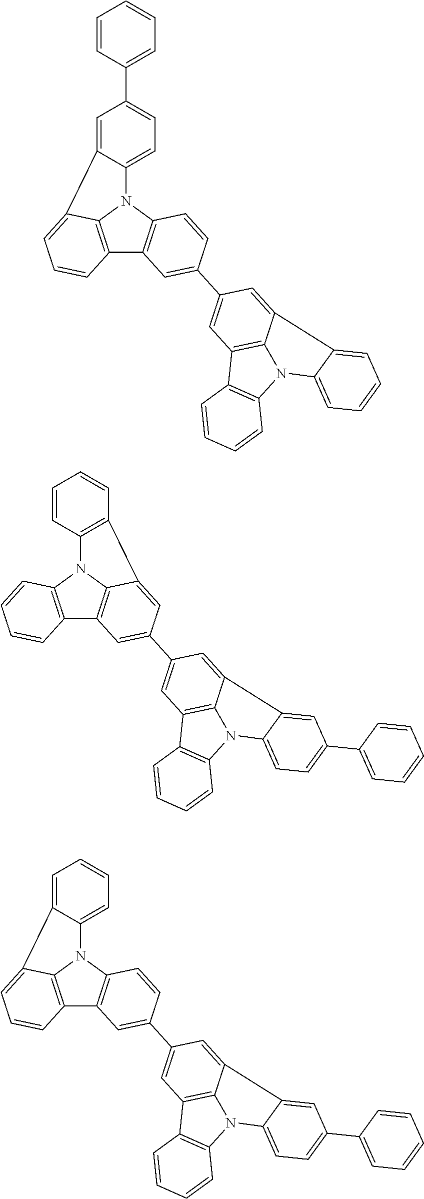 [SMILES: c1ccc(-c2ccc3c(c2)c2cccc4c5cc(-c6cc7c8ccccc8n8c9ccccc9c(c6)c78)ccc5n3c24)cc1 ; c1ccc(-c2ccc3c(c2)c2cc(-c4cc5c6ccccc6n6c7ccccc7c(c4)c56)cc4c5ccccc5n3c42)cc1 ; c1ccc(-c2ccc3c(c2)c2cc(-c4ccc5c(c4)c4cccc6c7ccccc7n5c64)cc4c5ccccc5n3c42)cc1]

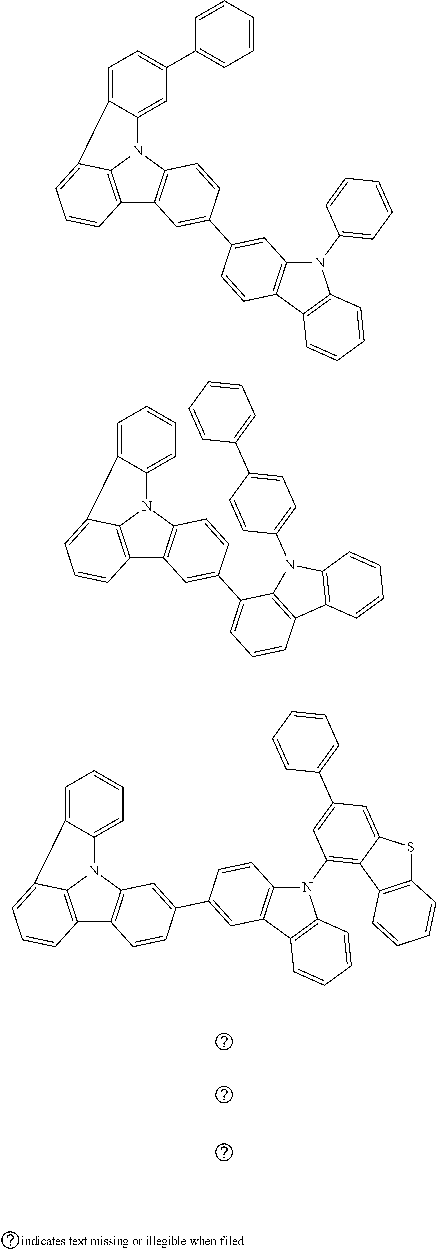 [SMILES: c1ccc(-c2ccc3c4cccc5c6cc(-c7ccc8c9ccccc9n(-c9ccccc9)c8c7)ccc6n(c3c2)c54)cc1 ; c1ccc(-c2ccc(-n3c4ccccc4c4cccc(-c5ccc6c(c5)c5cccc7c8ccccc8n6c75)c43)cc2)cc1 ; c1ccc(-c2cc(-n3c4ccccc4c4cc(-c5ccc6c7cccc8c9ccccc9n(c6c5)c87)ccc43)c3c(c2)sc2ccccc23)cc1]

Preferred bridged carbazoles are the structures of the following formula (14):

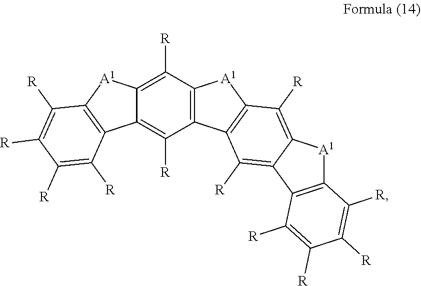

where A¹ and R have the definitions given above and A¹ is preferably the same or different at each instance and is selected from the group consisting of NR¹, where R¹ is an aromatic or heteroaromatic ring system which has 5 to 24 aromatic ring atoms and may be substituted by one or more R² radicals, and C(R¹)₂.

Preferred dibenzofuran derivatives are the compounds of the following formula (15):

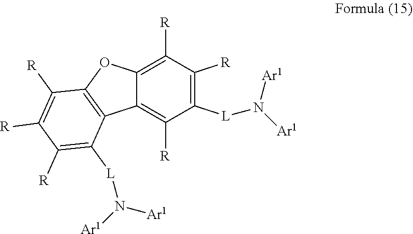

L is a single bond or an aromatic or heteroaromatic ring system which has 5 to 24 aromatic ring atoms and may be substituted by one or more R radicals;
where the oxygen may also be replaced by sulfur, so as to form a dibenzothiophene, and R and Ar¹ have the definitions given above. It is also possible here for the two Ar¹ groups that bind to the same nitrogen atom, or for one Ar¹ group and one L group that bind to the same nitrogen atom, to be bonded to one another, for example to give a carbazole.

Examples of suitable dibenzofuran derivatives are the compounds depicted below.

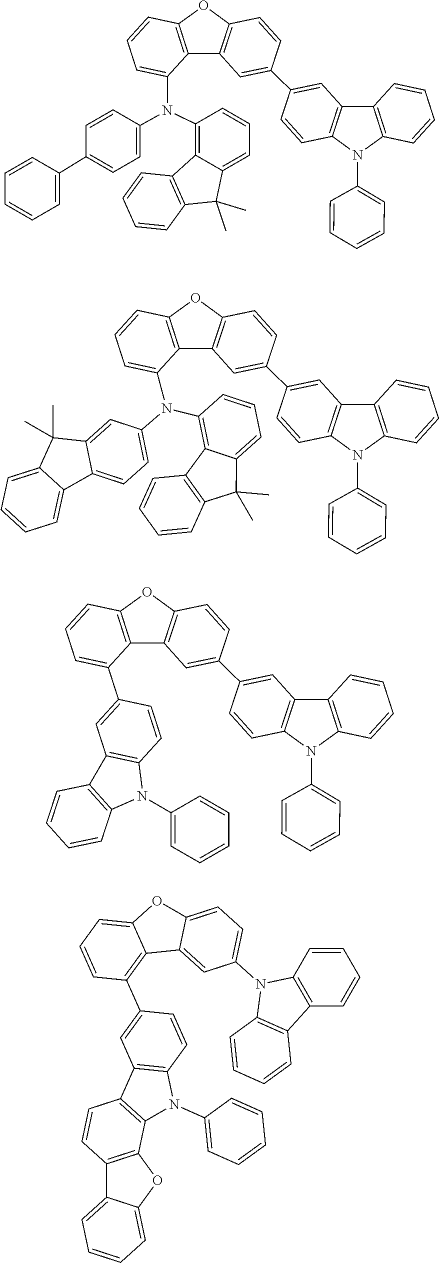 [SMILES: CC1(C)c2ccccc2-c2c(N(c3ccc(-c4ccccc4)cc3)c3cccc4oc5ccc(-c6ccc7c(c6)c6ccccc6n7-c6ccccc6)cc5c34)cccc21 ; CC1(C)c2ccccc2-c2ccc(N(c3cccc4c3-c3ccccc3C4(C)C)c3cccc4oc5ccc(-c6ccc7c(c6)c6ccccc6n7-c6ccccc6)cc5c34)cc21 ; c1ccc(-n2c3ccccc3c3cc(-c4ccc5oc6cccc(-c7ccc8c(c7)c7ccccc7n8-c7ccccc7)c6c5c4)ccc32)cc1 ; c1ccc(-n2c3ccc(-c4cccc5oc6ccc(-n7c8ccccc8c8ccccc87)cc6c45)cc3c3ccc4c5ccccc5oc4c32)cc1]

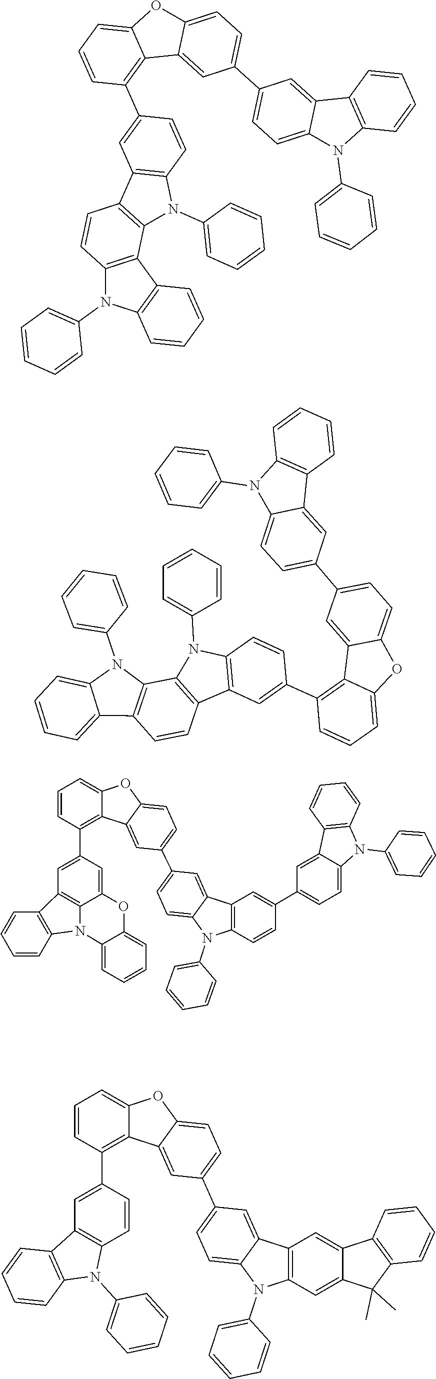

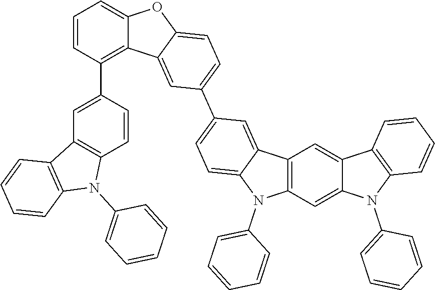 [SMILES: c1ccc(-n2c3ccccc3c3cc(-c4cccc5oc6ccc(-c7ccc8c(c7)c7cc9c%10ccccc%10n(-c%10ccccc%10)c9cc7n8-c7ccccc7)cc6c45)ccc32)cc1]

Preferred carbazoleamines are the structures of the following formulae (15), (16) and (17):

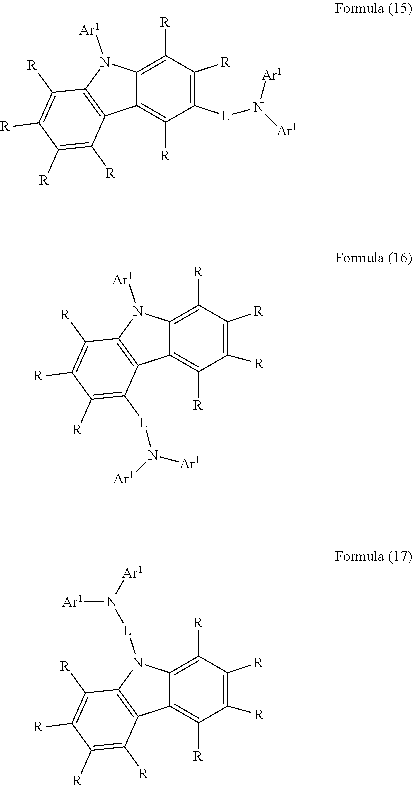

where L, R and Ar¹ have the definitions given above.

Examples of suitable carbazoleamine derivatives are the compounds depicted below.

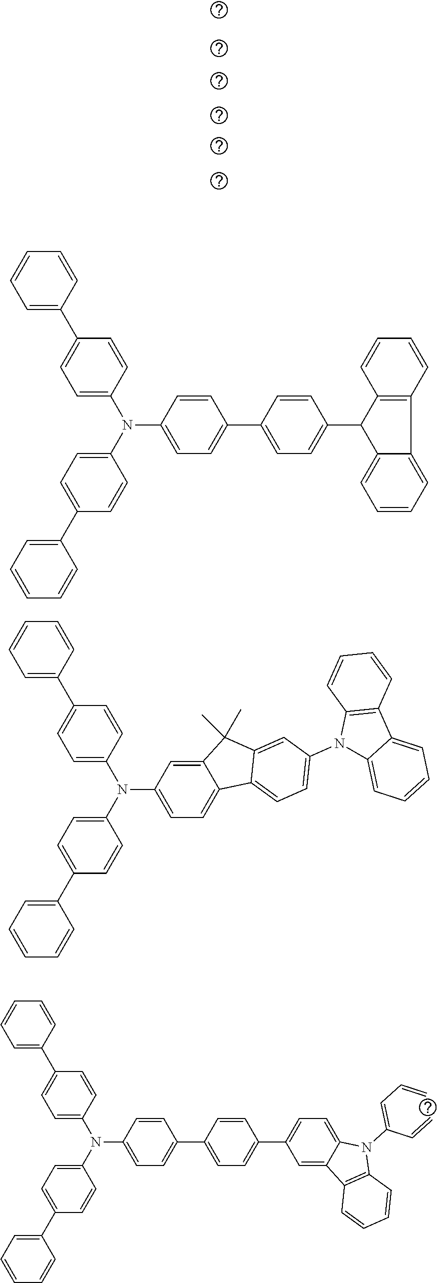

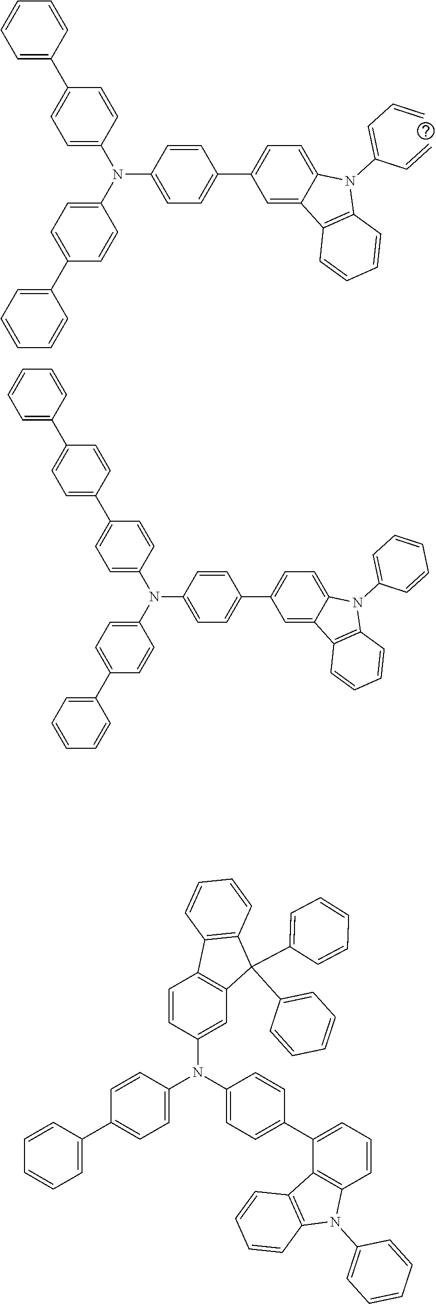

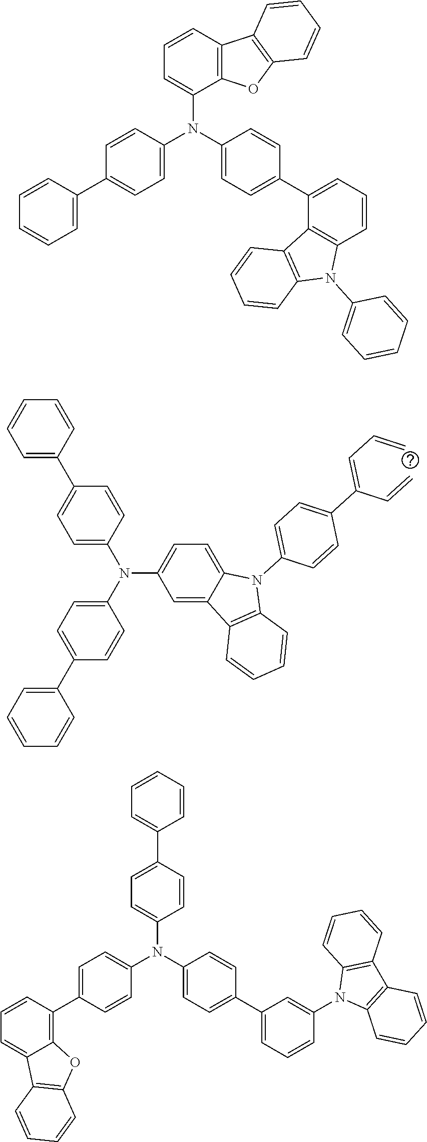 [SMILES: c1ccc(-c2ccc(N(c3ccc(-c4cccc5c4c4ccccc4n5-c4ccccc4)cc3)c3cccc4c3oc3ccccc34)cc2)cc1 ; C=C/C=C(\C=C)c1ccc(-n2c3ccccc3c3cc(N(c4ccc(-c5ccccc5)cc4)c4ccc(-c5ccccc5)cc4)ccc32)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

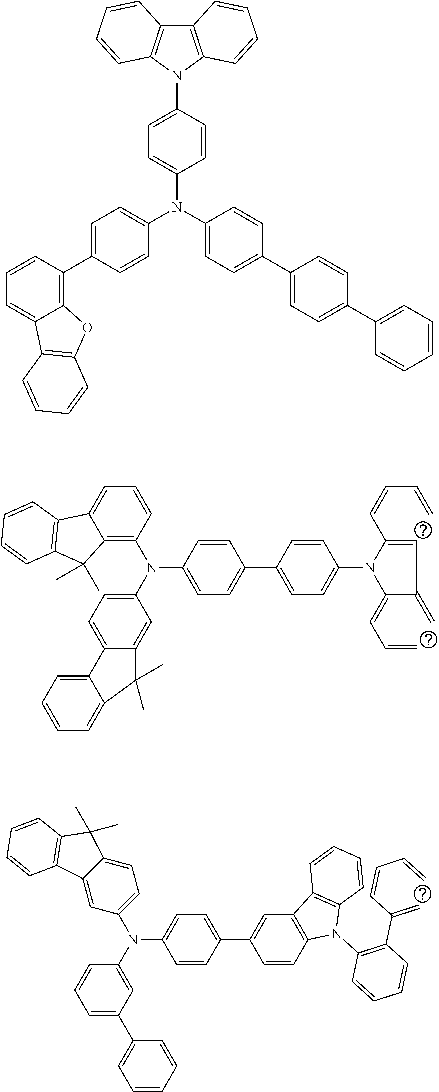 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; C=C/C=C\c1cc(=C)/c(=C\C=C)n1-c1ccc(-c2ccc(N(c3ccc4c(c3)C(C)(C)c3ccccc3-4)c3cccc4c3C(C)(C)c3ccccc3-4)cc2)cc1 ; C=C/C=C\C(=C)c1ccccc1-n1c2ccccc2c2cc(-c3ccc(N(c4cccc(-c5ccccc5)c4)c4ccc5c(c4)-c4ccccc4C5(C)C)cc3)ccc21]

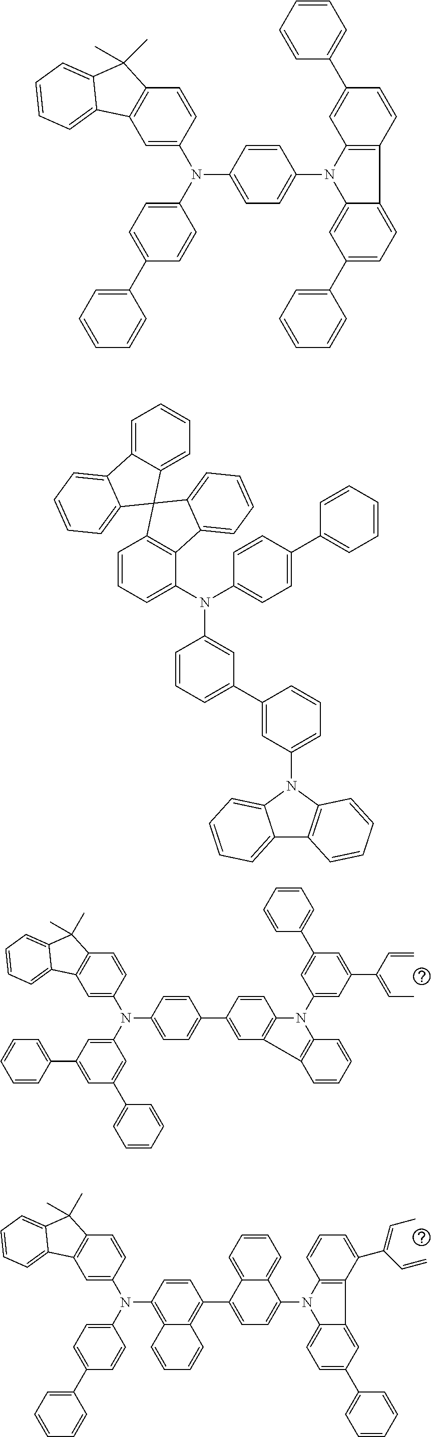

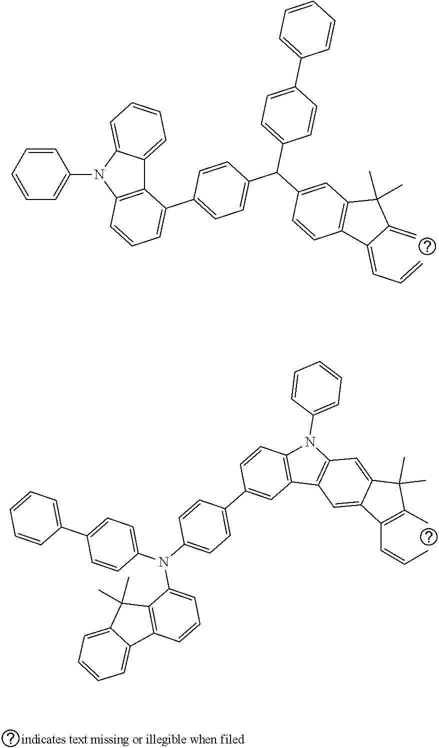 [SMILES: C=C/C=C1\C(=C)C(C)(C)c2cc(C(c3ccc(-c4ccccc4)cc3)c3ccc(-c4cccc5c4c4ccccc4n5-c4ccccc4)cc3)ccc21 ; C/C=C\C1=C(C)C(C)(C)c2cc3c(cc21)c1cc(-c2ccc(N(c4ccc(-c5ccccc5)cc4)c4cccc5c4C(C)(C)c4ccccc4-5)cc2)ccc1n3-c1ccccc1]

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186 and WO 2018/041769, WO 2019/020538, WO 2018/178001, WO 2019/115423 and WO 2019/158453. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Examples of phosphorescent dopants are adduced below.

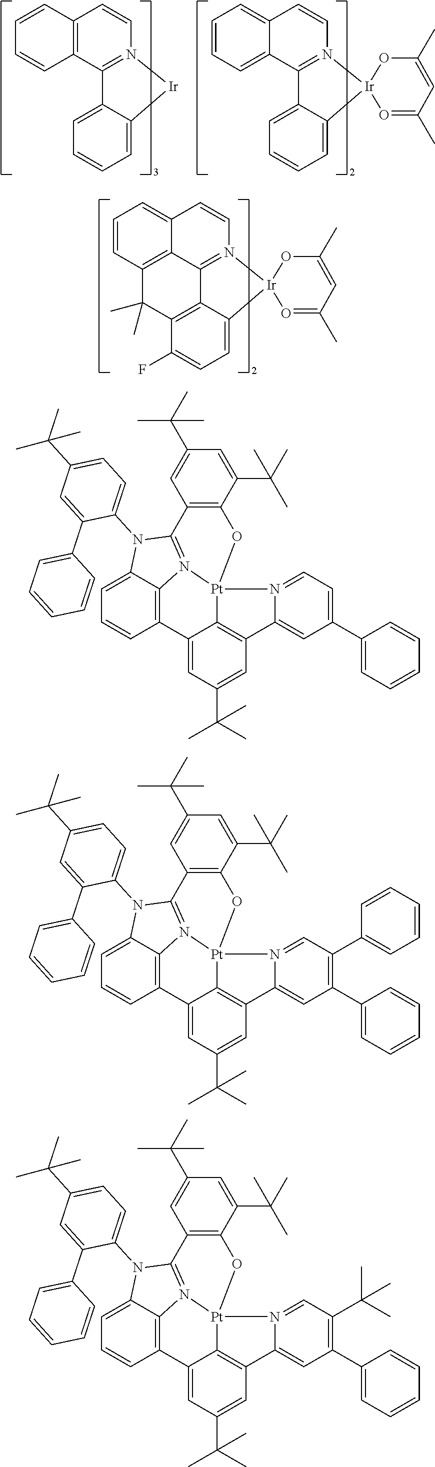

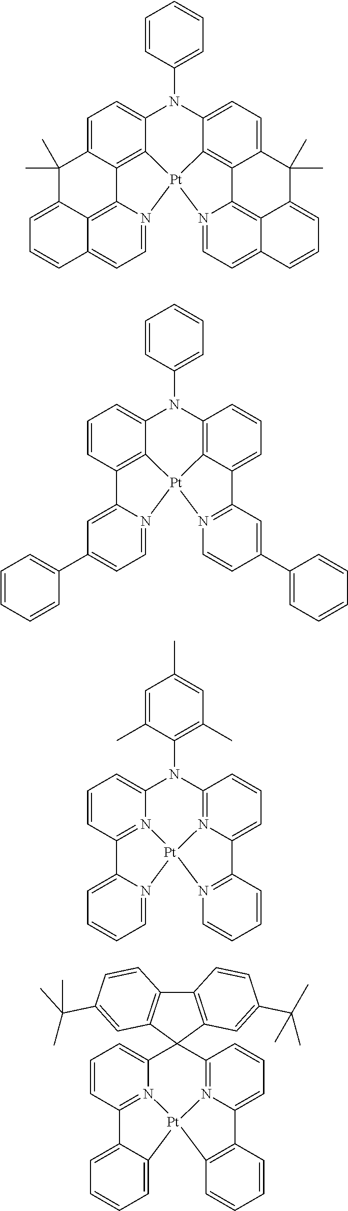

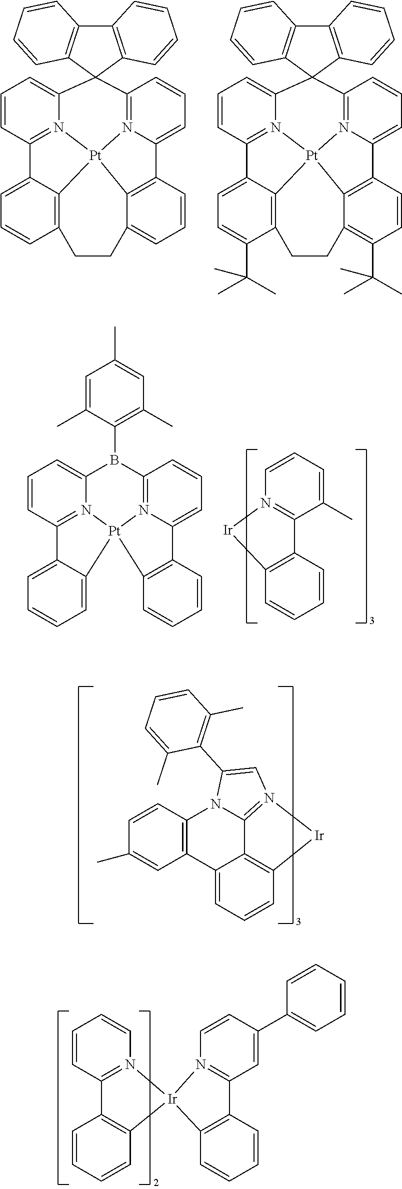

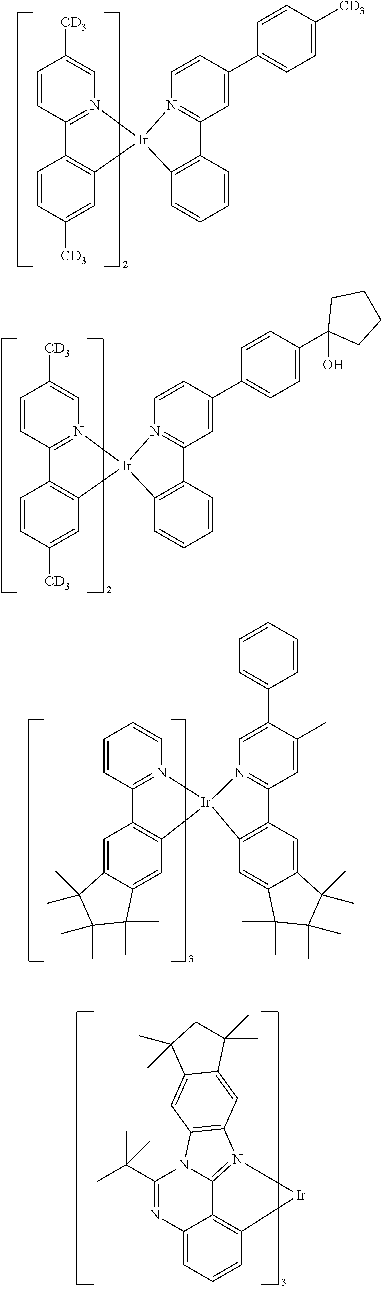

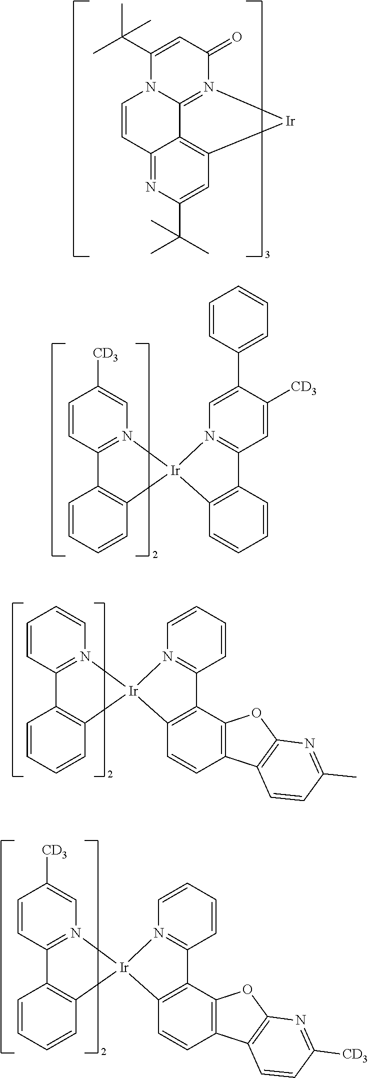

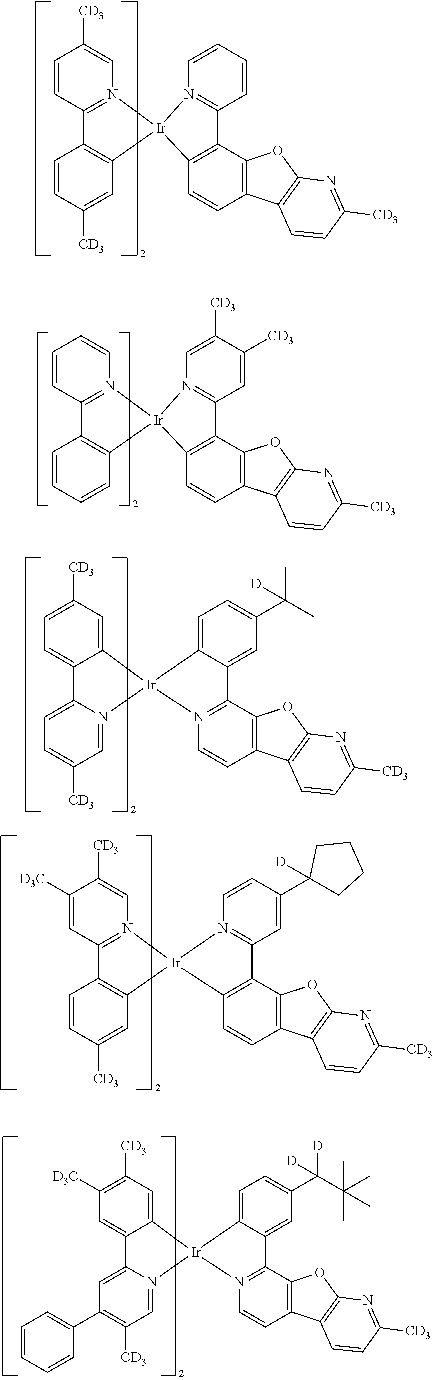

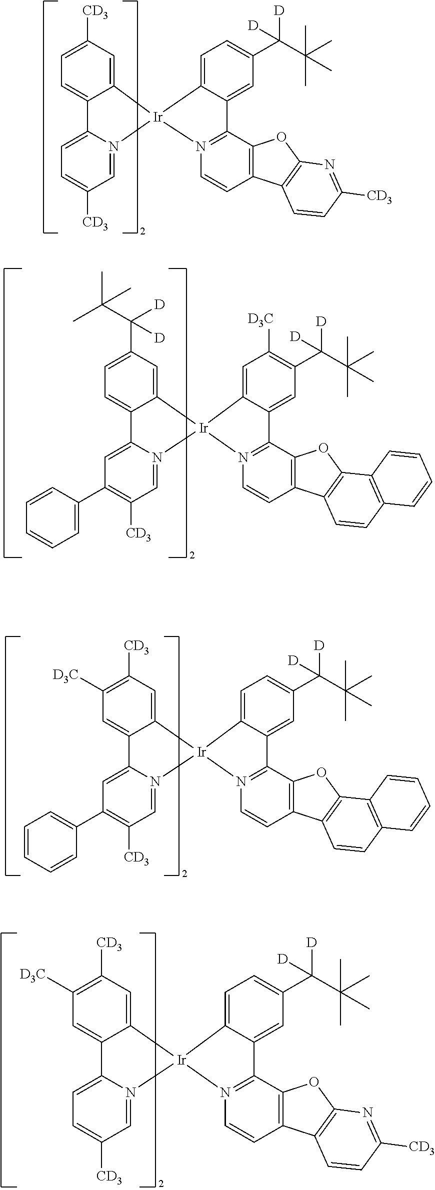

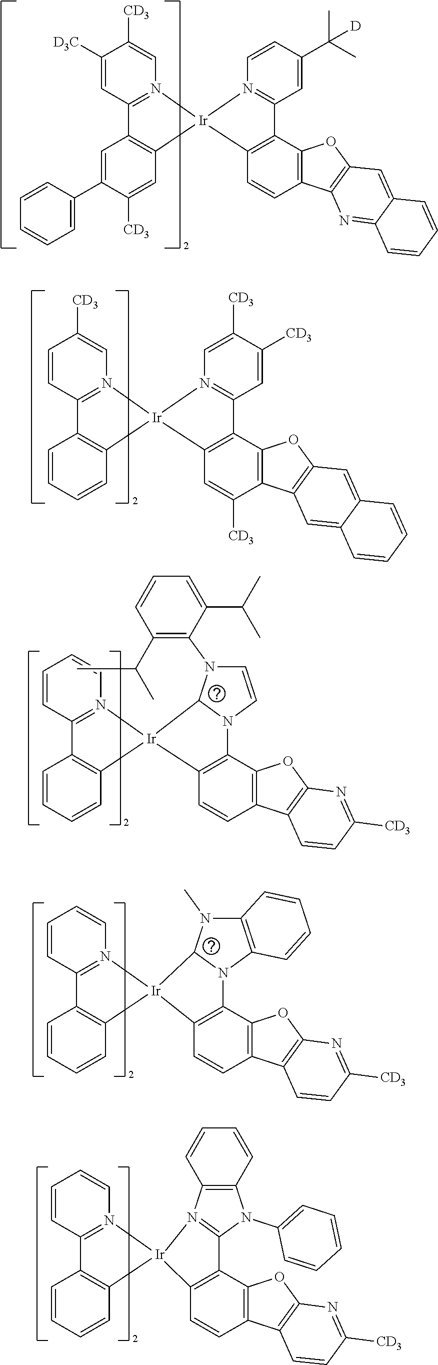

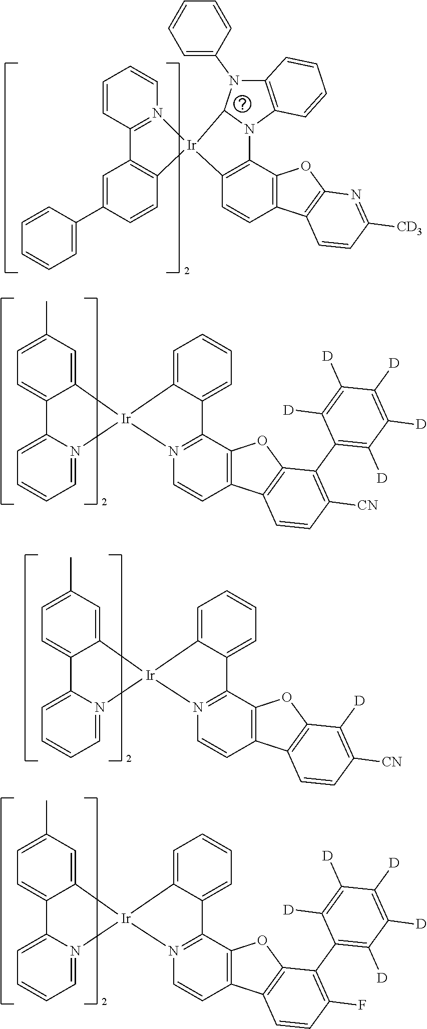

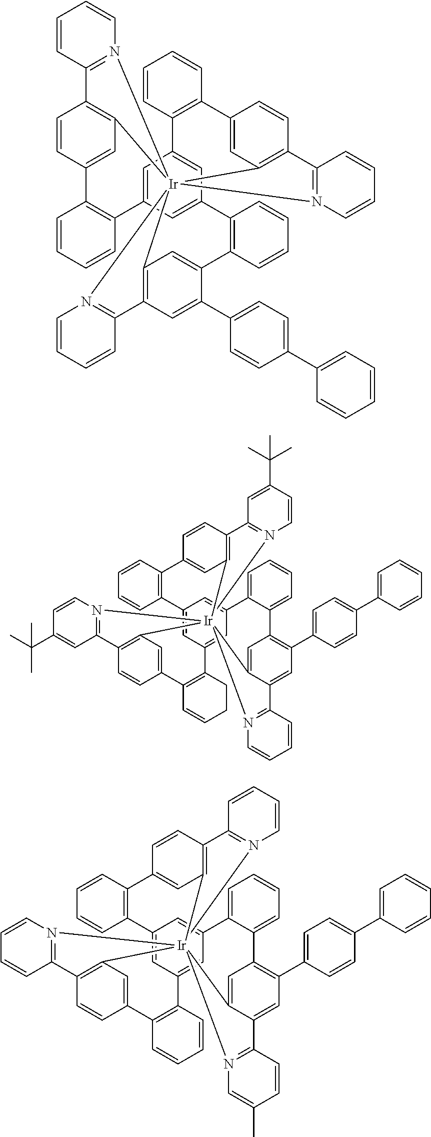

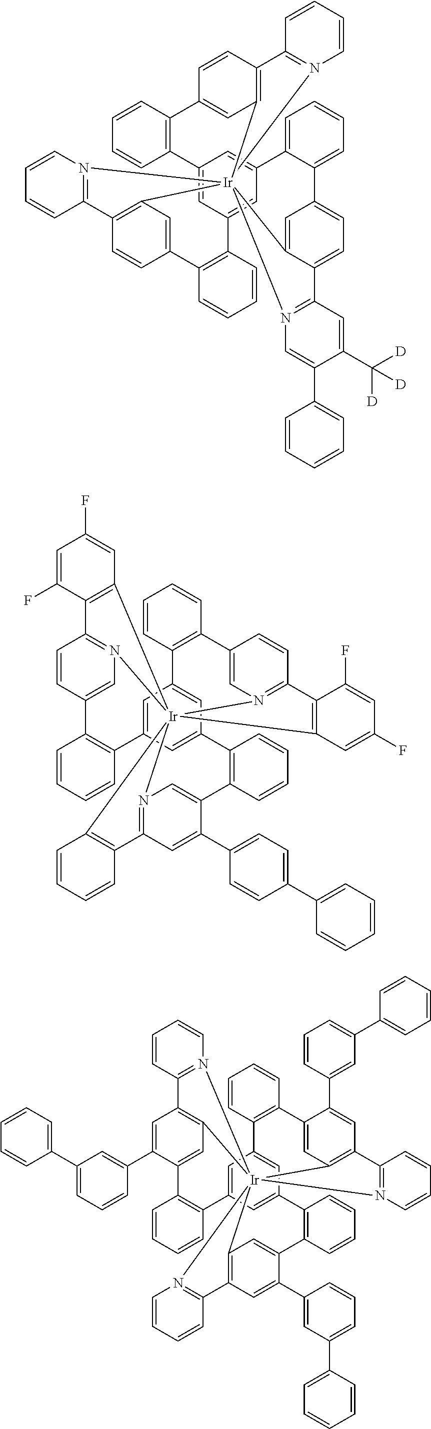

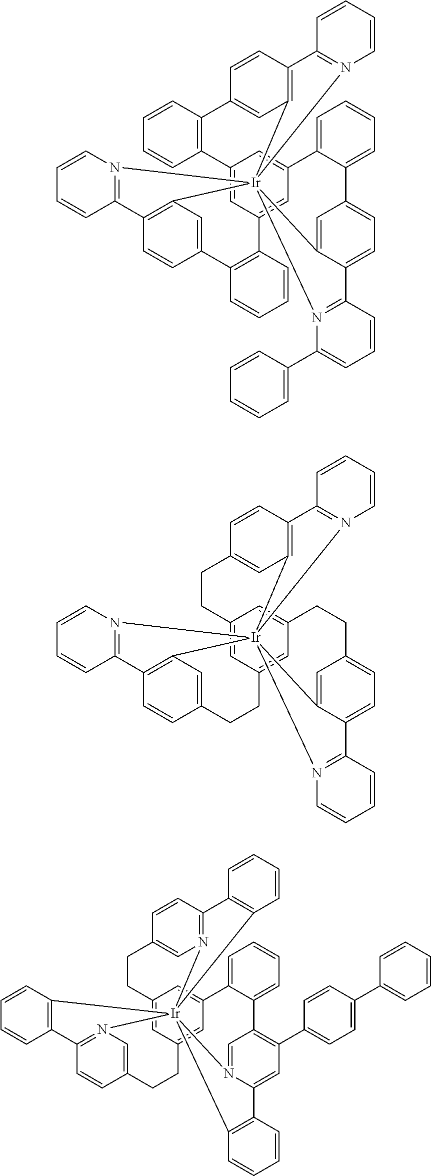

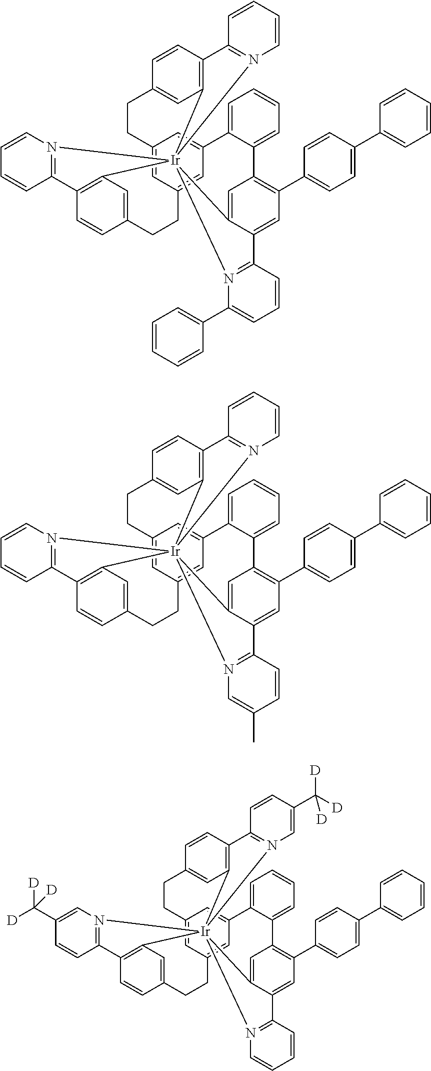

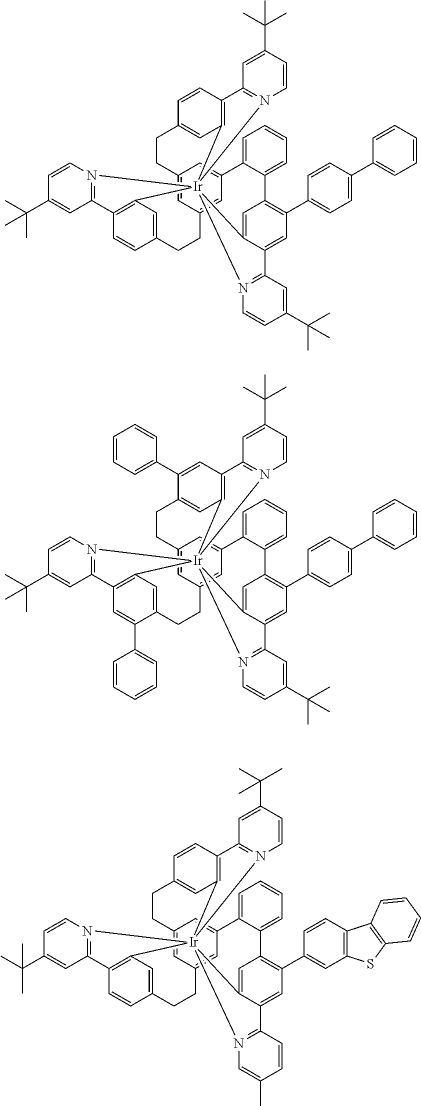

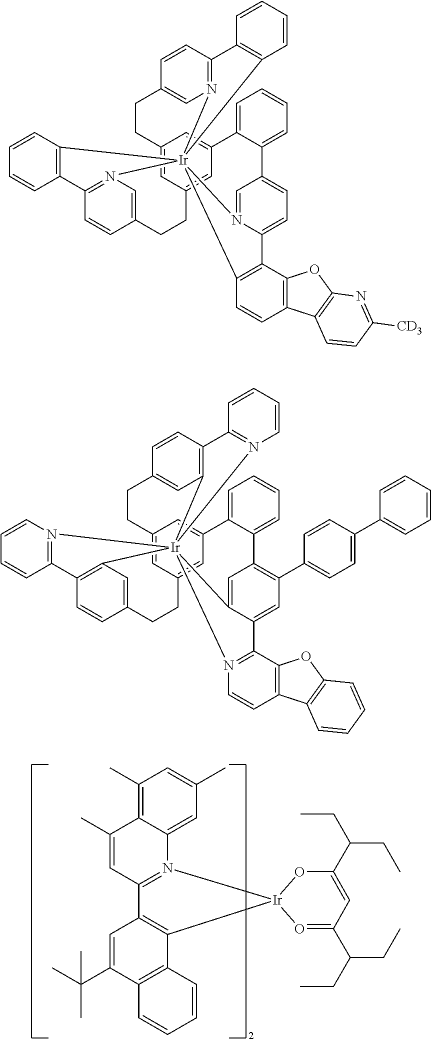

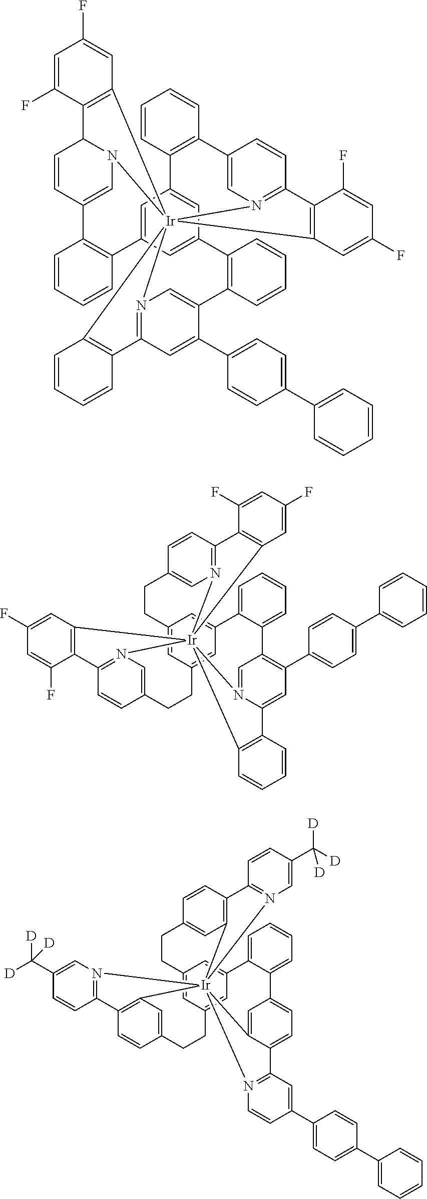

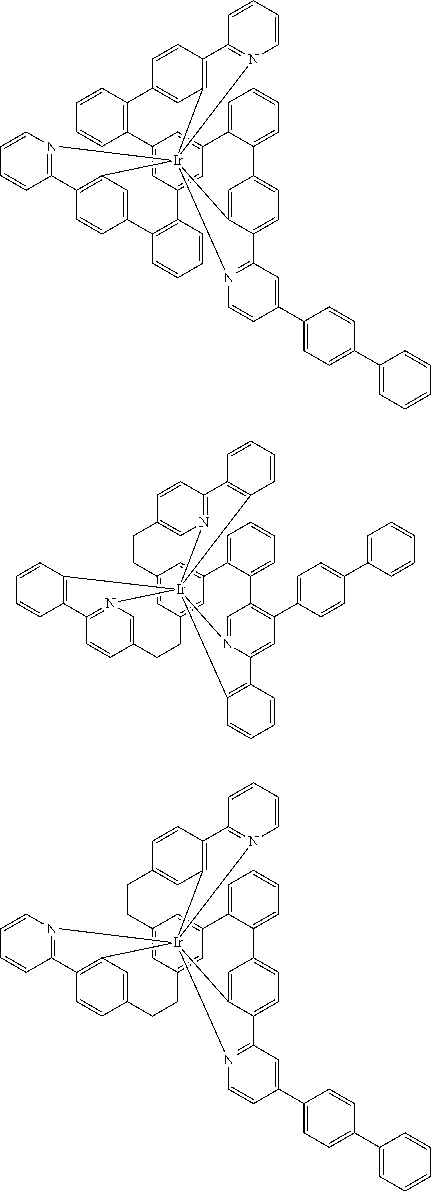

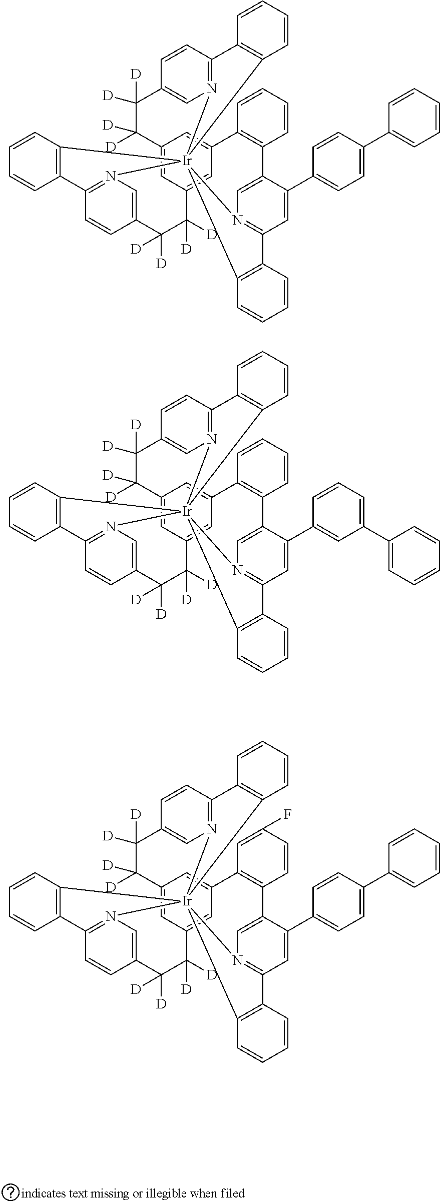

In the further layers of the organic electroluminescent device of the invention, it is possible to use any materials as typically used according to the prior art. The person skilled in the art will therefore be able, without exercising inventive skill, to use any materials known for organic electroluminescent devices in combination with the compounds of formula (1) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapor deposition.

These methods are known in general terms to those skilled in the art and can be applied by those skilled in the art without exercising inventive skill to organic electroluminescent devices comprising the compounds of formula (1).

The materials of the invention and the organic electroluminescent devices of the invention are notable for one or more of the following surprising advantages over the prior art:
1. OLEDs containing the compounds of formula (1) as matrix material for phosphorescent emitters lead to long lifetimes. This is especially true when the compounds are used as matrix material for a phosphorescent emitter. In particular, the OLEDs show an improved lifetime compared to OLEDs with matrix materials that likewise contain a lactam fused to a carbazole, but do not have a second carbazole fused to the lactam.
2. OLEDs containing the compounds of formula (1) lead to high efficiencies. This is especially true when the compounds are used as matrix material for a phosphorescent emitter.
3. OLEDs containing the compounds of formula (1) lead to low operating voltages. This is especially true when the compounds are used as matrix material for a phosphorescent emitter.
4. The compounds of the invention may also be used with very good properties in an electron transport layer, including in combination with a fluorescent emission layer, or in a hole blocker layer.

The invention is illustrated in detail by the examples which follow, without any intention of restricting it thereby. The person skilled in the art will be able to use the information given to execute the invention over the entire scope disclosed and produce further inventive electronic devices without exercising inventive skill.

### SYNTHESIS EXAMPLES

The syntheses which follow, unless stated otherwise, are conducted under a protective gas atmosphere in dried solvents. The compounds of the invention can be prepared by means of synthesis methods known to those skilled in the art.

### a) 2,7-Bis(2-chloroanilino)fluoren-9-one

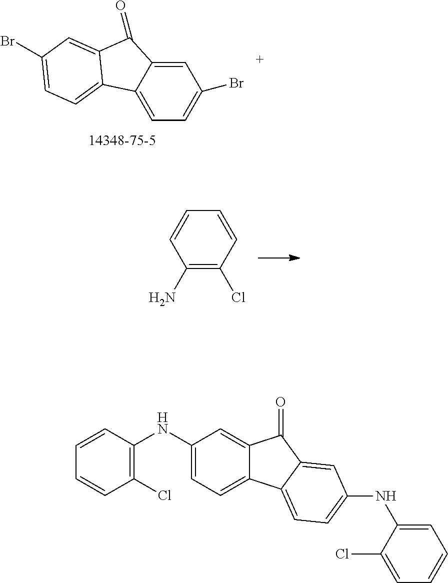 [SMILES: O=C1c2cc(Br)ccc2-c2ccc(Br)cc21 ; Nc1ccccc1Cl ; O=C1c2cc(Nc3ccccc3Cl)ccc2-c2ccc(Nc3ccccc3Cl)cc21]

23 g (70 mmol) of 2,7-dibromofluoren-9-one, 17.9 g (140 mmol) of 2-chloroaniline, 68.2 g (710 mmol) of sodium tert-butoxide, 613 mg (3 mmol) of palladium(II) acetate and 3.03 g (5 mmol) of dppf are dissolved in 1.3 l of toluene and stirred under reflux for 5 h. The reaction mixture is cooled down to room temperature, extended with toluene and filtered through Celite. The filtrate is concentrated under reduced pressure and the residue is crystallized from toluene/heptane. The product is isolated as a colorless solid.

Yield: 21 g (48 mmol), 70% of theory.

The following compounds can be prepared analogously:

| | Reactant 1 | Reactant 2 | Product | Yield |
|---|---|---|---|---|
| 1a | 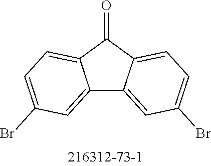 [SMILES: O=C1c2ccc(Br)cc2-c2cc(Br)ccc21] | 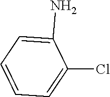 | 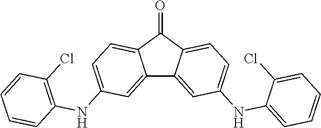 [SMILES: O=C1c2ccc(Nc3ccccc3Cl)cc2-c2cc(Nc3ccccc3Cl)ccc21] | 79% |
| 2a | 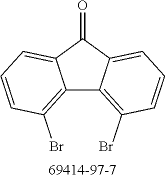 [SMILES: O=C1c2cccc(Br)c2-c2c(Br)cccc21] | 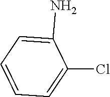 | 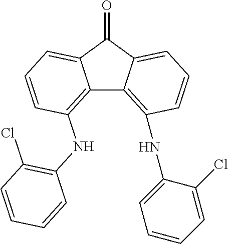 [SMILES: O=C1c2cccc(Nc3ccccc3Cl)c2-c2c(Nc3ccccc3Cl)cccc21] | 58% |
| 3a | 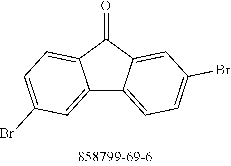 [SMILES: O=C1c2cc(Br)ccc2-c2cc(Br)ccc21] | 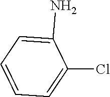 | 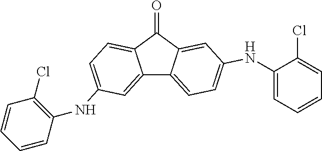 [SMILES: O=C1c2cc(Nc3ccccc3Cl)ccc2-c2cc(Nc3ccccc3Cl)ccc21] | 78% |
| 4a | 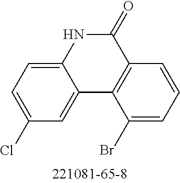 [SMILES: O=c1[nH]c2ccc(Cl)cc2c2c(Br)cccc12] | 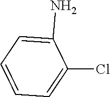 | 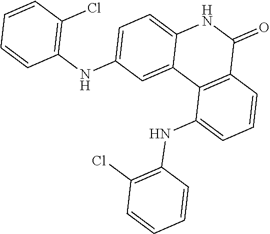 [SMILES: O=c1[nH]c2ccc(Nc3ccccc3Cl)cc2c2c(Nc3ccccc3Cl)cccc12] | 65% |
| 4a | 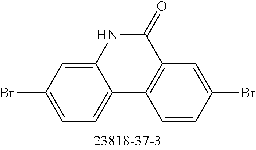 [SMILES: O=c1[nH]c2cc(Br)ccc2c2ccc(Br)cc12] | 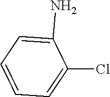 | 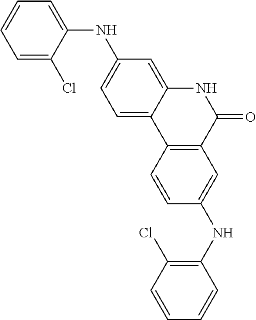 [SMILES: O=c1[nH]c2cc(Nc3ccccc3Cl)ccc2c2ccc(Nc3ccccc3Cl)cc12] | 82% |
| 6a | 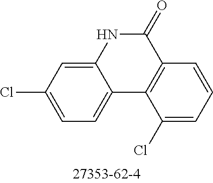 [SMILES: O=c1[nH]c2cc(Cl)ccc2c2c(Cl)cccc12] | 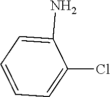 | 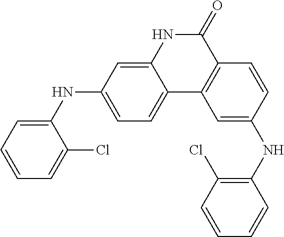 [SMILES: O=c1[nH]c2cc(Nc3ccccc3Cl)ccc2c2cc(Nc3ccccc3Cl)ccc12] | 56% |
| 7a | 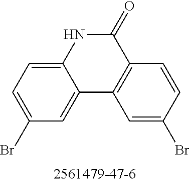 [SMILES: O=c1[nH]c2ccc(Br)cc2c2cc(Br)ccc12] | 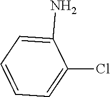 | 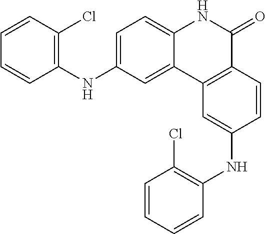 [SMILES: O=c1[nH]c2ccc(Nc3ccccc3Cl)cc2c2cc(Nc3ccccc3Cl)ccc12] | 77% |
| 8a | 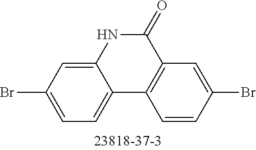 [SMILES: O=c1[nH]c2cc(Br)ccc2c2ccc(Br)cc12] | 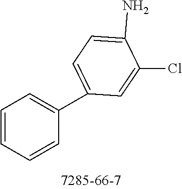 [SMILES: Nc1ccc(-c2ccccc2)cc1Cl] | 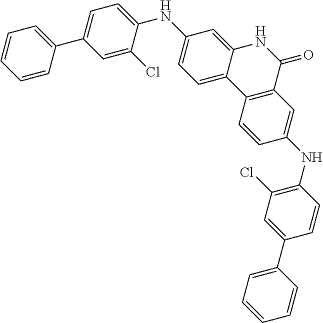 [SMILES: O=c1[nH]c2cc(Nc3ccc(-c4ccccc4)cc3Cl)ccc2c2ccc(Nc3ccc(-c4ccccc4)cc3Cl)cc12] | 84% |
| 9a | 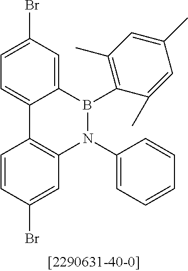 [SMILES: Cc1cc(C)c(B2c3cc(Br)ccc3-c3ccc(Br)cc3N2c2ccccc2)c(C)c1] | 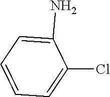 | 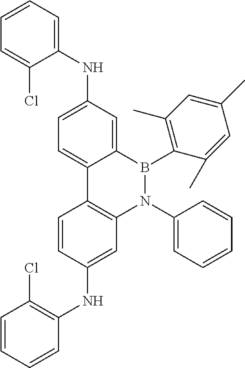 [SMILES: Cc1cc(C)c(B2c3cc(Nc4ccccc4Cl)ccc3-c3ccc(Nc4ccccc4Cl)cc3N2c2ccccc2)c(C)c1] | 71% |

### b) Cyclization

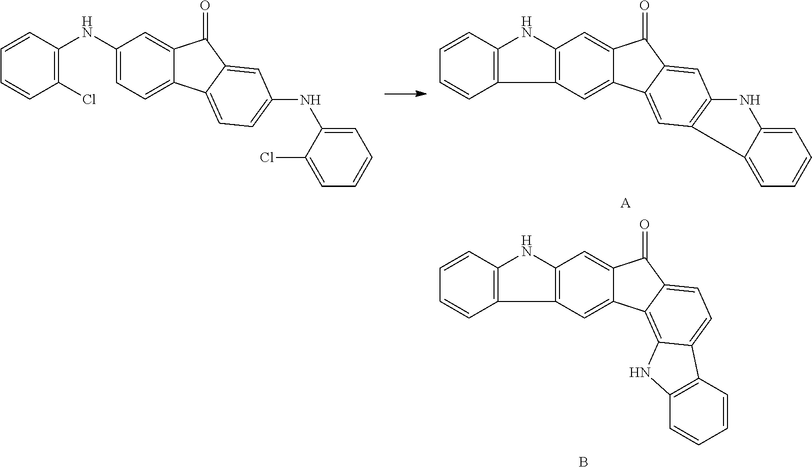

43 g (100 mmol) of 2,7-bis(2-chloroanilino)fluoren-9-one, 56 g (409 mmol) of potassium carbonate, 4.5 g (12 mmol) of tricyclohexylphosphine tetrafluoroborate and 1.38 g (6 mmol) of palladium(II) acetate are suspended in 500 ml of dimethylacetamide and stirred under reflux for 6 hours. After cooling, 300 ml of water and 400 ml of dichloromethane are added to the reaction mixture, which is stirred for a further 30 min, the organic phase is removed, the latter is filtered through a short Celite bed, and then the solvent is removed under reduced pressure. The crude product is subjected to hot extraction with toluene and recrystallized from toluene. The product is isolated as a beige solid.

Yield: 23 g (64 mmol), 66% of theory.

The following compounds can be prepared analogously:

| | Reactant 1 | Product (a) | Product (b) | Yield |
|---|---|---|---|---|
| 1b | 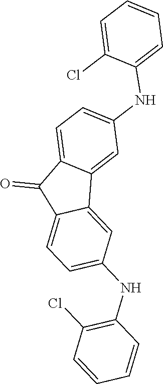 [SMILES: O=C1c2ccc(Nc3ccccc3Cl)cc2-c2cc(Nc3ccccc3Cl)ccc21] | 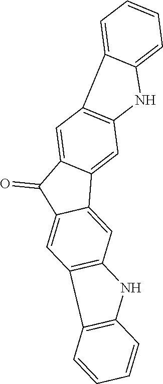 [SMILES: O=C1c2cc3c(cc2-c2cc4[nH]c5ccccc5c4cc21)[nH]c1ccccc13] | 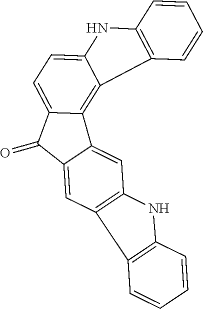 [SMILES: O=C1c2cc3c(cc2-c2c1ccc1[nH]c4ccccc4c21)[nH]c1ccccc13] | 56%/ 28% |
| 2b | 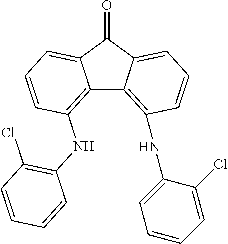 [SMILES: O=C1c2cccc(Nc3ccccc3Cl)c2-c2c(Nc3ccccc3Cl)cccc21] | 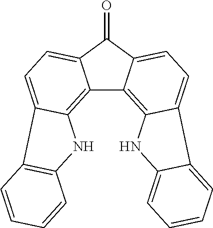 [SMILES: O=C1c2ccc3c([nH]c4ccccc43)c2-c2c1ccc1c2[nH]c2ccccc21] | | 64% |
| 3b | 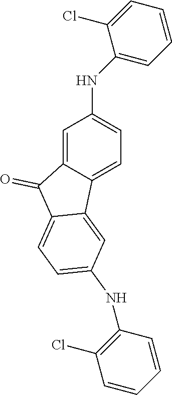 [SMILES: O=C1c2cc(Nc3ccccc3Cl)ccc2-c2cc(Nc3ccccc3Cl)ccc21] | 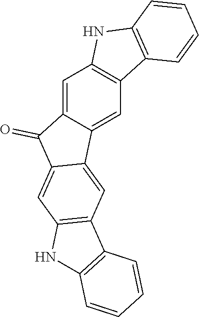 [SMILES: O=C1c2cc3[nH]c4ccccc4c3cc2-c2cc3c(cc21)[nH]c1ccccc13] | 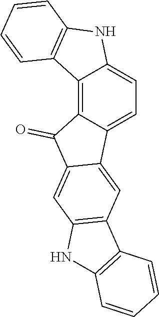 [SMILES: O=C1c2cc3[nH]c4ccccc4c3cc2-c2ccc3[nH]c4ccccc4c3c21] | 53%/ 21% |
| 4b | 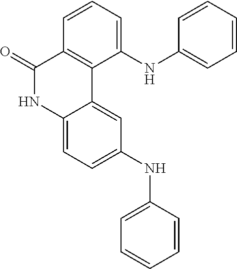 [SMILES: O=c1[nH]c2ccc(Nc3ccccc3)cc2c2c(Nc3ccccc3)cccc12] | 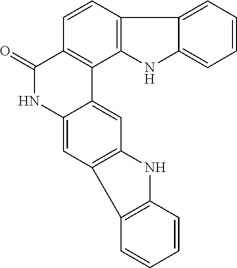 [SMILES: O=c1[nH]c2cc3c(cc2c2c1ccc1c4ccccc4[nH]c12)[nH]c1ccccc13] | | 60% |
| 5b | 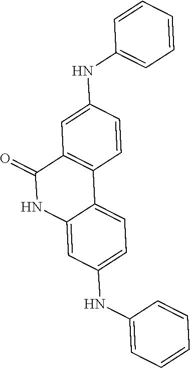 [SMILES: O=c1[nH]c2cc(Nc3ccccc3)ccc2c2ccc(Nc3ccccc3)cc12] | 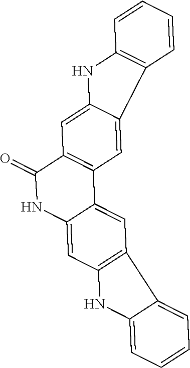 [SMILES: O=c1[nH]c2cc3[nH]c4ccccc4c3cc2c2cc3c(cc12)[nH]c1ccccc13] | 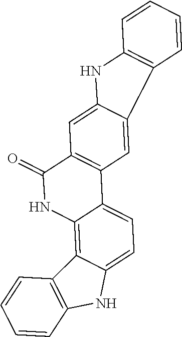 [SMILES: O=c1[nH]c2c(ccc3[nH]c4ccccc4c32)c2cc3c(cc12)[nH]c1ccccc13] | 59%/ 23% |
| 6b | 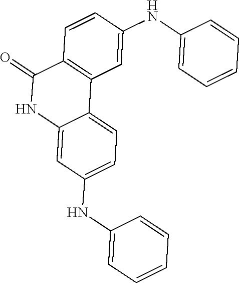 [SMILES: O=c1[nH]c2cc(Nc3ccccc3)ccc2c2cc(Nc3ccccc3)ccc12] | 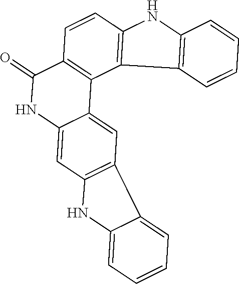 [SMILES: O=c1[nH]c2cc3[nH]c4ccccc4c3cc2c2c1ccc1[nH]c3ccccc3c12] | 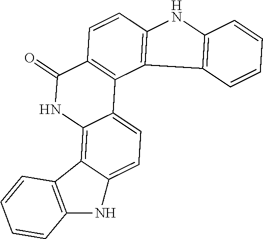 [SMILES: O=c1[nH]c2c(ccc3[nH]c4ccccc4c32)c2c1ccc1[nH]c3ccccc3c12] | 58%/ 20% |
| 7b | 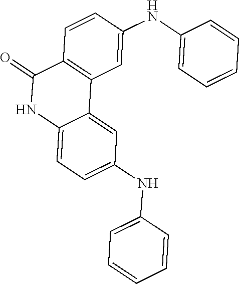 [SMILES: O=c1[nH]c2ccc(Nc3ccccc3)cc2c2cc(Nc3ccccc3)ccc12] | 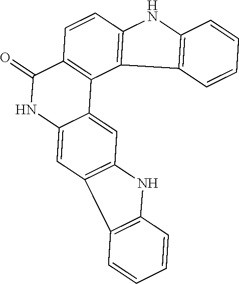 [SMILES: O=c1[nH]c2cc3c(cc2c2c1ccc1[nH]c4ccccc4c12)[nH]c1ccccc13] | | 63% |
| 8b | 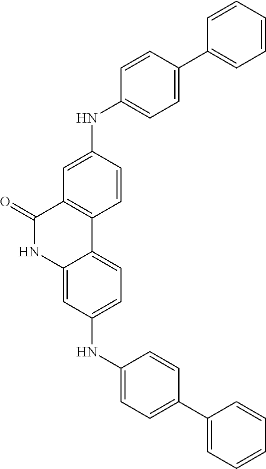 [SMILES: O=c1[nH]c2cc(Nc3ccc(-c4ccccc4)cc3)ccc2c2ccc(Nc3ccc(-c4ccccc4)cc3)cc12] | 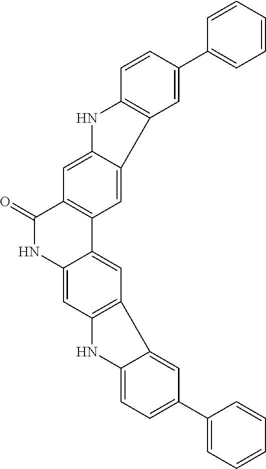 [SMILES: O=c1[nH]c2cc3[nH]c4ccc(-c5ccccc5)cc4c3cc2c2cc3c(cc12)[nH]c1ccc(-c2ccccc2)cc13] | 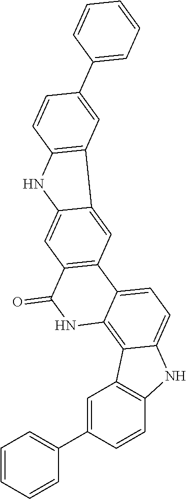 [SMILES: O=c1[nH]c2c(ccc3[nH]c4ccc(-c5ccccc5)cc4c32)c2cc3c(cc12)[nH]c1ccc(-c2ccccc2)cc13] | 61%/ 12% |
| 9b | 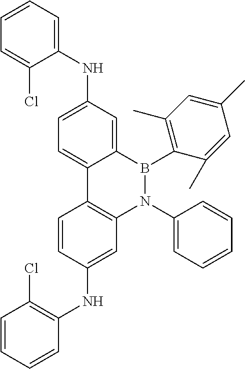 [SMILES: Cc1cc(C)c(B2c3cc(Nc4ccccc4Cl)ccc3-c3ccc(Nc4ccccc4Cl)cc3N2c2ccccc2)c(C)c1] | 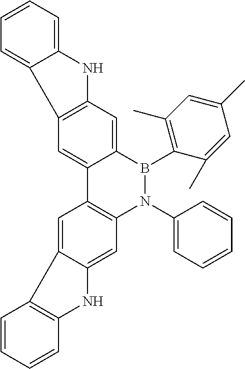 [SMILES: Cc1cc(C)c(B2c3cc4[nH]c5ccccc5c4cc3-c3cc4c(cc3N2c2ccccc2)[nH]c2ccccc24)c(C)c1] | 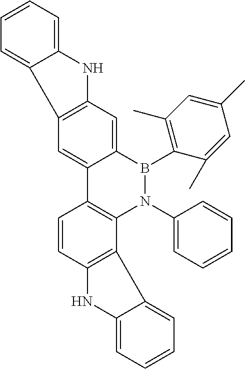 [SMILES: Cc1cc(C)c(B2c3cc4[nH]c5ccccc5c4cc3-c3ccc4[nH]c5ccccc5c4c3N2c2ccccc2)c(C)c1] | 58%/ 14% |

### c) 2,7-Bis(2-nitrophenyl)fluoren-9-one

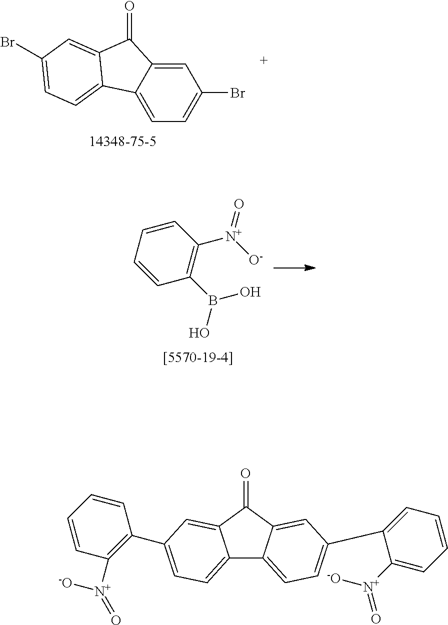

To a well-stirred, degassed suspension of 31 g (185 mmol) of B-(2-nitrophenyl)benzeneboronic acid, 20.2 g (60 mmol) of 2,7-dibromofluoren-9-one and 66.5 g (212.7 mmol) of potassium carbonate in a mixture of 250 ml of water and 250 ml of THE is added 1.7 g (1.49 mmol) of Pd(PPh₃)₄, and the mixture is heated under reflux for 17 h. After cooling, the organic phase is removed, washed three times with 200 ml of water and once with 200 ml of saturated aqueous sodium chloride solution, dried over magnesium sulfate and concentrated to dryness by rotary evaporation. The gray residue is recrystallized from hexane. The precipitated crystals are filtered off with suction, washed with a little MeOH and dried under reduced pressure.

Yield: 22.6 g (53 mmol), 90% of theory

The following compounds can be prepared analogously:

| | Reactant 1 | Reactant 2 | Product | Yield |
|---|---|---|---|---|
| 1c | 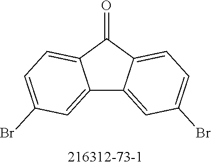 [SMILES: O=C1c2ccc(Br)cc2-c2cc(Br)ccc21] | 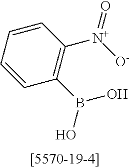 [SMILES: O=[N+]([O-])c1ccccc1B(O)O] | 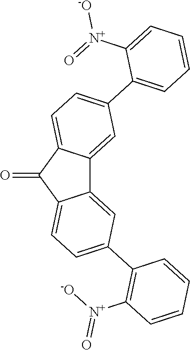 | 73% |
| 2c | 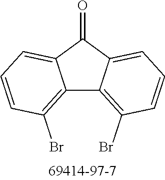 [SMILES: O=C1c2cccc(Br)c2-c2c(Br)cccc21] | 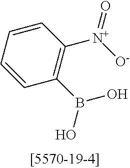 [SMILES: O=[N+]([O-])c1ccccc1B(O)O] | 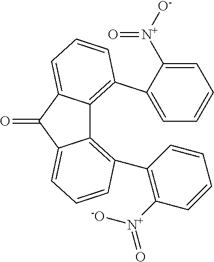 | 51% |
| 3c | 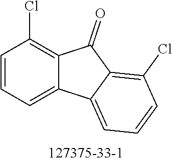 [SMILES: O=C1c2c(Cl)cccc2-c2cccc(Cl)c21] | 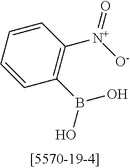 [SMILES: O=[N+]([O-])c1ccccc1B(O)O] | 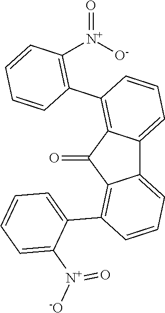 | 63% |
| 4c | 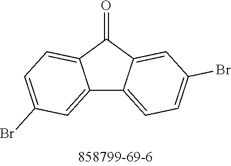 [SMILES: O=C1c2cc(Br)ccc2-c2cc(Br)ccc21] | 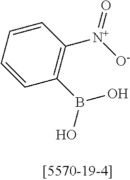 [SMILES: O=[N+]([O-])c1ccccc1B(O)O] | 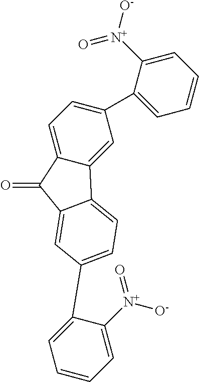 [SMILES: O=C1c2cc(-c3ccccc3[N+](=O)[O-])ccc2-c2cc(-c3ccccc3[N+](=O)[O-])ccc21] | 70% |
| 5c | 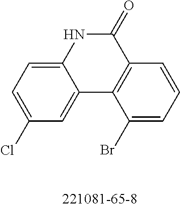 [SMILES: O=c1[nH]c2ccc(Cl)cc2c2c(Br)cccc12] | 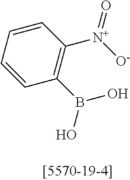 [SMILES: O=[N+]([O-])c1ccccc1B(O)O] | 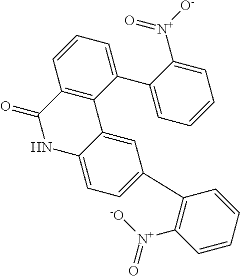 | 54% |
| 6c | 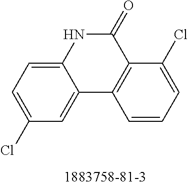 [SMILES: O=c1[nH]c2ccc(Cl)cc2c2cccc(Cl)c12] | 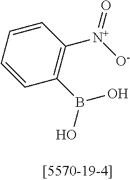 [SMILES: O=[N+]([O-])c1ccccc1B(O)O] | 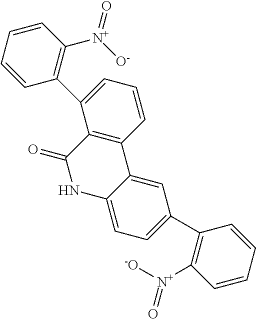 | 69% |
| 7c | 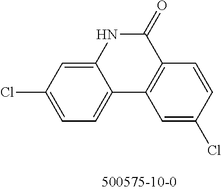 [SMILES: O=c1[nH]c2cc(Cl)ccc2c2cc(Cl)ccc12] | 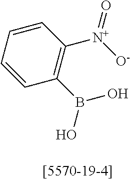 [SMILES: O=[N+]([O-])c1ccccc1B(O)O] | 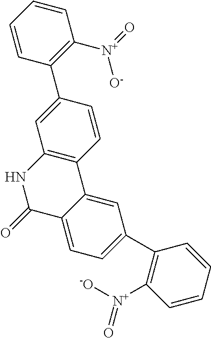 | 68% |
| 8c | 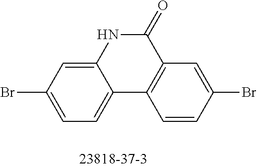 [SMILES: O=c1[nH]c2cc(Br)ccc2c2ccc(Br)cc12] | 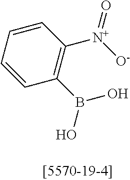 [SMILES: O=[N+]([O-])c1ccccc1B(O)O] | 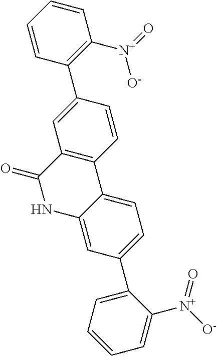 [SMILES: O=c1[nH]c2cc(-c3ccccc3[N+](=O)[O-])ccc2c2ccc(-c3ccccc3[N+](=O)[O-])cc12] | 80% |
| 9c | 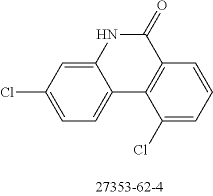 [SMILES: O=c1[nH]c2cc(Cl)ccc2c2c(Cl)cccc12] | 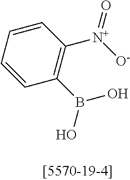 [SMILES: O=[N+]([O-])c1ccccc1B(O)O] | 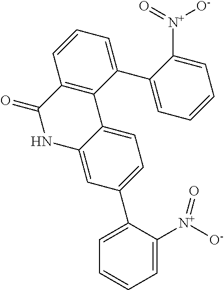 [SMILES: O=c1[nH]c2cc(-c3ccccc3[N+](=O)[O-])ccc2c2c(-c3ccccc3[N+](=O)[O-])cccc12] | 57% |
| 10c | 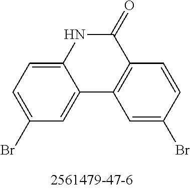 [SMILES: O=c1[nH]c2ccc(Br)cc2c2cc(Br)ccc12] | 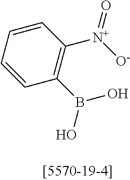 [SMILES: O=[N+]([O-])c1ccccc1B(O)O] | 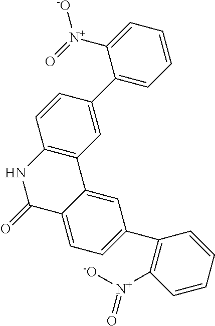 | 82% |
| 11c | 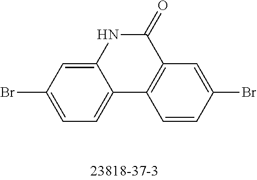 [SMILES: O=c1[nH]c2cc(Br)ccc2c2ccc(Br)cc12] | 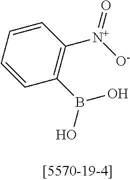 [SMILES: O=[N+]([O-])c1ccccc1B(O)O] | 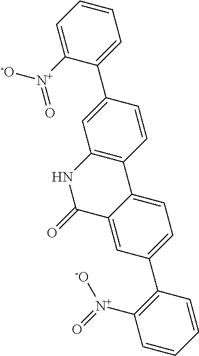 | 84% |
| 12c | 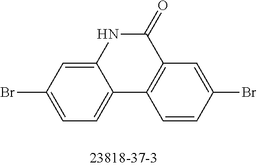 [SMILES: O=c1[nH]c2cc(Br)ccc2c2ccc(Br)cc12] | 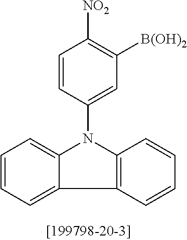 [SMILES: O=[N+]([O-])c1ccc(-n2c3ccccc3c3ccccc32)cc1B(O)O] | 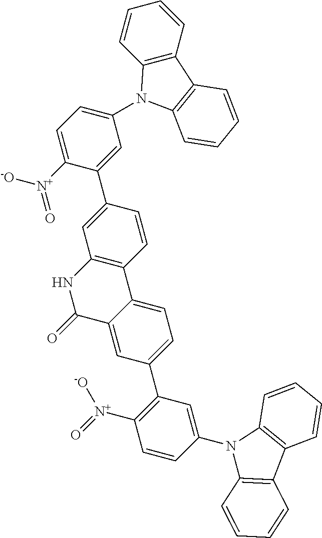 | 76% |
| 13c | 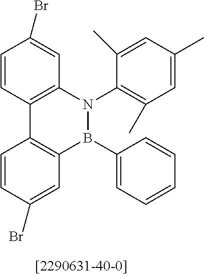 [SMILES: Cc1cc(C)c(N2B(c3ccccc3)c3cc(Br)ccc3-c3ccc(Br)cc32)c(C)c1] | 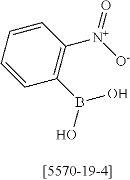 [SMILES: O=[N+]([O-])c1ccccc1B(O)O] | 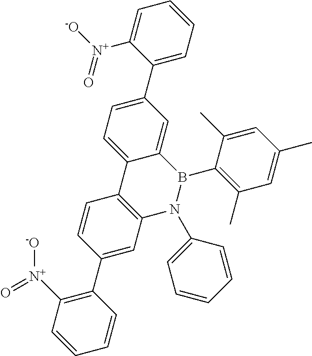 | 53% |

### d) Carbazole Synthesis

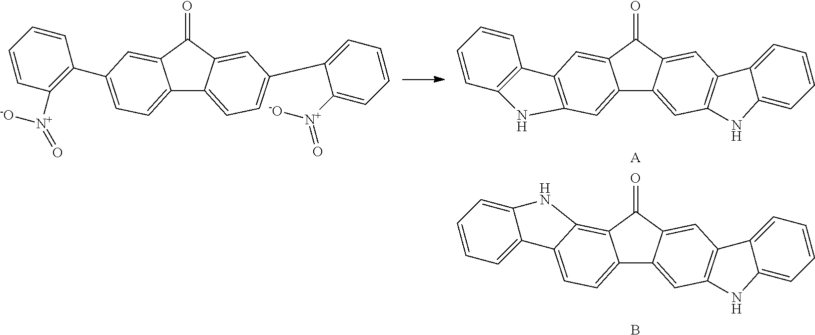

A mixture of 52 g (125 mmol) of 2,7-bis(2-nitrophenyl)fluoren-9-one and 146 ml (834 mmol) of triethyl phosphite is heated under reflux for 12 h. Subsequently, the rest of the triethyl phosphite is distilled off (72-76° C./9 mmHg). Water/MeOH (1:1) is added to the residue, and the solids are filtered off and recrystallized.

Yield: 33 g (92 mmol), 75% of theory.

The following compounds can be prepared analogously:

| | Reactant 1 | Product (a) | Product (b) | Yield |
|---|---|---|---|---|
| 1d | 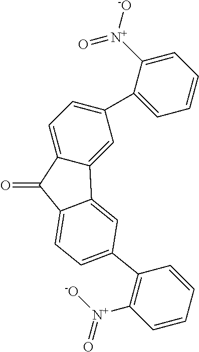 | 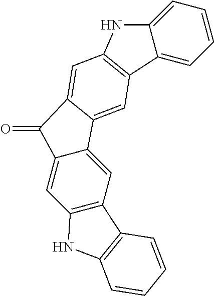 [SMILES: O=C1c2cc3[nH]c4ccccc4c3cc2-c2cc3c(cc21)[nH]c1ccccc13] | 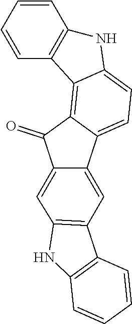 [SMILES: O=C1c2cc3[nH]c4ccccc4c3cc2-c2ccc3[nH]c4ccccc4c3c21] | 61%/15% |
| 2d | 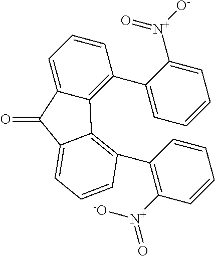 | 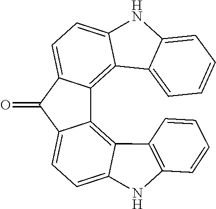 [SMILES: O=C1c2ccc3[nH]c4ccccc4c3c2-c2c1ccc1[nH]c3ccccc3c21] | | 73% |
| 3d | 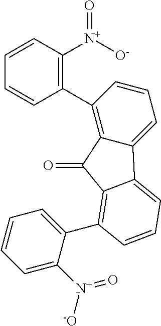 | 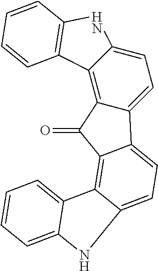 [SMILES: O=C1c2c(ccc3[nH]c4ccccc4c23)-c2ccc3[nH]c4ccccc4c3c21] | | 71% |
| 4d | 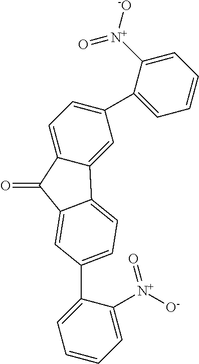 | 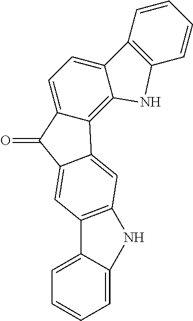 [SMILES: O=C1c2cc3c(cc2-c2c1ccc1c2[nH]c2ccccc21)[nH]c1ccccc13] | 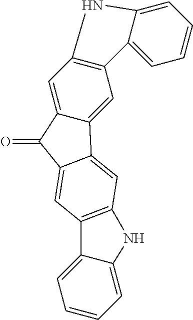 [SMILES: O=C1c2cc3c(cc2-c2cc4c(cc21)[nH]c1ccccc14)[nH]c1ccccc13] | 56%/26% |
| 5d | 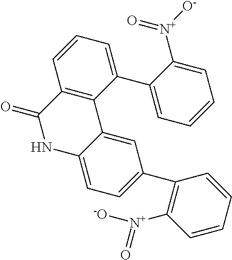 | 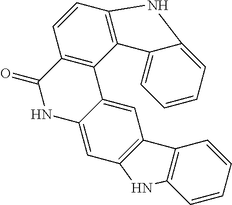 [SMILES: O=c1[nH]c2cc3[nH]c4ccccc4c3cc2c2c1ccc1[nH]c3ccccc3c12] | | 72% |
| 6d | 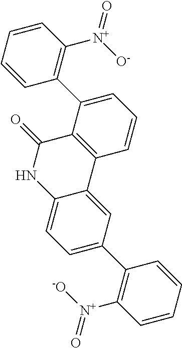 | 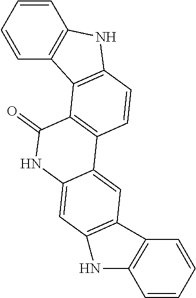 [SMILES: O=c1[nH]c2cc3[nH]c4ccccc4c3cc2c2ccc3[nH]c4ccccc4c3c12] | 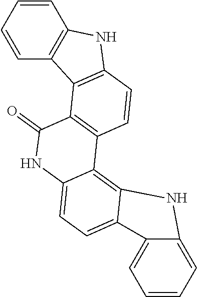 [SMILES: O=c1[nH]c2ccc3c4ccccc4[nH]c3c2c2ccc3[nH]c4ccccc4c3c12] | 54%/30% |
| 7d | 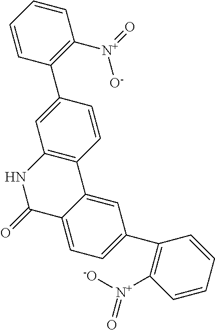 | 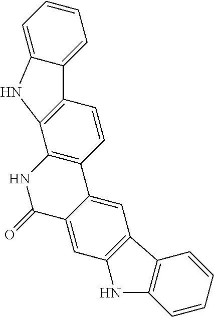 [SMILES: O=c1[nH]c2c(ccc3c4ccccc4[nH]c32)c2cc3c(cc12)[nH]c1ccccc13] | 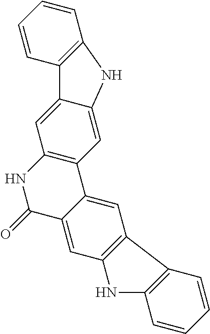 [SMILES: O=c1[nH]c2cc3c(cc2c2cc4c(cc12)[nH]c1ccccc14)[nH]c1ccccc13] | 52%/28% |
| 8d | 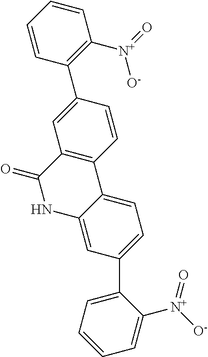 [SMILES: O=c1[nH]c2cc(-c3ccccc3[N+](=O)[O-])ccc2c2ccc(-c3ccccc3[N+](=O)[O-])cc12] | 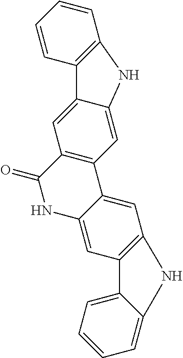 [SMILES: O=c1[nH]c2cc3c(cc2c2cc4[nH]c5ccccc5c4cc12)[nH]c1ccccc13] | 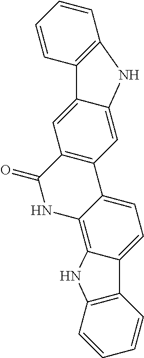 [SMILES: O=c1[nH]c2c(ccc3c4ccccc4[nH]c32)c2cc3[nH]c4ccccc4c3cc12] | 55%/14% |
| 9d | 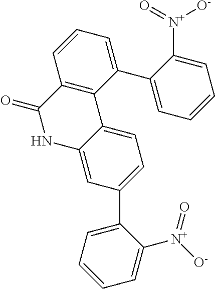 [SMILES: O=c1[nH]c2cc(-c3ccccc3[N+](=O)[O-])ccc2c2c(-c3ccccc3[N+](=O)[O-])cccc12] | 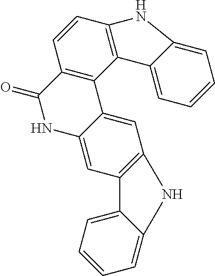 [SMILES: O=c1[nH]c2cc3c(cc2c2c1ccc1[nH]c4ccccc4c12)[nH]c1ccccc13] | | 64% |
| 10d | 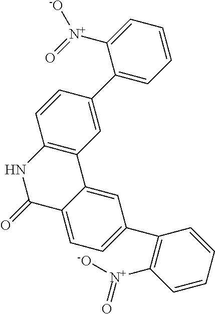 | 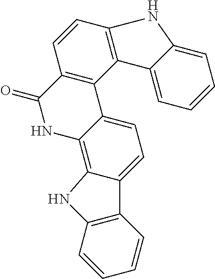 [SMILES: O=c1[nH]c2c(ccc3c4ccccc4[nH]c32)c2c1ccc1[nH]c3ccccc3c12] | | 67% |
| 11d | 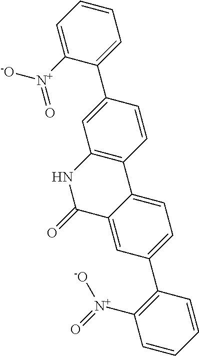 | 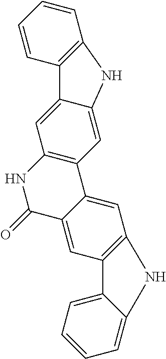 [SMILES: O=c1[nH]c2cc3c(cc2c2cc4[nH]c5ccccc5c4cc12)[nH]c1ccccc13] | 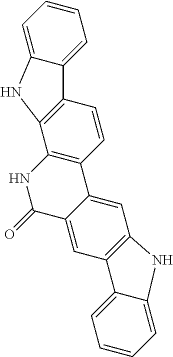 [SMILES: O=c1[nH]c2c(ccc3c4ccccc4[nH]c32)c2cc3[nH]c4ccccc4c3cc12] | 57%/21% |
| 12d | 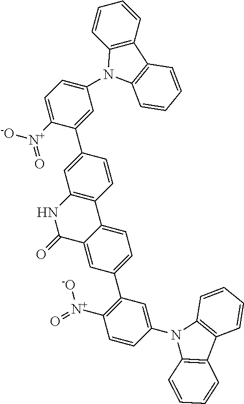 | 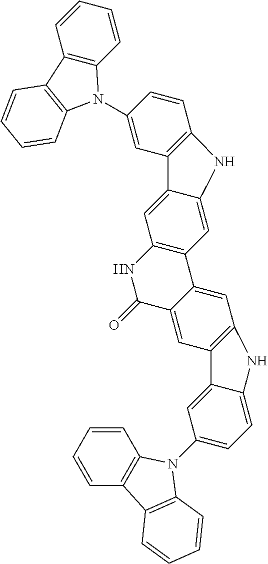 [SMILES: O=c1[nH]c2cc3c(cc2c2cc4[nH]c5ccc(-n6c7ccccc7c7ccccc76)cc5c4cc12)[nH]c1ccc(-n2c4ccccc4c4ccccc42)cc13] | 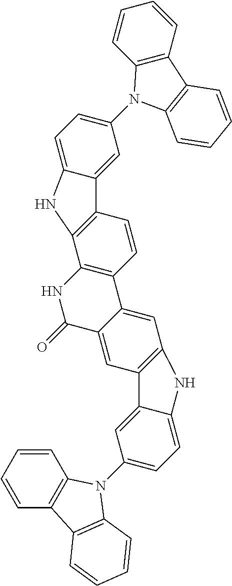 [SMILES: O=c1[nH]c2c(ccc3c4cc(-n5c6ccccc6c6ccccc65)ccc4[nH]c32)c2cc3[nH]c4ccc(-n5c6ccccc6c6ccccc65)cc4c3cc12] | 42%/24% |
| 13d | 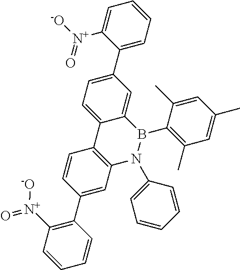 | 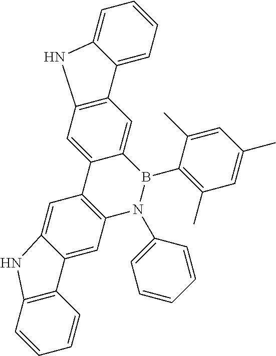 [SMILES: Cc1cc(C)c(B2c3cc4c(cc3-c3cc5[nH]c6ccccc6c5cc3N2c2ccccc2)[nH]c2ccccc24)c(C)c1] | 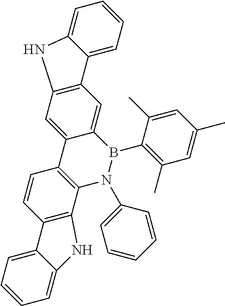 [SMILES: Cc1cc(C)c(B2c3cc4c(cc3-c3ccc5c([nH]c6ccccc65)c3N2c2ccccc2)[nH]c2ccccc24)c(C)c1] | 32%/12% |

### g) Ketoxime Synthesis

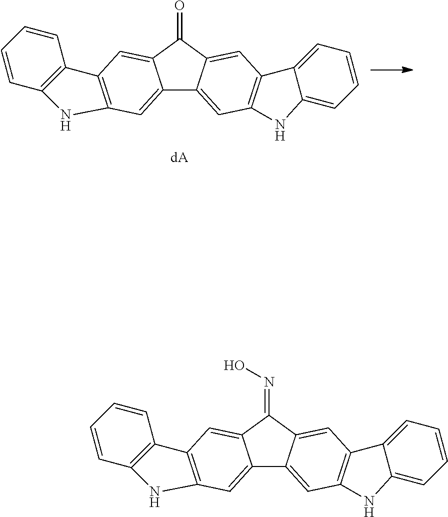 [SMILES: O=C1c2cc3c(cc2-c2cc4[nH]c5ccccc5c4cc21)[nH]c1ccccc13 ; "dA" ; ON=C1c2cc3c(cc2-c2cc4[nH]c5ccccc5c4cc21)[nH]c1ccccc13]

To an initial charge of 56 g (151 mmol) of compound (dA) in 300 ml pyridine/200 methanol is then added 20.5 g of hydroxylammonium chloride in portions, and then the mixture is heated at 60° C. for 3.5 hours.

After the reaction has ended, the precipitated solids are filtered off with suction and washed with water and 1 M HCl, and then with methanol.

The yield is 51 g (137 mmol), corresponding to 88% of theory.

The following compounds can be prepared analogously:

| | Reactant 1 | Product | Yield |
|---|---|---|---|
| 1g | 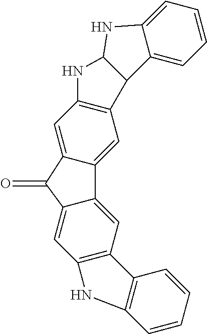 [SMILES: O=C1c2cc3c(cc2-c2cc4c(cc21)[nH]c1ccccc14)C1c2ccccc2NC1N3] | 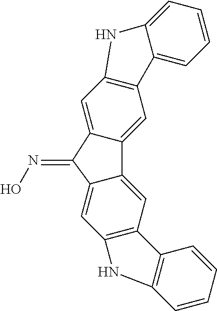 [SMILES: ON=C1c2cc3[nH]c4ccccc4c3cc2-c2cc3c(cc21)[nH]c1ccccc13] | 82% |
| 2g | 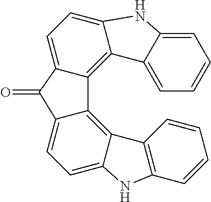 [SMILES: O=C1c2ccc3[nH]c4ccccc4c3c2-c2c1ccc1[nH]c3ccccc3c21] | 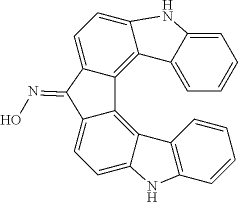 [SMILES: ON=C1c2ccc3[nH]c4ccccc4c3c2-c2c1ccc1[nH]c3ccccc3c21] | 82% |
| 3g | 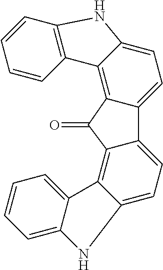 [SMILES: O=C1c2c(ccc3[nH]c4ccccc4c23)-c2ccc3[nH]c4ccccc4c3c21] | 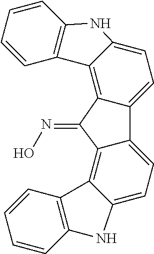 [SMILES: ON=C1c2c(ccc3[nH]c4ccccc4c23)-c2ccc3[nH]c4ccccc4c3c21] | 77% |

### h) Lactam Synthesis (Beckmann Rearrangement)

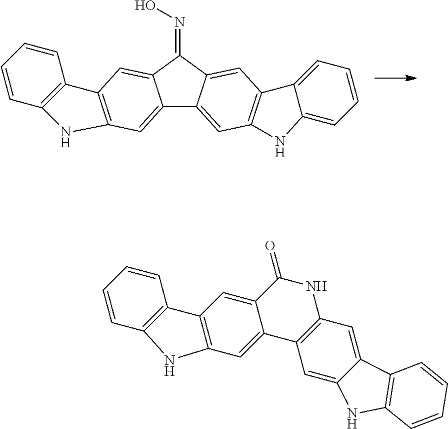 [SMILES: ON=C1c2cc3c(cc2-c2cc4[nH]c5ccccc5c4cc21)[nH]c1ccccc13 ; O=c1[nH]c2cc3c(cc2c2cc4[nH]c5ccccc5c4cc12)[nH]c1ccccc13]

An initial charge of 48.5 g (130 mmol) of compound (g) in 300 ml of polyphosphoric acid is ultimately heated to 170° C. for 12 hours. After the reaction has ended, the mixture is added to ice, extracted with ethyl acetate, separated and concentrated. The precipitated solids are filtered off with suction and washed with ethanol. The isomers are separated by chromatography.

The yield is 44.6 g (119 mmol), corresponding to 89% of theory.

The following compounds are prepared in an analogous manner:

| | Reactant 1 | Product (a) | Yield |
|---|---|---|---|
| 1h | 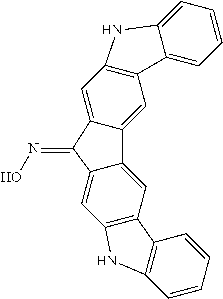 [SMILES: ON=C1c2cc3[nH]c4ccccc4c3cc2-c2cc3c(cc21)[nH]c1ccccc13] | 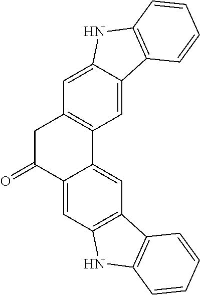 [SMILES: O=C1Cc2cc3[nH]c4ccccc4c3cc2-c2cc3c(cc21)[nH]c1ccccc13] | 66% |
| 2h | 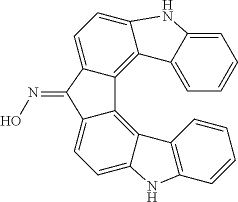 [SMILES: ON=C1c2ccc3[nH]c4ccccc4c3c2-c2c1ccc1[nH]c3ccccc3c21] | 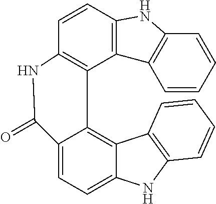 [SMILES: O=c1[nH]c2ccc3[nH]c4ccccc4c3c2c2c1ccc1[nH]c3ccccc3c12] | 68% |
| 3h | 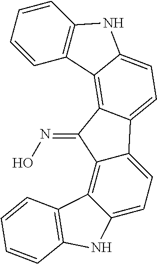 [SMILES: ON=C1c2c(ccc3[nH]c4ccccc4c23)-c2ccc3[nH]c4ccccc4c3c21] | 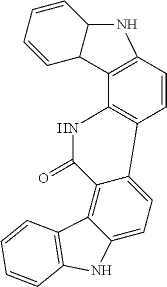 [SMILES: O=c1[nH]c2c3c(ccc2c2ccc4[nH]c5ccccc5c4c12)NC1C=CC=CC31] | 71% |

### i) Buchwald Reaction

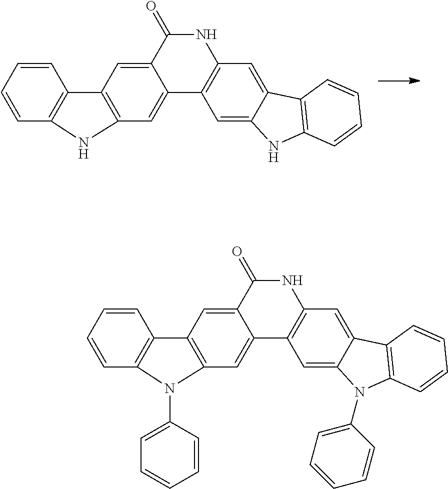 [SMILES: O=c1[nH]c2cc3c(cc2c2cc4[nH]c5ccccc5c4cc12)[nH]c1ccccc13 ; O=c1[nH]c2cc3c4ccccc4n(-c4ccccc4)c3cc2c2cc3c(cc12)c1ccccc1n3-c1ccccc1]

8.9 g (24 mmol, 1.00 eq) of compound (h) and 7.8 g (50 mmol, 2.00 eq) of bromobenzene are dissolved in 400 ml of toluene under an argon atmosphere. 1.0 g (5 mmol) of tri-tert-butylphosphine is added and the mixture is stirred under an argon atmosphere. 0.6 g (2 mmol) of Pd(OAc)₂ is added and the mixture is stirred under an argon atmosphere, and then 9.5 g (99 mmol) of sodium tert-butoxide are added. The reaction mixture is stirred under reflux for 24 h. After cooling, the organic phase is separated, washed three times with 200 ml of water, dried over MgSO₄ and filtered, and the solvent is removed under reduced pressure. The residue is purified by column chromatography using silica gel (eluent: DCM/heptane (1:3)). The residue is subjected to hot extraction with toluene and recrystallized from toluene/n-heptane and finally sublimed under high vacuum.

The yield is 10.4 g (19.8 mmol), corresponding to 83% of theory.

The following compounds are prepared in an analogous manner:

| | Reactant 1 | Reactant 2 | Product | Yield |
|---|---|---|---|---|
| 1i | 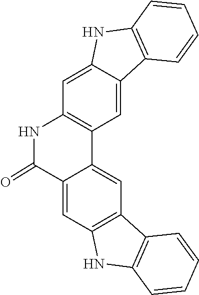 [SMILES: O=c1[nH]c2cc3[nH]c4ccccc4c3cc2c2cc3c(cc12)[nH]c1ccccc13] | 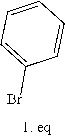 | 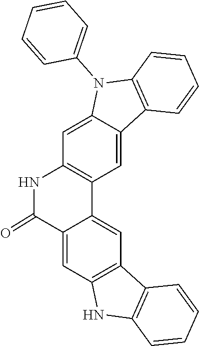 [SMILES: O=c1[nH]c2cc3c(cc2c2cc4c(cc12)[nH]c1ccccc14)c1ccccc1n3-c1ccccc1] | 76% |
| 2i | 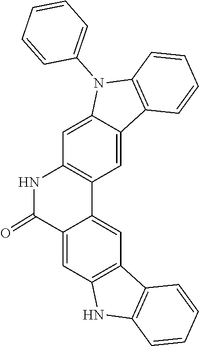 [SMILES: O=c1[nH]c2cc3c(cc2c2cc4c(cc12)[nH]c1ccccc14)c1ccccc1n3-c1ccccc1] | 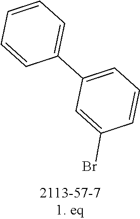 [SMILES: Brc1cccc(-c2ccccc2)c1] | 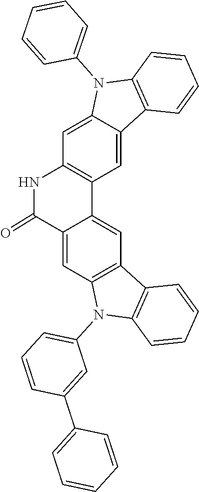 [SMILES: O=c1[nH]c2cc3c(cc2c2cc4c5ccccc5n(-c5cccc(-c6ccccc6)c5)c4cc12)c1ccccc1n3-c1ccccc1] | 73% |
| 3i | 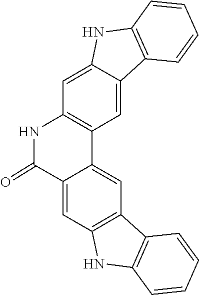 [SMILES: O=c1[nH]c2cc3[nH]c4ccccc4c3cc2c2cc3c(cc12)[nH]c1ccccc13] | 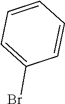 | 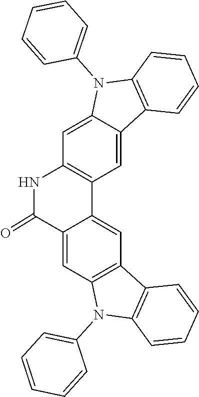 [SMILES: O=c1[nH]c2cc3c(cc2c2cc4c5ccccc5n(-c5ccccc5)c4cc12)c1ccccc1n3-c1ccccc1] | 76% |
| 4i | 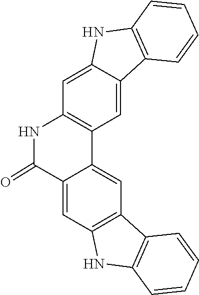 [SMILES: O=c1[nH]c2cc3[nH]c4ccccc4c3cc2c2cc3c(cc12)[nH]c1ccccc13] | 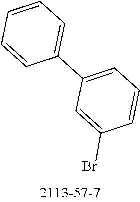 [SMILES: Brc1cccc(-c2ccccc2)c1] | 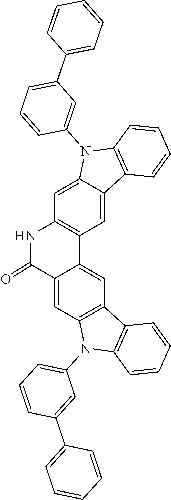 [SMILES: O=c1[nH]c2cc3c(cc2c2cc4c5ccccc5n(-c5cccc(-c6ccccc6)c5)c4cc12)c1ccccc1n3-c1cccc(-c2ccccc2)c1] | 70% |
| 5i | 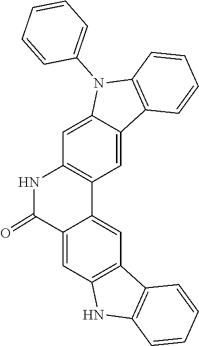 [SMILES: O=c1[nH]c2cc3c(cc2c2cc4c(cc12)[nH]c1ccccc14)c1ccccc1n3-c1ccccc1] | 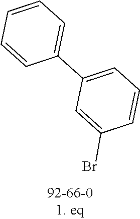 [SMILES: Brc1cccc(-c2ccccc2)c1] | 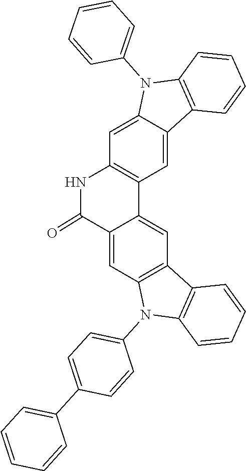 [SMILES: O=c1[nH]c2cc3c(cc2c2cc4c5ccccc5n(-c5ccc(-c6ccccc6)cc5)c4cc12)c1ccccc1n3-c1ccccc1] | 80% |
| 6i | 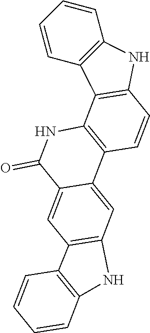 [SMILES: O=c1[nH]c2c(ccc3[nH]c4ccccc4c32)c2cc3[nH]c4ccccc4c3cc12] | 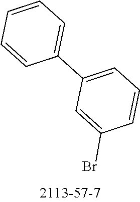 [SMILES: Brc1cccc(-c2ccccc2)c1] | 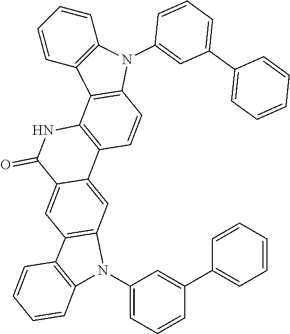 [SMILES: O=c1[nH]c2c(ccc3c2c2ccccc2n3-c2cccc(-c3ccccc3)c2)c2cc3c(cc12)c1ccccc1n3-c1cccc(-c2ccccc2)c1] | 80% |
| 7i | 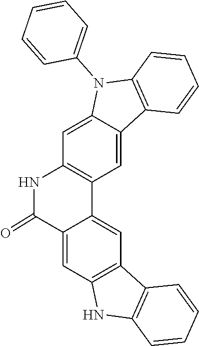 [SMILES: O=c1[nH]c2cc3c(cc2c2cc4c(cc12)[nH]c1ccccc14)c1ccccc1n3-c1ccccc1] | 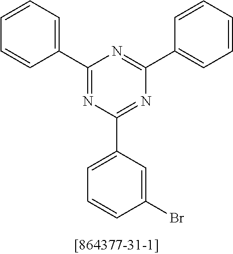 [SMILES: Brc1cccc(-c2nc(-c3ccccc3)nc(-c3ccccc3)n2)c1] | 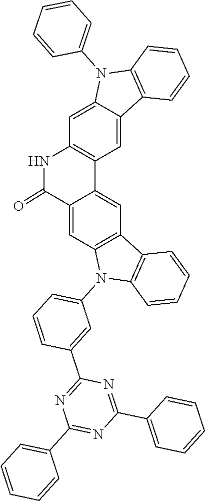 [SMILES: O=c1[nH]c2cc3c(cc2c2cc4c5ccccc5n(-c5cccc(-c6nc(-c7ccccc7)nc(-c7ccccc7)n6)c5)c4cc12)c1ccccc1n3-c1ccccc1] | 76% |
| 8i | 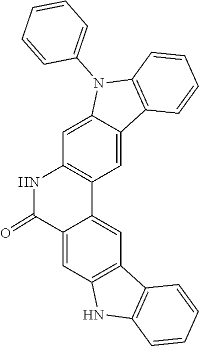 [SMILES: O=c1[nH]c2cc3c(cc2c2cc4c(cc12)[nH]c1ccccc14)c1ccccc1n3-c1ccccc1] | 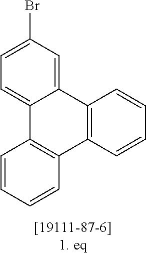 [SMILES: Brc1ccc2c3ccccc3c3ccccc3c2c1] | 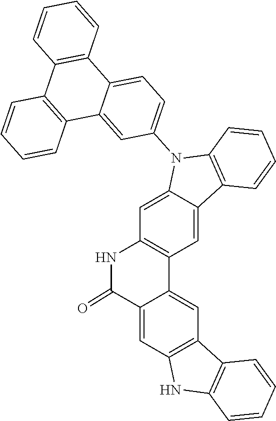 [SMILES: O=c1[nH]c2cc3c(cc2c2cc4c(cc12)[nH]c1ccccc14)c1ccccc1n3-c1ccc2c3ccccc3c3ccccc3c2c1] | 68% |
| 9i | 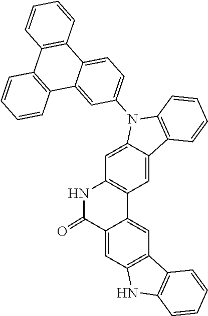 [SMILES: O=c1[nH]c2cc3c(cc2c2cc4c(cc12)[nH]c1ccccc14)c1ccccc1n3-c1ccc2c3ccccc3c3ccccc3c2c1] | 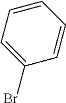 | 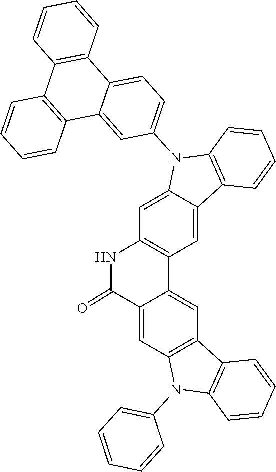 [SMILES: O=c1[nH]c2cc3c(cc2c2cc4c5ccccc5n(-c5ccccc5)c4cc12)c1ccccc1n3-c1ccc2c3ccccc3c3ccccc3c2c1] | 72% |
| 10i | 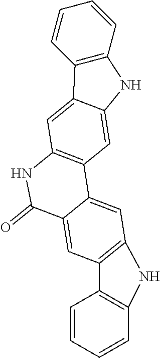 [SMILES: O=c1[nH]c2cc3c(cc2c2cc4[nH]c5ccccc5c4cc12)[nH]c1ccccc13] | 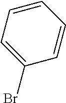 | 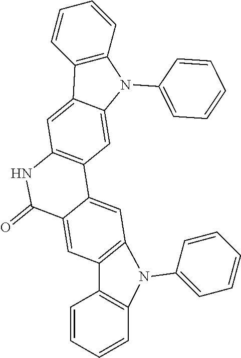 [SMILES: O=c1[nH]c2cc3c4ccccc4n(-c4ccccc4)c3cc2c2cc3c(cc12)c1ccccc1n3-c1ccccc1] | 71% |
| 11i | 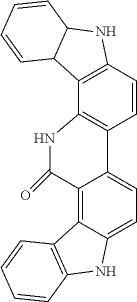 [SMILES: O=c1[nH]c2c3c(ccc2c2ccc4[nH]c5ccccc5c4c12)NC1C=CC=CC31] | 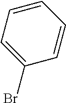 | 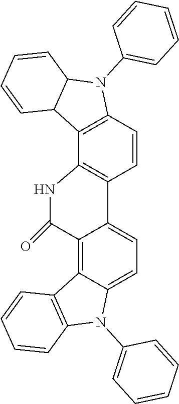 [SMILES: O=c1[nH]c2c3c(ccc2c2ccc4c(c5ccccc5n4-c4ccccc4)c12)N(c1ccccc1)C1C=CC=CC31] | 65% |
| 12i | 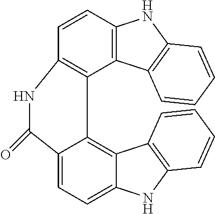 [SMILES: O=c1[nH]c2ccc3[nH]c4ccccc4c3c2c2c1ccc1[nH]c3ccccc3c12] | 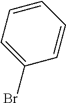 | 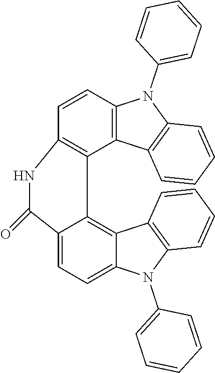 [SMILES: O=c1[nH]c2ccc3c(c4ccccc4n3-c3ccccc3)c2c2c1ccc1c2c2ccccc2n1-c1ccccc1] | 63% |
| 13i | 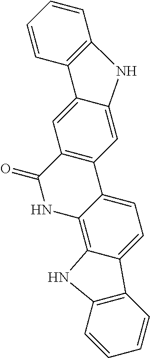 [SMILES: O=c1[nH]c2c(ccc3c4ccccc4[nH]c32)c2cc3[nH]c4ccccc4c3cc12] | 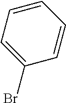 | 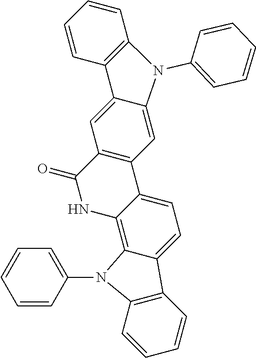 [SMILES: O=c1[nH]c2c(ccc3c4ccccc4n(-c4ccccc4)c32)c2cc3c(cc12)c1ccccc1n3-c1ccccc1] | 61% |
| 14i | 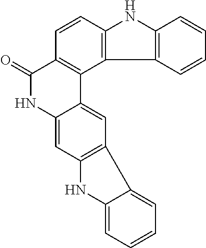 [SMILES: O=c1[nH]c2cc3[nH]c4ccccc4c3cc2c2c1ccc1[nH]c3ccccc3c12] | 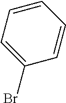 | 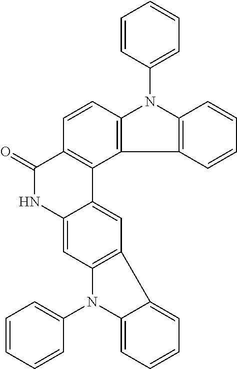 [SMILES: O=c1[nH]c2cc3c(cc2c2c1ccc1c2c2ccccc2n1-c1ccccc1)c1ccccc1n3-c1ccccc1] | 69% |
| 15i | 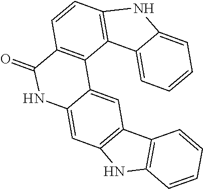 [SMILES: O=c1[nH]c2cc3[nH]c4ccccc4c3cc2c2c1ccc1[nH]c3ccccc3c12] | 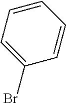 | 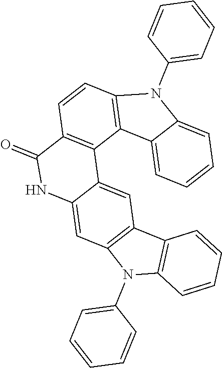 [SMILES: O=c1[nH]c2cc3c(cc2c2c1ccc1c2c2ccccc2n1-c1ccccc1)c1ccccc1n3-c1ccccc1] | |
| 16i | 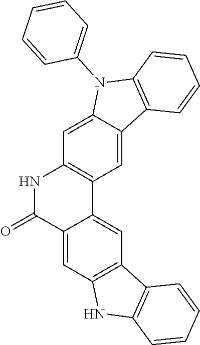 [SMILES: O=c1[nH]c2cc3c(cc2c2cc4c(cc12)[nH]c1ccccc14)c1ccccc1n3-c1ccccc1] | 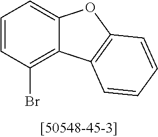 [SMILES: Brc1cccc2oc3ccccc3c12] | 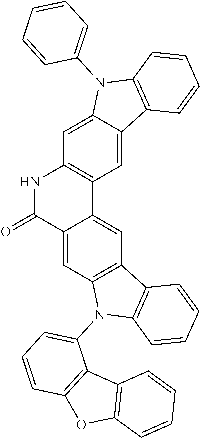 [SMILES: O=c1[nH]c2cc3c(cc2c2cc4c5ccccc5n(-c5cccc6oc7ccccc7c56)c4cc12)c1ccccc1n3-c1ccccc1] | |
| 17i | 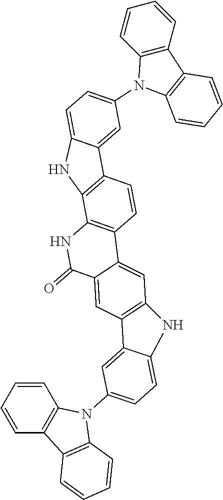 [SMILES: O=c1[nH]c2c(ccc3c4cc(-n5c6ccccc6c6ccccc65)ccc4[nH]c32)c2cc3[nH]c4ccc(-n5c6ccccc6c6ccccc65)cc4c3cc12] | 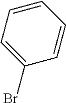 | 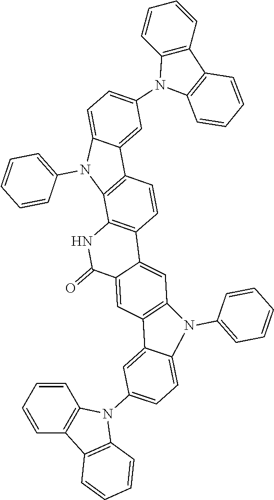 [SMILES: O=c1[nH]c2c(ccc3c4cc(-n5c6ccccc6c6ccccc65)ccc4n(-c4ccccc4)c32)c2cc3c(cc12)c1cc(-n2c4ccccc4c4ccccc42)ccc1n3-c1ccccc1] | |
| 18i | 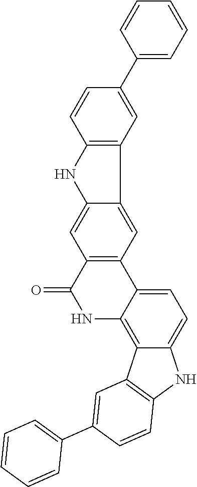 [SMILES: O=c1[nH]c2c(ccc3[nH]c4ccc(-c5ccccc5)cc4c32)c2cc3c(cc12)[nH]c1ccc(-c2ccccc2)cc13] | 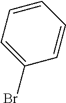 | 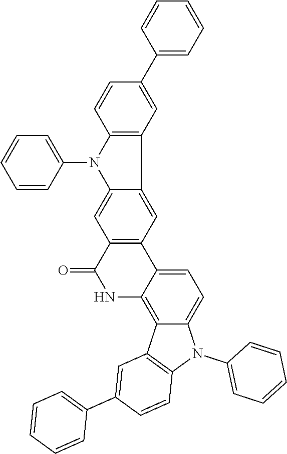 [SMILES: O=c1[nH]c2c(ccc3c2c2cc(-c4ccccc4)ccc2n3-c2ccccc2)c2cc3c4cc(-c5ccccc5)ccc4n(-c4ccccc4)c3cc12] | 66% |
| 19i | 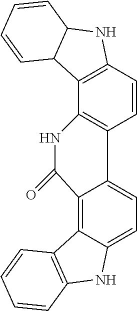 [SMILES: O=c1[nH]c2c3c(ccc2c2ccc4[nH]c5ccccc5c4c12)NC1C=CC=CC31] | 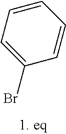 | 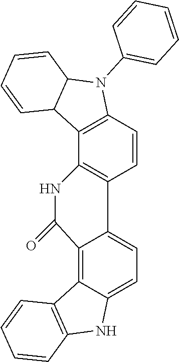 [SMILES: O=c1[nH]c2c3c(ccc2c2ccc4[nH]c5ccccc5c4c12)N(c1ccccc1)C1C=CC=CC31] | 60% |
| 20i | 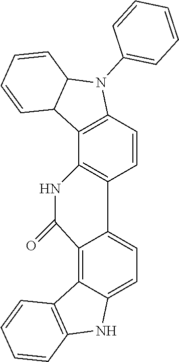 [SMILES: O=c1[nH]c2c3c(ccc2c2ccc4[nH]c5ccccc5c4c12)N(c1ccccc1)C1C=CC=CC31] | 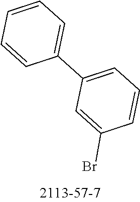 [SMILES: Brc1cccc(-c2ccccc2)c1] | 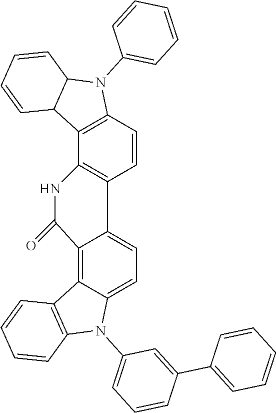 [SMILES: O=c1[nH]c2c3c(ccc2c2ccc4c(c5ccccc5n4-c4cccc(-c5ccccc5)c4)c12)N(c1ccccc1)C1C=CC=CC31] | 76% |
| 21i | 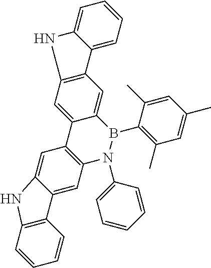 [SMILES: Cc1cc(C)c(B2c3cc4c(cc3-c3cc5[nH]c6ccccc6c5cc3N2c2ccccc2)[nH]c2ccccc24)c(C)c1] | 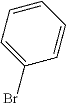 | 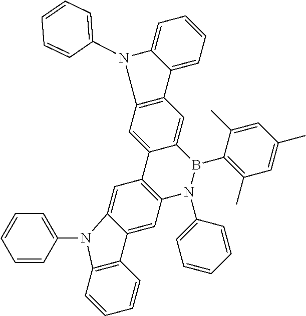 [SMILES: Cc1cc(C)c(B2c3cc4c5ccccc5n(-c5ccccc5)c4cc3-c3cc4c(cc3N2c2ccccc2)c2ccccc2n4-c2ccccc2)c(C)c1] | 62% |
| 22i | 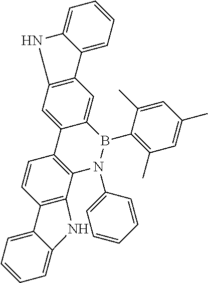 [SMILES: Cc1cc(C)c(B2c3cc4c(cc3-c3ccc5c([nH]c6ccccc65)c3N2c2ccccc2)[nH]c2ccccc24)c(C)c1] | 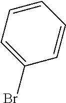 | 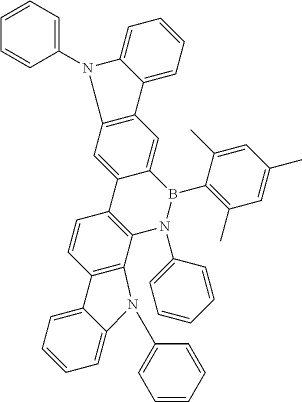 [SMILES: Cc1cc(C)c(B2c3cc4c5ccccc5n(-c5ccccc5)c4cc3-c3ccc4c5ccccc5n(-c5ccccc5)c4c3N2c2ccccc2)c(C)c1] | 56% |
| 23i | 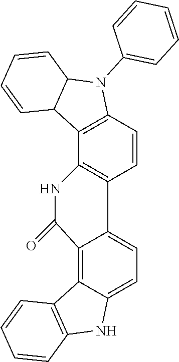 [SMILES: O=c1[nH]c2c3c(ccc2c2ccc4[nH]c5ccccc5c4c12)N(c1ccccc1)C1C=CC=CC31] | 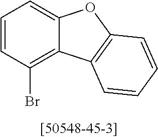 [SMILES: Brc1cccc2oc3ccccc3c12] | 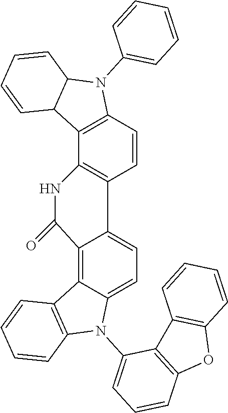 [SMILES: O=c1[nH]c2c3c(ccc2c2ccc4c(c5ccccc5n4-c4cccc5oc6ccccc6c45)c12)N(c1ccccc1)C1C=CC=CC31] | 59% |
| 24i | 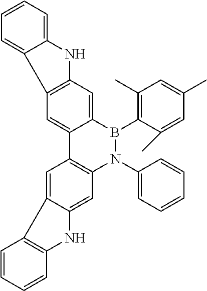 [SMILES: Cc1cc(C)c(B2c3cc4[nH]c5ccccc5c4cc3-c3cc4c(cc3N2c2ccccc2)[nH]c2ccccc24)c(C)c1] | 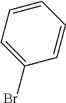 | 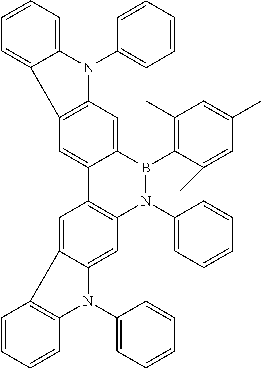 [SMILES: Cc1cc(C)c(B2c3cc4c(cc3-c3cc5c6ccccc6n(-c6ccccc6)c5cc3N2c2ccccc2)c2ccccc2n4-c2ccccc2)c(C)c1] | 47% |
| 25i | 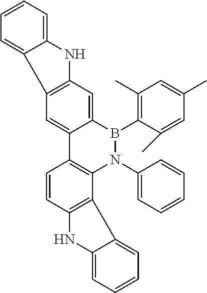 [SMILES: Cc1cc(C)c(B2c3cc4[nH]c5ccccc5c4cc3-c3ccc4[nH]c5ccccc5c4c3N2c2ccccc2)c(C)c1] | 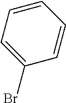 | 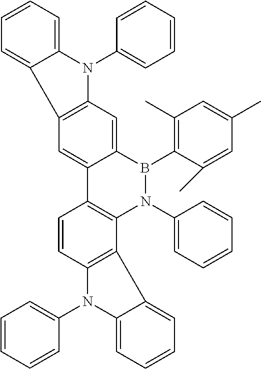 [SMILES: Cc1cc(C)c(B2c3cc4c(cc3-c3ccc5c(c3N2c2ccccc2)c2ccccc2n5-c2ccccc2)c2ccccc2n4-c2ccccc2)c(C)c1] | 45% |

### j) Ullmann Reaction

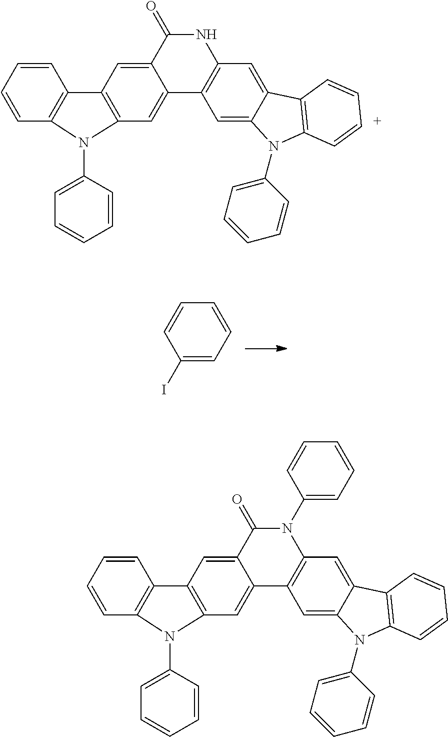 [SMILES: O=c1[nH]c2cc3c4ccccc4n(-c4ccccc4)c3cc2c2cc3c(cc12)c1ccccc1n3-c1ccccc1 ; Ic1ccccc1 ; O=c1c2cc3c4ccccc4n(-c4ccccc4)c3cc2c2cc3c(cc2n1-c1ccccc1)c1ccccc1n3-c1ccccc1]

An initial charge of 26.2 g (50 mmol, 1.00 eq.) of compound (i), 22.6 ml (256 mmol, 5.2 eq.) of iodobenzene and 14.4 g of potassium carbonate (104.2 mmol, 2.10 eq.) of potassium carbonate in 440 ml of dried DMF is inertized under argon. Subsequently, 1.24 g (5.4 mmol, 0.11 eq) of 1,3-di(2-pyridyl)propane-1,3-dione and 104 g (5.4 mmol, 0.11 eq) of copper(I) iodide are added and the mixture is heated at 140° C. for three days. After the reaction has ended, the mixture is concentrated cautiously on a rotary evaporator, and the precipitated solids are filtered off with suction and washed with water and ethanol. The crude product is purified twice by means of a hot extractor (toluene/heptane 1:1), and the solids obtained are recrystallized from toluene. After sublimation, 18.5 g (31 mmol, 62%) of the desired target compound is obtained.

The following compounds can be prepared analogously:

| Ex. | Reactant 1 | Reactant 1 | Product | Yield |
|---|---|---|---|---|
| 1j | 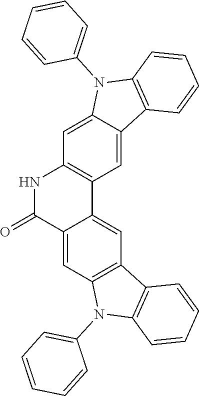 [SMILES: O=c1[nH]c2cc3c(cc2c2cc4c5ccccc5n(-c5ccccc5)c4cc12)c1ccccc1n3-c1ccccc1] | 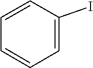 | 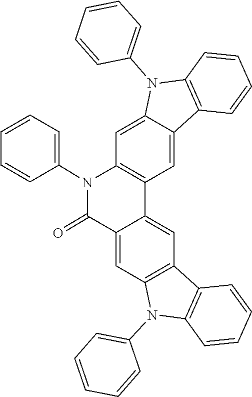 [SMILES: O=c1c2cc3c(cc2c2cc4c5ccccc5n(-c5ccccc5)c4cc2n1-c1ccccc1)c1ccccc1n3-c1ccccc1] | 77% |
| 2j | 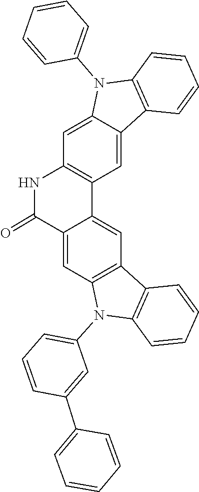 [SMILES: O=c1[nH]c2cc3c(cc2c2cc4c5ccccc5n(-c5cccc(-c6ccccc6)c5)c4cc12)c1ccccc1n3-c1ccccc1] | 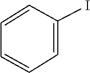 | 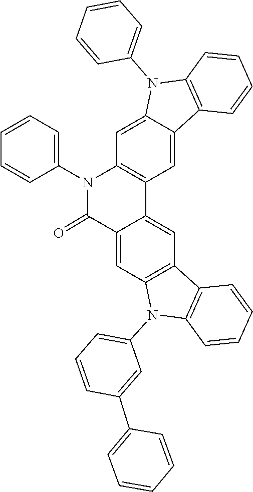 [SMILES: O=c1c2cc3c(cc2c2cc4c5ccccc5n(-c5ccccc5)c4cc2n1-c1ccccc1)c1ccccc1n3-c1cccc(-c2ccccc2)c1] | 64% |
| 3j | 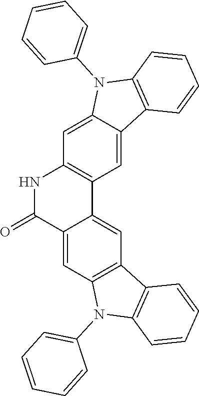 [SMILES: O=c1[nH]c2cc3c(cc2c2cc4c5ccccc5n(-c5ccccc5)c4cc12)c1ccccc1n3-c1ccccc1] | 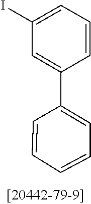 [SMILES: Ic1cccc(-c2ccccc2)c1] | 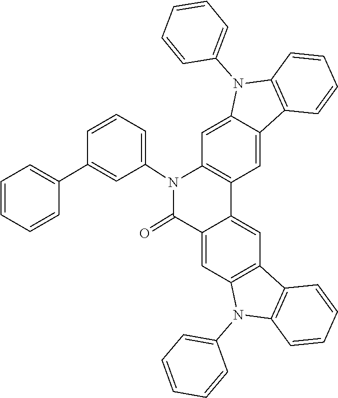 [SMILES: O=c1c2cc3c(cc2c2cc4c5ccccc5n(-c5ccccc5)c4cc2n1-c1cccc(-c2ccccc2)c1)c1ccccc1n3-c1ccccc1] | 61% |
| 4j | 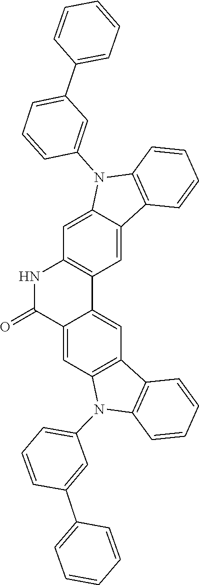 [SMILES: O=c1[nH]c2cc3c(cc2c2cc4c5ccccc5n(-c5cccc(-c6ccccc6)c5)c4cc12)c1ccccc1n3-c1cccc(-c2ccccc2)c1] | 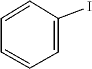 | 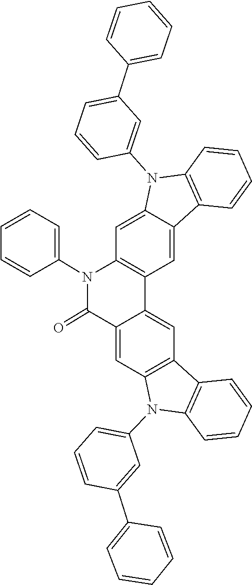 [SMILES: O=c1c2cc3c(cc2c2cc4c5ccccc5n(-c5cccc(-c6ccccc6)c5)c4cc2n1-c1ccccc1)c1ccccc1n3-c1cccc(-c2ccccc2)c1] | 55% |
| 5j | 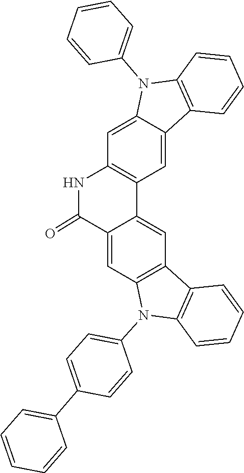 [SMILES: O=c1[nH]c2cc3c(cc2c2cc4c5ccccc5n(-c5ccc(-c6ccccc6)cc5)c4cc12)c1ccccc1n3-c1ccccc1] | 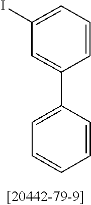 [SMILES: Ic1cccc(-c2ccccc2)c1] | 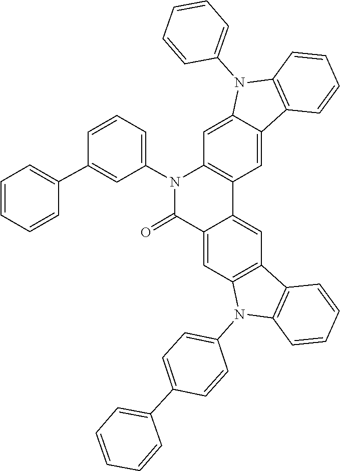 [SMILES: O=c1c2cc3c(cc2c2cc4c5ccccc5n(-c5ccccc5)c4cc2n1-c1cccc(-c2ccccc2)c1)c1ccccc1n3-c1ccc(-c2ccccc2)cc1] | 63% |
| 6j | 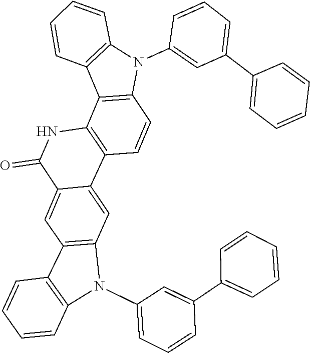 [SMILES: O=c1[nH]c2c(ccc3c2c2ccccc2n3-c2cccc(-c3ccccc3)c2)c2cc3c(cc12)c1ccccc1n3-c1cccc(-c2ccccc2)c1] | 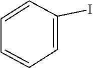 | 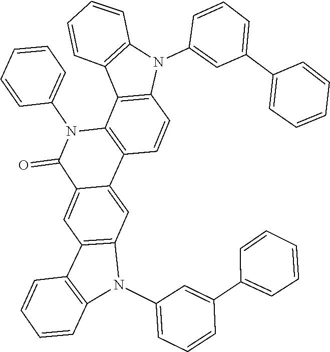 [SMILES: O=c1c2cc3c4ccccc4n(-c4cccc(-c5ccccc5)c4)c3cc2c2ccc3c(c4ccccc4n3-c3cccc(-c4ccccc4)c3)c2n1-c1ccccc1] | 66% |
| 7j | 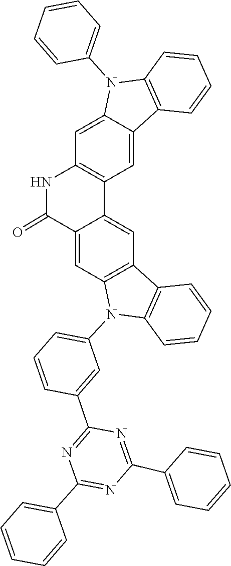 [SMILES: O=c1[nH]c2cc3c(cc2c2cc4c5ccccc5n(-c5cccc(-c6nc(-c7ccccc7)nc(-c7ccccc7)n6)c5)c4cc12)c1ccccc1n3-c1ccccc1] | 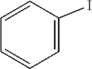 | 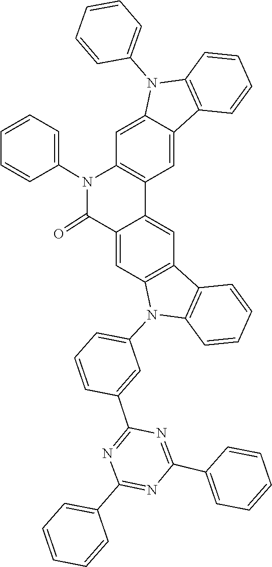 [SMILES: O=c1c2cc3c(cc2c2cc4c5ccccc5n(-c5ccccc5)c4cc2n1-c1ccccc1)c1ccccc1n3-c1cccc(-c2nc(-c3ccccc3)nc(-c3ccccc3)n2)c1] | 73% |
| 8j | 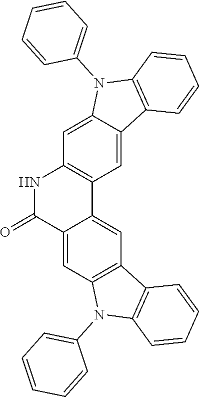 [SMILES: O=c1[nH]c2cc3c(cc2c2cc4c5ccccc5n(-c5ccccc5)c4cc12)c1ccccc1n3-c1ccccc1] | 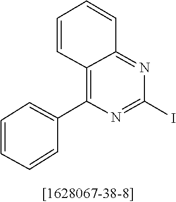 [SMILES: Ic1nc(-c2ccccc2)c2ccccc2n1] | 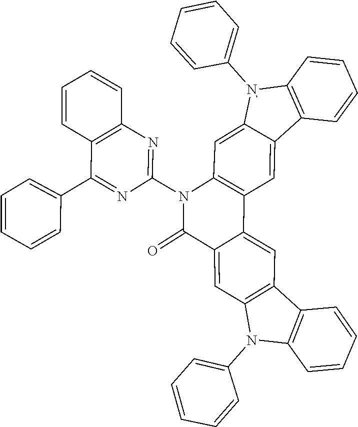 | 65% |
| 9j | 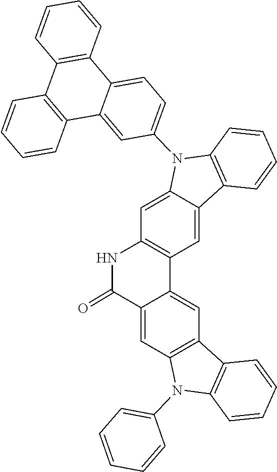 [SMILES: O=c1[nH]c2cc3c(cc2c2cc4c5ccccc5n(-c5ccccc5)c4cc12)c1ccccc1n3-c1ccc2c3ccccc3c3ccccc3c2c1] | 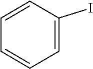 | 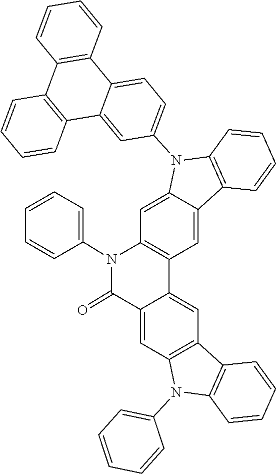 [SMILES: O=c1c2cc3c(cc2c2cc4c5ccccc5n(-c5ccc6c7ccccc7c7ccccc7c6c5)c4cc2n1-c1ccccc1)c1ccccc1n3-c1ccccc1] | 66% |
| 10j | 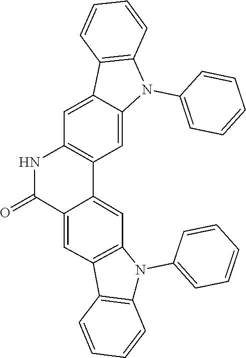 [SMILES: O=c1[nH]c2cc3c4ccccc4n(-c4ccccc4)c3cc2c2cc3c(cc12)c1ccccc1n3-c1ccccc1] | 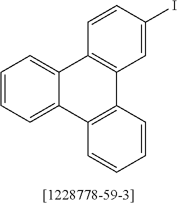 [SMILES: Ic1ccc2c3ccccc3c3ccccc3c2c1] | 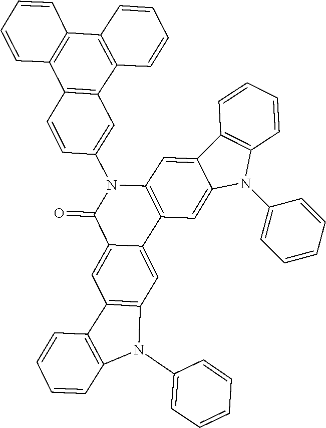 [SMILES: O=c1c2cc3c4ccccc4n(-c4ccccc4)c3cc2c2cc3c(cc2n1-c1ccc2c4ccccc4c4ccccc4c2c1)c1ccccc1n3-c1ccccc1] | 68% |
| 11j | 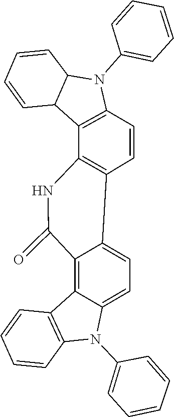 [SMILES: O=c1[nH]c2c3c(ccc2c2ccc4c(c5ccccc5n4-c4ccccc4)c12)N(c1ccccc1)C1C=CC=CC31] | 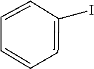 | 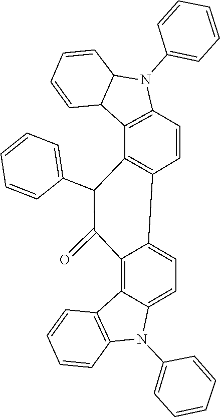 [SMILES: O=C1c2c(ccc3c2c2ccccc2n3-c2ccccc2)-c2ccc3c(c2C1c1ccccc1)C1C=CC=CC1N3c1ccccc1] | 64% |
| 12j | 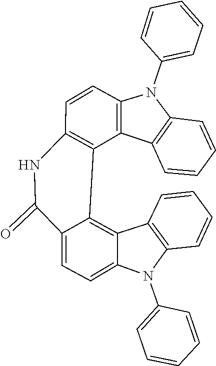 [SMILES: O=c1[nH]c2ccc3c(c4ccccc4n3-c3ccccc3)c2c2c1ccc1c2c2ccccc2n1-c1ccccc1] | 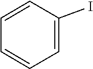 | 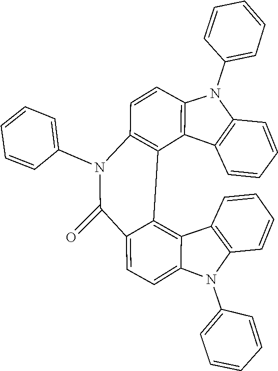 [SMILES: O=c1c2ccc3c(c4ccccc4n3-c3ccccc3)c2c2c3c4ccccc4n(-c4ccccc4)c3ccc2n1-c1ccccc1] | 67% |
| 13j | 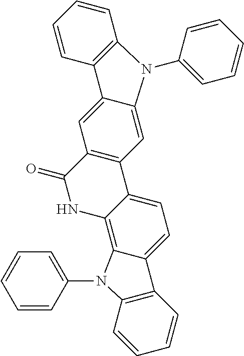 [SMILES: O=c1[nH]c2c(ccc3c4ccccc4n(-c4ccccc4)c32)c2cc3c(cc12)c1ccccc1n3-c1ccccc1] | 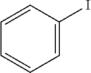 | 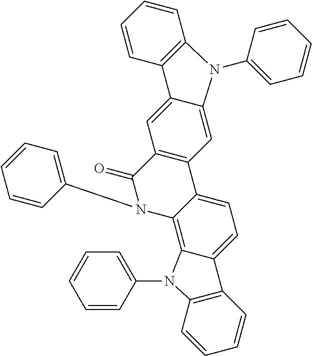 [SMILES: O=c1c2cc3c4ccccc4n(-c4ccccc4)c3cc2c2ccc3c4ccccc4n(-c4ccccc4)c3c2n1-c1ccccc1] | 44% |
| 14j | 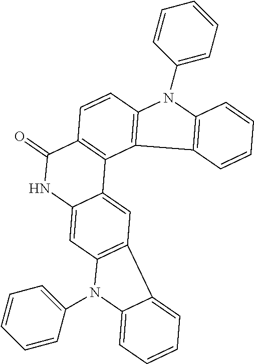 [SMILES: O=c1[nH]c2cc3c(cc2c2c1ccc1c2c2ccccc2n1-c1ccccc1)c1ccccc1n3-c1ccccc1] | 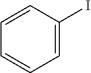 | 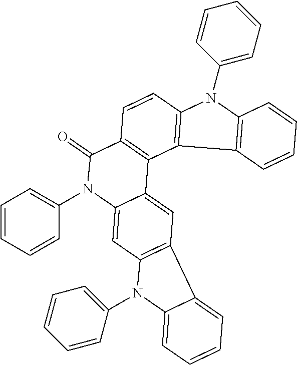 [SMILES: O=c1c2ccc3c(c4ccccc4n3-c3ccccc3)c2c2cc3c4ccccc4n(-c4ccccc4)c3cc2n1-c1ccccc1] | 71% |
| 15j | 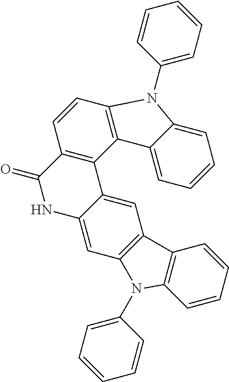 [SMILES: O=c1[nH]c2cc3c(cc2c2c1ccc1c2c2ccccc2n1-c1ccccc1)c1ccccc1n3-c1ccccc1] | 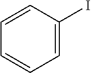 | 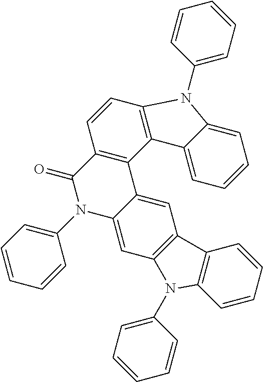 [SMILES: O=c1c2ccc3c(c4ccccc4n3-c3ccccc3)c2c2cc3c4ccccc4n(-c4ccccc4)c3cc2n1-c1ccccc1] | 70% |
| 16j | 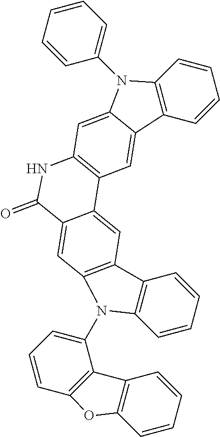 [SMILES: O=c1[nH]c2cc3c(cc2c2cc4c5ccccc5n(-c5cccc6oc7ccccc7c56)c4cc12)c1ccccc1n3-c1ccccc1] | 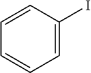 | 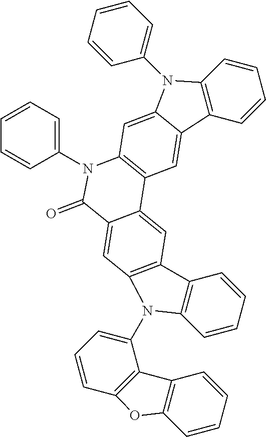 [SMILES: O=c1c2cc3c(cc2c2cc4c5ccccc5n(-c5ccccc5)c4cc2n1-c1ccccc1)c1ccccc1n3-c1cccc2oc3ccccc3c12] | 52% |
| 17j | 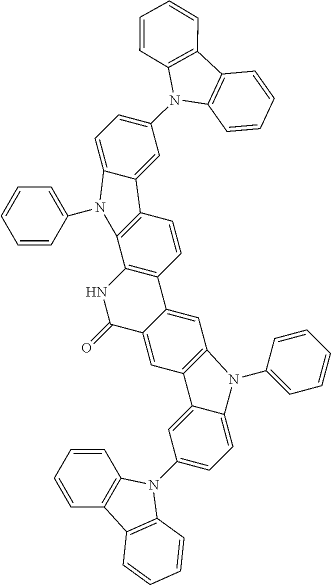 [SMILES: O=c1[nH]c2c(ccc3c4cc(-n5c6ccccc6c6ccccc65)ccc4n(-c4ccccc4)c32)c2cc3c(cc12)c1cc(-n2c4ccccc4c4ccccc42)ccc1n3-c1ccccc1] | 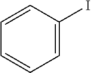 | 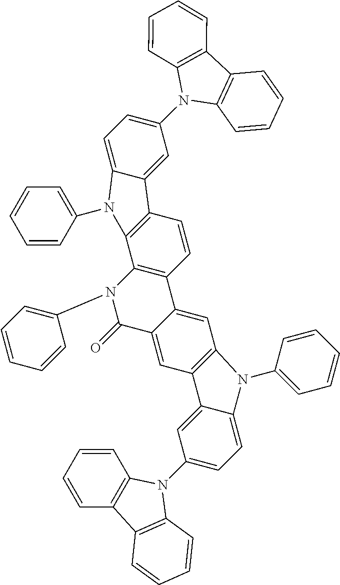 [SMILES: O=c1c2cc3c4cc(-n5c6ccccc6c6ccccc65)ccc4n(-c4ccccc4)c3cc2c2ccc3c4cc(-n5c6ccccc6c6ccccc65)ccc4n(-c4ccccc4)c3c2n1-c1ccccc1] | 41% |
| 18j | 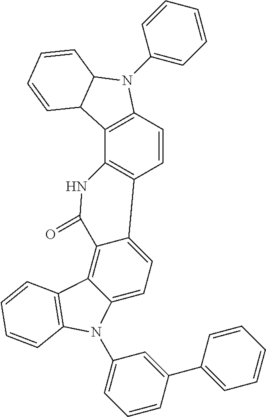 [SMILES: O=c1[nH]c2c3c(ccc2c2ccc4c(c5ccccc5n4-c4cccc(-c5ccccc5)c4)c12)N(c1ccccc1)C1C=CC=CC31] | 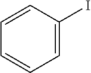 | 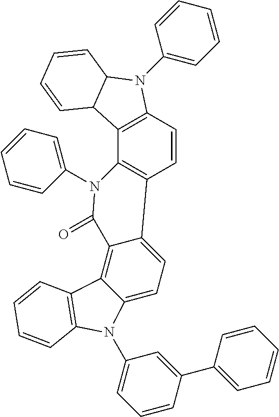 [SMILES: O=c1c2c(ccc3c2c2ccccc2n3-c2cccc(-c3ccccc3)c2)c2ccc3c(c2n1-c1ccccc1)C1C=CC=CC1N3c1ccccc1] | 72% |
| 19j | 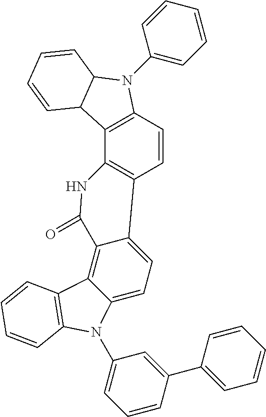 [SMILES: O=c1[nH]c2c3c(ccc2c2ccc4c(c5ccccc5n4-c4cccc(-c5ccccc5)c4)c12)N(c1ccccc1)C1C=CC=CC31] | 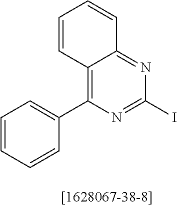 [SMILES: Ic1nc(-c2ccccc2)c2ccccc2n1] | 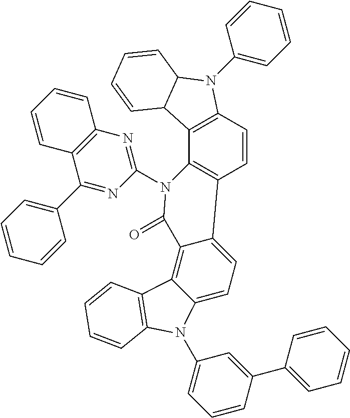 [SMILES: O=c1c2c(ccc3c2c2ccccc2n3-c2cccc(-c3ccccc3)c2)c2ccc3c(c2n1-c1nc(-c2ccccc2)c2ccccc2n1)C1C=CC=CC1N3c1ccccc1] | 74% |
| 20j | 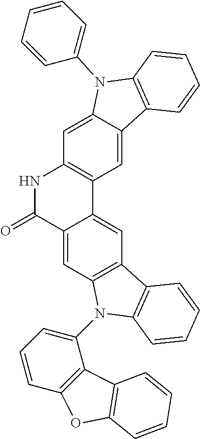 [SMILES: O=c1[nH]c2cc3c(cc2c2cc4c5ccccc5n(-c5cccc6oc7ccccc7c56)c4cc12)c1ccccc1n3-c1ccccc1] | 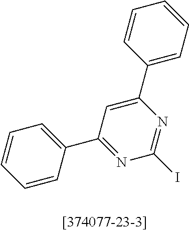 [SMILES: Ic1nc(-c2ccccc2)cc(-c2ccccc2)n1] | 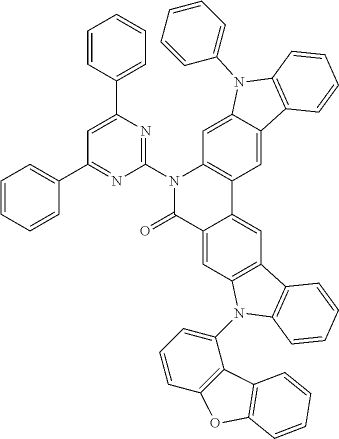 [SMILES: O=c1c2cc3c(cc2c2cc4c5ccccc5n(-c5ccccc5)c4cc2n1-c1nc(-c2ccccc2)cc(-c2ccccc2)n1)c1ccccc1n3-c1cccc2oc3ccccc3c12] | 63% |
| 21j | 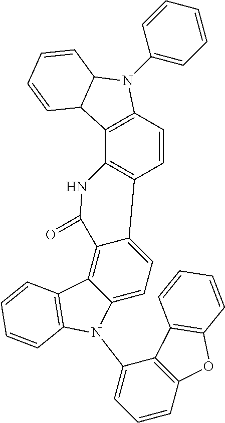 [SMILES: O=c1[nH]c2c3c(ccc2c2ccc4c(c5ccccc5n4-c4cccc5oc6ccccc6c45)c12)N(c1ccccc1)C1C=CC=CC31] | 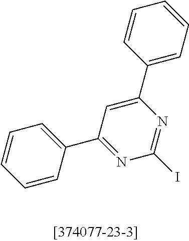 [SMILES: Ic1nc(-c2ccccc2)cc(-c2ccccc2)n1] | 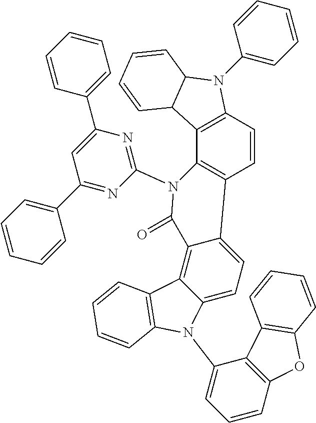 [SMILES: O=c1c2c(ccc3c2c2ccccc2n3-c2cccc3oc4ccccc4c23)c2ccc3c(c2n1-c1nc(-c2ccccc2)cc(-c2ccccc2)n1)C1C=CC=CC1N3c1ccccc1] | 60% |
| 22j | 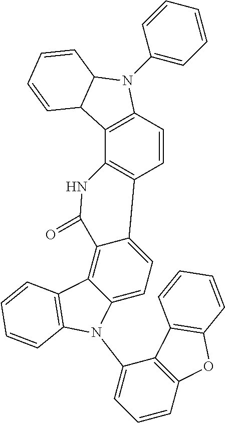 [SMILES: O=c1[nH]c2c3c(ccc2c2ccc4c(c5ccccc5n4-c4cccc5oc6ccccc6c45)c12)N(c1ccccc1)C1C=CC=CC31] | 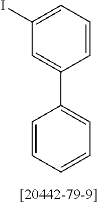 [SMILES: Ic1cccc(-c2ccccc2)c1] | 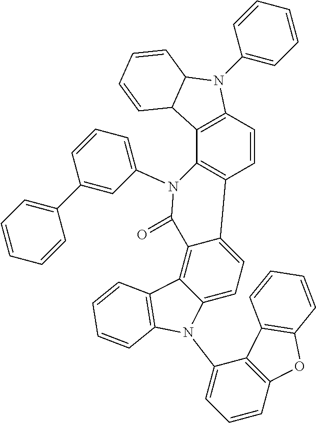 [SMILES: O=c1c2c(ccc3c2c2ccccc2n3-c2cccc3oc4ccccc4c23)c2ccc3c(c2n1-c1cccc(-c2ccccc2)c1)C1C=CC=CC1N3c1ccccc1] | 62% |

### k) Nucleophilic Substitution

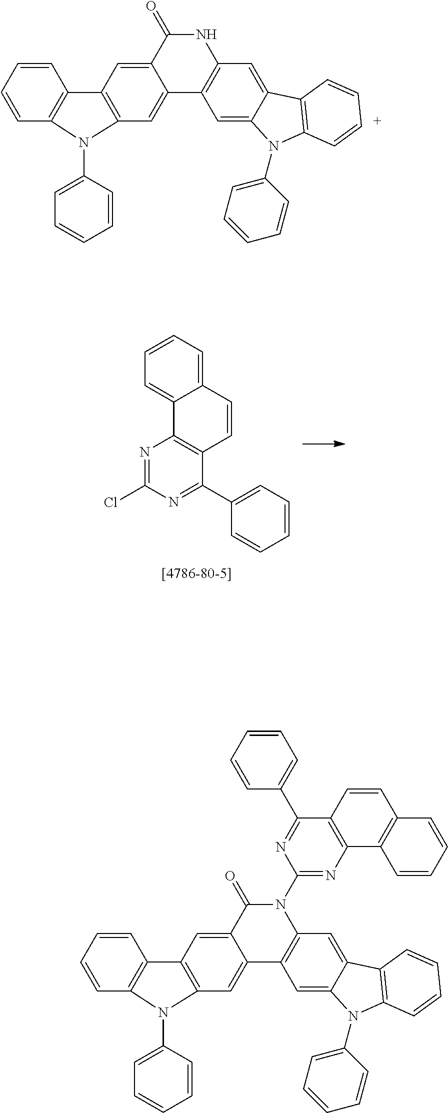 [SMILES: O=c1[nH]c2cc3c4ccccc4n(-c4ccccc4)c3cc2c2cc3c(cc12)c1ccccc1n3-c1ccccc1 ; Clc1nc(-c2ccccc2)c2ccc3ccccc3c2n1 ; O=c1c2cc3c4ccccc4n(-c4ccccc4)c3cc2c2cc3c(cc2n1-c1nc(-c2ccccc2)c2ccc4ccccc4c2n1)c1ccccc1n3-c1ccccc1]

32 g (61 mmol) of compound (i) is dissolved in 300 ml of dimethylformamide under a protective gas atmosphere, and 3 g of NaH, 60% in mineral oil, (75 mmol) is added. After 1 h at room temperature, a solution of 24 g (63 mmol) of 2-chloro-4-phenylbenzo[h]quinazoline in 150 ml of dimethylformamide is added dropwise. The reaction mixture is then stirred at room temperature for 12 h. After this time, the reaction mixture is poured onto ice and extracted three times with dichloromethane. The combined organic phases are dried over Na₂SO₄ and concentrated. The residue is recrystallized from toluene.

Yield: 38 g (48 mmol), 80% of theory

The following compounds can be prepared analogously:

| | Reactant 1 | Reactant 2 | Product | Yield |
|---|---|---|---|---|
| 1i | 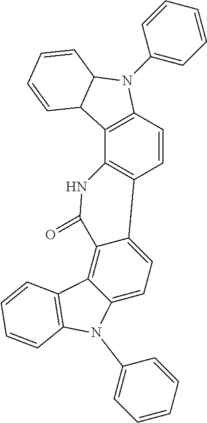 [SMILES: O=c1[nH]c2c3c(ccc2c2ccc4c(c5ccccc5n4-c4ccccc4)c12)N(c1ccccc1)C1C=CC=CC31] | 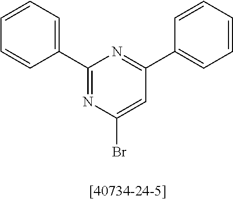 [SMILES: Brc1cc(-c2ccccc2)nc(-c2ccccc2)n1] | 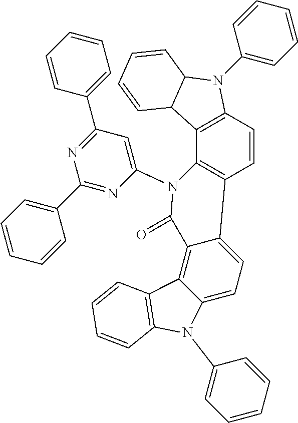 [SMILES: O=c1c2c(ccc3c2c2ccccc2n3-c2ccccc2)c2ccc3c(c2n1-c1cc(-c2ccccc2)nc(-c2ccccc2)n1)C1C=CC=CC1N3c1ccccc1] | 67% |
| 2i | 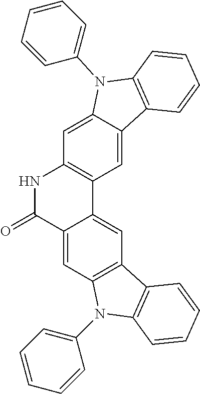 [SMILES: O=c1[nH]c2cc3c(cc2c2cc4c5ccccc5n(-c5ccccc5)c4cc12)c1ccccc1n3-c1ccccc1] | 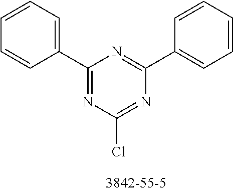 [SMILES: Clc1nc(-c2ccccc2)nc(-c2ccccc2)n1] | 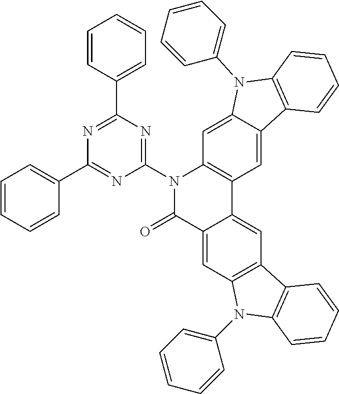 [SMILES: O=c1c2cc3c(cc2c2cc4c5ccccc5n(-c5ccccc5)c4cc2n1-c1nc(-c2ccccc2)nc(-c2ccccc2)n1)c1ccccc1n3-c1ccccc1] | 70% |
| 3i | 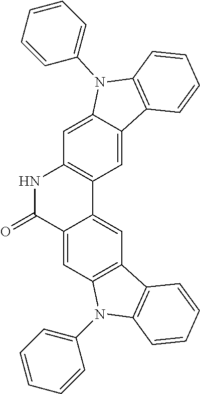 [SMILES: O=c1[nH]c2cc3c(cc2c2cc4c5ccccc5n(-c5ccccc5)c4cc12)c1ccccc1n3-c1ccccc1] | 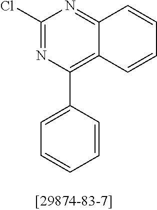 [SMILES: Clc1nc(-c2ccccc2)c2ccccc2n1] | 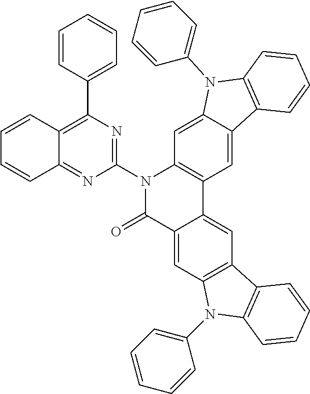 [SMILES: O=c1c2cc3c(cc2c2cc4c5ccccc5n(-c5ccccc5)c4cc2n1-c1nc(-c2ccccc2)c2ccccc2n1)c1ccccc1n3-c1ccccc1] | 68% |
| 4i | 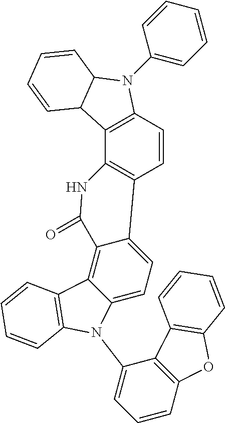 [SMILES: O=c1[nH]c2c3c(ccc2c2ccc4c(c5ccccc5n4-c4cccc5oc6ccccc6c45)c12)N(c1ccccc1)C1C=CC=CC31] | 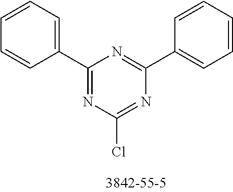 [SMILES: Clc1nc(-c2ccccc2)nc(-c2ccccc2)n1] | 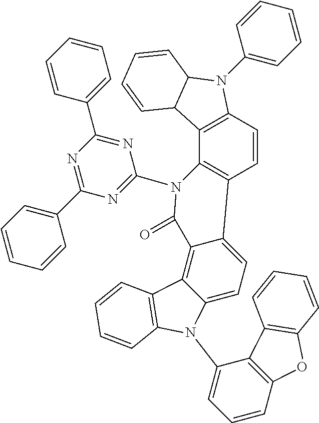 [SMILES: O=c1c2c(ccc3c2c2ccccc2n3-c2cccc3oc4ccccc4c23)c2ccc3c(c2n1-c1nc(-c2ccccc2)nc(-c2ccccc2)n1)C1C=CC=CC1N3c1ccccc1] | 65% |
| 5i | 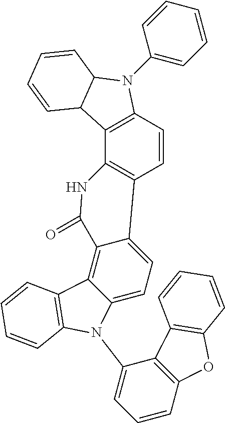 [SMILES: O=c1[nH]c2c3c(ccc2c2ccc4c(c5ccccc5n4-c4cccc5oc6ccccc6c45)c12)N(c1ccccc1)C1C=CC=CC31] | 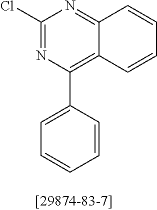 [SMILES: Clc1nc(-c2ccccc2)c2ccccc2n1] | 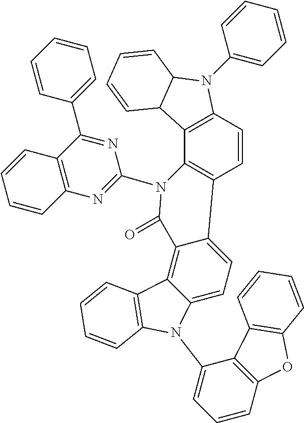 [SMILES: O=c1c2c(ccc3c2c2ccccc2n3-c2cccc3oc4ccccc4c23)c2ccc3c(c2n1-c1nc(-c2ccccc2)c2ccccc2n1)C1C=CC=CC1N3c1ccccc1] | 63% |

### Example 1: Production of the OLEDs

Examples E1 to E21 which follow present the use of the compounds of the invention in OLEDs.

Pretreatment for examples E1-E21: Glass plates coated with structured ITO (indium tin oxide) of thickness 50 nm are treated prior to coating, first with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 1. The materials required for production of the OLEDs are shown in table 2. The data of the OLEDs are listed in table 3.

All materials are applied by thermal vapor deposition in a vacuum chamber. In this case, the emission layer always consists of at least one matrix material (host material) and an emitting dopant (emitter) which is added to the matrix material(s) in a particular proportion by volume by co-evaporation. Details given in such a form as 2b:BisC1:TEG1 (45%:45%:10%) mean here that the material 2b is present in the layer in a proportion by volume of 45%, BisC1 in a proportion by volume of 45% and TEG1 in a proportion by volume of 10%. Analogously, the electron transport layer may also consist of a mixture of two materials.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. Electroluminescence spectra are determined at a luminance of 1000 cd/m², and these are used to calculate the CIE 1931 x and y color coordinates. The parameter U1000 in table 3 refers to the voltage which is required for a luminance of 1000 cd/m². EQE1000 denotes the external quantum efficiency which is attained at 1000 cd/m².

### Use of Mixtures of the Invention in OLEDs

The material combinations of the invention can be used in the emission layer in phosphorescent OLEDs. Inventive compounds 1j, 3j, 5j, 6j and 11j are used in examples E1 to E13 as h-type (hole-transporting) matrix for green emitters in the emission layer, and compounds 7j, 8j, 20j, 2i and 4i are used in examples E14 to E18 as e-type (electron-transporting) matrix for green emitters in the emission layer, and compound 11j is used in example E19 as hole conductor for green matrix material in the emission layer, and 8j is used in examples E20 and E21 as red matrix material in the emission layer.

The inventive compounds are used in combination with h-type matrices such as BisC1 (h-type) or TZ5 (e-type) in examples E2 to E18 or as a single host (E1, E19, E21).

The inventive compound 8j is used as red matrix material in the emission layer as a single host and in combination with compound BisC2 in examples E20 and E21.

TABLE 1
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| V1 | HATCN | SpMA1 | SpMA2 | TZ5V1::TEG1 | ST2 | ST2:LiQ | LiQ 1 nm |
| | 5 nm | 125 nm | 10 nm | (46%:47%:7%) | 10 nm | (50%:50%) 30 nm | |
| | | | | 30 nm | | | |
| V2 | HATCN | SpMA1 | SpMA2 | TZ5:V2:TEG1 | ST2 | ST2:LiQ | LiQ 1 nm |
| | 5 nm | 125 nm | 10 nm | (46%:47%:7%) | 10 nm | (50%:50%) 30 nm | |
| | | | | 30 nm | | | |
| V3 | HATCN | SpMA1 | SpMA2 | TZ5:V3:TEG1 | ST2 | ST2:LiQ | LiQ 1 nm |
| | 5 nm | 125 nm | 10 nm | (46%:47%:7%) | 10 nm | (50%:50%) 30 nm | |
| | | | | 30 nm | | | |
| V4 | HATCN | SpMA1 | SpMA2 | V4:BisC1:TEG1 | ST2 | ST2:LiQ | LiQ 1 nm |
| | 5 nm | 125 nm | 10 nm | (46%:47%:7%) | 10 nm | (50%:50%) 30 nm | |
| | | | | 30 nm | | | |
| V5 | HATCN | SpMA1 | SpMA2 | V4:TEG1 | ST2 | ST2:LiQ | LiQ 1 nm |
| | 5 nm | 125 nm | 10 nm | (97%:3%) 35 nm | 10 nm | (50%:50%) 30 nm | |
| E1 | HATCN | SpMA1 | SpMA2 | 1j:TEG1 | ST2 | ST2:LiQ | LiQ 1 nm |
| | 5 nm | 125 nm | 10 nm | (97%:3%) 35 nm | 10 nm | (50%:50%) 30 nm | |
| E2 | HATCN | SpMA1 | SpMA2 | TZ5:1j:TEG1 | ST2 | ST2:LiQ | LiQ 1 nm |
| | 5 nm | 125 nm | 10 nm | (97%:3%) 35 nm | 10 nm | (50%:50%) 30 nm | |
| E3 | HATCN | SpMA1 | SpMA2 | TZ3:1j:TEG1 | ST2 | ST2:LiQ | LiQ 1 nm |
| | 5 nm | 125 nm | 10 nm | (46%:47%:7%) | 10 nm | (50%:50%) 30 nm | |
| | | | | 30 nm | | | |
| E4 | HATCN | SpMA1 | SpMA2 | TZ4:1j:TEG1 | ST2 | ST2:LiQ | LiQ 1 nm |
| | 5 nm | 125 nm | 10 nm | (46%:47%:7%) | 10 nm | (50%:50%) 30 nm | |
| | | | | 30 nm | | | |
| E5 | HATCN | SpMA1 | SpMA2 | TZ5:1j:TEG1 | ST2 | ST2:LiQ | LiQ 1 nm |
| | 5 nm | 125 nm | 10 nm | (46%:47%:7%) | 10 nm | (50%:50%) 30 nm | |
| | | | | 30 nm | | | |
| E6 | HATCN | SpMA1 | SpMA2 | TZ6:1j:TEG1 | ST2 | ST2:LiQ | LiQ 1 nm |
| | 5 nm | 125 nm | 10 nm | (46%:47%:7%) | 10 nm | (50%:50%) 30 nm | |
| | | | | 30 nm | | | |
| E7 | HATCN | SpMA1 | SpMA2 | TZ8:1j:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E8 | HATCN | SpMA1 | SpMA2 | TZ2:3j:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E9 | HATCN | SpMA1 | SpMA2 | TZ5:5j:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E10 | HATCN | SpMA1 | SpMA2 | TZ5:6j:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E11 | HATCN | SpMA1 | SpMA2 | TZ3:6j:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E12 | HATCN | SpMA1 | SpMA2 | TZ7:11j:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E13 | HATCN | SpMA1 | SpMA2 | TZ5:11j:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E14 | HATCN | SpMA1 | SpMA2 | 7j:BisC1:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E15 | HATCN | SpMA1 | SpMA2 | 8j:BisC1:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E16 | HATCN | SpMA1 | SpMA2 | 20j:BisC1:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E17 | HATCN | SpMA1 | SpMA2 | 2i:BisC1:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E18 | HATCN | SpMA1 | SpMA2 | 4i:BisC1:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E19 | HATCN | SpMA1 | 11j | Tz4:BisC1:TEG1 | ST2 | ST2:LiQ | LiQ 1 nm |
| | 5 nm | 125 nm | 10 nm | ((46%:47%:7%) | 10 nm | (50%:50%) 30 nm | |
| | | | | 30 nm | | | |
| E20 | HATCN | SpMA1 | SpMA2 | 8j:BisC1:TER1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E21 | HATCN | SpMA1 | SpMA2 | 8j:TER1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |
| E22 | HATCN | SpMA1 | SpMA2 | TZ5:22i:TEG1 | ST2 | ST2:LiQ | LiQ 1 nm |
| | 5 nm | 230 nm | 20 nm | (46%:47%:7%) | 10 nm | (50%:50%) 30 nm | |
| | | | | 30 nm | | | |

TABLE 2
Structural formulae of the materials for OLEDs

| | |
|---|---|
| 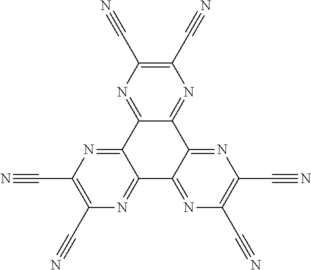 [SMILES: N#Cc1nc2c3nc(C#N)c(C#N)nc3c3nc(C#N)c(C#N)nc3c2nc1C#N] | HATCN |
| 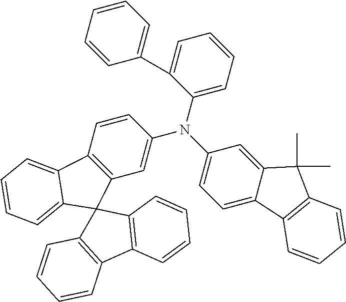 [SMILES: CC1(C)c2ccccc2-c2ccc(N(c3ccc4c(c3)C3(c5ccccc5-c5ccccc53)c3ccccc3-4)c3ccccc3-c3ccccc3)cc21] | SpMA1 |
| 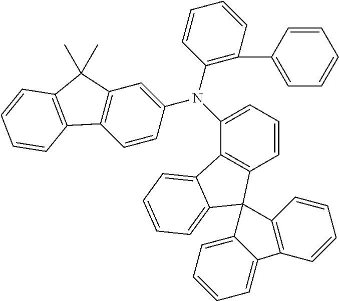 [SMILES: CC1(C)c2ccccc2-c2ccc(N(c3ccccc3-c3ccccc3)c3cccc4c3-c3ccccc3C43c4ccccc4-c4ccccc43)cc21] | SpMA2 |
| 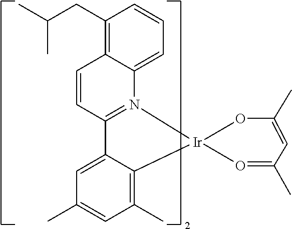 | TER1 |
| 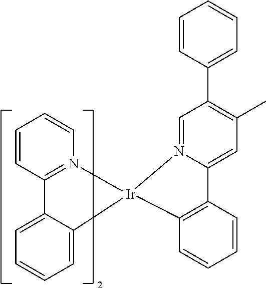 | TEG1 |
| 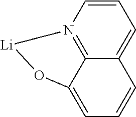 | LiQ |
| 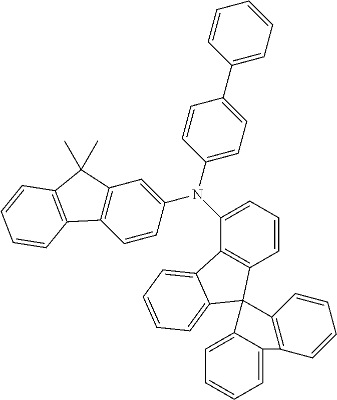 [SMILES: CC1(C)c2ccccc2-c2ccc(N(c3ccc(-c4ccccc4)cc3)c3cccc4c3-c3ccccc3C43c4ccccc4-c4ccccc43)cc21] | SpMA3 |
| 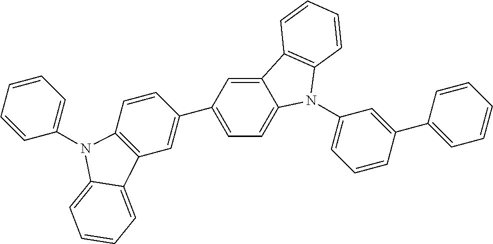 [SMILES: c1ccc(-c2cccc(-n3c4ccccc4c4cc(-c5ccc6c(c5)c5ccccc5n6-c5ccccc5)ccc43)c2)cc1] | BisC1 |
| 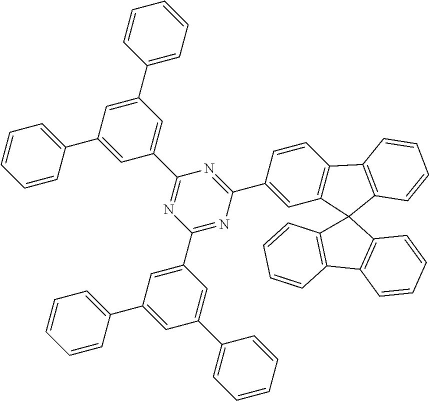 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] | ST2 |
| 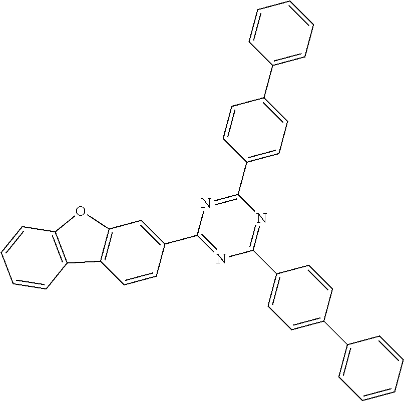 [SMILES: c1ccc(-c2ccc(-c3nc(-c4ccc(-c5ccccc5)cc4)nc(-c4ccc5c(c4)oc4ccccc45)n3)cc2)cc1] | TZ2 |
| 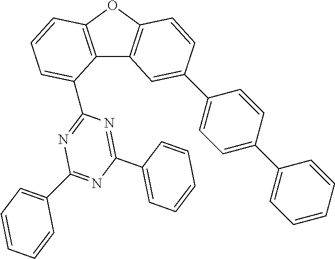 [SMILES: c1ccc(-c2ccc(-c3ccc4oc5cccc(-c6nc(-c7ccccc7)nc(-c7ccccc7)n6)c5c4c3)cc2)cc1] | TZ3 |
| 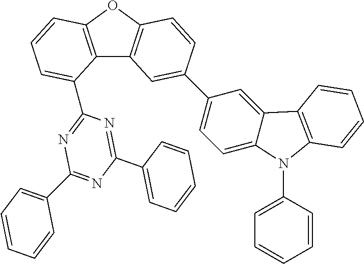 [SMILES: c1ccc(-c2nc(-c3ccccc3)nc(-c3cccc4oc5ccc(-c6ccc7c(c6)c6ccccc6n7-c6ccccc6)cc5c34)n2)cc1] | TZ4 |
| 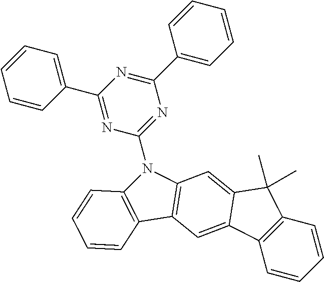 [SMILES: CC1(C)c2ccccc2-c2cc3c4ccccc4n(-c4nc(-c5ccccc5)nc(-c5ccccc5)n4)c3cc21] | TZ5 |
| 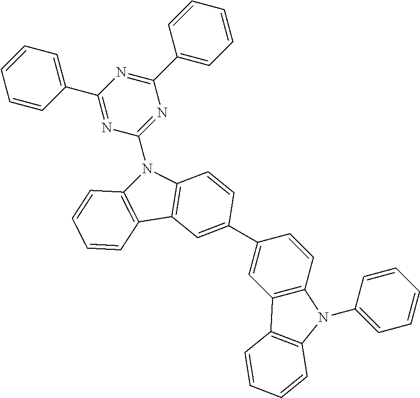 [SMILES: c1ccc(-c2nc(-c3ccccc3)nc(-n3c4ccccc4c4cc(-c5ccc6c(c5)c5ccccc5n6-c5ccccc5)ccc43)n2)cc1] | TZ6 |
| 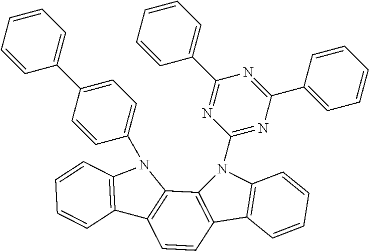 [SMILES: c1ccc(-c2ccc(-n3c4ccccc4c4ccc5c6ccccc6n(-c6nc(-c7ccccc7)nc(-c7ccccc7)n6)c5c43)cc2)cc1] | TZ7 |
| 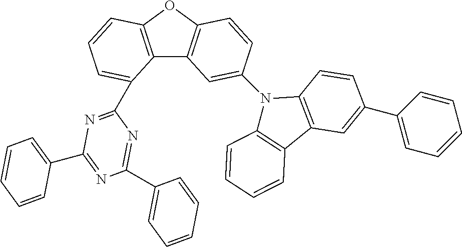 [SMILES: c1ccc(-c2ccc3c(c2)c2ccccc2n3-c2ccc3oc4cccc(-c5nc(-c6ccccc6)nc(-c6ccccc6)n5)c4c3c2)cc1] | TZ8 |
| 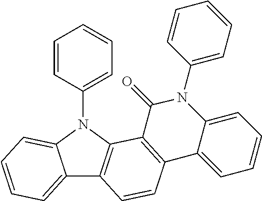 [SMILES: O=c1c2c(ccc3c4ccccc4n(-c4ccccc4)c32)c2ccccc2n1-c1ccccc1] | V1 |
| 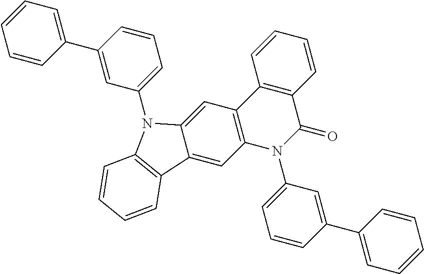 [SMILES: O=c1c2ccccc2c2cc3c(cc2n1-c1cccc(-c2ccccc2)c1)c1ccccc1n3-c1cccc(-c2ccccc2)c1] | V2 |
| 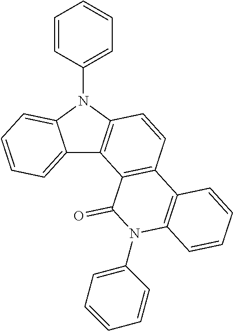 [SMILES: O=c1c2c(ccc3c2c2ccccc2n3-c2ccccc2)c2ccccc2n1-c1ccccc1] | V3 |
| 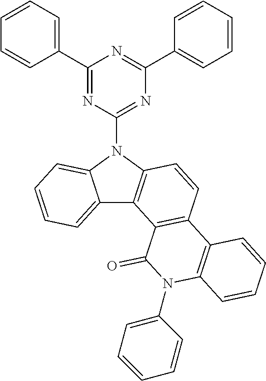 [SMILES: O=c1c2c(ccc3c2c2ccccc2n3-c2nc(-c3ccccc3)nc(-c3ccccc3)n2)c2ccccc2n1-c1ccccc1] | V4 |
| 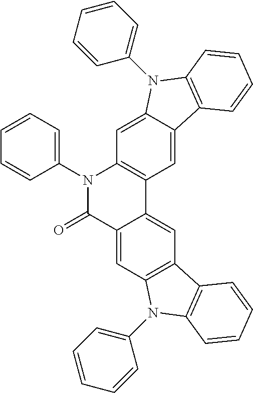 [SMILES: O=c1c2cc3c(cc2c2cc4c5ccccc5n(-c5ccccc5)c4cc2n1-c1ccccc1)c1ccccc1n3-c1ccccc1] | 1j |
| 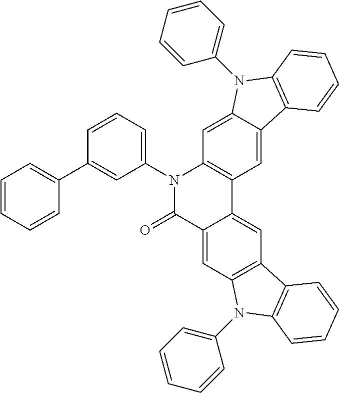 [SMILES: O=c1c2cc3c(cc2c2cc4c5ccccc5n(-c5ccccc5)c4cc2n1-c1cccc(-c2ccccc2)c1)c1ccccc1n3-c1ccccc1] | 3j |
| 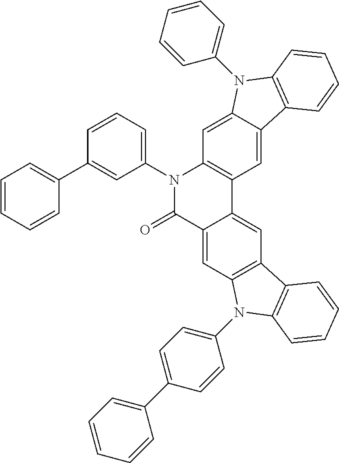 [SMILES: O=c1c2cc3c(cc2c2cc4c5ccccc5n(-c5ccccc5)c4cc2n1-c1cccc(-c2ccccc2)c1)c1ccccc1n3-c1ccc(-c2ccccc2)cc1] | 5j |
| 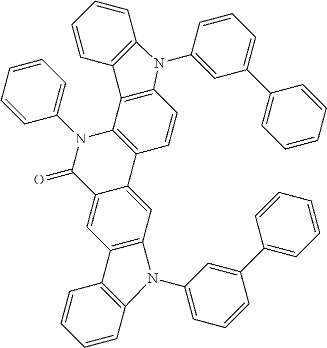 [SMILES: O=c1c2cc3c4ccccc4n(-c4cccc(-c5ccccc5)c4)c3cc2c2ccc3c(c4ccccc4n3-c3cccc(-c4ccccc4)c3)c2n1-c1ccccc1] | 6j |
| 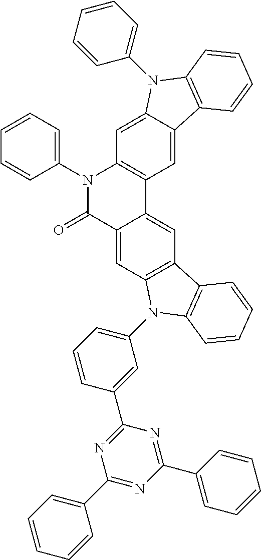 [SMILES: O=c1c2cc3c(cc2c2cc4c5ccccc5n(-c5ccccc5)c4cc2n1-c1ccccc1)c1ccccc1n3-c1cccc(-c2nc(-c3ccccc3)nc(-c3ccccc3)n2)c1] | 7j |
| 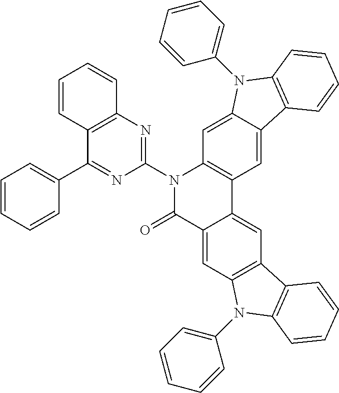 [SMILES: O=c1c2cc3c(cc2c2cc4c5ccccc5n(-c5ccccc5)c4cc2n1-c1nc(-c2ccccc2)c2ccccc2n1)c1ccccc1n3-c1ccccc1] | 8j |
| 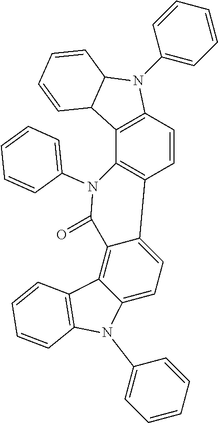 [SMILES: O=c1c2c(ccc3c2c2ccccc2n3-c2ccccc2)c2ccc3c(c2n1-c1ccccc1)C1C=CC=CC1N3c1ccccc1] | 11j |
| 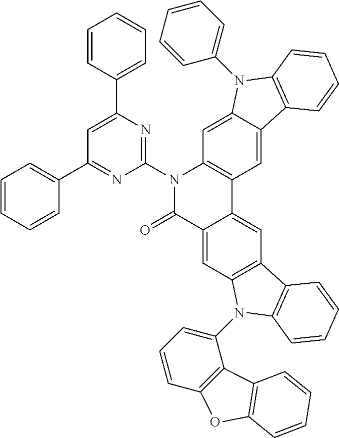 [SMILES: O=c1c2cc3c(cc2c2cc4c5ccccc5n(-c5ccccc5)c4cc2n1-c1nc(-c2ccccc2)cc(-c2ccccc2)n1)c1ccccc1n3-c1cccc2oc3ccccc3c12] | 20j |
| 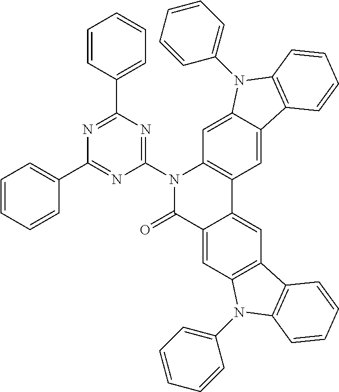 [SMILES: O=c1c2cc3c(cc2c2cc4c5ccccc5n(-c5ccccc5)c4cc2n1-c1nc(-c2ccccc2)nc(-c2ccccc2)n1)c1ccccc1n3-c1ccccc1] | 2i |
| 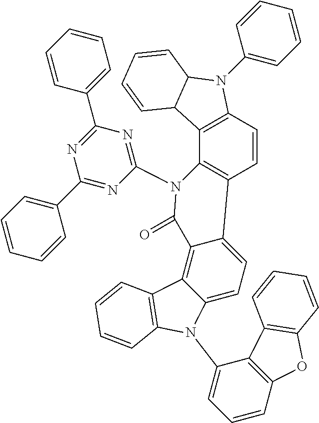 [SMILES: O=c1c2c(ccc3c2c2ccccc2n3-c2cccc3oc4ccccc4c23)c2ccc3c(c2n1-c1nc(-c2ccccc2)nc(-c2ccccc2)n1)C1C=CC=CC1N3c1ccccc1] | 4i |

TABLE 3
Data of the OLEDs

| | U1000 | EQE 1000 | CIE x/y at | j0 | L1 | LT |
|---|---|---|---|---|---|---|
| Ex. | (V) | (%) | 1000 cd/m² | (mA/cm²) | (%) | (h) |
| V1 | 4.4 | 15.0 | 0.32/0.64 | 20 | 80 | 203 |
| V2 | 4.3 | 14.1 | 0.32/0.63 | 20 | 80 | 212 |
| V3 | 4.2 | 15.3 | 0.33/0.63 | 20 | 80 | 200 |
| V4 | 4.0 | 16.0 | 0.32/0.63 | 20 | 80 | 270 |
| V5 | 4.5 | 15.0 | 0.32/0.63 | 20 | 80 | 252 |
| E1 | 3.8 | 16.8 | 0.32/0.64 | 20 | 80 | 360 |
| E2 | 3.4 | 19.2 | 0.33/0.63 | 20 | 80 | 540 |
| E3 | 3.3 | 18.4 | 0.33/0.64 | 20 | 80 | 501 |
| E4 | 3.2 | 18.5 | 0.31/0.64 | 20 | 80 | 487 |
| E5 | 3.4 | 18.2 | 0.33/0.63 | 20 | 80 | 488 |
| E6 | 3.3 | 18.1 | 0.33/0.63 | 20 | 80 | 485 |
| E7 | 3.3 | 18.5 | 0.32/0.64 | 20 | 80 | 449 |
| E8 | 3.2 | 18.0 | 0.31/0.64 | 20 | 80 | 440 |
| E9 | 3.4 | 17.4 | 0.32/0.63 | 20 | 80 | 461 |
| E10 | 3.2 | 18.9 | 0.33/0.64 | 20 | 80 | 453 |
| E11 | 3.3 | 18.4 | 0.31/0.63 | 20 | 80 | 420 |
| E12 | 3.0 | 19.1 | 0.31/0.64 | 20 | 80 | 522 |
| E13 | 3.1 | 17.3 | 0.32/0.64 | 20 | 80 | 513 |
| E14 | 3.4 | 18.5 | 0.33/0.63 | 20 | 80 | 380 |
| E15 | 3.2 | 18.3 | 0.32/0.64 | 20 | 80 | 595 |
| E16 | 3.2 | 18.5 | 0.31/0.63 | 20 | 80 | 375 |
| E17 | 3.1 | 18.6 | 0.32/0.64 | 20 | 80 | 530 |
| E18 | 3.2 | 18.0 | 0.32/0.63 | 20 | 80 | 380 |
| E19 | 3.1 | 17.9 | 0.32/0.63 | 20 | 80 | 360 |
| E20 | 3.3 | 24.0 | 0.66/0.34 | 20 | 95 | 285 |
| E21 | 3.4 | 22.1 | 0.66/0.33 | 20 | 95 | 220 |
| E22 | 3.5 | 18.4 | 0.33/0.64 | 20 | 80 | 385 |

1. A compound of formula (1) 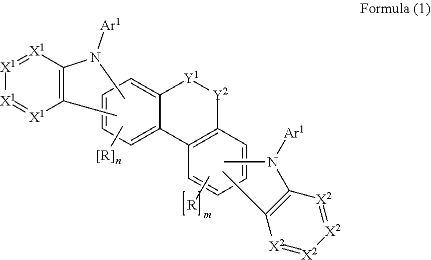
wherein the symbols used are as follows:
X¹ is the same or different at each instance and is CR or N, with the proviso that not more than two X¹ groups are N;
X² is the same or different at each instance and is CR or N, with the proviso that not more than two X² groups are N;
Y¹ is the same or different at each instance and is C═O, C═S, BR^{a}, NR^{a}, S, O, S═O, SO₂, PR^{a} or POR^{a};
Y² is the same or different at each instance and is C═O, C═S, NR^{a}, NR^{a}, S, O, S═O, SO₂, PR^{a} or POR^{a};
wherein:
Y¹ is not the same as Y²;
Ar¹ is the same or different at each instance and is an aromatic or heteroaromatic ring system which has 5 to 60 aromatic ring atoms and may be substituted in each case by one or more R¹ radicals, wherein one Ar¹ radical together with an R or R^{a} radical may form a ring system that may be substituted by one or more R¹ radicals;
R, R^{a} is the same or different at each instance and is H, D, F, Cl, Br, I, CN, NO₂, N(Ar^{a})₂, N(R¹)₂, C(═O)N(Ar^{a})₂, C(═O)N(R¹)₂, C(Ar^{a})₃, C(R¹)₃, Si(Ar^{a})₃, Si(R¹)₃, B(Ar^{a})₂, B(R¹)₂, C(═O)Ar^{a}, C(═O)R¹, P(═O)(Ar^{a})₂, P(═O)(R¹)₂, P(Ar^{a})₂, P(R¹)₂, S(═O)Ar^{a}, S(═O)R¹, S(═O)₂Ar^{a}, S(═O)₂R¹, OSO₂Ar^{a}, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, wherein the alkyl, alkoxy, thioalkoxy, alkenyl or alkynyl group may be substituted in each case by one or more R¹ radicals, wherein one or more nonadjacent CH₂ groups may be replaced by R¹C═CR¹, C≡C, Si(R¹)₂, C═O, C═S, C═Se, C═NR¹, C(═O)O—, C(═O)NR¹—, NR¹, P(═O)(R¹), O, S, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two or more R and/or R^{a} radicals together may form an aliphatic, heteroaliphatic, aromatic or heteroaromatic ring system which may be substituted by one or more R¹ radicals; or an R or R^{a} radical together with an Ar¹ radical may form a ring system that may be substituted by one or more R¹ radicals;
Ar^{a} is the same or different at each instance and is an aromatic or heteroaromatic ring system which has 5 to 60 aromatic ring atoms and may be substituted by one or more R¹ radicals; at the same time, it is possible for two Ar^{a} radicals bonded to the same carbon atom, silicon atom, nitrogen atom, phosphorus atom or boron atom also to be joined together via a bridge by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹;
n, m is the same or different at each instance and is 0, 1 or 2;
R¹ is the same or different at each instance and is H, D, F, Cl, Br, I, CN, NO₂, N(R²)₂, C(═O)R², P(═O)(R²)₂, P(R²)₂, B(R²)₂, C(R²)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by R²C═CR², C≡C, Si(R²)₂, C═O, C═S, C═Se, C═NR², C(═O)O, C(═O)NR², NR², P(═O)(R²), O, S, SO or SO₂ and wherein one or more hydrogen atoms may be replaced by D, F, Cl, Br, I, CN or NO₂, or an aromatic or heteroaromatic ring system which has 5 to 60 aromatic ring atoms, each of which may be substituted by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, where two or more R¹ radicals together may form an aliphatic, heteroaliphatic, aromatic or heteroaromatic ring system that may be substituted by one or more R² radicals;
R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, C, Br, I or CN and which may be substituted by one or more alkyl groups each having 1 to 4 carbon atoms; at the same time, two or more substituents R² together may form a ring system.

2. A compound as claimed in claim 1, wherein X¹ is CR and/or X² is CR.

3. A compound as claimed in claim 1, and which is selected from the formulae (1-2a) to (1-2d), wherein the symbols have the definitions given in claim 1:

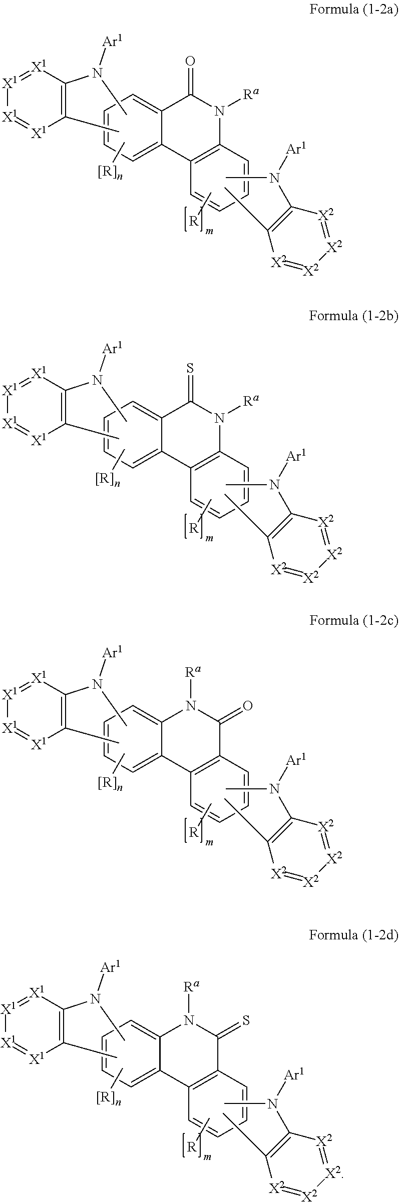

4. A compound as claimed in claim 1, and which is selected from formulae (2-1a) to (2-36a), wherein the symbols have the definitions given in claim 1:

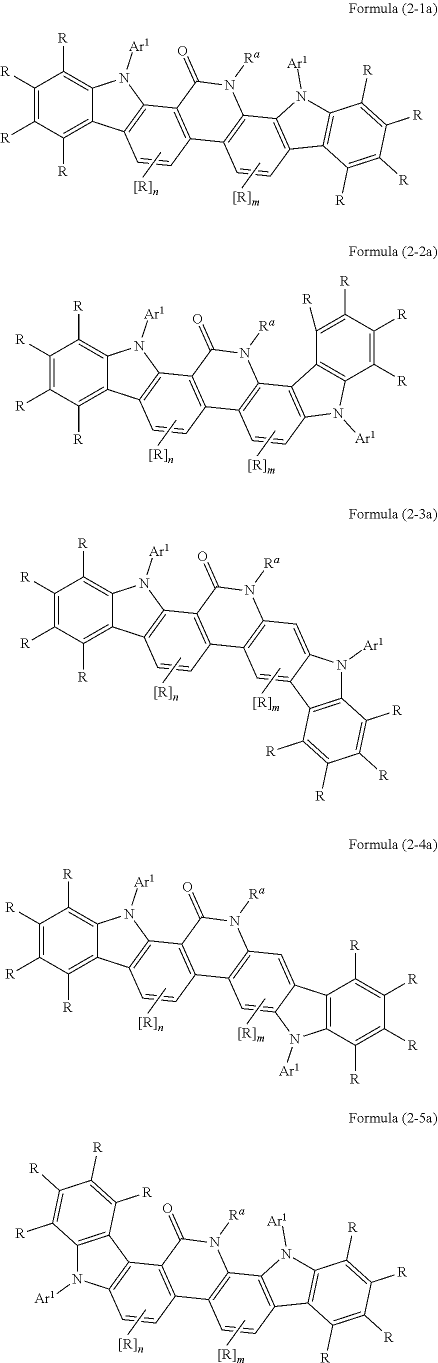

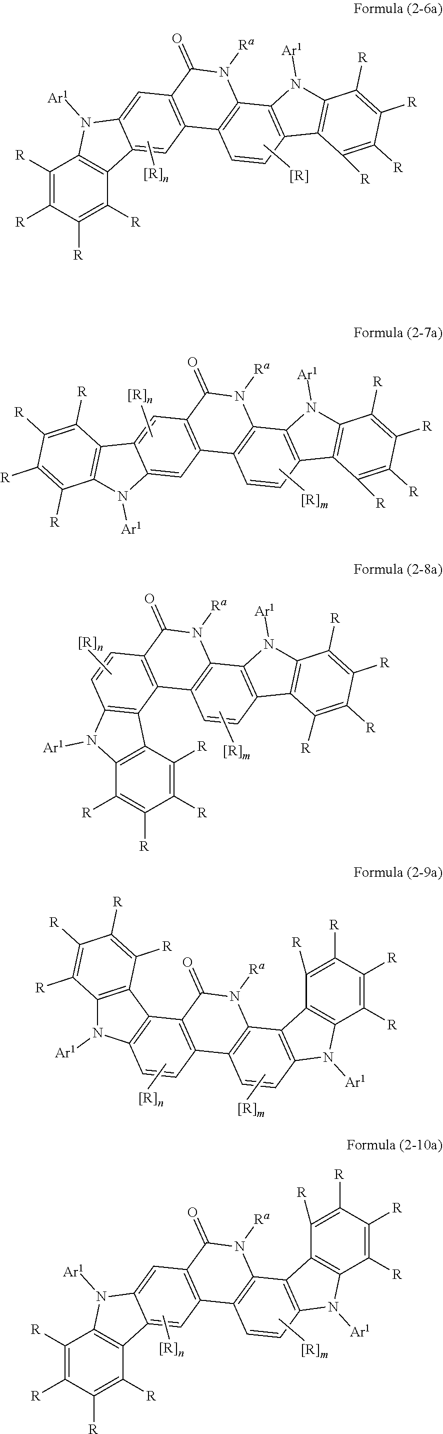

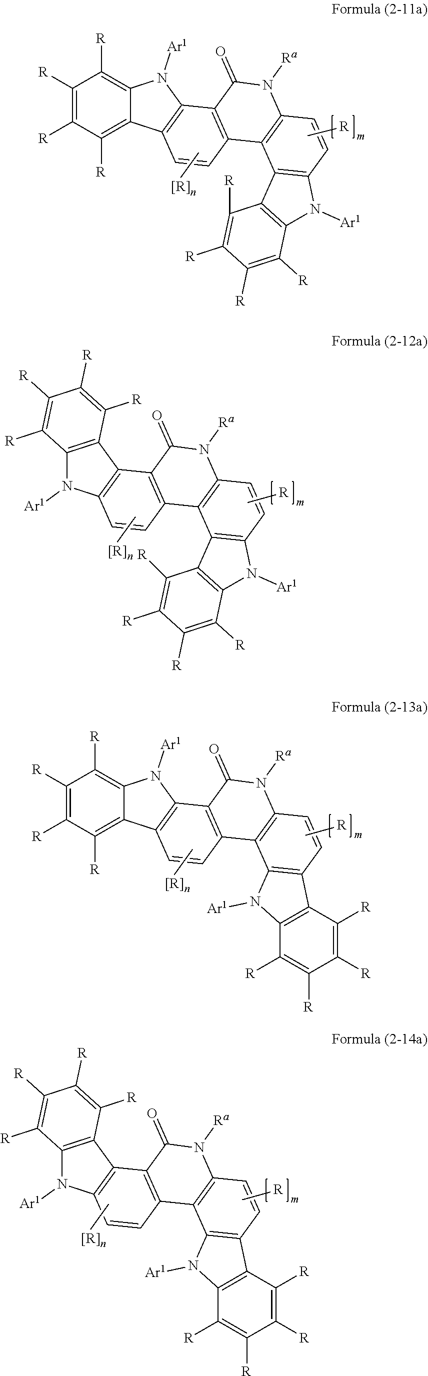

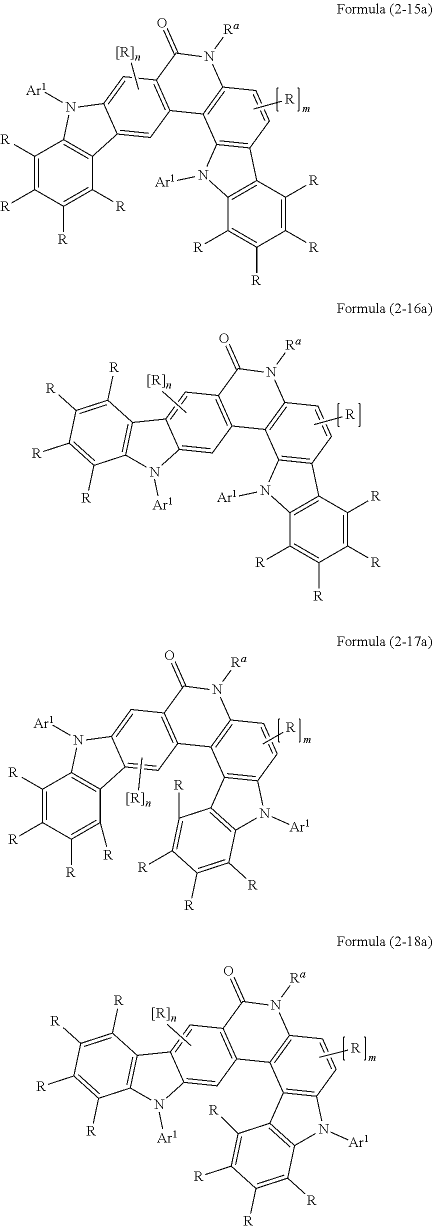

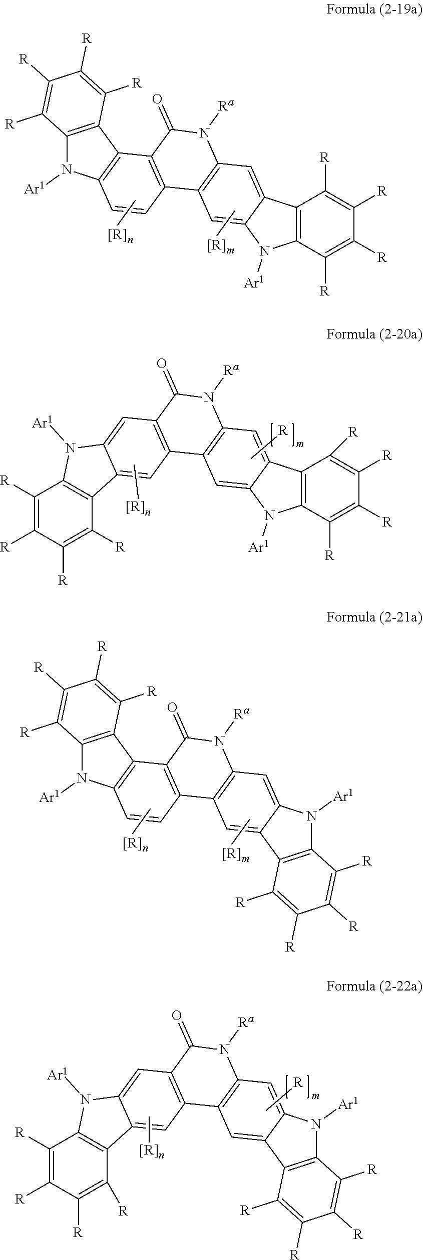

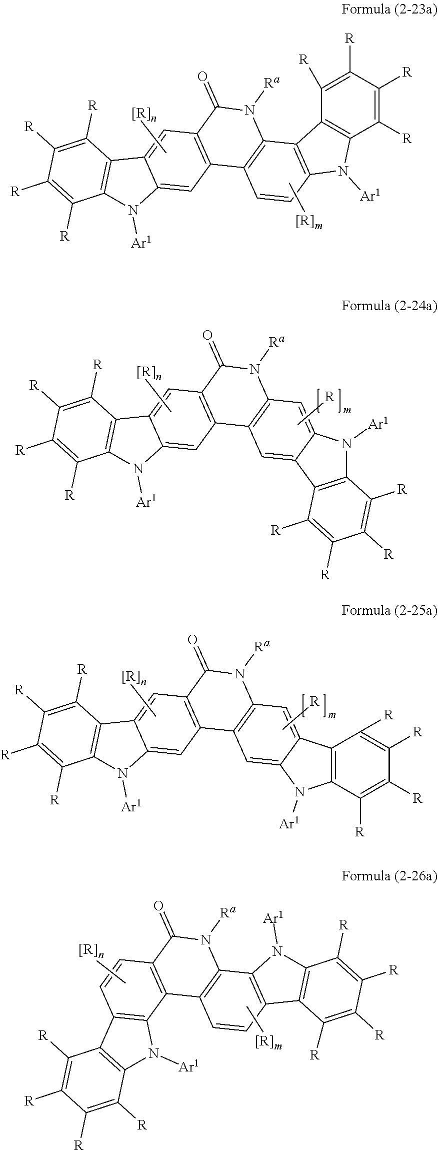

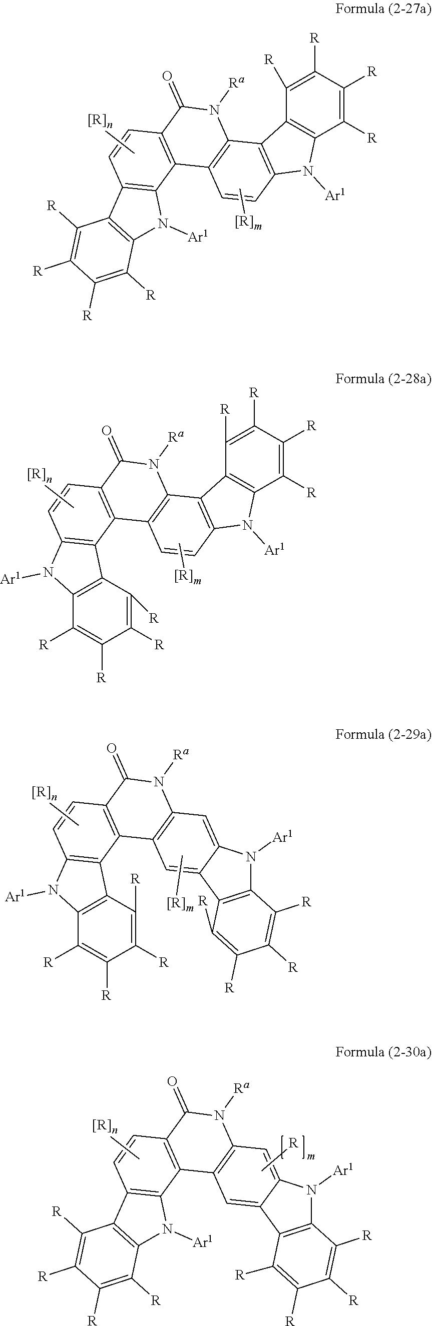

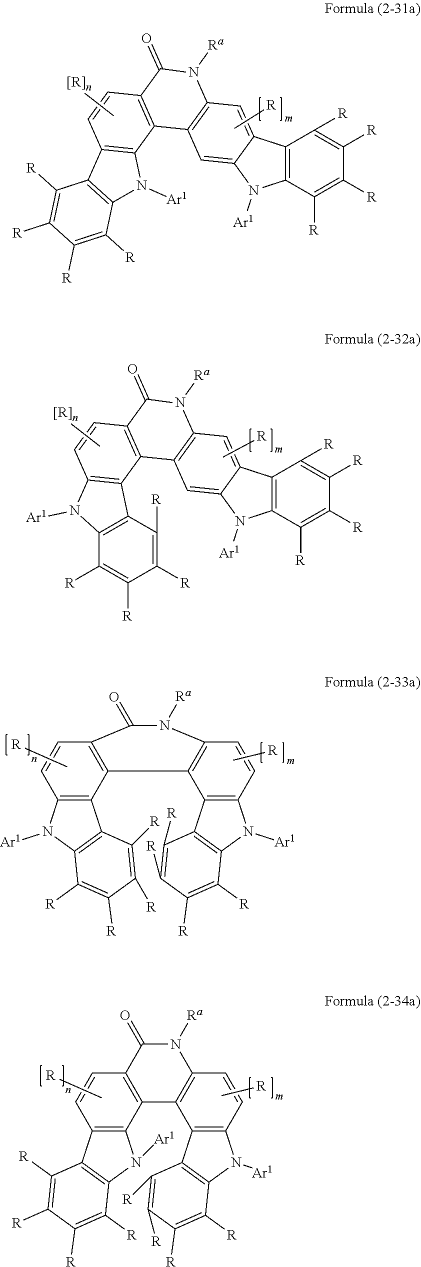

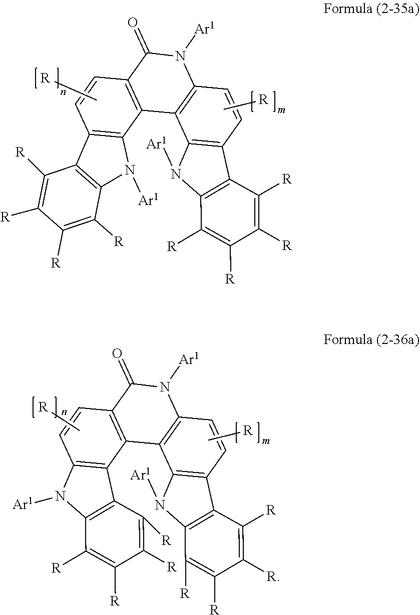

5. A compound as claimed in claim 1, and which is selected from the compounds of the formulae (2-1 b) to (2-72b)

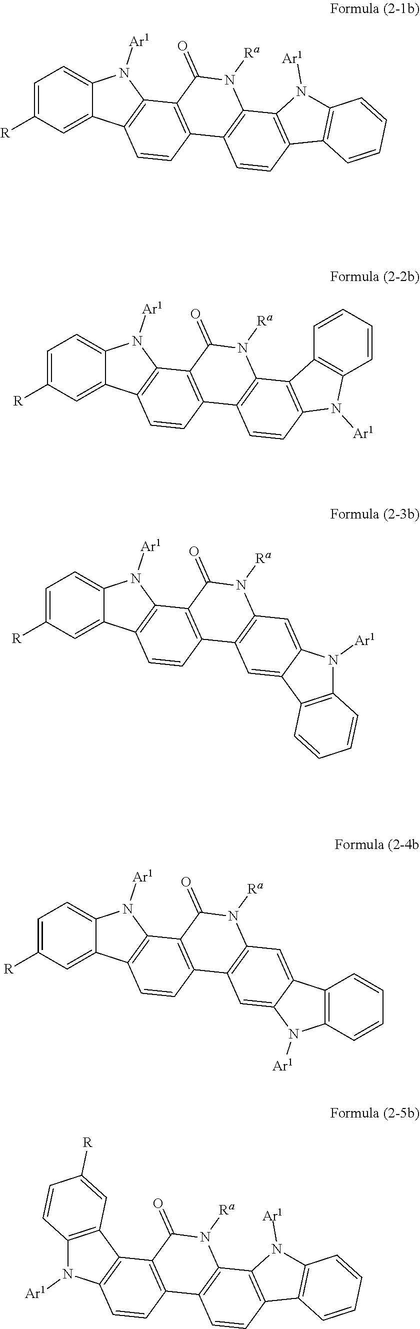

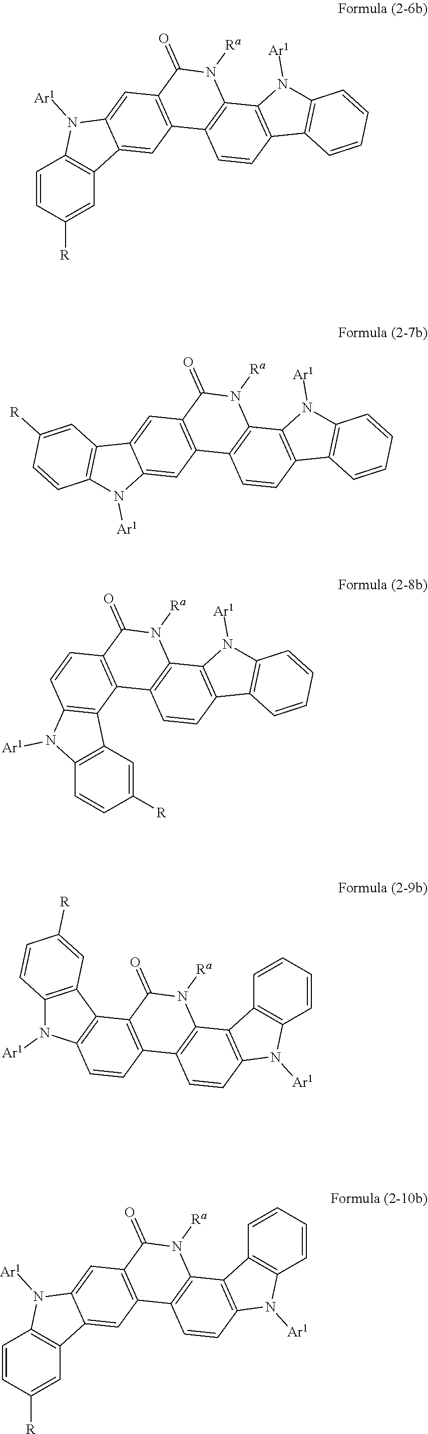

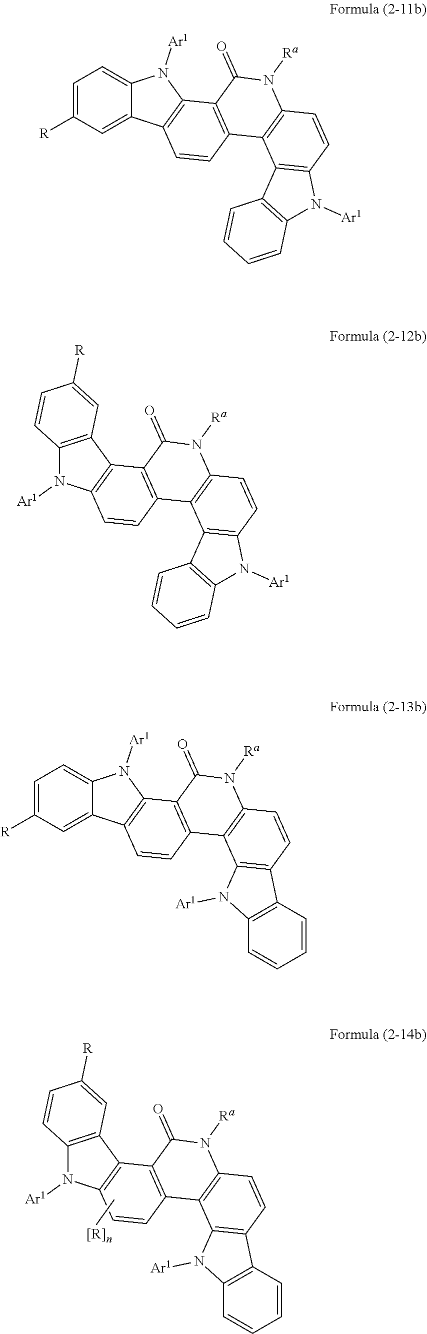

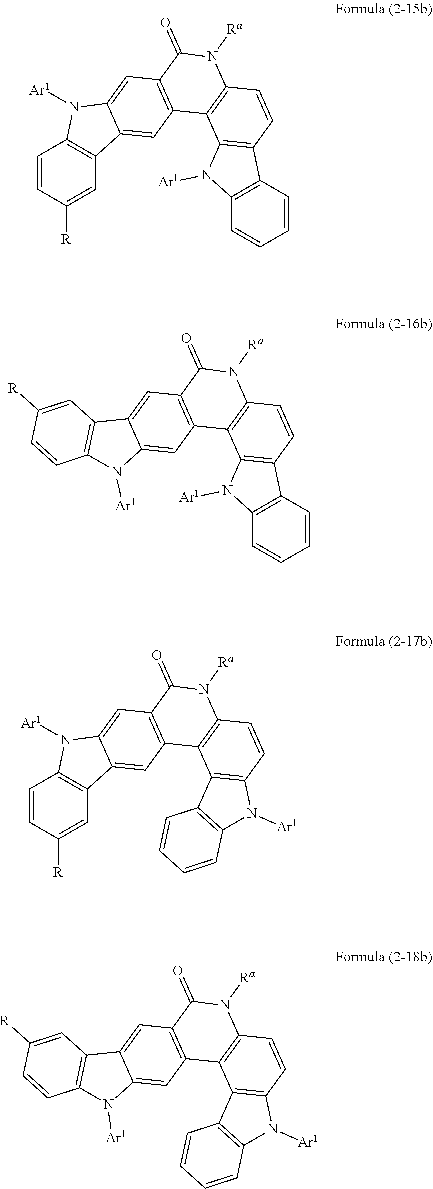

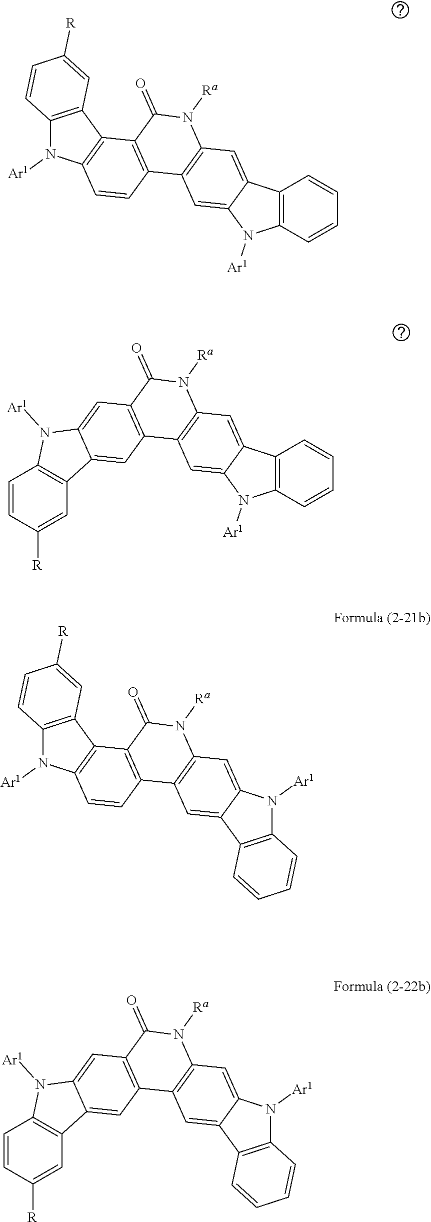

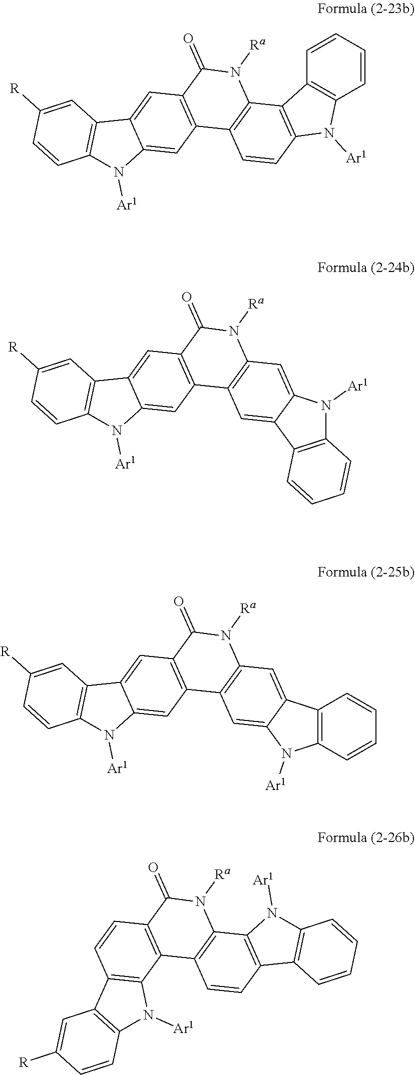

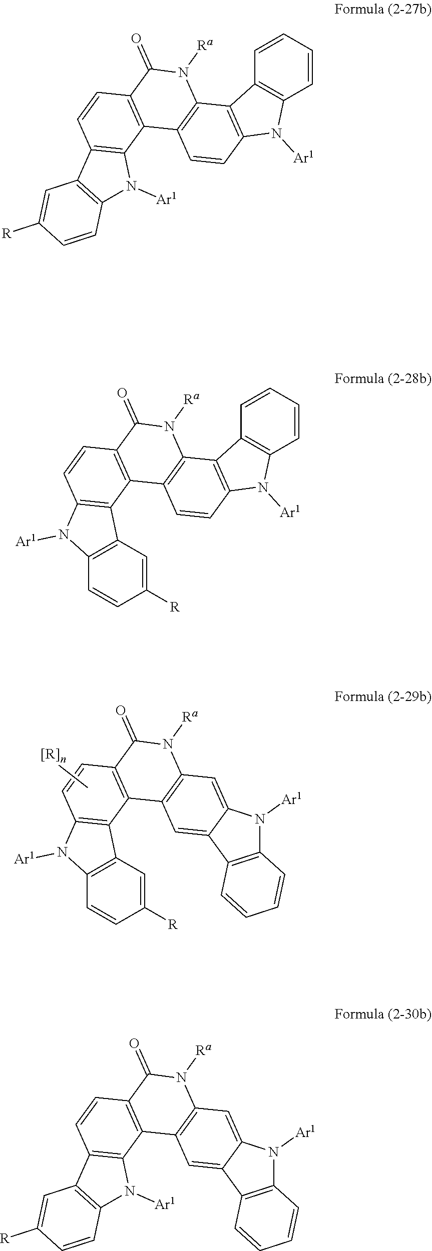

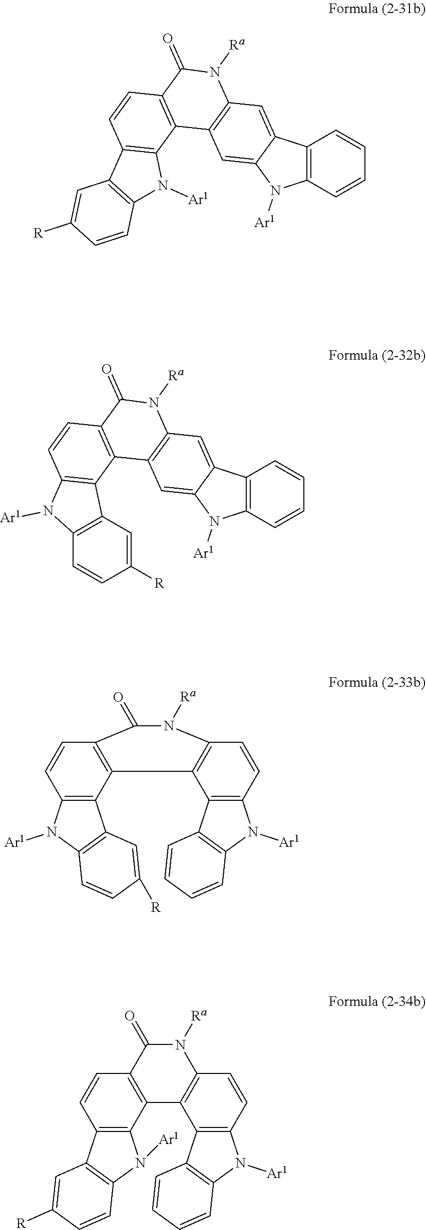

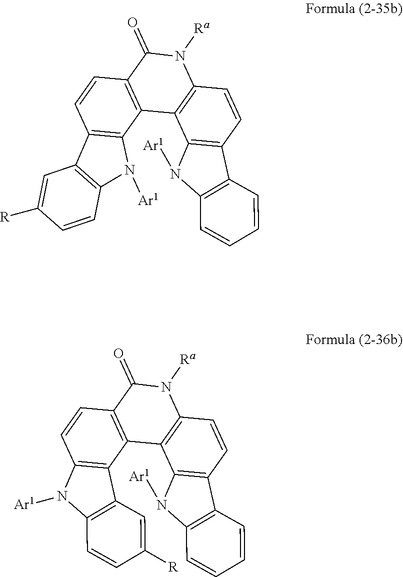

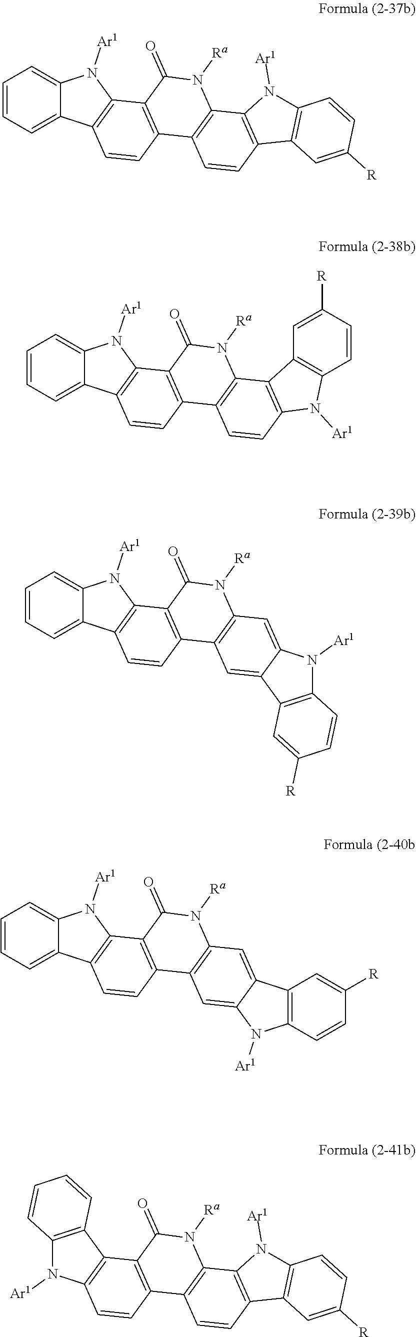

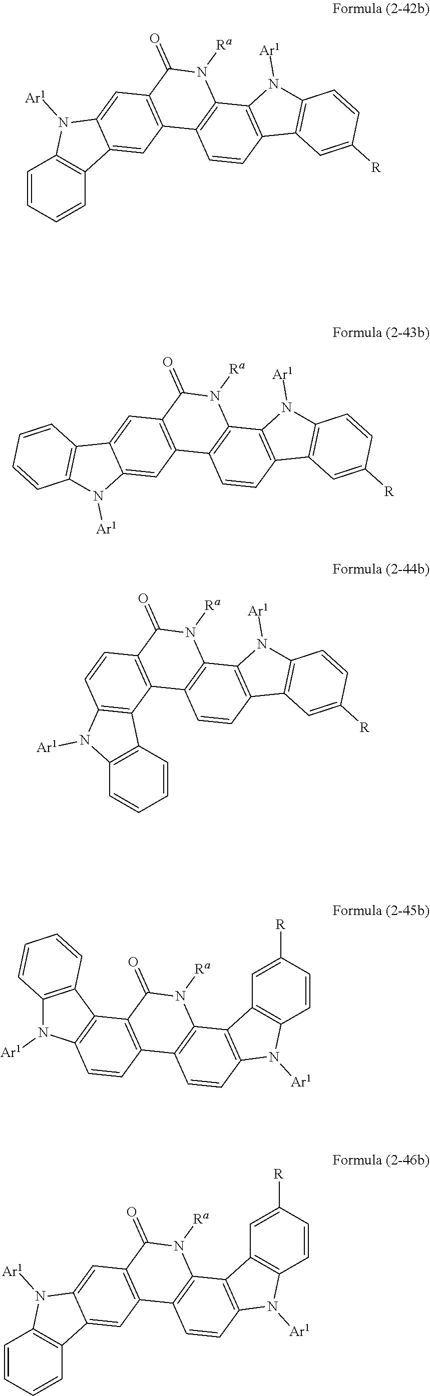

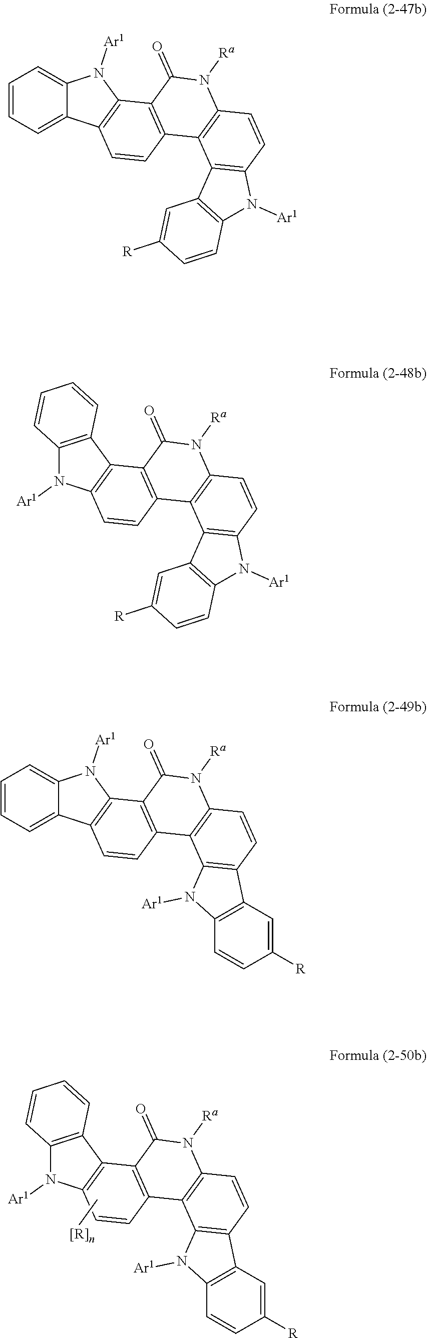

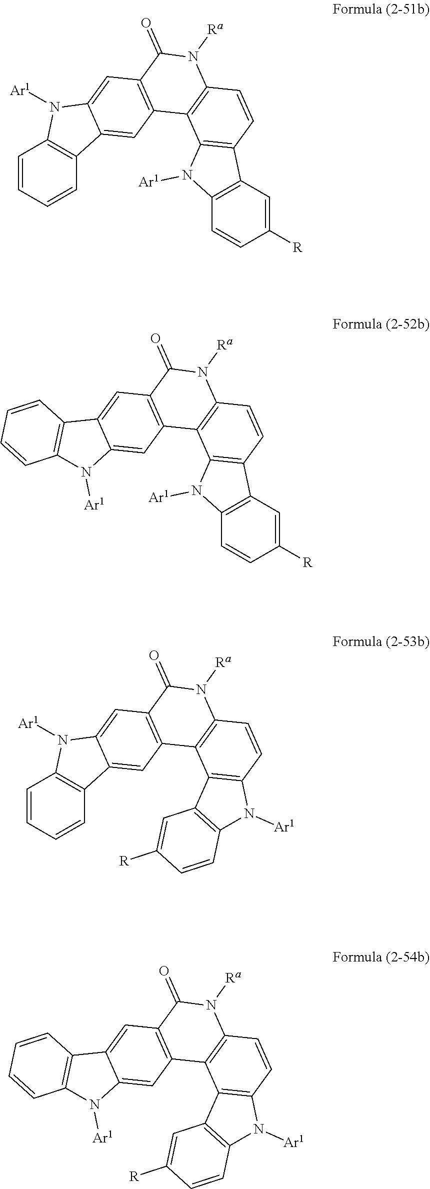

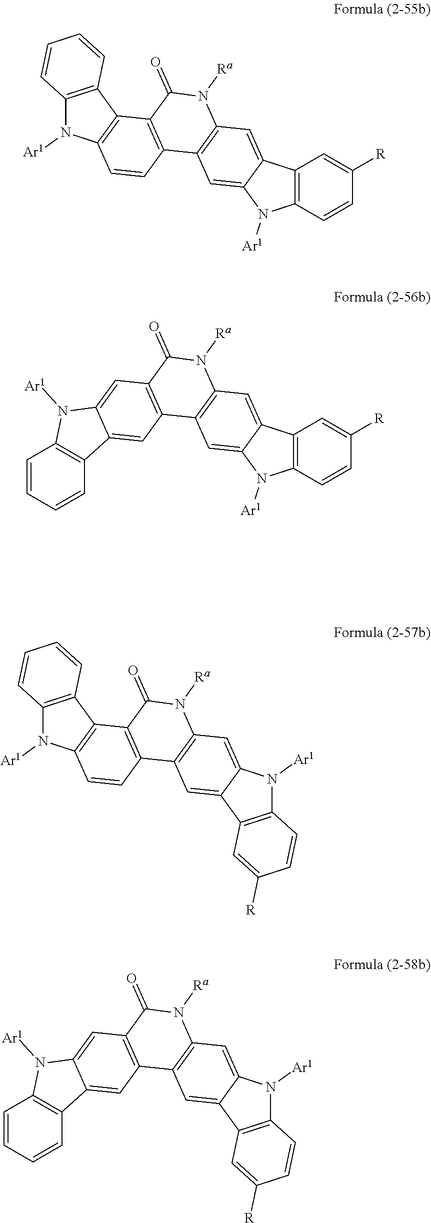

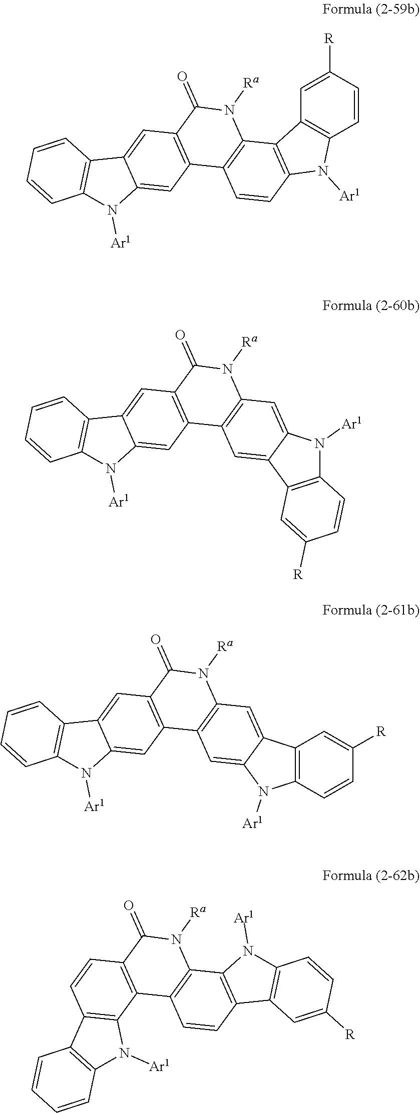

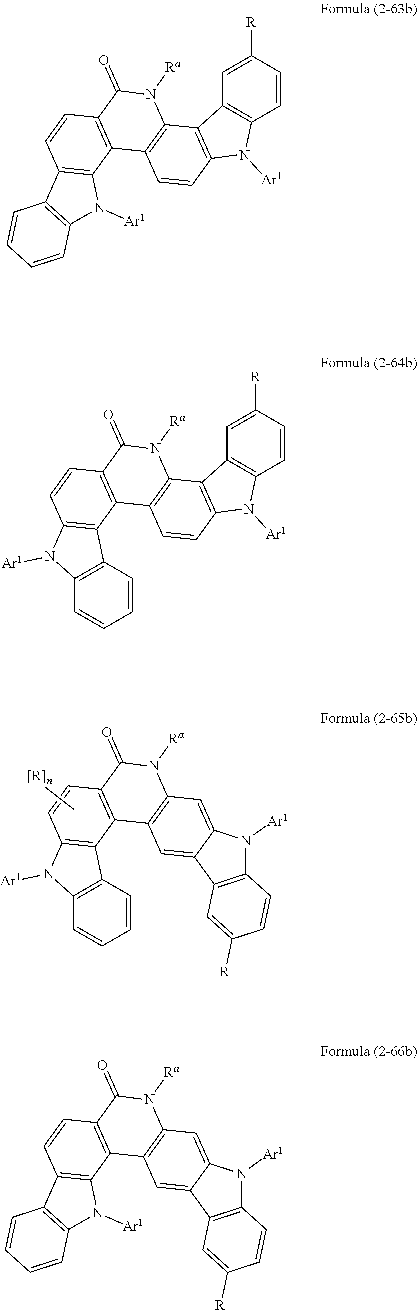

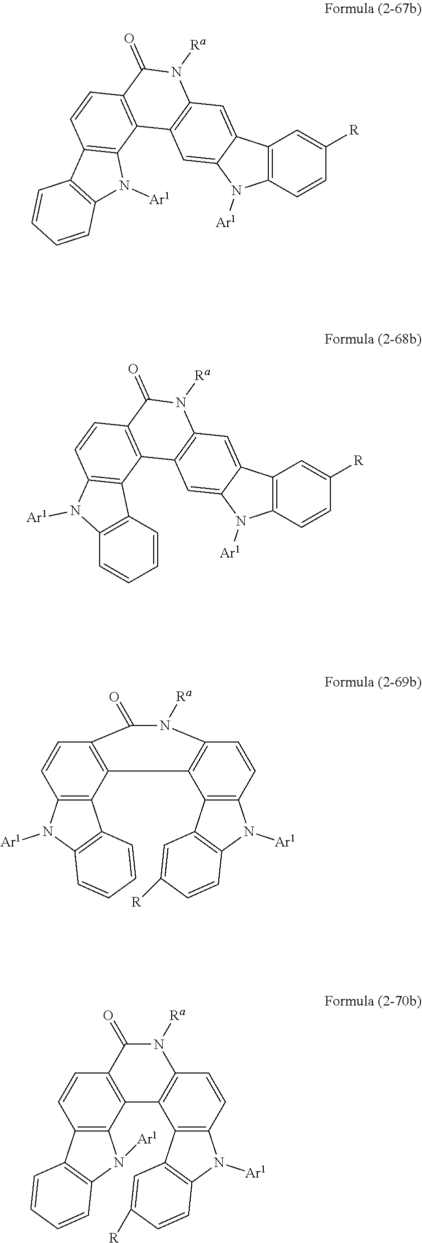

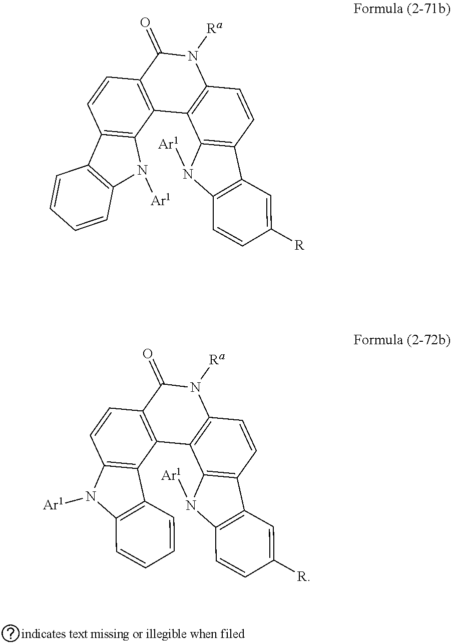

6. A process for preparing a compound as claimed in claim 1, which comprises the steps of:
(a) synthesizing a biscarbazole base skeleton proceeding from a functionalized fluorenone or the functionalized central ring system via coupling and ring closure reactions;
(b) introducing the substituent Ar¹ by a coupling reaction or nucleophilic substitution;
(c) in the case of the fluorenone precursor, forming the central ring containing Y¹ and Y² via a rearrangement reaction;
(d) introducing the substituent R^{a} for the sequence (a), (b) and (c) by a coupling reaction or nucleophilic substitution.

7. A formulation comprising at least one compound as claimed in claim 1 and at least one further compound.

8. An electronic device comprising at least one compound as claimed in claim 1, wherein the electronic device is selected from the group consisting of organic electroluminescent devices, organic integrated circuits, organic field-effect transistors, organic thin-film transistors, organic light-emitting transistors, organic solar cells, organic optical detectors, organic photoreceptors, organic field-quench devices, light-emitting electrochemical cells and organic laser diodes.

9. The electronic device as claimed in claim 8, which is an organic electroluminescent device, wherein the device comprises an anode, a cathode and at least one emitting layer, wherein at least one organic layer that may be an emitting layer, hole transport layer, electron transport layer, hole blocker layer, electron blocker layer or other functional layer comprises the at least one compound.
